SEQUENCE LISTING (357 sequences)

SEQ ID NO: 1 - DNA, 72 bp, Homo sapiens
aaatattggctattattatttttatcacaccatctcgtgagtctcatcatctcatgaaatagtgcatgagaa

SEQ ID NO: 2 - DNA, 72 bp, Homo sapiens
gtttcagtggtgggatcatgtctttatcagaagctcgtgaaggaatgttgcttttcttagtcatgtaggaac

SEQ ID NO: 3 - DNA, 72 bp, Homo sapiens
agcagtttgtataaacacaaataataggaagtaatcgaattgaaaactaatccaaaactgctttttgaatgg

SEQ ID NO: 4 - DNA, 72 bp, Homo sapiens
aggtgggagaaactcttcaggccaagagtttgagacgagcctgggcaacatagcaagaccctatctctataa

SEQ ID NO: 5 - DNA, 72 bp, Homo sapiens
tggtgaaaagggaatggaaattggatgtaaggatacgagtttccttttttttttttttttgagacagagtat

SEQ ID NO: 6 - DNA, 72 bp, Homo sapiens
attcctagaaaactgtattaaactgattgctagcacgtatgtgtatggattcactgtgggacttgtacagac

SEQ ID NO: 7 - DNA, 72 bp, Homo sapiens
ttttccctttataccaagaggatgtctgattaactcgatgtataaaaggactgataacaaaaataagcatca

SEQ ID NO: 8 - DNA, 72 bp, Homo sapiens
gggtggattgcttgagctcaagaattcaagaccaacgtgggcagcatagcaagactccctacaaaaaaaata

SEQ ID NO: 9 - DNA, 72 bp, Homo sapiens
cacatgcacatgtatgtttattgcagcactattcacgatagcagacttggaaccaacccaaatgtccatcaa

SEQ ID NO: 10 - DNA, 72 bp, Homo sapiens
gagttcattccccatccagttaggtcaagttagaacgagggttgccatccagttaggtcaagttaaaatgag

SEQ ID NO: 11 - DNA, 72 bp, Homo sapiens
ccttccactgataaccatcaaggtaacattgcaaacgtgttagactatggcataaaggcaaccacaggtaca

SEQ ID NO: 12 - DNA, 72 bp, Homo sapiens
ggccaaggcaggcagatcacttgaggtcaggagttcgagatcagtctagccaacatggtgaaacccagtctc

SEQ ID NO: 13 - DNA, 72 bp, Homo sapiens
gtcccagagattctggtatgttgtgtctttgttctcgttggtttcaaagagcatctttatttctgctttcat

SEQ ID NO: 14 - DNA, 72 bp, Homo sapiens
tctcctcctagattatataaaaagattgtattccacgtgctgaatcaaaacacagttaacttggtgagatca

SEQ ID NO: 15 - DNA, 72 bp, Homo sapiens
cctgcacttcctggccctccatgcttgggcatggacgtgtgatatggtttggctgtgtccccacccaaatct

SEQ ID NO: 16 - DNA, 72 bp, Homo sapiens
acatgtgccatgttggtttgctgcacccatcaactcgtcatttacattaggtatttctcctaacactatccc

SEQ ID NO: 17 - DNA, 72 bp, Homo sapiens
gtcagagtgcttgtgcccaaaactaagtcataccacgtacttaagtacacagatcttagagtcagagtgctt

SEQ ID NO: 18 - DNA, 72 bp, Homo sapiens
cccagccttagggtgtcctttttatactttgttttcgttaacagtgtcaaaaattagttggctttaagtatt

SEQ ID NO: 19 - DNA, 72 bp, Homo sapiens
ccattttgtgtaaaatctgccatggacaatatgtacgtgaatgaacatggctatgttccacattattttggg

SEQ ID NO: 20 - DNA, 72 bp, Homo sapiens
gtaacttaacacaatagatgtttatttcttactcacgtaaagtctaataggtgccaagacagataaggttct

SEQ ID NO: 21 - DNA, 72 bp, Homo sapiens
atttagacaaaggtatattcagcctgttttatgtacgaagcactgtactgatccctgcagaagacaaaatca

SEQ ID NO: 22 - DNA, 72 bp, Homo sapiens
agctgtgtgctggaggctgccagtgctcaacaaatcgtgcttgcacttttcactgtgctcaggtgaagtaca

SEQ ID NO: 23 - DNA, 72 bp, Homo sapiens
tgcccaggtctggcctcttgtttcaagtcacagctcgttgaaaacattaaaaaaaaaaaaaacaaaccttga

SEQ ID NO: 24 - DNA, 72 bp, Homo sapiens
acaaaaattcatcagatttaataaagttgtctattcgaagatagggacttttttcttttttaaaaattaaat

SEQ ID NO: 25 - DNA, 72 bp, Homo sapiens
aggatggctgggctccagtgtctctggagtggcttcgagtccactgctcctggaaggcttcatcccattggc

SEQ ID NO: 26 - DNA, 72 bp, Homo sapiens
agatatgactggaaaacattttctcccattgtgtacgtgtcttttcacttacttggtgacatcctttagagc

SEQ ID NO: 27 - DNA, 72 bp, Homo sapiens
cacattgtcaaaattggtggtgggtgagaaacagtcgtgggttctagttcatctttatgaattcccatttgt

SEQ ID NO: 28 - DNA, 72 bp, Homo sapiens
ccccatgacctagtcacctccccaaaggccccagtcgacttgggaattaggatttcaacctatacattttgg

SEQ ID NO: 29 - DNA, 72 bp, Homo sapiens
atataagcaggcagaaaaatgtgaaaagagaaacacgtctagctgcccagtatacatctttctcccatgctg

SEQ ID NO: 30 - DNA, 72 bp, Homo sapiens
caaagtcatttttaattataaactttgaatatgttcgtatttatttagttatttaatgcttatttaaaaatg

SEQ ID NO: 31 - DNA, 72 bp, Homo sapiens
ctacaaaccaagcacaccaaggatttctggagccacgagaagtggagcaagaaagaggcattggttcatgaa

SEQ ID NO: 32 - DNA, 72 bp, Homo sapiens
gagtgcagccattttaaagtatcaagccaggtgttcgtaacaggcacttcataagtggaatattttattttg

SEQ ID NO: 33 - DNA, 72 bp, Homo sapiens
gaggagacttttgatattgttctatttatctttatcgtcacattttttcaggcagtaactatatgtaaaaga

SEQ ID NO: 34 - DNA, 72 bp, Homo sapiens
ccacactactcaaagtagctgttccccaaactgttcgttacccttacactaagagataagaagcttgatcca

SEQ ID NO: 35 - DNA, 72 bp, Homo sapiens
aaaaaagaaaaaaaagtagtcttatagattaattacgtaattaaccattagcaaacacaatacagcctgaga

SEQ ID NO: 36 - DNA, 72 bp, Homo sapiens
agatcaagaccatcctggccaacatggtgaaacctcgtctctactaaaaatacaaaaattagctgggcatgg

SEQ ID NO: 37 - DNA, 72 bp, Homo sapiens
cactcctcccagacacaagagctagtcaatggtgtcgtgtgtcccttcaaggcaaatactacttgtaatagt

SEQ ID NO: 38 - DNA, 72 bp, Homo sapiens
taaggttcattgtgggccatcttagaggctatctacgagtggatcattactttttattatcattatttattt

SEQ ID NO: 39 - DNA, 72 bp, Homo sapiens
agcccagctaagtttttattattcttttgtagacacgtgatcttgctatgttgcccaggctggtcttaaaca

SEQ ID NO: 40 - DNA, 72 bp, Homo sapiens
cctaatccaatagtactggtgtccttataagaagacgagattaggacagagacacctacagaaggaaggctg

SEQ ID NO: 41 - DNA, 72 bp, Homo sapiens
tgatatcccctttatcattttttattgtgtctattcgatttttctctcttttcttctttattagtctggcta

SEQ ID NO: 42 - DNA, 72 bp, Homo sapiens
ttctaccagaggtacaaagaggagctggtaccattcgttctgaaactattccagtcaatagaaagagaggga

SEQ ID NO: 43 - DNA, 72 bp, Homo sapiens
ctgggttcaagcaatcctcttgcctcagcctccctcgtagctgaaactacaggcatatgccaccatgcccaa

SEQ ID NO: 44 - DNA, 72 bp, Homo sapiens
ttagagttgccagagttcttgcactggctctttctcgtctatgtaggctgatgttcctttaatctttgaagt

SEQ ID NO: 45 - DNA, 72 bp, Homo sapiens
gagacaggatctcactacattacccaggctggtctcgaactcttggcctcaagtgatcctcctgcctcagcc

SEQ ID NO: 46 - DNA, 72 bp, Homo sapiens
agtattgatacccctgctctcttttggttattattcgtataaactatccttttttatactttcactttcaac

SEQ ID NO: 47 - DNA, 72 bp, Homo sapiens
gtgtgtatatatatgtgtgtgtgtatatatacacacgtatatatatatatttaactgattcttgtgccttag

SEQ ID NO: 48 - DNA, 72 bp, Homo sapiens
atttcaatgcataaaactaagaaagtagatcaagacgataatacaattttcagttgtatatttttgttttag

SEQ ID NO: 49 - DNA, 72 bp, Homo sapiens
aacaacctgggcaacatggtgaaactctgtctctacgaaaaaaaaaaaaaattagctggatgtggtggtgtg

SEQ ID NO: 50 - DNA, 72 bp, Homo sapiens
aagtatcttattaatatttttaaaatacttgattacgtgttaaaatgatggtattttgaatatactggatta

SEQ ID NO: 51 - DNA, 72 bp, Homo sapiens
acatacaccattgaaatagacaaatgttactttttcgtacctacccctattcctctaagtacctgttgttaa

SEQ ID NO: 52 - DNA, 72 bp, Homo sapiens
caggctgatggaaacatgacatggagttggcctgacgttgctgactttgaaaatggagaaaggggccaagag

SEQ ID NO: 53 - DNA, 72 bp, Homo sapiens
cctgtaggcaagcataagaaatgagcagctactaacgtttgaaatcctttgctatcccatgcaaagttacat

SEQ ID NO: 54 - DNA, 72 bp, Homo sapiens
agtagggagatatgtcatcacatattcctgggatacgtaaactataactcaaactatataagaggaaaattg

SEQ ID NO: 55 - DNA, 72 bp, Homo sapiens
tttttgctattgtgaatagtgctgcaataaacatacgtgtgcatgtgtctttattgtagcatgatttataat

SEQ ID NO: 56 - DNA, 72 bp, Homo sapiens
gttatttcagtaacacttgtgtttattgcaactgacgtgattgcaggagctgcacagggcacttgtccatcc

SEQ ID NO: 57 - DNA, 72 bp, Homo sapiens
aagtattgttcttaagaaatgttcagtctgttcaacgatttgagcccctttctattgactctccaggagtca

SEQ ID NO: 58 - DNA, 72 bp, Homo sapiens
acagtcaaatatgccccttcttaaaaacaaacaaacgaacagacaaacaaatccctctcttcagtgtatatc

SEQ ID NO: 59 - DNA, 72 bp, Homo sapiens
tggatattagaaaaaatatcacaagggggtgtatacgactcctgagatattgggagtaacatcattctctcc

SEQ ID NO: 60 - DNA, 72 bp, Homo sapiens
aggaccacctatccaagactatgggaggcctgagacgattgcagaacatctgctagtataaacttcaagaat

SEQ ID NO: 61 - DNA, 72 bp, Homo sapiens
atgttagctataggatttccatatatggcctttatcgtgttgtggtacattccttctatacctaatttgttc

SEQ ID NO: 62 - DNA, 72 bp, Homo sapiens
ggcattatgtaagagtcaaattttattcctctccacgaagatatccagttttcctaacactatttattgaag

SEQ ID NO: 63 - DNA, 72 bp, Homo sapiens
cctgggacagcctgggttttgtttctccttcctttcgaagcagaatgttcttcaaagcttttcccagtgagt

SEQ ID NO: 64 - DNA, 72 bp, Homo sapiens
ccatttatgacaatatggatgaatctagaggacatcgtggtaagtgaaataagccagacacagaaagacaag

SEQ ID NO: 65 - DNA, 72 bp, Homo sapiens
tcatcaatcaccactgtttcagtgcagaacattttcgtcttcccaaaaagaaacccctcagtaatcactccc

SEQ ID NO: 66 - DNA, 72 bp, Homo sapiens
tgggattcagtttttgaaatgaaacactgagccttcgatgaccttcctgtacatgtgaaagcacacctgtct

SEQ ID NO: 67 - DNA, 72 bp, Homo sapiens
ctcacatggtgccctgcactgccaagacaagtgaacgatacagtaaggatggctaaaggtgacctcagaaac

SEQ ID NO: 68 - DNA, 72 bp, Homo sapiens
atatttttaaaagcataaatatttaggcatactaacgatagtcagatataagtcatgaacagacaagctgaa

SEQ ID NO: 69 - DNA, 72 bp, Homo sapiens
aagagatgggtagaatagaaacaacttgaaaaacacgttttaagatatcatctatgagagcttccccaactt

SEQ ID NO: 70 - DNA, 72 bp, Homo sapiens
tgactccaccaaggcaaggaagtcatcaaaagggacgtggggagtgtggggaaaaaatacataaatcatggg

SEQ ID NO: 71 - DNA, 72 bp, Homo sapiens
gagatgtgaggtgtcattctattcatcatgttcttcgttgcttgaatactctcagcatttgttttctggaaa

SEQ ID NO: 72 - DNA, 72 bp, Homo sapiens
aagaaactccagcatatttacatcttttatgtctacgatccactcactttcagagtttccaaagactgaatt

SEQ ID NO: 73 - DNA, 72 bp, Homo sapiens
cattgtctgtttttaaatttgagataaaattgtcacgaaaatataagacaaacagggaaatctaattttctg

SEQ ID NO: 74 - DNA, 72 bp, Homo sapiens
tccccattctcctctcatataaggctaccacagaacgtattttctagggccctccatcttttgattccctaa

SEQ ID NO: 75 - DNA, 72 bp, Homo sapiens
aatagtttaatggttattatacagatatgttttatcgttttcttggagaatgttgactattttagctttcaa

SEQ ID NO: 76 - DNA, 72 bp, Homo sapiens
taactggagaacacacttattactcataaagcagacgaagcaaaagtagacatttgacatataataaaacaa

SEQ ID NO: 77 - DNA, 72 bp, Homo sapiens
tagtccatcagttattcagtagcctaattttgattcgaatgcacttcactggtttagtacccaggtcattgc

SEQ ID NO: 78 - DNA, 72 bp, Homo sapiens
gtcacaggtcctcatgagaattggaggggacaagacgtccaaatcatatcaaaacttgacagagttttcatt

SEQ ID NO: 79 - DNA, 72 bp, Homo sapiens
tttcttactacaaattttcctgtcatttcctatttcgacctcttttatctaagcctggaatgcagtcagcac

SEQ ID NO: 80 - DNA, 72 bp, Homo sapiens
gcaaggatgtctcctctcacactccttttcaatatcgtactagaagttctagctgatacaataagacaagaa

SEQ ID NO: 81 - DNA, 72 bp, Mouse
tgatctactcatgcagaaggcaggcctgcaagtatcgtagctacacagagtaaaaccaacatccagcaataa

SEQ ID NO: 82 - DNA, 72 bp, Mouse
tagtggagcatgtatccttattacatcccttattacgagatagcatttgaaatgtaaatgaagaaaatatct

SEQ ID NO: 83 - DNA, 72 bp, Mouse
cctatcatatgcctgaaaagcacttacaacagactcgagttgctcttgactttgtcctactacacttgcttc

SEQ ID NO: 84 - DNA, 72 bp, Mouse
gctataacatattcagagggtaagtcccatattttcgtgtttctaatcaatgatgagagaataaagactcct

SEQ ID NO: 85 - DNA, 72 bp, Mouse
aaacaaattcaaagacaaaaaccacatgatcatctcgttagatgcagaaaaagcatttgacaagatccaaca

SEQ ID NO: 86 - DNA, 72 bp, Mouse
gatttcagaggaaaacactttctctgtcttgtactcgtccaggtgataaactcctactttgaaatcctattg

SEQ ID NO: 87 - DNA, 72 bp, Mouse
catgtctttctcattagttgttaagaaattgtcttcgttctgcatacaatttggccactaaaaattgcatca

SEQ ID NO: 88 - DNA, 72 bp, Mouse
aattctaaggggcaaagtgtccacactttggtcttcgttcttcttgagtttcatgtgttttgcaaattgtat

SEQ ID NO: 89 - DNA, 72 bp, Mouse
taaaaataggctttttaaggttaagaaaatcctttcgtaaaattgaggttgatttatccagagtctagaaac

SEQ ID NO: 90 - DNA, 72 bp, Mouse
atacatgaggacatttagcttctcttttgggtcttcgattttatttcaatgatcaacctgtctgtttctgta

SEQ ID NO: 91 - DNA, 72 bp, Mouse
aacttttagattgtttatttgtgtctggagacattcgattttaccacacagcaccttcttttccttcatcat

SEQ ID NO: 92 - DNA, 72 bp, Mouse
tttattcacagggattacttcttttcctttatctacgtttctgtgaatgtctttaatatttttatacttcta

SEQ ID NO: 93 - DNA, 72 bp, Mouse
ctgacctccactttagtcagctcttggctcaagcacgtaccactgtgaaagcaaaacagatggtcagtaagt

SEQ ID NO: 94 - DNA, 72 bp, Mouse
tctgtaagaggtcatcttttacactaaatagaattcgttcctgattttaagcaaactactgtagccaaagcc

SEQ ID NO: 95 - DNA, 72 bp, Mouse
gcaatcaccatcaaaattccaactcaattcttcaacgaattagaaagagcaatctgcaaattcatctggaac

SEQ ID NO: 96 - DNA, 72 bp, Mouse
tgagtttcatgtgtttaggaaattgtatcttatatcgtgggtatcctaggttttgggctagtatccacttat

SEQ ID NO: 97 - DNA, 72 bp, Mouse
ttcttttctgttattatcttttgaagggctggattcgtggaaagataatgtgtgaattttgttttgtagtgg

SEQ ID NO: 98 - DNA, 72 bp, Mouse
actctagcaagcctgtcttagcattagttatgcaacgtcaactggcctcaaagttactgagatttgctgcag

SEQ ID NO: 99 - DNA, 72 bp, Mouse
gctttacaaggtaagtctggccttgaactttctaacgaaattcaagacagtctatcagaagtaaagtgggga

SEQ ID NO: 100 - DNA, 72 bp, Mouse
ttttcaggtacttctcagccatttggtattcctcacgtgagaattctttgtttagctctgagcacaattttt

SEQ ID NO: 101 - DNA, 72 bp, Mouse
atcaaataagtcactttacatctcttccctggtaacgactacaaaattccatacttctaagagccacagaga

SEQ ID NO: 102 - DNA, 72 bp, Mouse
ataaatgtggaattatatgtacatataaatggatacgttatccaaattaaaaattcaagacccaagaaatac

SEQ ID NO: 103 - DNA, 72 bp, Mouse
attccagataaatttgcagattgccctttctaattcgttgaagaattgagttggaattttgatggggattgt

SEQ ID NO: 104 - DNA, 72 bp, Mouse
gcaatacccatcaaaattccaaatcaattcttcaacgaattagaaggagcaatttgcaaattcatctggaat

SEQ ID NO: 105 - DNA, 72 bp, Mouse
atgctacttttgtgctacttcagcattcattttaacgttttcttcaactttcttaatgtttgtttctcaaag

SEQ ID NO: 106 - DNA, 72 bp, Mouse
aatctcaagataaaatataaaattgtactccaattcgtttgtcaagagaacataaattcaagcaatgctccc

SEQ ID NO: 107 - DNA, 72 bp, Mouse
aatagaatattcatccccaatgcattcttaagactcgtgatattagtgagaaaaatatagtatggaagactc

SEQ ID NO: 108 - DNA, 72 bp, Mouse
aaaatacttctagctatttattgctgtgcctcaaacgatcctaaaacatgacaacataaaacagcagcattt

SEQ ID NO: 109 - DNA, 72 bp, Mouse
tcataccagtgtaaaatatagttgtgcaaaaatatcgtttgtcatctgtctctaaaattcctattatgacaa

SEQ ID NO: 110 - DNA, 72 bp, Mouse
ggtgcacagaacaggagctttgcatataaactcaacgtggtggtgacaacaggcaaaatccttgaaaaggac

SEQ ID NO: 111 - DNA, 72 bp, Mouse
ctaccctaccccctacacacacacacacacacacacgagagagagagagagagagagggagagagagagaga

SEQ ID NO: 112 - DNA, 72 bp, Mouse
agagcattatgcacctttaaacatttgttctctcacgacccttcattttggtaacacttaaacacttgatgt

SEQ ID NO: 113 - DNA, 72 bp, Mouse
ctaccacagtcatttttataaaggacatggtctgtcgagtaaccaactttgcatccattcagcatgcctttc

SEQ ID NO: 114 - DNA, 72 bp, Mouse
aatgaaataaaagtccatgtcctaccttaaaaggacgtagtcttgaataaacaaacatttaaaagacacata

SEQ ID NO: 115 - DNA, 72 bp, Mouse
tttaaagtgaatctctaacaatatttagaatgaatcgaaattcagtcaaactaatgaagcctgagatacaaa

SEQ ID NO: 116 - DNA, 72 bp, Mouse
aattatcttatagaggagaaagtagagaagagtctcgaagatattggcacaagggaaaacttcctgaactac

SEQ ID NO: 117 - DNA, 72 bp, Mouse
tttaaaactgaactgaactgctaatatcctgacaacgaatattgaacttgtacccaaagagctgtttctaaa

SEQ ID NO: 118 - DNA, 72 bp, Mouse
taatttaaaaaactgaaagaaactaagaaaaaaaacgtgaggaatgtatatatatatatatatatatatata

SEQ ID NO: 119 - DNA, 36 bp, Artificial Sequence
  Extension Probe
aaatattaactattattatttttatcacaccatctc

SEQ ID NO: 120 - DNA, 36 bp, Artificial Sequence
  Extension Probe
atttcaataataaaatcatatctttatcaaaaactc

SEQ ID NO: 121 - DNA, 36 bp, Artificial Sequence
  Extension Probe
aacaatttatataaacacaaataataaaaaataatc

SEQ ID NO: 122 - DNA, 36 bp, Artificial Sequence
  Extension Probe
aaataaaaaaaactcttcaaaccaaaaatttaaaac

SEQ ID NO: 123 - DNA, 36 bp, Artificial Sequence
  Extension Probe
taataaaaaaaaaataaaaattaaatataaaaatac

SEQ ID NO: 124 - DNA, 36 bp, Artificial Sequence
  Extension Probe
attcctaaaaaactatattaaactaattactaacac

SEQ ID NO: 125 - DNA, 36 bp, Artificial Sequence
  Extension Probe
ttttccctttataccaaaaaaatatctaattaactc

SEQ ID NO: 126 - DNA, 36 bp, Artificial Sequence
  Extension Probe
aaataaattacttaaactcaaaaattcaaaaccaac

SEQ ID NO: 127 - DNA, 36 bp, Artificial Sequence
  Extension Probe
cacatacacatatatatttattacaacactattcac

SEQ ID NO: 128 - DNA, 36 bp, Artificial Sequence
  Extension Probe
aaattcattccccatccaattaaatcaaattaaaac

SEQ ID NO: 129 - DNA, 36 bp, Artificial Sequence
  Extension Probe
ccttccactaataaccatcaaaataacattacaaac

SEQ ID NO: 130 - DNA, 36 bp, Artificial Sequence
  Extension Probe
aaccaaaacaaacaaatcacttaaaatcaaaaattc

SEQ ID NO: 131 - DNA, 36 bp, Artificial Sequence
  Extension Probe
atcccaaaaattctaatatattatatctttattctc

SEQ ID NO: 132 - DNA, 36 bp, Artificial Sequence
  Extension Probe
tctcctcctaaattatataaaaaaattatattccac

SEQ ID NO: 133 - DNA, 36 bp, Artificial Sequence
  Extension Probe
cctacacttcctaaccctccatacttaaacataaac

SEQ ID NO: 134 - DNA, 36 bp, Artificial Sequence
  Extension Probe
acatataccatattaatttactacacccatcaactc

SEQ ID NO: 135 - DNA, 36 bp, Artificial Sequence
  Extension Probe
atcaaaatacttatacccaaaactaaatcataccac

SEQ ID NO: 136 - DNA, 36 bp, Artificial Sequence
  Extension Probe
cccaaccttaaaatatcctttttatactttattttc

SEQ ID NO: 137 - DNA, 36 bp, Artificial Sequence
  Extension Probe
ccattttatataaaatctaccataaacaatatatac

SEQ ID NO: 138 - DNA, 36 bp, Artificial Sequence
  Extension Probe
ataacttaacacaataaatatttatttcttactcac

SEQ ID NO: 139 - DNA, 36 bp, Artificial Sequence
  Extension Probe
atttaaacaaaaatatattcaacctattttatatac

SEQ ID NO: 140 - DNA, 36 bp, Artificial Sequence
  Extension Probe
aactatatactaaaaactaccaatactcaacaaatc

SEQ ID NO: 141 - DNA, 36 bp, Artificial Sequence
  Extension Probe
tacccaaatctaacctcttatttcaaatcacaactc

SEQ ID NO: 142 - DNA, 36 bp, Artificial Sequence
  Extension Probe
acaaaaattcatcaaatttaataaaattatctattc

SEQ ID NO: 143 - DNA, 36 bp, Artificial Sequence
  Extension Probe
aaaataactaaactccaatatctctaaaataacttc

SEQ ID NO: 144 - DNA, 36 bp, Artificial Sequence
  Extension Probe
aaatataactaaaaaacattttctcccattatatac

SEQ ID NO: 145 - DNA, 36 bp, Artificial Sequence
  Extension Probe
cacattatcaaaattaataataaataaaaaacaatc

SEQ ID NO: 146 - DNA, 36 bp, Artificial Sequence
  Extension Probe
ccccataacctaatcacctccccaaaaaccccaatc

SEQ ID NO: 147 - DNA, 36 bp, Artificial Sequence
  Extension Probe
atataaacaaacaaaaaaatataaaaaaaaaaacac

SEQ ID NO: 148 - DNA, 36 bp, Artificial Sequence
  Extension Probe
caaaatcatttttaattataaactttaaatatattc

SEQ ID NO: 149 - DNA, 36 bp, Artificial Sequence
  Extension Probe
ctacaaaccaaacacaccaaaaatttctaaaaccac

SEQ ID NO: 150 - DNA, 36 bp, Artificial Sequence
  Extension Probe
aaatacaaccattttaaaatatcaaaccaaatattc

SEQ ID NO: 151 - DNA, 36 bp, Artificial Sequence
  Extension Probe
aaaaaaacttttaatattattctatttatctttatc

SEQ ID NO: 152 - DNA, 36 bp, Artificial Sequence
  Extension Probe
ccacactactcaaaataactattccccaaactattc

SEQ ID NO: 153 - DNA, 36 bp, Artificial Sequence
  Extension Probe
aaaaaaaaaaaaaaaataatcttataaattaattac

SEQ ID NO: 154 - DNA, 36 bp, Artificial Sequence
  Extension Probe
aaatcaaaaccatcctaaccaacataataaaacctc

SEQ ID NO: 155 - DNA, 36 bp, Artificial Sequence
  Extension Probe
cactcctcccaaacacaaaaactaatcaataatatc

SEQ ID NO: 156 - DNA, 36 bp, Artificial Sequence
  Extension Probe
taaaattcattataaaccatcttaaaaactatctac

SEQ ID NO: 157 - DNA, 36 bp, Artificial Sequence
  Extension Probe
aacccaactaaatttttattattcttttataaacac

SEQ ID NO: 158 - DNA, 36 bp, Artificial Sequence
  Extension Probe
cctaatccaataatactaatatccttataaaaaaac

SEQ ID NO: 159 - DNA, 36 bp, Artificial Sequence
  Extension Probe
taatatcccctttatcattttttattatatctattc

SEQ ID NO: 160 - DNA, 36 bp, Artificial Sequence
  Extension Probe
ttctaccaaaaatacaaaaaaaaactaataccattc

SEQ ID NO: 161 - DNA, 36 bp, Artificial Sequence
  Extension Probe
ctaaattcaaacaatcctcttacctcaacctccctc

SEQ ID NO: 162 - DNA, 36 bp, Artificial Sequence
  Extension Probe
ttaaaattaccaaaattcttacactaactctttctc

SEQ ID NO: 163 - DNA, 36 bp, Artificial Sequence
  Extension Probe
aaaacaaaatctcactacattacccaaactaatctc

SEQ ID NO: 164 - DNA, 36 bp, Artificial Sequence
  Extension Probe
aatattaatacccctactctcttttaattattattc

SEQ ID NO: 165 - DNA, 36 bp, Artificial Sequence
  Extension Probe
atatatatatatatatatatatatatatatacacac

SEQ ID NO: 166 - DNA, 36 bp, Artificial Sequence
  Extension Probe
atttcaatacataaaactaaaaaaataaatcaaaac

SEQ ID NO: 167 - DNA, 36 bp, Artificial Sequence
  Extension Probe
aacaacctaaacaacataataaaactctatctctac

SEQ ID NO: 168 - DNA, 36 bp, Artificial Sequence
  Extension Probe
aaatatcttattaatatttttaaaatacttaattac

SEQ ID NO: 169 - DNA, 36 bp, Artificial Sequence
  Extension Probe
acatacaccattaaaataaacaaatattactttttc

SEQ ID NO: 170 - DNA, 36 bp, Artificial Sequence
  Extension Probe
caaactaataaaaacataacataaaattaacctaac

SEQ ID NO: 171 - DNA, 36 bp, Artificial Sequence
  Extension Probe
cctataaacaaacataaaaaataaacaactactaac

SEQ ID NO: 172 - DNA, 36 bp, Artificial Sequence
  Extension Probe
aataaaaaaatatatcatcacatattcctaaaatac

SEQ ID NO: 173 - DNA, 36 bp, Artificial Sequence
  Extension Probe
tttttactattataaataatactacaataaacatac

SEQ ID NO: 174 - DNA, 36 bp, Artificial Sequence
  Extension Probe
attatttcaataacacttatatttattacaactaac

SEQ ID NO: 175 - DNA, 36 bp, Artificial Sequence
  Extension Probe
aaatattattcttaaaaaatattcaatctattcaac

SEQ ID NO: 176 - DNA, 36 bp, Artificial Sequence
  Extension Probe
acaatcaaatataccccttcttaaaaacaaacaaac

SEQ ID NO: 177 - DNA, 36 bp, Artificial Sequence
  Extension Probe
taaatattaaaaaaaatatcacaaaaaaatatatac

SEQ ID NO: 178 - DNA, 36 bp, Artificial Sequence
  Extension Probe
aaaaccacctatccaaaactataaaaaacctaaaac

SEQ ID NO: 179 - DNA, 36 bp, Artificial Sequence
  Extension Probe
atattaactataaaatttccatatataacctttatc

SEQ ID NO: 180 - DNA, 36 bp, Artificial Sequence
  Extension Probe
aacattatataaaaatcaaattttattcctctccac

SEQ ID NO: 181 - DNA, 36 bp, Artificial Sequence
  Extension Probe
cctaaaacaacctaaattttatttctccttcctttc

SEQ ID NO: 182 - DNA, 36 bp, Artificial Sequence
  Extension Probe
ccatttataacaatataaataaatctaaaaaacatc

SEQ ID NO: 183 - DNA, 36 bp, Artificial Sequence
  Extension Probe
tcatcaatcaccactatttcaatacaaaacattttc

SEQ ID NO: 184 - DNA, 36 bp, Artificial Sequence
  Extension Probe
taaaattcaatttttaaaataaaacactaaaccttc

SEQ ID NO: 185 - DNA, 36 bp, Artificial Sequence
  Extension Probe
ctcacataataccctacactaccaaaacaaataaac

SEQ ID NO: 186 - DNA, 36 bp, Artificial Sequence
  Extension Probe
atatttttaaaaacataaatatttaaacatactaac

SEQ ID NO: 187 - DNA, 36 bp, Artificial Sequence
  Extension Probe
aaaaaataaataaaataaaaacaacttaaaaaacac

SEQ ID NO: 188 - DNA, 36 bp, Artificial Sequence
  Extension Probe
taactccaccaaaacaaaaaaatcatcaaaaaaaac

SEQ ID NO: 189 - DNA, 36 bp, Artificial Sequence
  Extension Probe
aaaatataaaatatcattctattcatcatattcttc

SEQ ID NO: 190 - DNA, 36 bp, Artificial Sequence
  Extension Probe
aaaaaactccaacatatttacatcttttatatctac

SEQ ID NO: 191 - DNA, 36 bp, Artificial Sequence
  Extension Probe
cattatctatttttaaatttaaaataaaattatcac

SEQ ID NO: 192 - DNA, 36 bp, Artificial Sequence
  Extension Probe
tccccattctcctctcatataaaactaccacaaaac

SEQ ID NO: 193 - DNA, 36 bp, Artificial Sequence
  Extension Probe
aataatttaataattattatacaaatatattttatc

SEQ ID NO: 194 - DNA, 36 bp, Artificial Sequence
  Extension Probe
taactaaaaaacacacttattactcataaaacaaac

SEQ ID NO: 195 - DNA, 36 bp, Artificial Sequence
  Extension Probe
taatccatcaattattcaataacctaattttaattc

SEQ ID NO: 196 - DNA, 36 bp, Artificial Sequence
  Extension Probe
atcacaaatcctcataaaaattaaaaaaaacaaaac

SEQ ID NO: 197 - DNA, 36 bp, Artificial Sequence
  Extension Probe
tttcttactacaaattttcctatcatttcctatttc

SEQ ID NO: 198 - DNA, 36 bp, Artificial Sequence
  Extension Probe
acaaaaatatctcctctcacactccttttcaatatc

SEQ ID NO: 199 - DNA, 36 bp, Artificial Sequence
  Extension Probe
taatctactcatacaaaaaacaaacctacaaatatc

SEQ ID NO: 200 - DNA, 36 bp, Artificial Sequence
  Extension Probe
taataaaacatatatccttattacatcccttattac

SEQ ID NO: 201 - DNA, 36 bp, Artificial Sequence
  Extension Probe
cctatcatatacctaaaaaacacttacaacaaactc

SEQ ID NO: 202 - DNA, 36 bp, Artificial Sequence
  Extension Probe
actataacatattcaaaaaataaatcccatattttc

SEQ ID NO: 203 - DNA, 36 bp, Artificial Sequence
  Extension Probe
aaacaaattcaaaaacaaaaaccacataatcatctc

SEQ ID NO: 204 - DNA, 36 bp, Artificial Sequence
  Extension Probe
aatttcaaaaaaaaacactttctctatcttatactc

SEQ ID NO: 205 - DNA, 36 bp, Artificial Sequence
  Extension Probe
catatctttctcattaattattaaaaaattatcttc

SEQ ID NO: 206 - DNA, 36 bp, Artificial Sequence
  Extension Probe
aattctaaaaaacaaaatatccacactttaatcttc

SEQ ID NO: 207 - DNA, 36 bp, Artificial Sequence
  Extension Probe
taaaaataaactttttaaaattaaaaaaatcctttc

SEQ ID NO: 208 - DNA, 36 bp, Artificial Sequence
  Extension Probe
atacataaaaacatttaacttctcttttaaatcttc

SEQ ID NO: 209 - DNA, 36 bp, Artificial Sequence
  Extension Probe
aacttttaaattatttatttatatctaaaaacattc

SEQ ID NO: 210 - DNA, 36 bp, Artificial Sequence
  Extension Probe
tttattcacaaaaattacttcttttcctttatctac

SEQ ID NO: 211 - DNA, 36 bp, Artificial Sequence
  Extension Probe
ctaacctccactttaatcaactcttaactcaaacac

SEQ ID NO: 212 - DNA, 36 bp, Artificial Sequence
  Extension Probe
tctataaaaaatcatcttttacactaaataaaattc

SEQ ID NO: 213 - DNA, 36 bp, Artificial Sequence
  Extension Probe
acaatcaccatcaaaattccaactcaattcttcaac

SEQ ID NO: 214 - DNA, 36 bp, Artificial Sequence
  Extension Probe
taaatttcatatatttaaaaaattatatcttatatc

SEQ ID NO: 215 - DNA, 36 bp, Artificial Sequence
  Extension Probe
ttcttttctattattatcttttaaaaaactaaattc

SEQ ID NO: 216 - DNA, 36 bp, Artificial Sequence
  Extension Probe
actctaacaaacctatcttaacattaattatacaac

SEQ ID NO: 217 - DNA, 36 bp, Artificial Sequence
  Extension Probe
actttacaaaataaatctaaccttaaactttctaac

SEQ ID NO: 218 - DNA, 36 bp, Artificial Sequence
  Extension Probe
ttttcaaatacttctcaaccatttaatattcctcac

SEQ ID NO: 219 - DNA, 36 bp, Artificial Sequence
  Extension Probe
atcaaataaatcactttacatctcttccctaataac

SEQ ID NO: 220 - DNA, 36 bp, Artificial Sequence
  Extension Probe
ataaatataaaattatatatacatataaataaatac

SEQ ID NO: 221 - DNA, 36 bp, Artificial Sequence
  Extension Probe
attccaaataaatttacaaattaccctttctaattc

SEQ ID NO: 222 - DNA, 36 bp, Artificial Sequence
  Extension Probe
acaatacccatcaaaattccaaatcaattcttcaac

SEQ ID NO: 223 - DNA, 36 bp, Artificial Sequence
  Extension Probe
atactacttttatactacttcaacattcattttaac

SEQ ID NO: 224 - DNA, 36 bp, Artificial Sequence
  Extension Probe
aatctcaaaataaaatataaaattatactccaattc

SEQ ID NO: 225 - DNA, 36 bp, Artificial Sequence
  Extension Probe
aataaaatattcatccccaatacattcttaaaactc

SEQ ID NO: 226 - DNA, 36 bp, Artificial Sequence
  Extension Probe
aaaatacttctaactatttattactatacctcaaac

SEQ ID NO: 227 - DNA, 36 bp, Artificial Sequence
  Extension Probe
tcataccaatataaaatataattatacaaaaatatc

SEQ ID NO: 228 - DNA, 36 bp, Artificial Sequence
  Extension Probe
aatacacaaaacaaaaactttacatataaactcaac

SEQ ID NO: 229 - DNA, 36 bp, Artificial Sequence
  Extension Probe
ctaccctaccccctacacacacacacacacacacac

SEQ ID NO: 230 - DNA, 36 bp, Artificial Sequence
  Extension Probe
aaaacattatacacctttaaacatttattctctcac

SEQ ID NO: 231 - DNA, 36 bp, Artificial Sequence
  Extension Probe
ctaccacaatcatttttataaaaaacataatctatc

SEQ ID NO: 232 - DNA, 36 bp, Artificial Sequence
  Extension Probe
aataaaataaaaatccatatcctaccttaaaaaaac

SEQ ID NO: 233 - DNA, 36 bp, Artificial Sequence
  Extension Probe
tttaaaataaatctctaacaatatttaaaataaatc

SEQ ID NO: 234 - DNA, 36 bp, Artificial Sequence
  Extension Probe
aattatcttataaaaaaaaaaataaaaaaaaatctc

SEQ ID NO: 235 - DNA, 36 bp, Artificial Sequence
  Extension Probe
tttaaaactaaactaaactactaatatcctaacaac

SEQ ID NO: 236 - DNA, 36 bp, Artificial Sequence
  Extension Probe
taatttaaaaaactaaaaaaaactaaaaaaaaaaac

SEQ ID NO: 237 - DNA, 58 bp, Artificial Sequence
  Adapter
aatgatacggcgaccaccgagatctacactctttccctacacgacgctcttccgatct

SEQ ID NO: 238 - DNA, 57 bp, Artificial Sequence
  Adapter
agatcggaagagcgtcgtgtagggaaagagtgtagatctcggtggtcgccgttcatt

SEQ ID NO: 239 - DNA, 72 bp, Homo sapiens
agactcttctgaggccctgggggctgtgacatttacgaggccaatgtataccttgagtctgttactaagata

SEQ ID NO: 240 - DNA, 72 bp, Homo sapiens
tattccatattatggacagccagttctgttcttctcgttcatattgcttgaactcaactcctacttggtcct

SEQ ID NO: 241 - DNA, 72 bp, Homo sapiens
cttgcagtcaagttgaagaaccagtgaatgacagccgttgcaggtgggtttcagaaactccctgagaatctc

SEQ ID NO: 242 - DNA, 72 bp, Homo sapiens
gtggctcttaaacccactggatcttctcagtggcccgtggtgccagccccagacagtggccaggcctccttg

SEQ ID NO: 243 - DNA, 72 bp, Homo sapiens
ggtagatggtttaggaagacagtgaagattttcaccgtgaaggaaatggagaaagatgcttgttagagatat

SEQ ID NO: 244 - DNA, 72 bp, Homo sapiens
ggggattcttcttttctgatggcctttagaatgagcgttggatcttcctgggtctcaagcctgcaggctttg

SEQ ID NO: 245 - DNA, 72 bp, Homo sapiens
agagatttgcaggcatggtaggcagatgaggaagccgtgacaaaagggaaatttgtgtgcctaagaagtctc

SEQ ID NO: 246 - DNA, 72 bp, Homo sapiens
aaggtgcaaaaattaaatcatgcatgcaaagcagtcgtaggtgctccatagtatgtggttagccttataatg

SEQ ID NO: 247 - DNA, 72 bp, Homo sapiens
gtcaagtccctgcccttgaatgtggtttgacctcccgaagtgagaaaacatgccaggaagcttgttacccac

SEQ ID NO: 248 - DNA, 72 bp, Homo sapiens
tttttctcactatggcatgcacctaatccttggtccgtgactgctaaagcagtagatttctatggccctttg

SEQ ID NO: 249 - DNA, 72 bp, Homo sapiens
tctcatggttttatttgaagctgaaatgaaatagccgtgaaaaaagcactgtaacttagagctatctcaatc

SEQ ID NO: 250 - DNA, 72 bp, Homo sapiens
atgactactgtagacactcttaaattccctgtcaacgtttcattatagcagcatcatctgtttgaaaatata

SEQ ID NO: 251 - DNA, 72 bp, Homo sapiens
tgcagaggacatgggcttcctcatcactgatgccacgagctcctcatgggtagacaggaccctgccagtgac

SEQ ID NO: 252 - DNA, 72 bp, Homo sapiens
cagtaaatacatcatgtgtcagatattgatgagaccgtggagaagaattaggcaaggtaatttgcataaaaa

SEQ ID NO: 253 - DNA, 72 bp, Homo sapiens
cctgaagcccataagtcatctcattagtatacaaacgtagtattatgccattacttttaatggcaaaaacca

SEQ ID NO: 254 - DNA, 72 bp, Homo sapiens
gtgggaagtcactaacactgagggagaaatggtcacgtcatgagagcatcacaaagaggtgaggtcacaggt

SEQ ID NO: 255 - DNA, 72 bp, Homo sapiens
actgtaagatcattcaccctaactcattccactttcgacatcctgttacttccagtattgtttattccttcc

SEQ ID NO: 256 - DNA, 72 bp, Homo sapiens
gtcacccaggagctaggacctggcatgggggcttccgactctgcccagtgcactgtctgtggctgagcttgt

SEQ ID NO: 257 - DNA, 72 bp, Homo sapiens
gttggccaggcttagctgagctaggctggagttaccgtctgcagtcagctagtgggttaactgggtctggct

SEQ ID NO: 258 - DNA, 72 bp, Homo sapiens
ggaatcatcaggaagctcctgtgggacagataacacgtgttcattgtataggtgagggagctaaggttcaga

SEQ ID NO: 259 - DNA, 72 bp, Homo sapiens
gtggagggaagggagaggctatgataaatgtccctcgtgtgccttaaggggacctggtaacttggtttcttt

SEQ ID NO: 260 - DNA, 72 bp, Homo sapiens
ggagcagggagggaggagggctgggggtgctggttcgtaaatgatactagcccagtgagaggcctccaggct

SEQ ID NO: 261 - DNA, 72 bp, Homo sapiens
gaaattcctcctggaactccagtgtctgctcctaccgacaggctccagcccaccctaaggattttggatttg

SEQ ID NO: 262 - DNA, 72 bp, Homo sapiens
actcagcaattccttgctaagacttacagatagcccgtactggtggctgttccagatatcttctctcttatt

SEQ ID NO: 263 - DNA, 72 bp, Homo sapiens
agatccttaattttctaacatcagcaaagtcccttcgtcacataaactgacattcacaggttctggacattc

SEQ ID NO: 264 - DNA, 72 bp, Homo sapiens
gaagtgactgagaccagatgatcaccactgggcaccgtggtctctgtagcaggctcagggagcccagggttg

SEQ ID NO: 265 - DNA, 72 bp, Homo sapiens
aggaatatgactttgtggcaaatgctttaacttggcgtaagagctaagtctggcattgctgcaattgaatgg

SEQ ID NO: 266 - DNA, 72 bp, Homo sapiens
tatttcttgttcttatctttctttttctctgacctcgttccagatatctttagagttgctgctatggggagc

SEQ ID NO: 267 - DNA, 72 bp, Homo sapiens
aagtatgtgccctttatcctcctggacatgagcagcgactttttttttttttttttttttttttgagatggt

SEQ ID NO: 268 - DNA, 72 bp, Homo sapiens
cattcttctaggatcaaattgtggcaataggagagcgtgctacagggcagctctttgctgcagtgttgcaga

SEQ ID NO: 269 - DNA, 72 bp, Homo sapiens
tggtaaacccttaggaagaaattagaaaaacatggcgtaagacaagaagtctctgtgaagggttgaagagtg

SEQ ID NO: 270 - DNA, 72 bp, Homo sapiens
aagtgttaattacctaatgaacaataactcagccacgagagaaatattcagtatgttatttactggagaagg

SEQ ID NO: 271 - DNA, 72 bp, Homo sapiens
gagcagagattctggaggaactgatccattgagcccgtagatagtggggcaagagcattccaggcaggagaa

SEQ ID NO: 272 - DNA, 72 bp, Homo sapiens
taactcatgttgttttccctgccttggaattctgccgtcctcctccctccctccccttgcaacacttaccca

SEQ ID NO: 273 - DNA, 72 bp, Homo sapiens
aatgcaaaatgtgcagttcaggctggcagaaggaacgaggctggaataggagccaacaggcttataataata

SEQ ID NO: 274 - DNA, 72 bp, Homo sapiens
cagatctgtattcctcatgaaaataaaacctctctcgacacactgtgtccttgtgggtttttagttttacta

SEQ ID NO: 275 - DNA, 72 bp, Homo sapiens
ataacatcctggaggggaactgactcctacaatgccgaaagagatctataccaagaacatggctctcacaga

SEQ ID NO: 276 - DNA, 72 bp, Homo sapiens
tggccttcagcattgaactaaataagcagtcatggcgaagtggccagaggatttgttcagtgtcatacttgc

SEQ ID NO: 277 - DNA, 72 bp, Homo sapiens
gaggggatccccaccaacctcttccacacctgccccgagtcaaggtcaagtccacattgctcctgtgcctct

SEQ ID NO: 278 - DNA, 72 bp, Homo sapiens
tctctagtagcacctcacatgactagtaagcccttcgaaggggtatgcacaccattggataccccttctcaa

SEQ ID NO: 279 - DNA, 72 bp, Homo sapiens
aagcaatgacatttgccaagagaaatgctcaggcccgtcctgtgggcactcattgctgcatcatgagaggcc

SEQ ID NO: 280 - DNA, 72 bp, Homo sapiens
atgagaaggtatgacatgaactaaatgacatttttcgtcattctggctgctgtagagagaatggaatagaag

SEQ ID NO: 281 - DNA, 72 bp, Homo sapiens
tgtcttactctgtggaaccttgcaaaagtgaagaacgttgaagggttatttagggcagctggctgatgtcaa

SEQ ID NO: 282 - DNA, 72 bp, Homo sapiens
ctgtgtatcagtaagtgggtgtgggtgtgtatattcgtgtgcatttcagtgtttgtctaagtgtttatgtgt

SEQ ID NO: 283 - DNA, 72 bp, Homo sapiens
ggtcctgtgtcttgcccacctgctctcctggtggccgtggctctggagaagtccccagccaggtccatgctc

SEQ ID NO: 284 - DNA, 72 bp, Homo sapiens
tgcagcctcacctaggcagggttagtgtgggaaggcgtgggaatcaccctgtgaccaagaacaaagaggaac

SEQ ID NO: 285 - DNA, 72 bp, Homo sapiens
tcctctcatattctaaatagctgagaaacagcctacgtgcaggtcagttgcactgcactgtgtgtgatagtg

SEQ ID NO: 286 - DNA, 72 bp, Homo sapiens
ttaacagtaaaaattcaacttcctaacactggccccgtgaacatctacatgttcattccattctcatcctct

SEQ ID NO: 287 - DNA, 72 bp, Homo sapiens
acacagccaaacttggaaagacaaatagtcattggcgaataaagcagagatctggattcaagtgaagtgaag

SEQ ID NO: 288 - DNA, 72 bp, Homo sapiens
aacttccatttcctcagtggcagttaaccacattccgtgctcagcacagagtatttttcttattgcagaaag

SEQ ID NO: 289 - DNA, 72 bp, Homo sapiens; identical to SEQ ID NO: 246

SEQ ID NO: 290 - DNA, 72 bp, Homo sapiens
aggtctgtcaggactccaccattttgacatgaccccgttttcccccacaatcccccttccaggaccccattg

SEQ ID NO: 291 - DNA, 72 bp, Homo sapiens
ggggtggaaatggtcagggtagacccaagagagcacgatgcctggatgatcagtttttgttagtcagtagtt

SEQ ID NO: 292 - DNA, 72 bp, Homo sapiens
aaagactactatgtagggtagcaatcccagctgggcgtggggactccattcccactccaaaccacaaaatga

SEQ ID NO: 293 - DNA, 72 bp, Homo sapiens
agcatcctacagccccacaagtacaggcccttgttcgaatgtgtcttacaaaaaggaataaatgaaaataag

SEQ ID NO: 294 - DNA, 72 bp, Homo sapiens
tgagccatggcacttttcccaattcaattttcactcgaaaactcaaagtgagataattgcctaggcaaaact

SEQ ID NO: 295 - DNA, 72 bp, Homo sapiens
ggcccaggttgggggaagctcctccaccaacctgtcgtgagccatgcccctccagtccatctgctcccactc

SEQ ID NO: 296 - DNA, 72 bp, Homo sapiens
cacaggtggtaaaaagaatttaccaagacagctgtcgtaaagaaaggcaggtttgagaaagtaggaaaatgc

SEQ ID NO: 297 - DNA, 72 bp, Homo sapiens
cgagtggttaagtcacctacccaagagccagcatgcgtggctctgggatttgaatcagatttgcctgattcc

SEQ ID NO: 298 - DNA, 72 bp, Homo sapiens
ttcactgcaatgcagaggatgggtttgaaattcaccgattccctagggttgccctggcctggcccatcagct

SEQ ID NO: 299 - DNA, 72 bp, Homo sapiens
taaatttgatttatttttaaattattttaatttgccgtaaatggccatttgtggctggtggccacaatattg

SEQ ID NO: 300 - DNA, 72 bp, Homo sapiens
ctggaaagtcaccacccaacccactcctgatgcagcgagacctgaggaaggggccagagatgcacagggtca

SEQ ID NO: 301 - DNA, 72 bp, Homo sapiens
agctgaactcttaaccacactgctctcctgcagggcgatgagcttgccatgcctcttggtcattccctaagg

SEQ ID NO: 302 - DNA, 72 bp, Homo sapiens
agggcatttcagcagcatactcaagattctacagacgactaagtagcagagccacagtttgaacccaggcag

SEQ ID NO: 303 - DNA, 72 bp, Homo sapiens
atactaagctttattaacatccaagtaactgtgtgcgtccctgtttggttttggggaaactggactgacagc

SEQ ID NO: 304 - DNA, 72 bp, Homo sapiens
tagtggagtacaagaattcctttctacaaatggtacgtgggaacaaagattgcattggcccactatgggctc

SEQ ID NO: 305 - DNA, 72 bp, Homo sapiens
tttatacccagtgattctgaagaaggcaatagaaccgtgtgaggaaaatgtaaaggcaccctgcaatgtggc

SEQ ID NO: 306 - DNA, 72 bp, Homo sapiens
cctgggctgttgctcttggctccataaagttcttacgtgtagttctgtagttatgacccagaaccaactccc

SEQ ID NO: 307 - DNA, 72 bp, Homo sapiens
ttgctatttgggttgtctgttatatgcagccaaaccgacccctaacagacacacatatagacaactcccatc

SEQ ID NO: 308 - DNA, 72 bp, Homo sapiens
cccctagggttcttaaaaggattctatgagttattcgttgaaagggtttgaatgagtactgacccatagtaa

SEQ ID NO: 309 - DNA, 72 bp, Homo sapiens
gatagcctgctggtcctaggagaagtatcagaagccgtggagcagagccacaccagccctgttgcagatcca

SEQ ID NO: 310 - DNA, 72 bp, Homo sapiens
atggaacaagcaaagccacatcaataggcaagttccgtagcagataaaagaggcttctggggctggaaccta

SEQ ID NO: 311 - DNA, 72 bp, Homo sapiens
gacccagcagggctggagactggcaattcactccccgtcatgccttcctggtggacacctgtttaggtgggc

SEQ ID NO: 312 - DNA, 72 bp, Homo sapiens
cctgggttcaaatcccagagttgccctttctagcccgtgacctctggggagccacttcacctctccaggtgt

SEQ ID NO: 313 - DNA, 72 bp, Homo sapiens
gcagctaagtgtgccattgacagagatggtaagaacgtagagtgggaaggggccttaaggtacttaatgctc

SEQ ID NO: 314 - DNA, 72 bp, Homo sapiens
ttcctggtaccttttgaagcagatgttctgctgcccgtgagagagaggcagctacagagcagctcatcatgt

SEQ ID NO: 315 - DNA, 72 bp, Homo sapiens
ccaaggtccctgctaagcactttccatgcattaaccgtggaacttcaagacaaccctgaggtataggtatta

SEQ ID NO: 316 - DNA, 72 bp, Homo sapiens
tctgctcccagccaccctctgggccagatggtccccgtgagcctggttctagcaattagctcagatattact

SEQ ID NO: 317 - DNA, 72 bp, Homo sapiens
atcatcagccttacaggccaggtgtgtccagacaccgaagctttggagggttctaagcagtggagccatgag

SEQ ID NO: 318 - DNA, 72 bp, Homo sapiens
aaagggtttcccagatacagaagttacactccagccgttgtgtttagtacactctggtttgtctatgagctc

SEQ ID NO: 319 - DNA, 72 bp, Homo sapiens
cttaccttcttcctacctcaatcagatgccactcacgattcccttgctctaggaatcctggattttcagctc

SEQ ID NO: 320 - DNA, 72 bp, Homo sapiens
actgttttctcctctgtgctctcaaaaccctttctcgtgactctactgaaaaactcctcattgcaaatcaga

SEQ ID NO: 321 - DNA, 72 bp, Homo sapiens
ttatagaaaagcaatatattttgtaaaatgaatgacgaatgcttccatgtatccaggaagagtactgtgtcc

SEQ ID NO: 322 - DNA, 72 bp, Homo sapiens
gatatcaattcaaagtcccaaatctcatctaaatccgtcacttcaaaagtccaaagtctccttgtctcagtc

SEQ ID NO: 323 - DNA, 72 bp, Homo sapiens
agggataagtttgtgatgaaaaaggcatggaagtgcgtcctgctaaggaaagttgatgagcaggagaagagg

SEQ ID NO: 324 - DNA, 72 bp, Homo sapiens
taaacagtgtgataaattgtgtgatttagttctgccgtggaggagaatattcacctgtgagtaagcaggtag

SEQ ID NO: 325 - DNA, 72 bp, Homo sapiens
ccaattatctgggtgccttaattaatccacagacccgtggcctgatctccctgagatcctaggaaacaataa

SEQ ID NO: 326 - DNA, 72 bp, Homo sapiens
gcatgagggatgtaaaggtgcattggagatgatttcgatcagcattctttaagatgttgtttacaaaggcaa

SEQ ID NO: 327 - DNA, 72 bp, Homo sapiens; identical to SEQ ID NO: 261

SEQ ID NO: 328 - DNA, 72 bp, Homo sapiens
ggttgtcctagagatgctgcagctgttggctgtgacgtggcttactccatgtacaggtgaatgtcagagatt

SEQ ID NO: 329 - DNA, 72 bp, Homo sapiens
gtttccagttgcccttcacactgactctccttggccgttgctgctgatgggtccatccttggcctacttacc

SEQ ID NO: 330 - DNA, 72 bp, Homo sapiens
ctctgaaagcagtgctgctatgaacatcacaggaccgtgtttcatgcctagaagtggcattgtgcattgcag

SEQ ID NO: 331 - DNA, 72 bp, Homo sapiens
cagggggcaactacctcttcatagcaaagcttcatcgttaagttcctggttctgggctattgtccctgtctc

SEQ ID NO: 332 - DNA, 72 bp, Homo sapiens
tttcaggtcattaagggctttacttattttgaatgcgtttattttgacaacaattaatgggttttgagcaga

SEQ ID NO: 333 - DNA, 72 bp, Homo sapiens
gcagctggaggagatgggaaggtgcaggtttgccccgtgatctgcagcacacaagatctgtgccagggactg

SEQ ID NO: 334 - DNA, 72 bp, Homo sapiens
acattctattttttttcactgccatgaggcccctccgtggtggatggggaaggggaagggggtcttcagatg

SEQ ID NO: 335 - DNA, 72 bp, Homo sapiens
ctaggtactatggtatgtgttttacaaagctcatccgttggcctctgcatcatctctgtcaaataagcactg

SEQ ID NO: 336 - DNA, 72 bp, Homo sapiens
actgaagtatgcatatggagttaggtgtgcttatgcgtgactcaactgtgtgtgggtagcaagatccatgtc

SEQ ID NO: 337 - DNA, 72 bp, Homo sapiens
gcaagtggatagctgaaaggctgggcagagtgacccgagggcctcatttagccctgggtagtgaatgcctgt

SEQ ID NO: 338 - DNA, 72 bp, Homo sapiens
cagcaatactttgactctgctagatcctataattccgaatcctaacaactactcctgtccttctcctgcttc

SEQ ID NO: 339 - DNA, 72 bp, Homo sapiens
ccttcttgatgatgccaaactttcttctgcacaggcgtggtaccatctgcaaagcatcaactactcagtgag

SEQ ID NO: 340 - DNA, 72 bp, Homo sapiens
attcagtttattcttactgtcctgtagagaggacacgaggatcagagaggttcagtttcttgcccagaatca

SEQ ID NO: 341 - DNA, 72 bp, Homo sapiens
ggaaggcagaagtgggtgtggaggtttcccatgagcgttggcttatgtgatgcttaattttaggtgacaact

SEQ ID NO: 342 - DNA, 72 bp, Homo sapiens
aagttaaaaggatggtgaagataagcatagaaagacgaggtttggctaagtaaaggttaaagttaaggcttg

SEQ ID NO: 343 - DNA, 72 bp, Homo sapiens
catttgatgctgttgtatttttgcttctttccttacgtccatctgcctccttccatctcccctcctagaaca

SEQ ID NO: 344 - DNA, 72 bp, Homo sapiens
taatttaatatgtgggtacctacctggagccctctcgttactttgccaggactcctccctccaaatctacca

SEQ ID NO: 345 - DNA, 72 bp, Homo sapiens
catgagatgggaggagcttgagtaactgaatgacccgtggagcagagcctgtcagcctcaaacacactgtac

SEQ ID NO: 346 - DNA, 72 bp, Homo sapiens
cctgtgctggagtttgacagcagtgaccagccagacgacctggatgagacaagggtcagtgcaaacaagacc

SEQ ID NO: 347 - DNA, 72 bp, Homo sapiens
agaaaaagaagaggatgcctgaggtggtgggaagacgtaggctctagcttcaggtgagcttggaaaagtcag

SEQ ID NO: 348 - DNA, 72 bp, Homo sapiens
gtgggtctgtatctccttttcaatgtgaatatgtacgagactatgaatagctaagtaaaggtgaaaagtccc

SEQ ID NO: 349 - DNA, 72 bp, Homo sapiens
taaatgtgatctgaggccacataaataaaagtattcgtttagaatcagggaggtggaagatcctgtgtacct

SEQ ID NO: 350 - DNA, 72 bp, Homo sapiens
cacacagcctctcacagtggtgtggcctggacacccgtttccttctcctttctcaggctgccctattcttgg

SEQ ID NO: 351 - DNA, 72 bp, Homo sapiens
tttattttagttctttttcagtgtcaggtgctcatcgtggtgtaaataacaattctgtgttaggcaggtttt

SEQ ID NO: 352 - DNA, 72 bp, Homo sapiens
cagtccccagaggtcaagttatctcaacctacaggcgttccagatgataacccagtaattttgcaacaaagg

SEQ ID NO: 353 - DNA, 72 bp, Homo sapiens
tgtgctcatgaaagaccctttcattcccatgtgatcgaataggaaagcaagtaggcctagaagctactgaca

SEQ ID NO: 354 - DNA, 72 bp, Homo sapiens
gggaataattttgaagagtataggaaaatgatgaccgagagaggggataattgttagactgatatccttgag

SEQ ID NO: 355 - DNA, 72 bp, Homo sapiens
agcccaagcttgtactgcaaggtggctgcaaggcccgacccaaatctagagcctgaccttgacctcatgggt

SEQ ID NO: 356 - DNA, 72 bp, Homo sapiens
gaaagtgtgctcagaggtttggataatgctcaaaccgtagcttgggtttgaattctcaaagaaagtgcttaa

SEQ ID NO: 357 - DNA, 72 bp, Homo sapiens
tgtctcattgaaacacattgctcatttattcctctcgtcatcctttgagacacagtcattattttccagatg

(19) United States
(12) Patent Application Publication
(10) Pub. No.: US 2020/0407802 A1
(43) Pub. Date: Dec. 31, 2020

(54) Measuring Replication-Associated DNA Methylation Loss

(71) Applicants: Van Andel Research Institute, Grand Rapids, MI (US); Cedars-Sinai Medical Center, Los Angeles, CA (US)

(72) Inventors: Benjamin P. Berman, Los Angeles, CA (US); Wanding Zhou, Grand Rapids, MI (US); Peter W. Laird, East Grand Rapids, MI (US); Jamie Lynn Endicott, Grand Rapids, MI (US); Hui Shen, East Grand Rapids, MI (US)

(21) Appl. No.: 16/977,565

(22) PCT Filed: Mar. 2, 2019
(86) PCT No.: PCT/IB2019/051689

§ 371 Date: Sep. 2, 2020

Related U.S. Application Data
(60) Provisional application No. 62/637,979, filed on Mar. 2, 2018.

(51) Int. Cl. C12Q 1/6886; G16B 20/30

(52) U.S. Cl. CPC C12Q 1/6886; C12Q 2600/154; G16B 20/30

(57) ABSTRACT

Provided are methods for measuring replication-associated genomic DNA methylation loss, using a Solo-WCGW DNA sequence motif (n₍ₓ₎WCpGWn₍ₓ₎; wherein W=A or T, n=A or G or C or T and excludes any CG dinucleotides, and x≥9) to filter the methylation data. Certain methods provide for measuring the mitotic/replicative history/age of a cell or tissue sample (e.g., cell/tissue type-specific mitotic history/age), for determining a chronological age of a cell or tissue, for determining increased risk for conditions associated with excessive replicative turnover or aging, for determining a cell-type or tissue-type-specific rate of replication-associated DNA methylation loss, and for determining replication-associated DNA methylation loss of a target cell in a sample containing multiple cell types The methods provide for improved structural determination of partially methylated domains (PMD) and for identification of common PMDs shared between normal tissue types, or specific to individual normal or diseased tissue types.

### CROSS-REFERENCE TO RELATED APPLICATIONS

This application claims priority to U.S. Provisional Application 62/637,979 filed on Mar. 2, 2018, the disclosure of which is considered part of the disclosure of this application and is hereby incorporated by reference in its entirety.

### FEDERAL FUNDING ACKNOWLEDGEMENT

This invention was made with government support under Grant Nos. U24 CA210969, U01 CA184826, and U24 CA143882, awarded by the National Institutes of Health, and RO1 CA170550, and RO1 HG006705 awarded by National Institutes of Health/National Cancer Institute. The government has certain rights in the invention.

### FIELD OF THE INVENTION

Aspects of the present invention relate generally to methods for measuring genomic DNA methylation loss, and more particularly to methods enabling measurement of genomic DNA methylation loss that is linked to cellular replicative/mitotic history. Additional aspects relate to methods for measuring mitotic turnover rate, chronological age of a cell or tissue, excessive replicative turnover, increased risk for conditions associated with excessive replicative turnover or aging, identification of subjects for increased surveillance, cancer screening, forensic analysis, etc.

### CROSS-REFERENCE TO RELATED APPLICATIONS

This application claims priority to U.S. Provisional Application 62/637,979 filed on Mar. 2, 2018, the disclosure of which is considered part of the disclosure of this application and is hereby incorporated by reference in its entirety.

### INCORPORATION OF SEQUENCE LISTING

The contents of the text file named “2019_03_01_SequenceListing ST25.txt” which was created on Mar. 1, 2019, and is 74.8 KB in size, are hereby incorporated by reference in their entirety.

### BACKGROUND

Loss of 5-methylcytosine in both benign and malignant neoplasms was discovered more than thirty years ago (1-4), yet the mechanisms that lead to this hypomethylation and its role in disease remain poorly understood. Genomic studies (5-9) established that hypomethylation occurs in only about half the genome, coinciding with megabase-scale domains of repressive chromatin characterized by low gene density, low GC-density, late replication timing, localization at the nuclear lamina, and Hi-C “B” domains (10,11). These regions were termed “Partially Methylated Domains” (PMDs), and were contrasted with “Highly Methylated Domains” (HMDs) that make up the remainder of the genome (12). PMDs have been confirmed as a common feature of most epithelial cancers (13), and other cancer types such as pediatric medulloblastoma (14).

Conflicting evidence suggests that PMD hypomethylation could provide tumors with a growth advantage or alternatively may represent only a side effect of cancer (15, 16). An understanding of the earliest origins of this process could help elucidate a potential role of PMD hypomethylation in cancer initiation, yet results in pre-cancer cell types have been conflicting. Since the 1980s, long-term cell culture has been known to result in significant DNA hypomethylation (17), which was later discovered to occur primarily in PMD domains (8, 12, 18, 19) and to accumulate stochastically in culture (20, 21). In primary uncultured tissues, one study showed the existence of PMDs in a few highly proliferative tissues such as peripheral white blood cells and placenta, but not in slowly dividing tissues like kidney, lung, or brain (9). Other studies have shown the presence of global hypomethylation in placenta (22) and more differentiated B cells (23) and T cells (24), but not in early stage B cells or T cells nor in myelocytes (23, 24). The largest whole-genome bisulfite sequencing (WGBS) study of normal tissues concluded that PMDs were undetectable in 17 of 19 human tissue types studied (34 of 37 total samples), with the only exceptions being placenta and pancreas (25). This reinforced the prevailing view that PMD hypomethylation may be restricted to a very limited set of normal cell types, or only initiated upon exposure to environmental factors such as carcinogens (26). Applicants and one other group detected a small degree of PMD hypomethylation in normal mucosa adjacent to colon tumors (5, 6), but could not rule out a pre-cancer “field effect” in these adjacent tissues.

There is a need to investigate the dynamics of hypomethylation across a large number of normal and malignant tissues, and to develop new methods to enable determination of whether there are PMDs shared by normal mammalian cells and cancer cells, to enable further definition of possible relationships between PMDs, other chromatin features, and genomic mutational processes.

### SUMMARY OF THE INVENTION

Particular aspects provide the largest and most diverse set of WGBS experiments to date, including new tumor and adjacent normal data from 8 common cancer types. By identifying a local sequence signature that defined the most strongly hypomethylated CpGs within PMDs, we were able to determine that most PMDs are shared by cancers and nearly all healthy human and mouse tissue types starting from fetal development. This allowed, for the first time, investigation of the dynamics of hypomethylation across a large number of normal and malignant tissues, and definition of the relationship between PMDs, other chromatin features, and genomic mutational processes.

In certain aspects, the present methods can be used to derive mitotic age for each tissue type separately, and derive a mapping for the corresponding tissue type/cell type. Such tissue/cell-type variation can be well controlled and exploited in cell-sorting based methods.

As disclosed and described herein, a set of 39 diverse primary tumors and 8 matched adjacent tissues was profiled using Whole-Genome Bisulfite Sequencing (WGBS), and analyzed them alongside 343 additional human and 206 mouse WGBS datasets. A local CpG sequence context associated with preferential hypomethylation in PMDs was identified. Surprisingly, analysis of CpGs in this context (“Solo-WCGWs”, disclosed herein) revealed previously undetected PMD hypomethylation in almost all healthy tissue types. PMD hypomethylation increased with age, beginning during fetal development, and appeared to track the accumulation of cell divisions. In cancer, PMD hypomethylation depth correlated with somatic mutation density and cell-cycle gene expression, consistent with its reflection of mitotic history, and suggesting its application as a mitotic clock.

According to particular aspects of the present invention, therefore, late replication leads to lifelong progressive methylation loss, which acts as a biomarker for cellular aging and which, according to additional aspects, contributes to oncogenesis.

Particular surprisingly effective aspects provide a method comprising: a) identifying a test cell or tissue sample for which a determination of replication-associated DNA methylation loss is desired; b) obtaining, at data processing apparatus, CpG dinucleotide sequence methylation data for genomic DNA derived from the test cell or test tissue sample, wherein the genomic DNA comprises highly methylated domains (HMD) and partially methylated domains (PMD), wherein each such CpG dinucleotide is the sole CpG dinucleotide sequence within a n(x)WCpGWn(x) genomic DNA sequence motif (Solo-WCGW motif) of at least one PMD, and wherein W=A or T, n=A or G or C or T, and x≥9; c) determining, at the data processing apparatus, based on the CpG dinucleotide sequence methylation data, a mean or average CpG dinucleotide methylation value, or a value related thereto, for a plurality of Solo-WCGW motif sequences of the at least one PMDs, to provide a measure of cellular replication-associated DNA methylation loss (e.g., compared to HMD), wherein the provided measure of replication-associated DNA methylation loss reflects a cumulative number of cell divisions or mitotic history; and d) based on the provided measure of replication-associated DNA methylation loss, reaching a conclusion, at the data processing apparatus, as to a condition or state of the test cell or tissue sample. In the methods, obtaining the genomic CpG dinucleotide sequence methylation data may comprise excluding at the data processing apparatus, from a larger set of genomic CpG methylation data, methylation data of CpG dinucleotide sequences not within the Solo-WCGW motif sequences of the at least one PMD. In the methods, obtaining the genomic CpG dinucleotide sequence methylation data may comprise excluding, at the data processing apparatus, from a larger set of genomic CpG methylation data, methylation data of non-intergenic Solo-WCGW motif sequences of the at least one PMD. In the methods, obtaining the genomic CpG dinucleotide sequence methylation data may comprise excluding, at the data processing apparatus, from a larger set of genomic CpG methylation data, methylation data of H3K36me3 histone marked Solo-WCGW motif sequences of the at least one PMDs. In the methods, obtaining the genomic CpG dinucleotide sequence methylation data may comprise excluding cell type invariant proxies for H3K36me3 histone marked Solo-WCGW motif sequences, such as those falling in transcribed gene bodies. In the methods, the plurality of Solo-WCGW motif sequences of the at least one PMDs may be located at one or more PMDs of a single chromosome. In the methods, the plurality of Solo-WCGW motif sequences of the at least one PMDs may be located between or among multiple chromosomes. In the methods, x may be a value selected from the group consisting of at least 9, at least 14, at least 19, at least 24, at least 29, at least 34, at least 39, at least 44, at least 49, at least 54, at least 59. In the methods, x may be a value in a range selected from the group consisting of about 9-49, 9-99, 9-149, 9-199, 14-49, 14-99, 14-149, 14-199, 19-49, 19-99, 19-149, 19-199, 24-49, 24-99, 24-149, 24-199, 29-49, 29-99, 29-149, 29-199, 34-49, 34-99, 34-149, 34-199, 39-49, 39-99, 39-149, 39-199, 44-49, 44-99, 44-149, 44-199, 49-99, 49-149, 49-199, 54-99, 54-149, 54-199, 59-99, 59-149, 59-199, and any subranges of the preceding ranges. In the methods, x may be 34±25 (e.g., in the range of 9-59). In the methods, x may be 34±15 (e.g., in the range of 19-49). In the methods, x may be 34 or about 34. In the methods, the Solo-WCGW motif may comprise the sequence n(x−1)mWCpGWGn(x−1), and wherein W=A or T, n=A or G or C or T, m=C or A, and x≥9 (with x varying as given above). In the methods, the Solo-WCGW motif may comprise the sequence n(x−1)CWCpGWGn(x−1), and wherein W=A or T, n=A or G or C or T, and x≥9 (with x varying as given above). In the methods, the at least one PMDs may be characterized, at least in part, by late replication timing and/or nuclear lamina localization, and/or Hi-C-defined heterochromatic “compartment B”. In the methods, the at least one PMDs may be, at least in part, defined by assessing, at the data processing apparatus, the CpG dinucleotide sequence methylation data of the Solo-WCGW motif sequences (e.g., at least in part defined by assessing, at the data processing apparatus, the standard deviation (SD) of the CpG dinucleotide sequence methylation data of the Solo-WCGW motif sequences across a set of samples, or by assessing, at the data processing apparatus, the covariance between multiple Solo-WCGW motif sequences across a set of samples). In the methods, the SD of solo-WCGW PMD hypomethylation may be bimodally distributed within 100-kb bins. In the methods, the at least one PMD may be: a common PMD shared between or among a plurality of different cell or tissue types; a common PMD shared between or among normal and cancer cell or tissue types; or a common PMD shared between most healthy mammalian tissue types starting from fetal development. In the methods, the at least one PMD may be a cell-type invariant PMD, or a cell-type-specific PMD. In the methods, the replication-associated DNA methylation loss may reflect a cell-type specific replicative/mitotic turnover rate. In the methods, the cumulative number of cell divisions, or the mitotic history, may be from an early stage of embryonic development. In the methods, the replication-associated DNA methylation loss may reflect the chronological age of the cell or tissue sample. In the methods, the cell or tissue sample may be a cancer cell or cancer tissue sample. In the methods, the genomic DNA derived from a cell or tissue sample may comprise genomic DNA derived from tissue biopsies, or cell-free DNA derived from blood or other non-invasive samples including but not limited to urine, stool, saliva, etc. In the methods, the plurality of Solo-WCGW motif sequences of the at least one PMDs may be a number selected from at least 5, at least 10, at least 100, at least 500, at least 1,000, at least 1,500, at least 2,000, at least 5,000, and at least 10,000 or greater. In the methods, obtaining CpG dinucleotide sequence methylation data may comprise obtaining CpG dinucleotide sequence methylation data from less than a complete genomic read. In the methods, obtaining CpG dinucleotide sequence methylation data may be from the genomic DNA of a single cell. In the methods, the amount of replication-associated DNA methylation loss may vary between cell types or tissue types, reflecting a cell-type or tissue-type specific rate of replication-associated DNA methylation loss. In the methods, the plurality of Solo-WCGW motif sequences of the at least one PMDs may comprise hypomethylation prone Solo-WCGW sequence motifs selected to minimize propeller twist DNA shape. In the methods, cell-type or tissue-type specific rates of replication-associated DNA methylation loss may be used to infer the presence of one or more highly replicative cell types within a sample containing multiple cell types. The methods may, for example, comprise inferring the presence of genomic DNA of a highly replicative target cell type within a sample containing genomic DNA of multiple cell types, based on a target cell-type specific rate of replication-associated DNA methylation loss.

Additional aspects provide a method for identification of replication-associated DNA methylation loss of a target cell type in a sample containing genomic DNA of multiple cell types, comprising: a) identifying a test sample containing genomic DNA of multiple cell types including genomic DNA of a target cell type; and b) determining, at data processing apparatus, for the genomic DNA from the test sample, replication-associated DNA methylation loss according to the methods disclosed herein, wherein the at least one PMD comprises a target cell-type specific PMD to provide a measure of target cell-type specific replication-associated DNA methylation loss. In the methods, the presence of genomic DNA of the target cell may be identified at the data processing apparatus based on the presence of the target cell-type specific replication-associated DNA methylation loss. In the methods, the at least one PMD may comprise a cell-type specific PMD for the target cell type, and for each of other cell types of the sample to provide a measure of cell-type specific replication-associated DNA methylation loss for the target cell, and for each of the other cell types of the sample. In the methods, the presence of the genomic DNA of the multiple cells types may be identified at the data processing apparatus based on the presence of the respective cell-type specific replication-associated DNA methylation losses. The methods may further comprise identification at the data processing apparatus of the most hypomethylated cell types in the sample, based on the respective cell-type specific replication-associated DNA methylation losses. In the methods, the genomic DNA may comprise genomic DNA derived from tissue biopsies, or cell-free DNA derived from blood or other non-invasive samples including but not limited to urine, stool, saliva, etc.

Additional aspects provide a method for providing a measure of a mitotic history/age of a cell or tissue sample, comprising: a) identifying a test cell or tissue sample for which a determination of mitotic history/age is desired; and b) determining, at data processing apparatus, for genomic DNA from the test cell or the test tissue sample, replication-associated DNA methylation loss according to the methods described herein to provide a measure of mitotic history/age for the test cell or test tissue (test mitotic age). The methods may further comprise comparing, at the data processing apparatus, the measure of mitotic history/age of the test cell or test tissue determined in step b) with one or more control mitotic history/age values obtained, using the same method used in step b), for genomic DNA of a normal matched cell/tissue having a known replicative history, and assigning a mitotic history/age to the test cell or the test tissue. In the methods, the normal matched cell/tissue having a known replicative history may comprise a primary cell line or an immortalized primary cell line, for which mitotic history/age has been calibrated with respect to passage number using the methods disclosed herein. In the methods, the determined mitotic history/age of the cell or the tissue may be a cell type-specific or tissue type-specific mitotic history/age.

Additional aspects provide a method for determining a chronological age of a cell or tissue sample, comprising: a) identifying a test cell or tissue sample for which a determination of chronological age is desired; b) determining, at data processing apparatus, for genomic DNA from the test cell or the test tissue sample, replication-associated DNA methylation loss according to the methods disclosed herein to provide a measure of mitotic history/age for the test cell or test tissue (test mitotic age); and c) determining a chronological age for the test cell or test tissue by comparing, at data the processing apparatus, the test mitotic age with one or more control mitotic age values obtained, using the same method used in a), for genomic DNA of a normal, cell-matched and/or tissue-matched control population calculated, at the data processing apparatus, over a chronological age range, and assigning a chronological age to the test cell or the test tissue. In the methods, the actual chronological age of the test cell or test sample may be known and may be less than the chronological age determined in step b), providing a measure of accelerated aging. The methods may be part of a forensic analysis.

Additional aspects provide a method for determining increased risk for conditions associated with excessive replicative turnover or aging, comprising: a) identifying a test cell or tissue sample for which a determining increased risk for conditions associated with excessive replicative turnover or aging is desired; b) measuring, at data processing apparatus, for genomic DNA from the test cell or the test tissue sample having a known chronological age, replication-associated DNA methylation loss according to the methods disclosed herein to provide a measure of mitotic age for the test cell or test tissue (test mitotic age); and c) determining that there is an increased risk for conditions associated with excessive replicative turnover or aging by comparing, at the data processing apparatus, the test mitotic age with control mitotic age values obtained, using the same method used in a), for the genomic DNA of a normal, cell-matched or tissue-matched control population having the same chronological age as the test cell or test tissue, and finding, at the data processing apparatus, that the test mitotic age is greater than the aged-matched control mitotic age. In the methods, the condition associated with excessive replicative turnover or aging may be selected from the group consisting of cancer, neurodegenerative disease, cardiovascular disease, gastrointestinal disease, auto-immune diseases, and progeria.

Additional aspects provide a method for determining increased risk of a subject for conditions associated with excessive replicative turnover or aging, comprising: a) determining, at data processing apparatus, replication-associated genomic DNA methylation loss for a test cell or test tissue of a test subject; and b) comparing, at the data processing apparatus, the replication-associated genomic DNA methylation loss determined in a) with that of an age-matched normal control cell or tissue; and c) based on the comparison in part b), concluding, at the data processing apparatus, that a subject having greater replication-associated genomic DNA methylation loss compared to that of the age-matched control is a subject having an increased risk for conditions associated with excessive replicative turnover or aging, wherein the replication-associated genomic DNA methylation loss is determined by the methods disclosed herein. In the methods, the condition associated with excessive replicative turnover or aging may be selected from the group consisting of cancer, neurodegenerative disease, cardiovascular disease, gastrointestinal disease, auto-immune diseases and progeria.

Yet additional aspects provide a method of assessing methylation maintenance in stem cells, comprising: identifying a test stem cell sample; determining, at data processing apparatus, a measure of replication-associated genomic DNA methylation loss by the method disclosed herein; and based on the measure of replication-associated genomic DNA methylation loss, concluding, at the data processing apparatus, the degree of methylation maintenance by comparison with a normal control stem cell methylation value. In the methods, the stem cell may be selected from the group consisting of embryonic stem cells (ESC), induced pluripotent stem cells (iPSC) and mesenchymal stem cells (MSCs).

Further aspects provide a method for structurally defining a partially methylated domain (PMD) of genomic DNA, comprising: a) identifying a genomic DNA for which at least one PMD structural determination is desired; b) obtaining, at the data processing apparatus, CpG dinucleotide sequence methylation data for the genomic DNA, wherein each such CpG dinucleotide is the sole CpG dinucleotide sequence within a n(x)WCpGWn(x) genomic DNA sequence motif (Solo-WCGW motif) of at least one PMD, and wherein W=A or T, n=A or G or C or T, and x≥9 (with x varying as givem above for the general methods); and c) determining, at the data processing apparatus, a PMD structure based on the CpG dinucleotide sequence methylation data. In the methods, the at least one PMD may be, at least in part, defined by assessing, at the data processing apparatus, the standard deviation (SD) of the CpG dinucleotide sequence methylation data of the Solo-WCGW motif sequences. In the methods, the SD of solo-WCGW PMD hypomethylation may be bimodally distributed within 100-kb bins.

Yet further aspects provide a method for developing a mitotic clock, including: (a) identifying a test cell for which a determination of a mitotic clock is desired; (b) providing conditions for the test cell to divide; (c) determining the number of effective cell divisions in the test cell at one or more timepoints; (d) obtaining, at data processing apparatus, CpG dinucleotide sequence methylation data for genomic DNA derived from the test cell at the timepoints, wherein the genomic DNA comprises highly methylated domains (HMD) and partially methylated domains (PMD), wherein each such CpG dinucleotide is the sole CpG dinucleotide sequence within a n(x)WCpGWn(x) genomic DNA sequence motif (Solo-WCGW motif) of at least one PMD, and wherein W=A or T, n=A or G or C or T, and x≥9; (e) based on the CpG dinucleotide sequence methylation data, determining, at the data processing apparatus, a mean or average CpG dinucleotide methylation value or a value related thereto at each of the timepoints for a plurality of Solo-WCGW motif sequences of the at least one PMDs, to provide a measure of cellular replication-associated DNA methylation loss at each of the timepoints; (f) correlating, at the data processing apparatus, the effective cell divisions at each of the timepoints with the measure of cellular replication-associated DNA methylation loss at each of the timepoints; and (g) if the correlation from correlating step is statistically significant, identifying the measure of cellular replication-associated DNA methylation loss as a mitotic clock.

In additional aspects, the correlating step may include calculating regression at the data processing apparatus and, for example, the regression calculation may be determined by an elastic net regression model or an independent regression model.

In yet further aspects, each of the one or more timepoints may be a cell passage in vitro or changes (e.g. increases) of a cell mass in vivo. In one aspect, the conditions for the division of the test cell may include passing the test cell to certain passage numbers, wherein the timepoints are the passages numbers.

In an additional aspect, the method may include extracting DNA at each passage number and performing bisulfate conversion and library preparation and/or, at the data processing apparatus, determining a passage number calibration curve.

Further, in one aspect, the determining step may include measuring the volume of the cell mass at the one or more timepoints, wherein a change (e.g., an increase) in the volume of the cell mass across the timepoints reflects an increase in the number of effective cell divisions.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A-C show, according to particular exemplary aspects, that Solo-WCGW CpGs are prone to hypomethylation.

FIGS. 2A-F show, according to particular exemplary aspects, that most PMDs are shared across cancer and normal tissues.

FIGS. 3A1-3A2, 3B-E show, according to particular exemplary aspects, that most PMDs are shared across developmental lineages in humans.

FIG. 4 shows, according to particular exemplary aspects, that most PMDs are shared across developmental lineages in mouse.

FIGS. 5A-C show, according to particular exemplary aspects, that PMD hypomethylation emerges during embryonic development.

FIGS. 6A-F show, according to particular exemplary aspects, that PMD hypomethylation is associated with chronological age.

FIGS. 7A-G show, according to particular exemplary aspects, that PMD hypomethylation is linked to mitotic cell division in cancer. samples (purity>=0.7), ordered by PMD-HMD methylation difference.

FIGS. 8A-G show, according to particular exemplary aspects, that replication timing and H3K36me3 contribute independently to methylation maintenance.

FIGS. 9A-C show, according to particular exemplary aspects, that using the solo-WCGW sequence motif a set of shared PMDs and HMDs was initially defined across the majority of the 49 core sample set using an existing Hidden Markov Model-based (HMM-based) method, MethPipe27.

FIGS. 10A1-10A3, 10B1-10B2 show, according to particular exemplary aspects, that the same sequence dependencies shown in FIG. 9, were consistent within all other tumor and adjacent normal samples in the core set, using either the WGBS data (FIG. 10A1-A3), or matched Illumina Infinium HumanMethylation450™ (HM450) microarray data (FIG. 10B1-B2).

FIGS. 11A-C show, according to particular exemplary aspects, that an additional 390 human and 206 mouse WGBS samples examined later exhibited the same hypomethylation pattern (FIG. 11A-B) as in FIGS. 9 and 10, with the exception of three germ cell samples (FIG. 11C).

FIGS. 12A-B show, according to particular exemplary aspects, that in addition to enhancing the PMD/HMD signal in high coverage WGBS data, solo-WCGW CpGs allowed accurate PMD structure to be determined with average genomic read coverage as low as 0.05× in down-sampled bulk WGBS data (FIG. 12a), and in low-coverage single-cell WGBS data (31) (FIG. 12b), providing for an application for low coverage or single-cell WGBS studies.

FIG. 13 shows, according to particular exemplary aspects, that there is an absence of bimodal distribution of cross-sample mean methylation for the core normal and tumor WGBS samples.

FIG. 14 shows, according to particular exemplary aspects, that PMDs classified using the presently disclosed SD-based method covered 95% of the base pairs in PMDs previously reported in colorectal cancer (6), and 93% of PMDs in the IMR90 fibroblast cell line (12).

FIGS. 15A-C show, according to particular exemplary aspects, methylation maintenance in embryonic and induced pluripotent stem cells.

FIGS. 16A-B show, according to particular exemplary aspects, that for five sample groups, the majority of PMDs defined by high-SD bins were substantially overlapping PMDs defined earlier from the core tumor group (FIG. 3E).

FIG. 17 shows, according to particular exemplary aspects, a multiscaled view of chromosome 17 (3-43 Mbp) Solo-WCGW methylation in different stages of mouse spermatogenesis from prospermatogonia to mature sperm.

FIG. 18 shows, according to particular exemplary aspects, the association of average PMD solo-WCGW CpG methylation with gestational age in mouse WGBS data sets stratified by tissue types.

FIG. 19 shows, according to particular exemplary aspects, the Solo-WCGW methylation average in common HMD and common PMD in 9,072 TCGA tumor samples from 33 tumor types.

FIG. 20 shows, according to particular exemplary aspects, subtype-stratification of Solo-WCGW methylation average in common HMD and common PMD in TCGA tumor samples from 10 cancer types.

FIGS. 21A-D show, according to particular exemplary aspects, that within TCGA tumors, higher genome-wide somatic mutation densities were found to be significantly associated with deeper PMD hypomethylation, suggesting that mitotic turnover may underlie both somatic mutation and PMD hypomethylation (FIG. 7B). This association was consistent using different purity thresholds (FIG. 13c), indicating that it was not the result of confounding due to differential detection sensitivity related to purity. PMD hypomethylation was also associated with somatic copy number aberration density (FIG. 21d).

FIG. 22 shows, according to particular exemplary aspects, the association of LINE-1 break points and PMD methylation (characterized by average of HM450 probes in common PMDs). Rho is Spearman's correlation coefficient. P-value was calculated using algorithm AS89 implemented in the R software.

FIGS. 23A-B show, according to particular exemplary aspects, that head and neck squamous cell carcinomas with NSD1 mutations, which exhibit significant reductions in H3K36me2 and H3K36me3 levels (57), have substantial loss of DNA methylation in the HMD compartment.

FIGS. 24A-D show, according to particular exemplary aspects, evidence supporting a model wherein hypomethylated solo-WCGWs within late replicating PMDs are protected from deamination and thus have a lower CpG to TpG mutation rate for both somatic mutations (from tumor sequencing) and de novo mutations in the human germline (from whole-genome trio sequencing).

FIG. 25 shows, according to particular exemplary aspects, first decile of the number of solo-WCGW CpGs in windows of different sizes that were used to segment the whole genome.

FIGS. 26A-B show, according to particular exemplary aspects, mRNA expression of DNMT3A and DNMT3B. Expression of DNMT3B in H1 hESC was higher than other cancer cell lines and primary tissues assayed in the ENCODE project by over ten-fold (FIG. 26a). Embryonic Carcinoma, sharing a similar early embryonic origin with ESCs, also had the highest expression of both DNMT3A and DNMT3B compared to other cancer types in TCGA (FIG. 26b).

FIGS. 27A-B show, according to particular exemplary aspects, a rank-based analysis of 792 genomic 100 kb bins from chromosome 16 (FIG. 5) was performed to measure the HMD/PMD structure in normal tissues at different developmental stages. The rank correlations had only minor variations between replica or closely related samples (FIG. 27a) and the patterns were stable when using bins from different chromosomes (FIG. 27b).

FIG. 28 shows, according to particular exemplary aspects, that certain specific sub-patterns that match the Solo-WCGW definition were found to be more predictive of replication-associated DNA methylation loss than the more general definition.

FIG. 29 shows, according to particular exemplary aspects, that DNA shape features were also found to be predictive of replication-associated DNA methylation loss. The upper panel shows a generic illustration (taken from 2004 Pearson Education, Inc., publishing as Bnjamin Cummings) of a propeller twist that results from bond rotation. The lower panel compares to extent of propeller twist at the CpG dinucleotide found in hypomethylation resistant Solo-WCGW motif sequences, to that found in hypomethylation prone Solo-WCGW motif sequences. Specifically, hypomethylation prone Solo-WCGW motif sequences were found to have a lower propeller twist DNA shape relative to hypomethylation resistant Solo-WCGW motif sequences.

FIGS. 30-1 to 30-16 show, according to particular exemplary aspects, Table 1. TCGA tumors and adjacent normal samples were sequenced using paired-end WGBS at ˜15× sequence depth, to compile a set of 40 core tumor samples and 9 core normal samples.

FIG. 31 is a heatmap showing beta values at solo-WCGW mitotic clock CpGs. CpGs are represented by rows; samples are represented by column. Independent replicates, when performed, are denoted by ‘subculture.’ Probes are ranked by descending cross-culture starting methylation value.

FIG. 32 shows cross-culture performance of solo-WCGW mitotic clock. Cell type (n=4) is denoted by color; donor ID (n=5) is denoted by shape. Starting PDL is normalized to elastic net performed on AG21839. Delta PDL (PDLend-PDLstart) is untransformed.

FIG. 33A is a density plot showing individual coefficient of correlation (r) by donor. Simple linear regression was performed at solo-WCGW probes with no missing values (n=9711). A population of strongly anti-correlating (r<−0.75) probes is consistently observed between all combinations of cell types and donors.

FIG. 33B is a density plot showing individual correlation coefficient (r2) by donor. An overlapping subpopulation of CpGs with r2>0.80 (n=75) was selected for further use as a mitotic clock.

FIG. 34 shows the distribution of independently-predictive probes (r2>0.80) by cell type. 75 CpGs individually strongly correlated in regression analyses were shared between all cell types and donors.

FIG. 35 shows the predictive performance of median beta value from refined solo-WCGW probeset (n=75) versus median beta value of all solo-WCGW CpGs (n=9711). Particularly for cell lines from older donors, reflecting older mitotic ages, the refined subset shows markedly-enhanced performance.

FIG. 36 is a heat map showing the top pan-tissue independently predictive probeset: 75 overlapping CpGs. CpGs are represented by rows; samples are represented by column. Independent replicates, when performed, are denoted by ‘subculture.’ Probes are ranked by descending cross-culture starting methylation value.

FIG. 37 is a density plot showing the predictive performance of median beta value of refined solo-WCGW probeset (n=75) from top independently-predictive probes. While overall pan-culture correlation is poor (−0.549), likely due to lack of standardization method for PDL, correlation of independent cultures is extremely high (<−0.977). Using this model, relative mitotic ages of cells from the same lineage can be compared with high accuracy, but with poor accuracy comparing cells of differing lineages.

FIG. 38 is a heatmap showing Hannum blood clock CpGs (n=71) for primary cell samples (n=116). CpGs are represented by rows; samples are represented by columns. Independent replicates, when performed, are denoted by ‘subculture.’ Probes are ranked by descending cross-culture starting methylation value. Hannum's clock estimates chronological age for adult whole blood samples and is not intended for the cells cultured. Accordingly, cross cell-type variation of behavior at some CpGs is observed, and methylation profiles are relatively stable, reflecting minor advances in chronological age over cell culture period. Missing values are denoted by gray cells.

FIG. 39 is a heatmap showing DNAm Age CpGs (n=334; 19 CpGs from model are absent from EPIC microarray) for primary cell samples (n=116). CpGs are represented by rows; samples are represented by column. Independent replicates, when performed, are denoted by ‘subculture.’ Probes are ranked by descending cross-culture starting methylation value. Horvath's DNAm Age clock estimates chronological age for all tissue types and ages. Some variation is observed between cell type. Methylation profiles are relatively stable, reflecting minor advances in chronological age over cell culture period.

FIG. 40 is a density plot showing DNAm Age versus PDL. As DNAm Age estimates chronological age, and culturing cells under pro-mitotic conditions does not imitate physiological aging, slight positive correlation of DNAm Age to PDL is expected. The relative acceleration of DNAm Age (50-69 years) of adult fibroblast AG16146 (donor age of 31 years) is unexpected, as is the deceleration of DNAm Age (8-12 years) of adult endothelial cell AG11182 (donor age of 15 years).

FIG. 41 is a heatmap showing Skin & Blood Clock CpGs (n=391) for primary cell samples (n=116). CpGs are represented by rows; samples are represented by column. Independent replicates, when performed, are denoted by ‘subculture.’ Probes are ranked by descending cross-culture starting methylation value. Horvath's Skin & Blood Clock clock estimates chronological age for highly-replicative skin and blood samples and is sensitive to cell culture. Accordingly, modest variation is observed across advancing PDL in neonatal and adult skin cultures; little variation is observed in non-skin cultures. Missing values are denoted by gray cells.

FIG. 42 is a density plot showing Skin & Blood Clock Age versus PDL. Horvath's Skin & Blood Clock clock estimates chronological age for highly-replicative skin and blood samples and is sensitive to cell culture. Both neonatal fibroblast cell lines were modeled with moderate- to high-accuracy, although performance on adult fibroblasts was inexplicably poor and anti-correlated. Predictive performance on other cell types was mixed. The chronological ages for non-neonatal cell lines were significant underestimations of donor ages.

FIG. 43 is a heatmap showing PhenoAge CpGs (n=513) for primary cell samples (n=116). CpGs are represented by rows; samples are represented by column. Independent replicates, when performed, are denoted by ‘subculture.’ Probes are ranked by descending cross-culture starting methylation value. Levine's PhenoAge methylation clock estimates biological age for all tissue samples and is not sensitive to cell culture. Accordingly, little variation is observed across advancing PDL in all cultures. The PhenoAge methylation profile for adult endothelial cells is markedly hypomethylated compared to other cell types.

FIG. 44 is a density plot showing PhenoAge (relative units) vs PDL. Highly-variable correlations and anticorrelations are observed by cell type and donor age.

FIG. 45 is a heatmap showing epiTOC CpGs (n=385) for primary cell samples (n=116). CpGs are represented by rows; samples are represented by column. Independent replicates, when performed, are denoted by ‘subculture.’ Probes are ranked by descending cross-culture starting methylation value. Yang's epiTOC clock estimates relative mitotic age for all tissues. Surprisingly, even in adult cell lines with presumably extensive mitotic histories, little change in methylation profile is observed. Missing values are denoted by gray cells.

FIG. 46 is a density plot showing epiTOC Mitotic Age (relative units) vs PDL. Although advancing PDL for the two neonatal fibroblast cultures was strongly- to highly-correlated with epiTOC mitotic age, this composite measurement was poorly correlated for all adult cultures.

### DETAILED DESCRIPTION OF THE INVENTION

According to particular surprising aspects of the present invention, four distinct features were identified that influence DNA methylation levels in large portions of the human and mouse genomes: First, the local sequence context of the CpG dinucleotide; second, the timing of DNA replication; third, the presence of the H3K36me3 histone mark; and fourth, the accumulated number of cell divisions.

According to additional aspects, the sequence context, replication timing, and H3K36me3 marks each confer differential susceptibility to replication-associated DNA methylation loss, and thus collectively shape PMD/HMD structure, while the degree of PMD hypomethylation is a function of the cumulative number of cell divisions from the earliest stages of embryonic development.

According to particular aspects, two local sequence features (CpG density and the WCGW sequence context) were shown to exert a strong influence on the rate of DNA methylation loss at individual CpGs within PMDs, and that these influences are consistent across cell types and species.

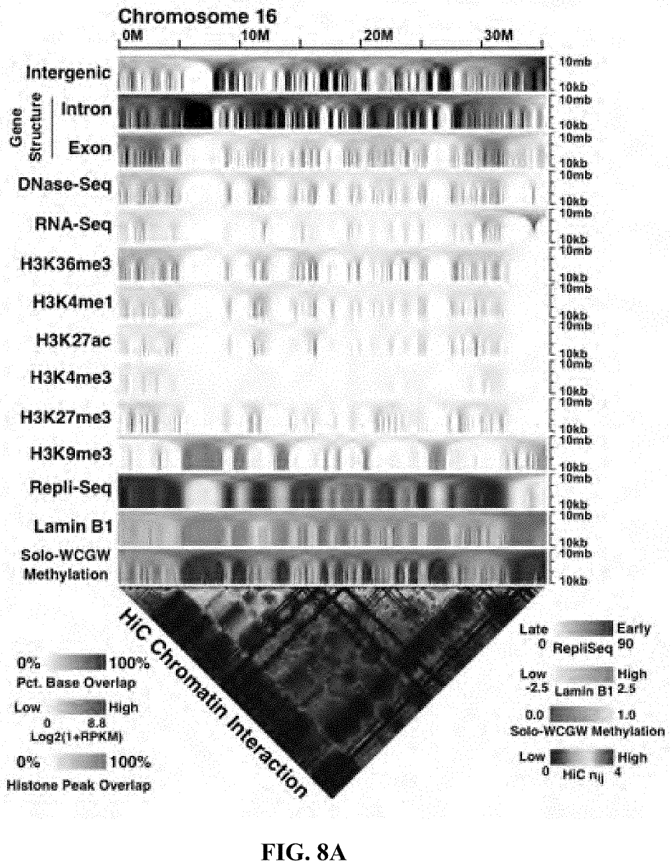

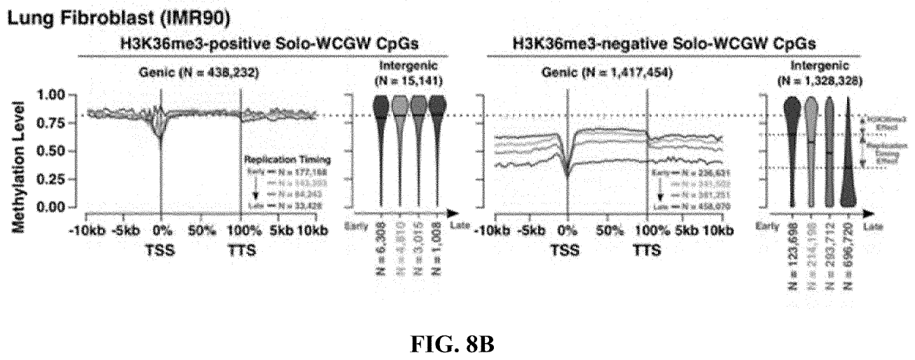

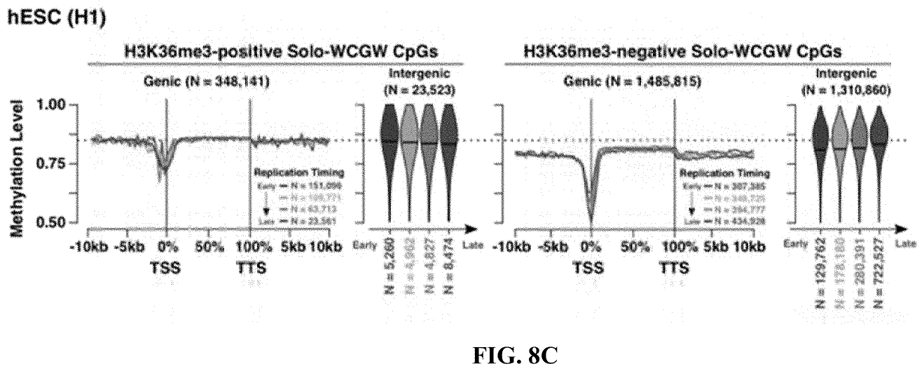

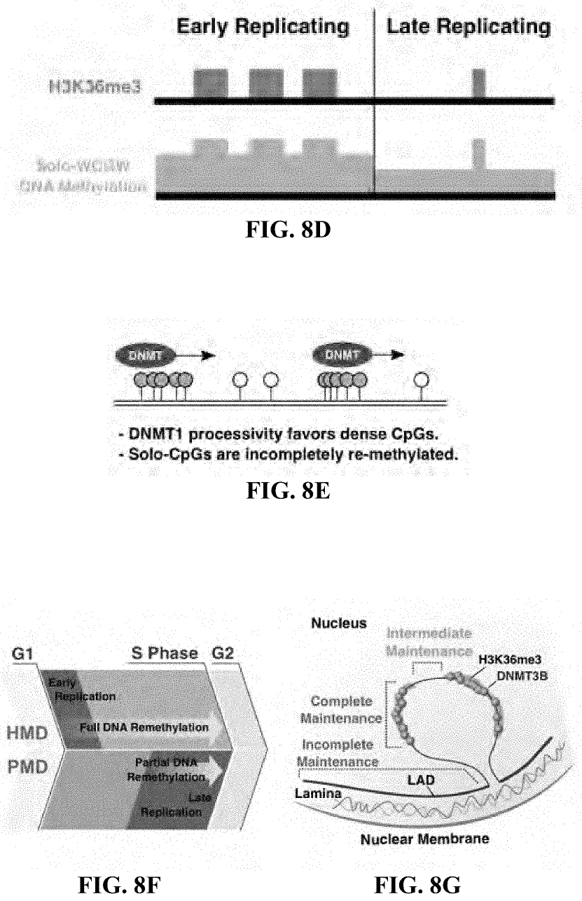

The bulk of DNA methylation maintenance is performed by DNMT1 and augmented by DNMT3A/B48. DNMT1 has been shown to act processively, with increased efficiency in the presence of multiple CpG sites in close proximity (49), a feature consistent with the poorer methylation maintenance of “solo” CpGs (FIG. 8e). Prior in vitro biochemical studies have yielded conflicting findings regarding the role of the immediate CpG flanking positions on DNMT1 activity, with one study suggesting higher affinity for G/C rich flanking sequences (50), and another suggesting higher affinity for A/T rich sequences (51).

According to additional aspects, the in vivo effects of a WCGW motif disclosed herein on methylation maintenance efficiency provide for careful mechanistic studies to identify the causative factor or factors.

According to further aspects, the Solo-WCGW signature, developed and disclosed herein, allowed for the improved analysis of HMD/PMD structure (and the shared PMD signatures) also disclosed herein, leading to better characterization of not just the “common PMDs” disclosed here, but also important classes of cell-type-specific PMDs (6, 7, 14, 52) (see working Example 10 below).

According to additional aspects of the present invention, most Solo-WCGW are not marked by H3K36me3, and replication timing was identified as the major determinant for methylation levels at these H3K36me3-negative CpGs. According to certain aspects, and while not being bound by mechanism, replication late in S phase provides the cell with less time for re-methylation of newly synthesized daughter strands during DNA replication (FIG. 8F). This is consistent with the mitotic clock-like PMD methylation loss disclosed herein specifically within late-replicating regions (FIG. 8F). This re-methylation window model is supported by a recent study that reconstructed methylation gains and losses at individual CpGs upon clonal expansions of individual somatic cells in culture (21), showing that progressive methylation loss was most pronounced at late-replicating domains. Further strengthening the re-methylation window model, biochemical studies have shown that re-methylation during mitosis is in fact relatively slow and not fully completed until after the S-G2 checkpoint (53, 54). Therefore, re-methylation efficiency is likely dependent on the time window between daughter strand synthesis and the beginning of M-phase.

According to yet additional aspects of the present invention, the presence of H3K36me3 overrides this late-replication associated methylation loss at Solo-WCGW CpGs (FIG. 8D. Without being bound by mechanism, genetic evidence suggests that maintenance of DNA methylation at H3K36me3-marked CpGs is mediated by the direct recruitment of DNMT3B to H3K36me3-marked nucleosomes (45, 55). The independent contributions of replication timing and H3K36me3 are consistent with earlier findings based on actively transcribed gene bodies (9), and help to resolve the long-standing paradox concerning positive associations between actively transcribed gene bodies and DNA methylation (56). According to further aspects, this would also explain why head and neck squamous cell carcinomas with NSD1 mutations, which exhibit significant reductions in H3K36me2 and H3K36me3 levels (57), have substantial loss of DNA methylation in the HMD compartment (FIG. 23B). It is important to note that the two major genomic contexts disclosed herein as contributing to hypomethylation, are strongly associated with specific nuclear territories (FIG. 8G). As the heterochromatin likely represents a distinct compartment separated by a physical boundary, we cannot rule out other compositional differences of this compartment contributing to the less efficient DNA methylation maintenance observed there.

A number of studies have identified specific CpGs predictive of chronological age (58-60) as well as gestation age at birth (61). However, these signatures are largely non-overlapping with PMDs, as shown in earlier work (26) and with the PMD solo-WCGWs identified here. According to particular aspects of the present invention, this is because the presently disclosed PMD hypomethylation captures underlying mitotic dynamics, which are only loosely associated with chronological age per se. Organismal aging and the associated physiological changes affect transcriptional regulation of various genes and pathways, and many or most of the loci identified on the basis of age alone (58-60) likely represent transcriptionally-coupled chromatin changes at these genes (for example, changes to Somatostatin which regulated growth hormone (58)). According to particular aspects, as shown herein, PMD hypomethylation is likely a more direct clock-like readout of mitotic age, which is generally correlated with chronological age but can be accelerated by environmental factors or processes that promote cell turnover, such as cellular damage, wounding, inflammation, etc.

DNA hypomethylation has long been proposed to allow the aberrant expression and transposition of retroelements that can play a role in cancer by inducing chromosomal aberrations at the point of insertion (62-66). Genetically engineered Dnmt1 hypomorphism in mouse was shown to cause lymphomas frequently harboring retrotranspon-induced Notchl activation events (43). Whole-genome sequencing has shown that approximately 50% of human tumors contain somatic retrotranspositions of LINE-1 elements, and that these often lead to structural alterations (39, 40, 67, 68) enriched within PMDs39. In one study, human lung tumors exhibiting mobilization of LINE-1 elements shared a common DNA hypomethylation signature (42).

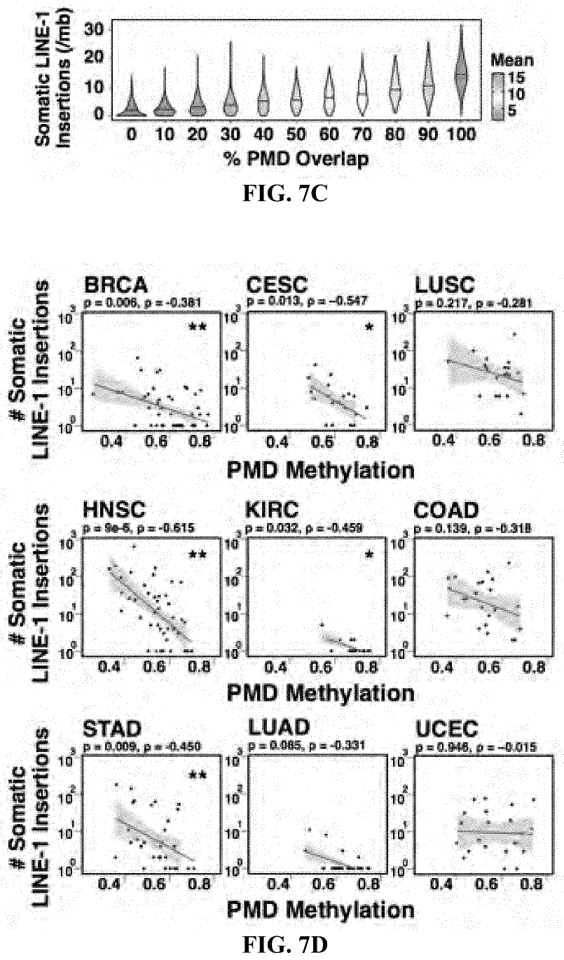

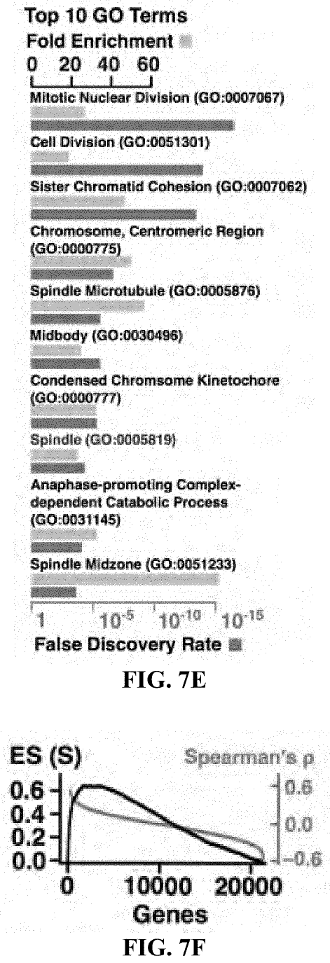

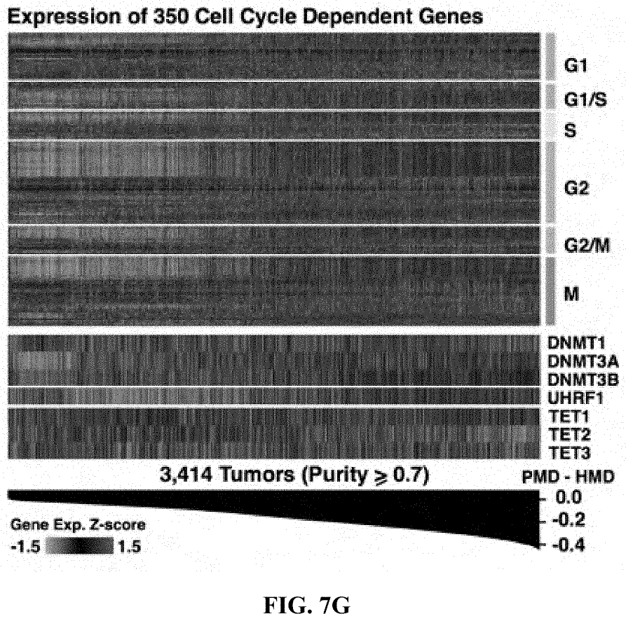

According to additional aspects of the present invention, as shown herein across a large TCGA cohort, tumors with higher degrees of PMD hypomethylation are more likely to have LINE-1 insertions, and these insertions are more likely to occur within PMDs (FIG. 7C-D). While this evidence is correlative in nature, and it is possible that LINE-1 activity is caused by a methylation-independent event, the new results presented herein are consistent with the genetic models cited above, and thus, according to particular aspects, LINE-1 insertion is accelerated by PMD hypomethylation.

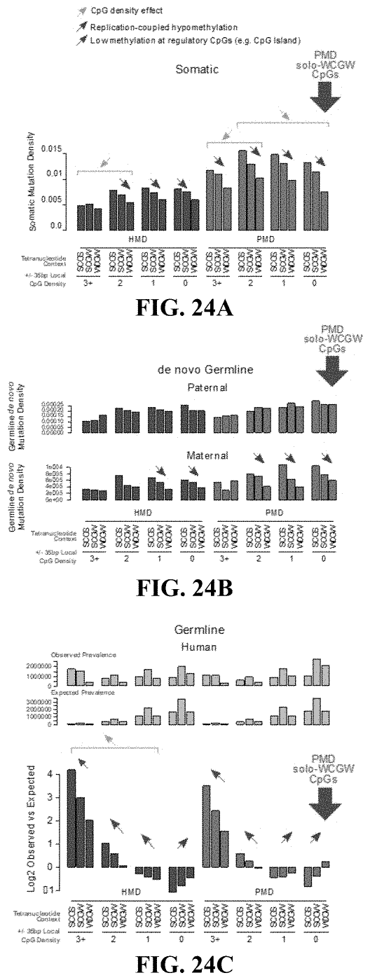

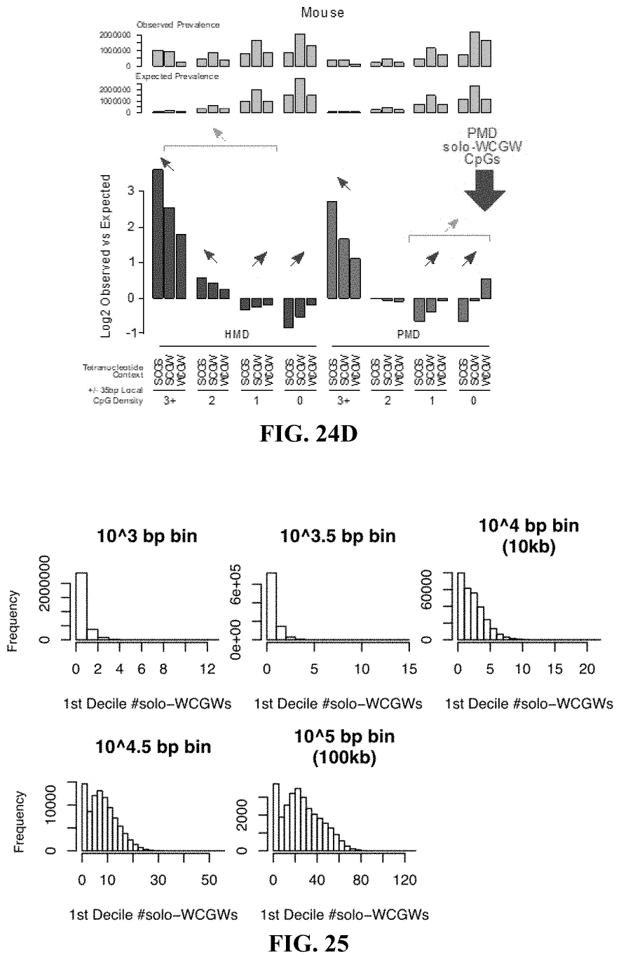

The methylation loss process described and disclosed herein affects a sizeable fraction of all CpGs in the genome, and thus could exert a significant influence on methylation-dependent mutational processes, most importantly CpG to TpG substitutions driven by methylation-dependent deamination of CpGs. This mutational signature accounts for a large fraction of single nucleotide mutations observed in both evolution and cancer, and thus systematic DNA methylation changes might be expected to influence the rate of these mutations. According to particular aspects, hypomethylated solo-WCGWs within late replicating PMDs are protected from deamination and thus have a lower CpG to TpG mutation rate. Indeed, we observed evidence in support of this model for both somatic mutations (from tumor sequencing) and de novo mutations in the human germline (from whole-genome trio sequencing) were observed herein (FIGS. 24A-D and working Example 13).

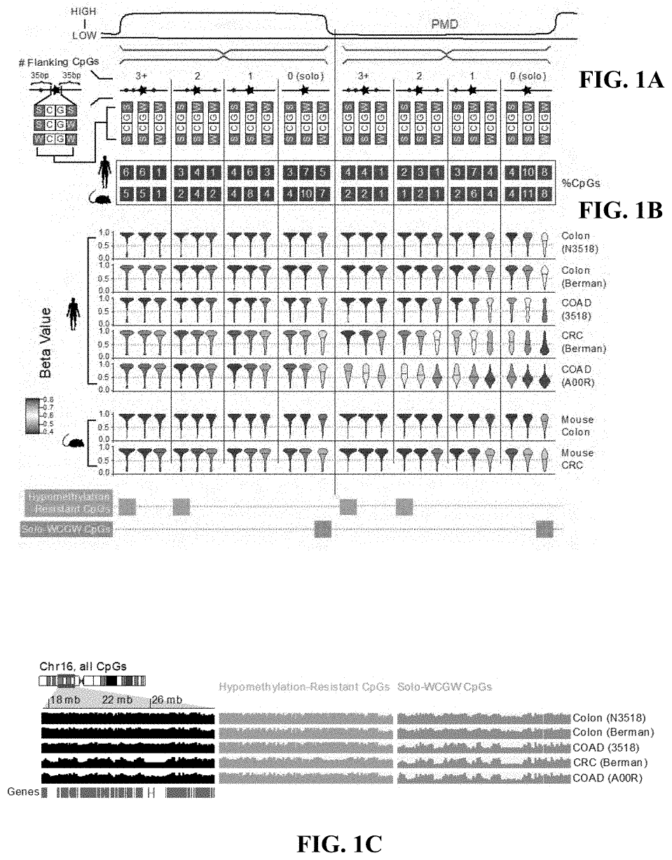

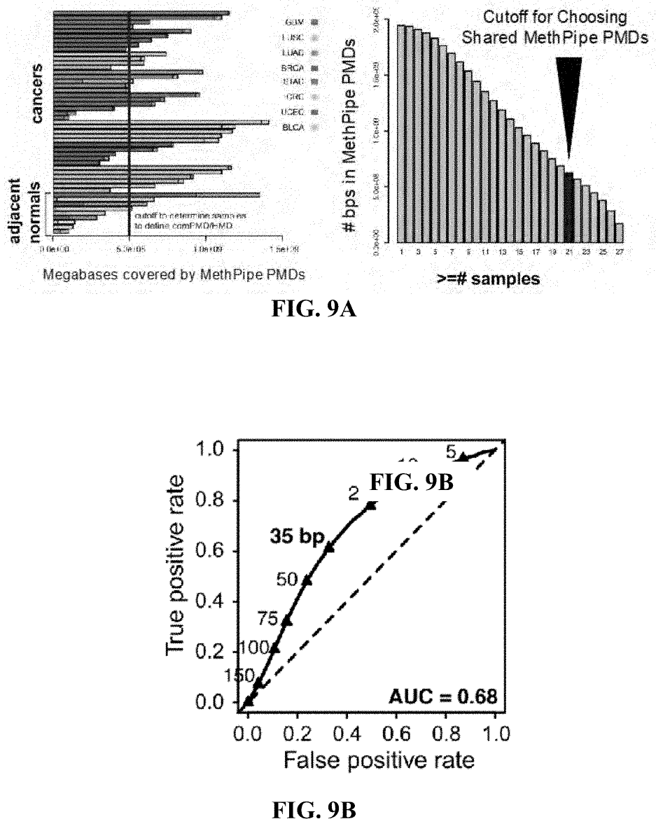

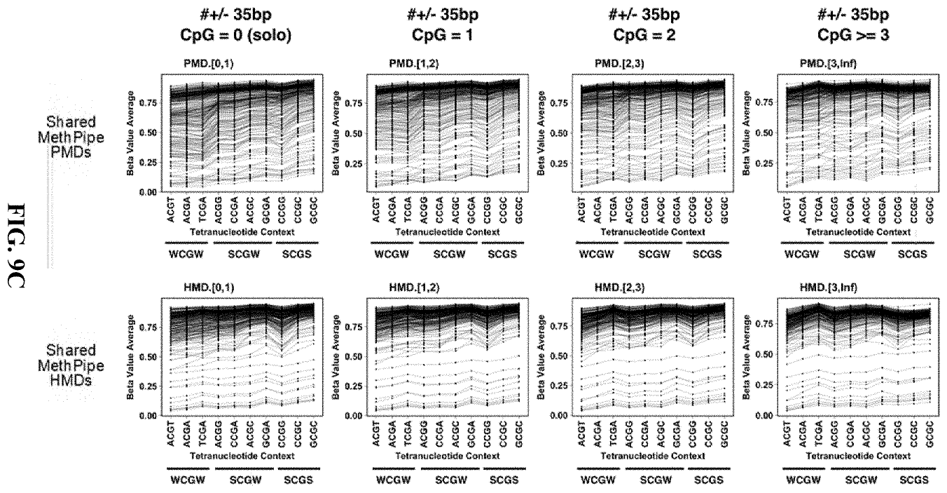

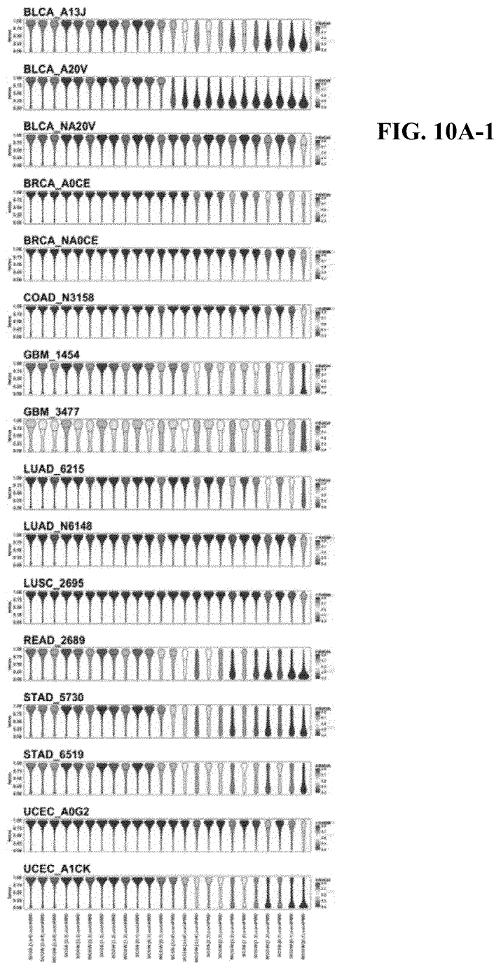

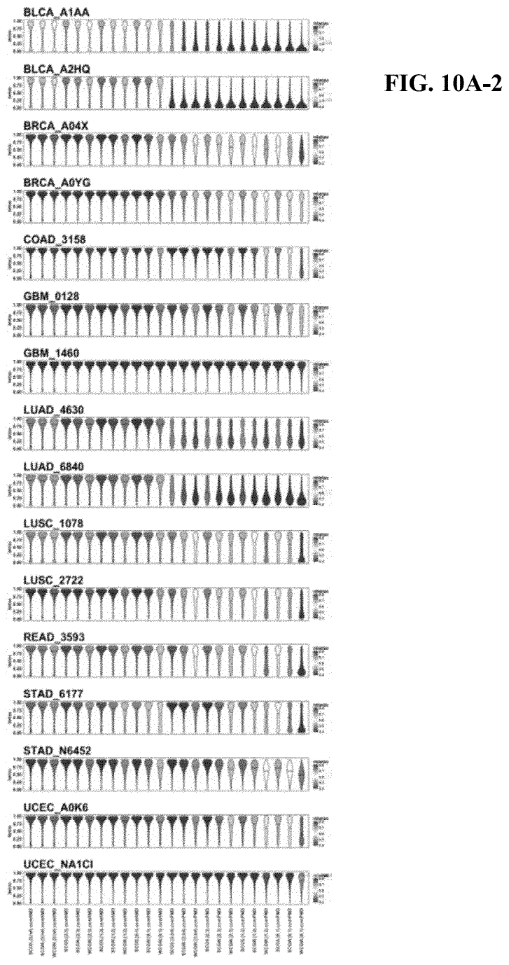

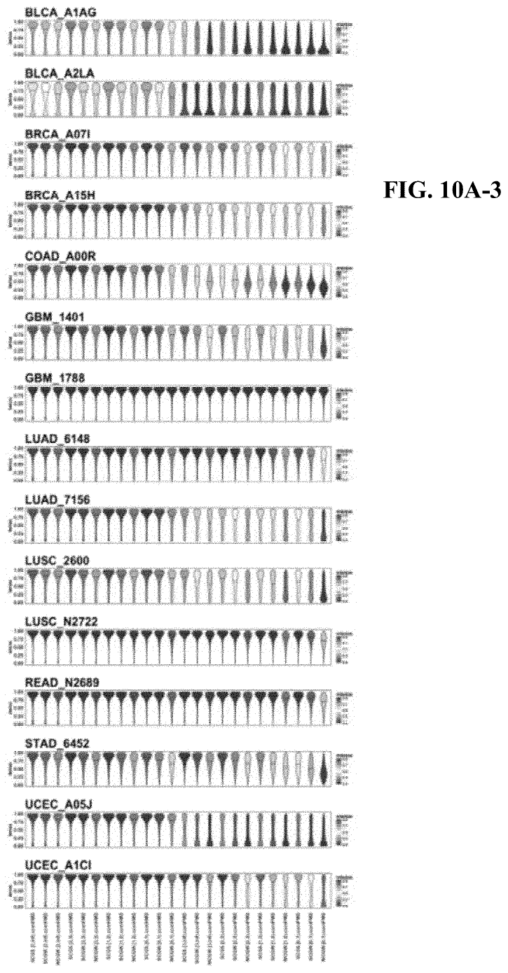

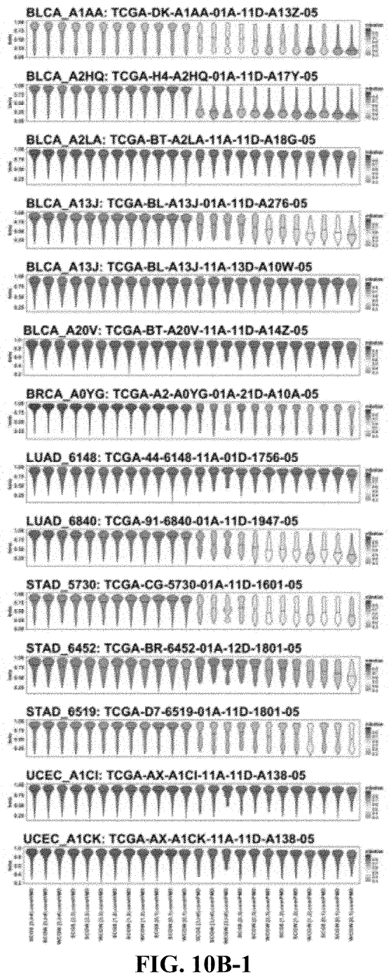

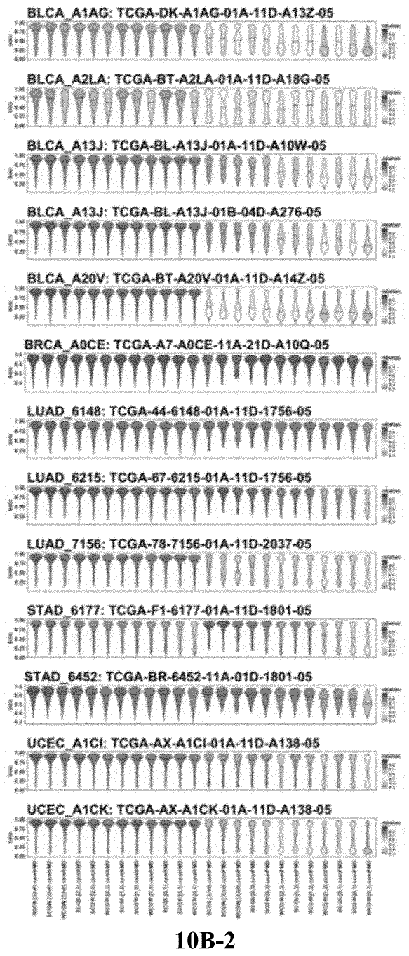

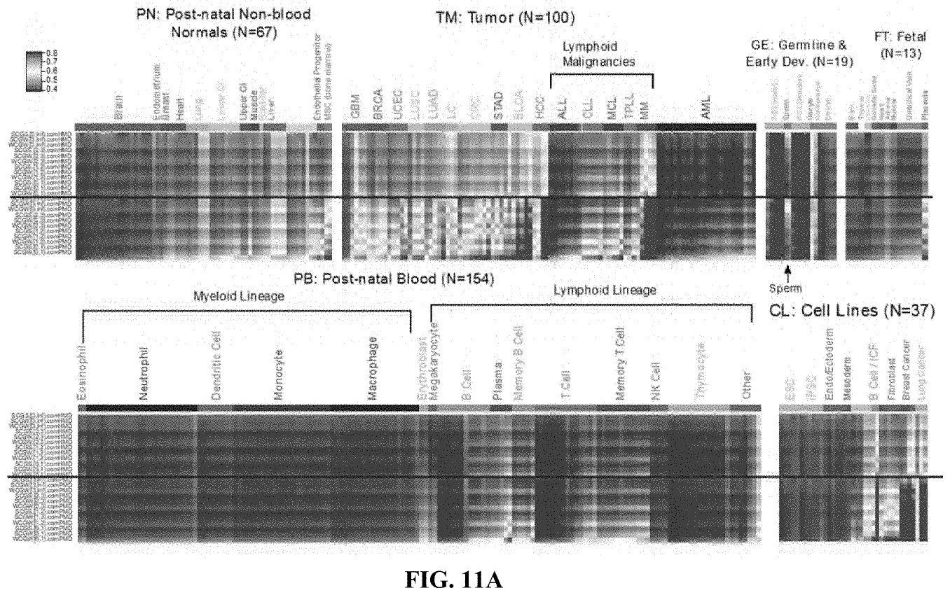

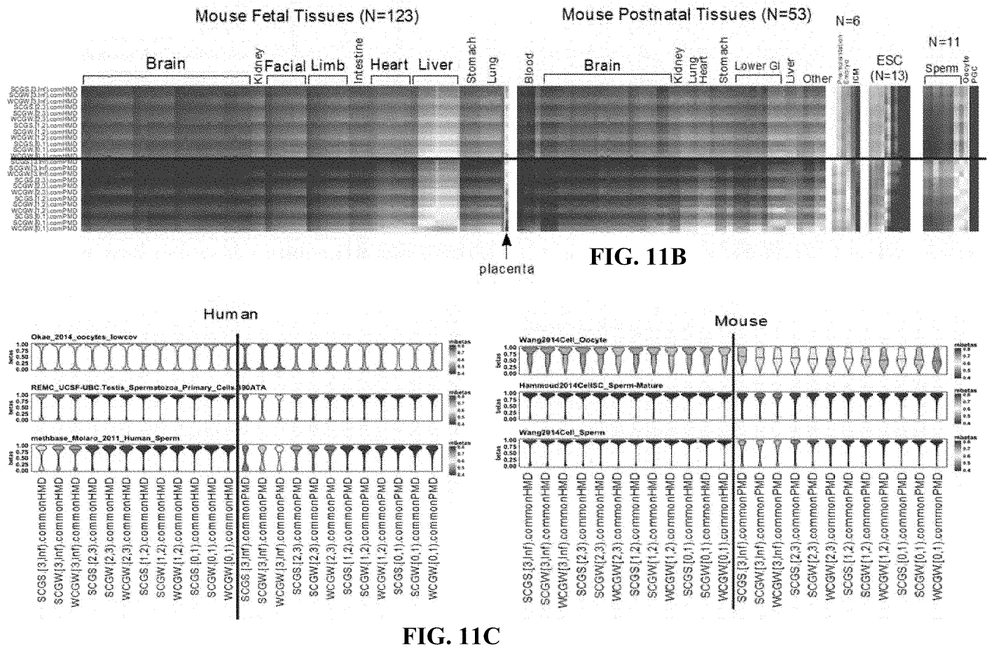

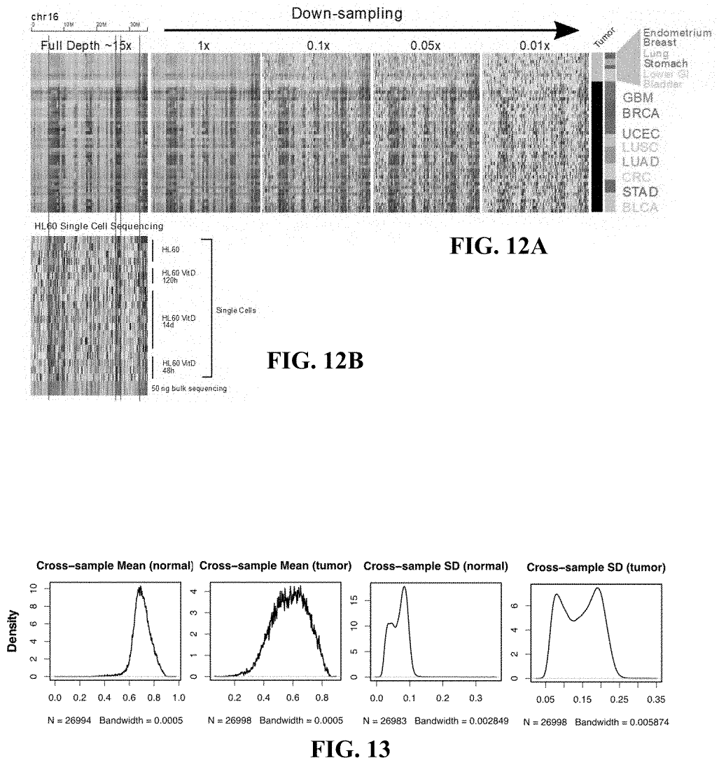

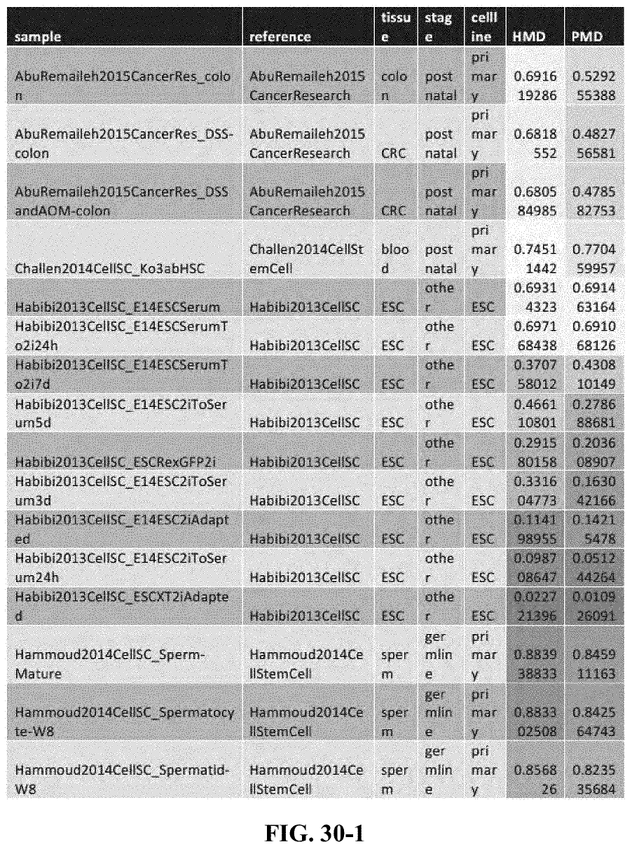

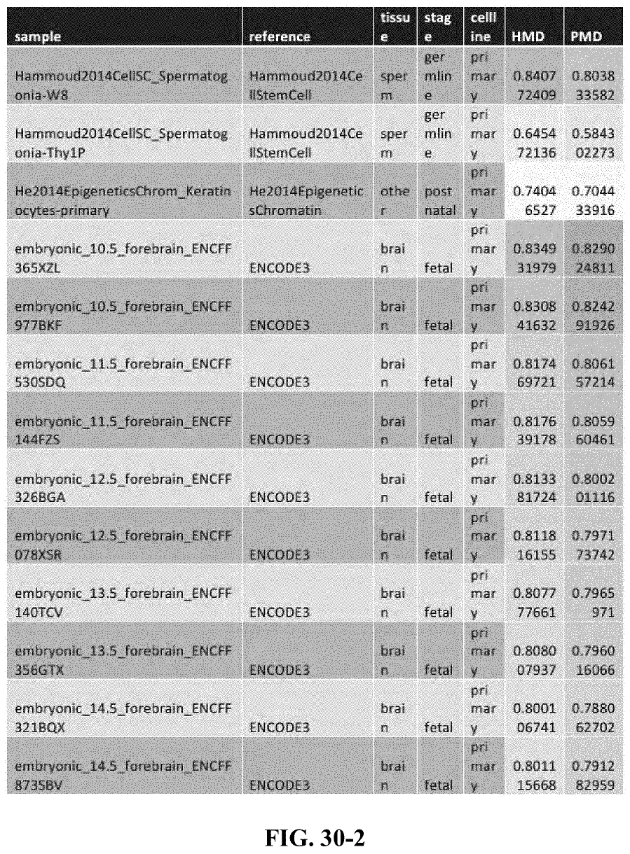

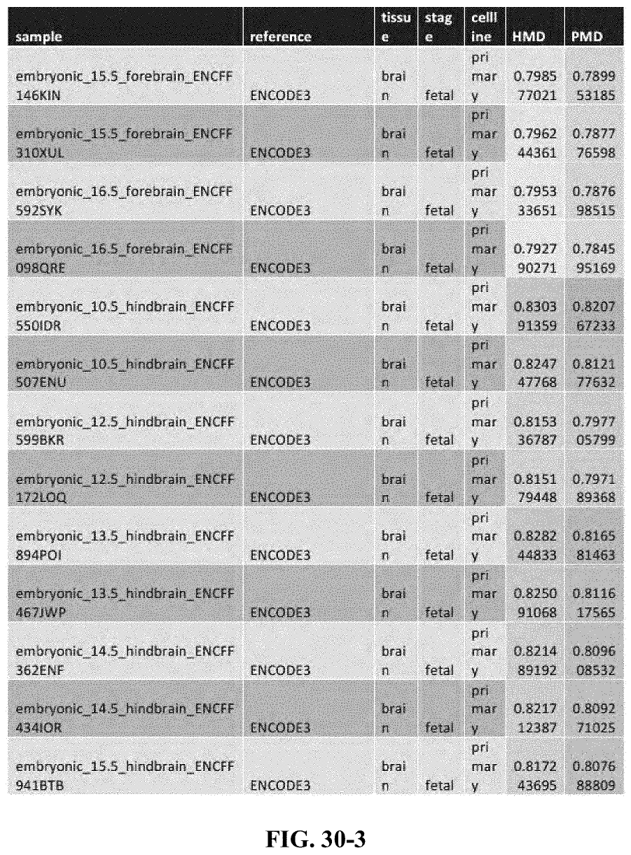

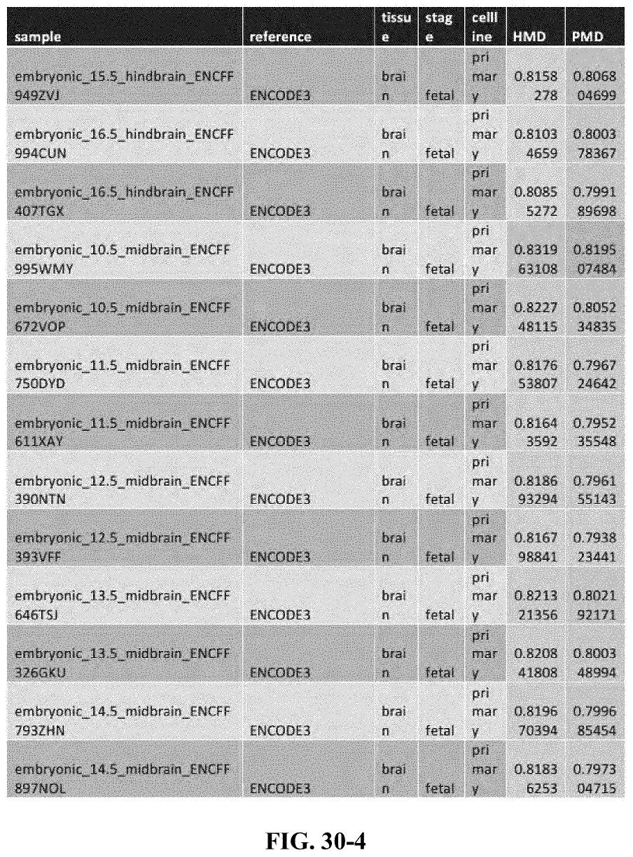

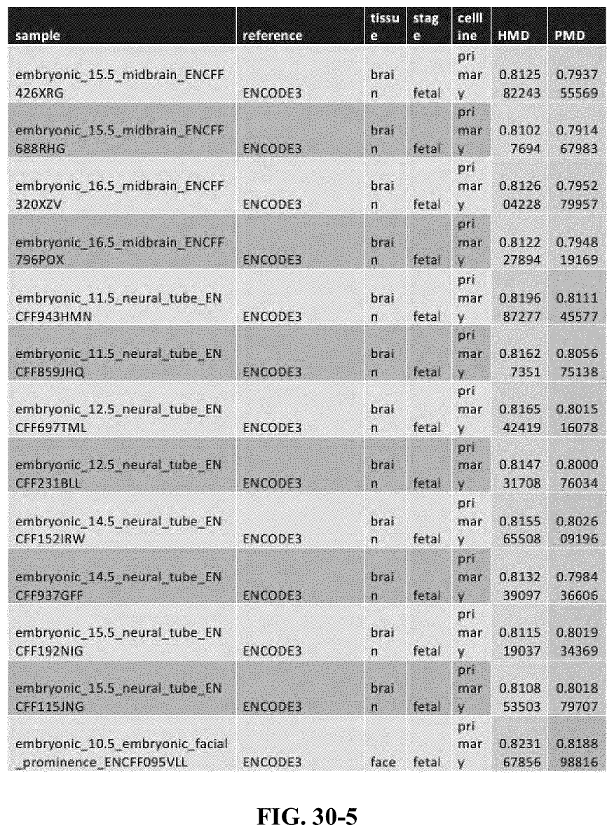

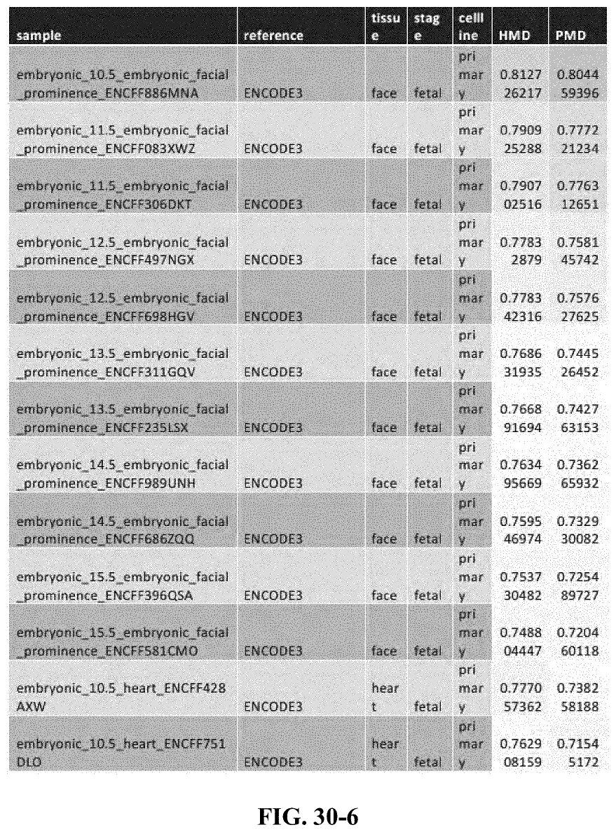

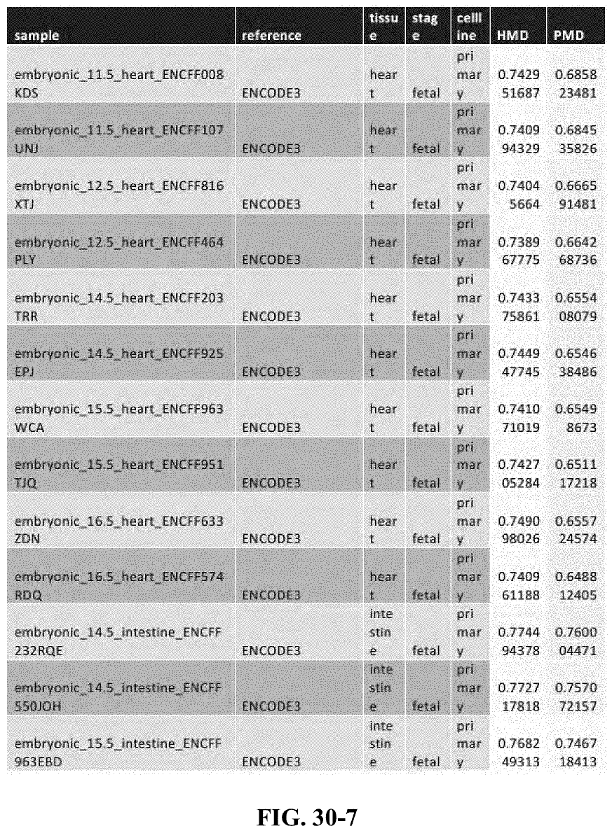

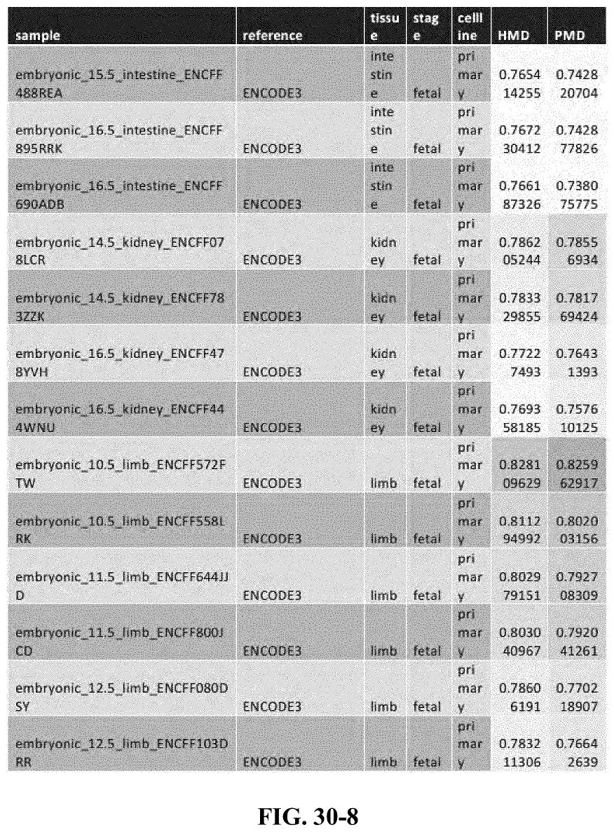

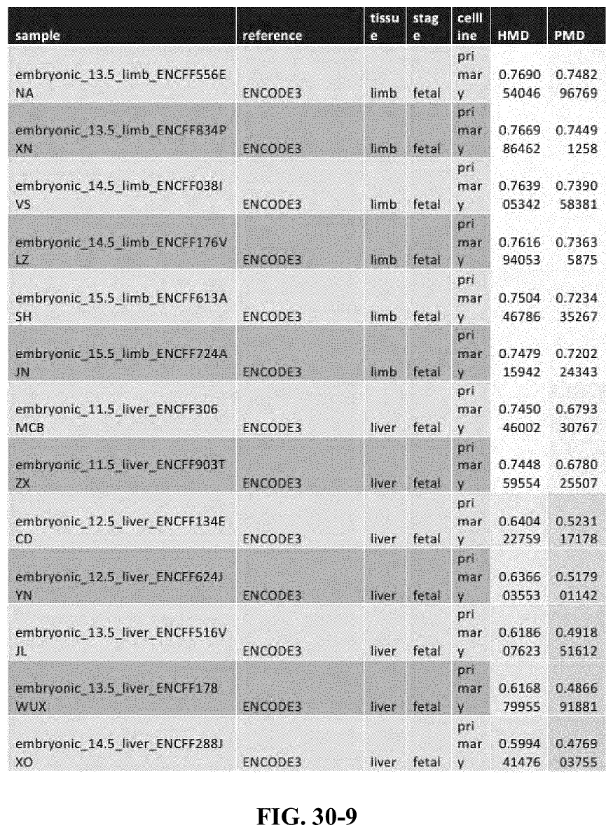

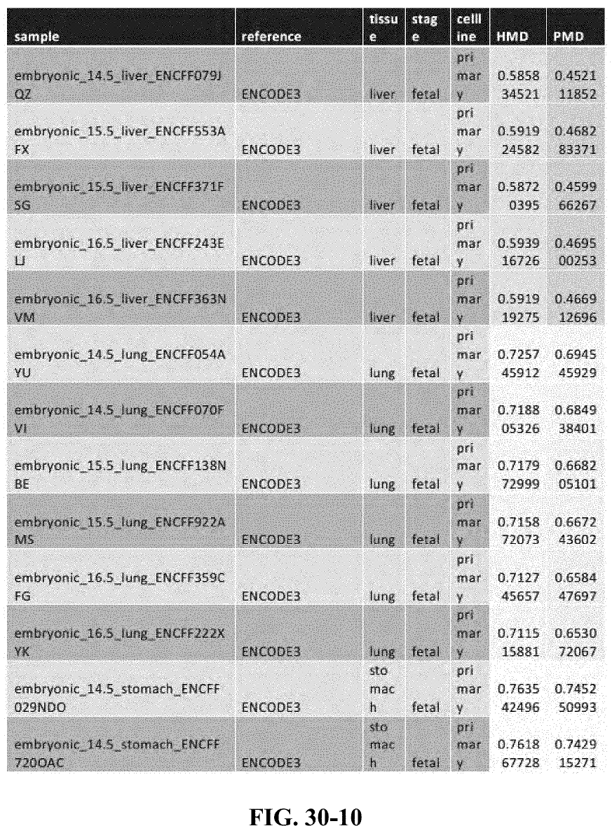

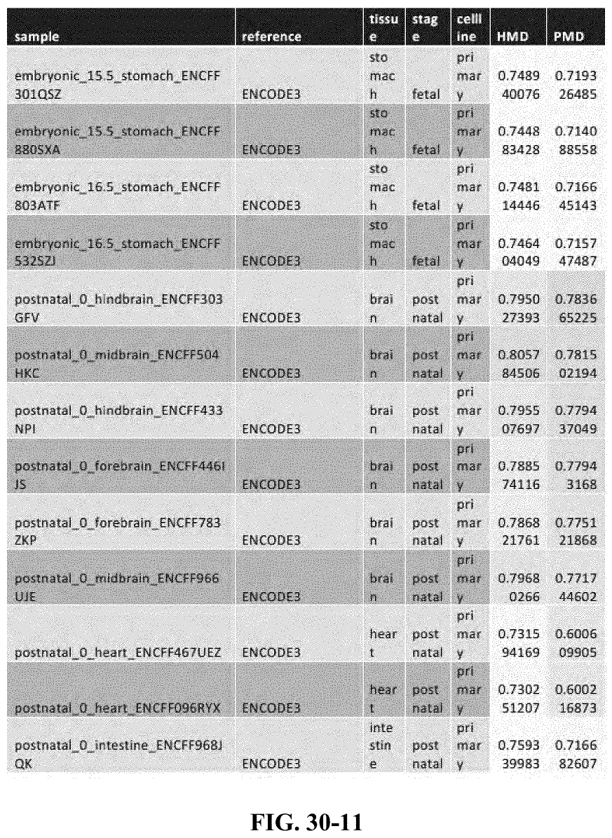

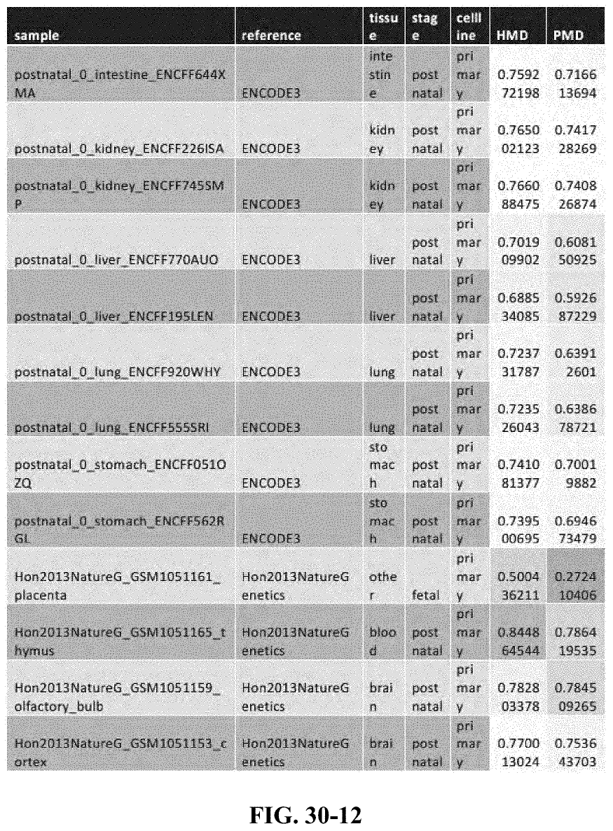

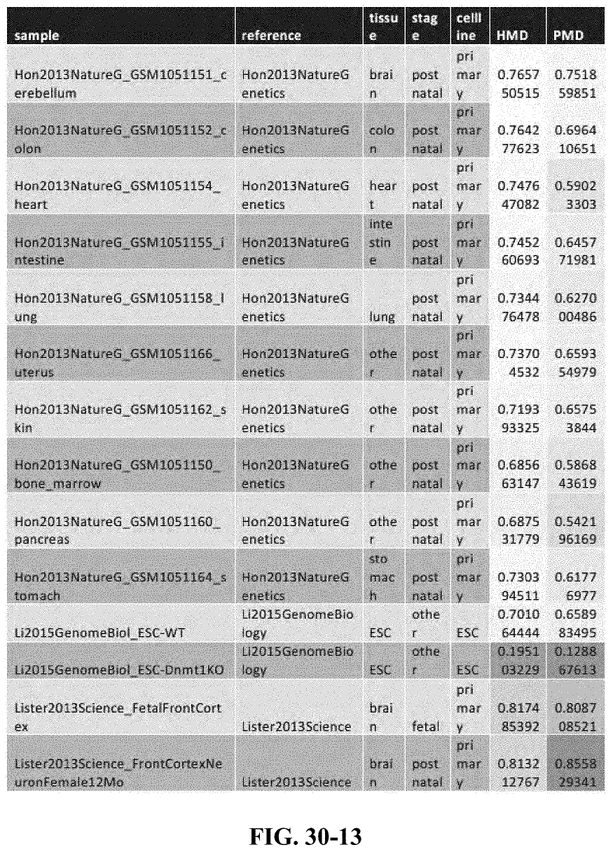

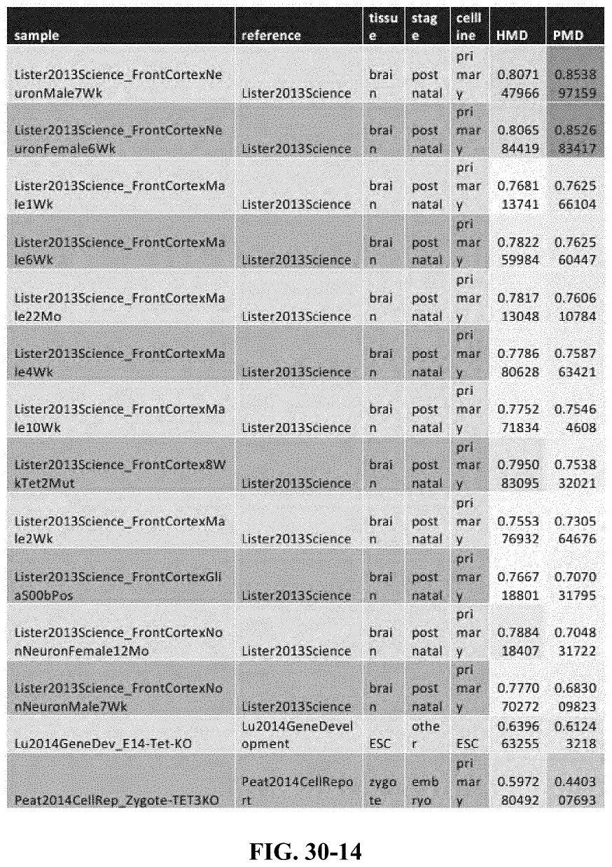

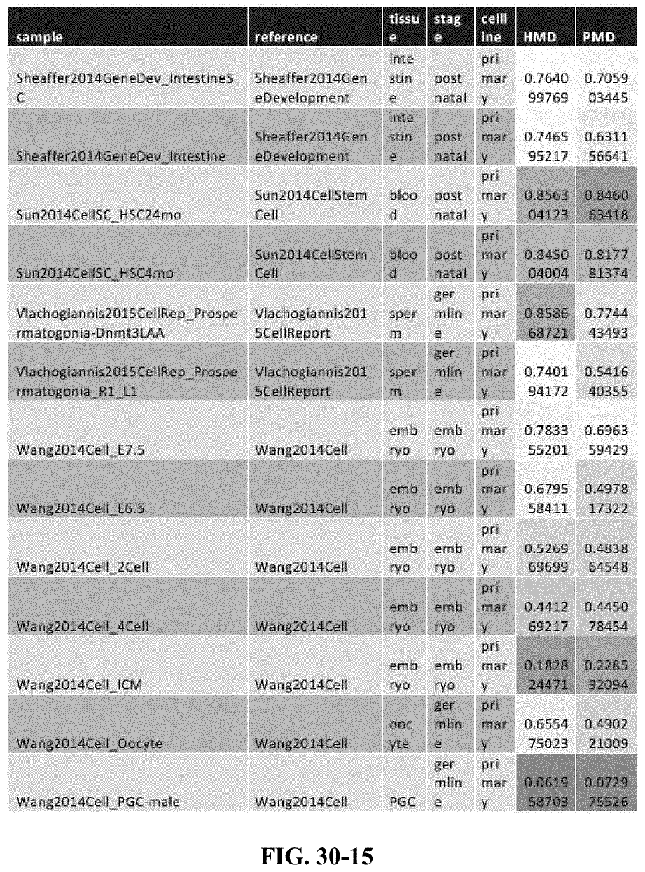

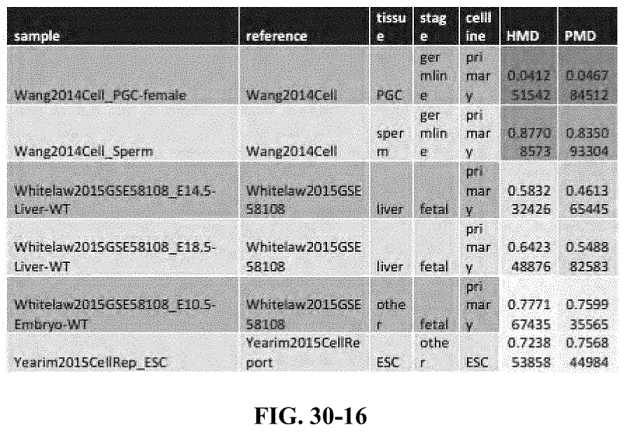

According to particular aspects, working Example 1 below describes the definition and use of a Solo-WCGW sequence motif having substantial utility for measuring genomic DNA methylation loss. Solo-WCGW CpGs were shown herein to be prone to hypomethylation. A set of shared partially methylated domains (PMDs) and highly methylated domains (HMDs) was initially defined across the majority of a 49 core sample set (40 core tumor samples and 9 core normal samples) (FIGS. 30-1 to 30-16; FIG. 9A). Low CpG density within windows of about +1-35 bp was found to be optimal for predicting PMD-specific hypomethylation (FIG. 9b). Additionally, CpGs flanked by an A or T (“W”) on both sides (WCGW tetranucleotides) were consistently more prone to DNA hypomethylation than those flanked by a C or G (“S”) on either (SCGW) or both (SCGS) sides (FIG. 1A; FIG. 9C). The most hypomethylation-prone sequence context was at CpGs with the combination of zero neighboring CpGs (“solo”) and the WCGW motif. These same sequence dependencies were consistent within all other tumor and adjacent normal samples in the core set, using either the WGBS data (FIG. 10A1-A3) or matched Illumina Infinium HumanMethylation450™ (HM450) microarray data (FIG. 10B1-B2). An additional 390 human and 206 mouse WGBS samples examined later exhibited the same pattern (FIGS. 11A and 11B), with the exception of three germ cell samples (FIG. 11C). While they represent only the extreme of a hypomethylation process that affects other CpGs, focusing on solo-WCGWs alone enhanced the signal of PMD/HMD structure, especially in normal adjacent tissues and weakly hypomethylated tumors such as COAD-3518 (FIG. 1C). In addition to enhancing the PMD/HMD signal in high coverage WGBS data, solo-WCGW CpGs allowed accurate PMD structure to be determined with average genomic read coverage as low as 0.05× in down-sampled bulk WGBS data (FIG. 12A), and in low-coverage single-cell WGBS data (31) (FIG. 12B), providing for an application for low coverage or single-cell WGBS studies.

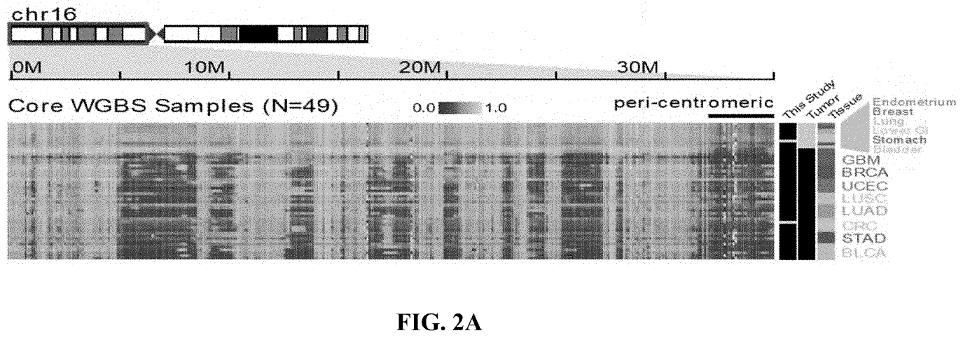

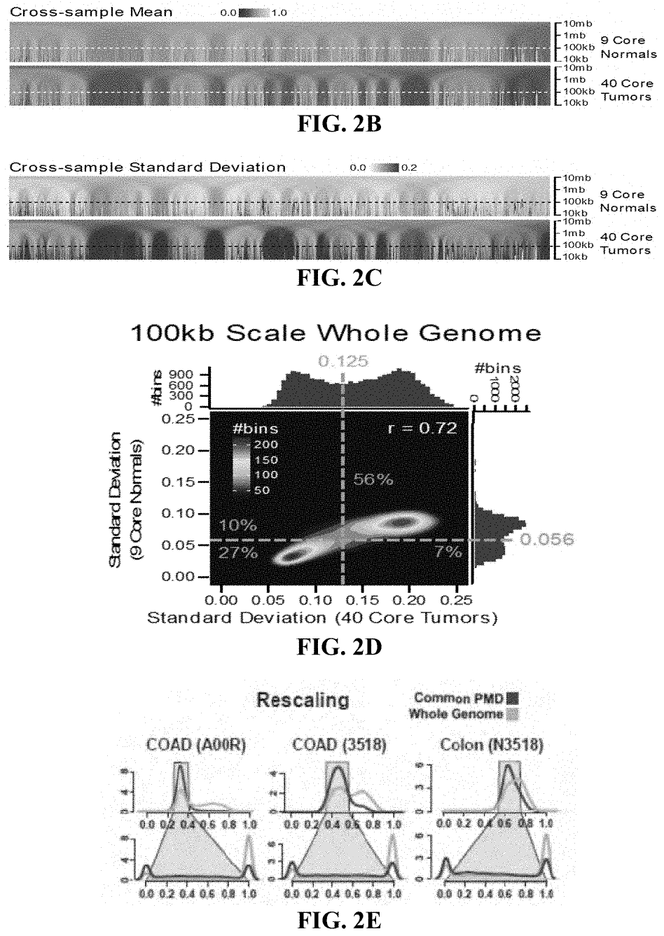

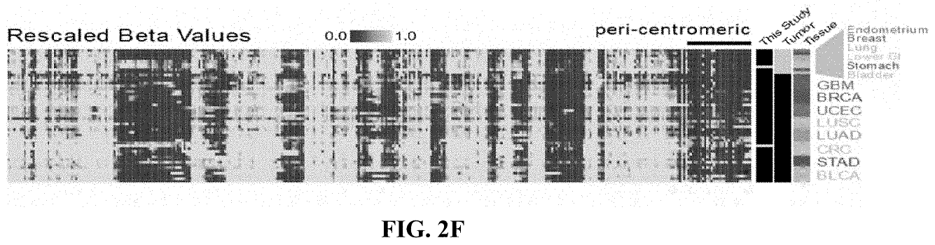

According to additional aspects, working Example 2 below describes data showing that most PMDs were shown to be shared across cancer and normal tissues. Genome-wide, standard deviation SD of solo-WCGW PMD hypomethylation was bimodally distributed within 100-kb bins in both normal and tumor core groups (FIGS. 2A 2C and 2D), unlike mean methylation (FIG. 13) and all other features examined (not shown). Using the bimodal SD peaks as a classifier resulted in a segmentation of the genome into HMDs and PMDs, and resulted in 100-kb bin classifications that were 83% concordant between the normal and tumor groups (FIG. 2D). This SD-based classification of PMDs allowed for rescaling of methylation values for individual samples based on their sample-specific degree of PMD hypomethylation (FIGS. 2E-F), further illustrating the high degree of concordance in PMD/HMD structure across tumor and normal samples.

According to additional aspects, working Example 3 below describes data showing that most PMDs where shown to be shared across developmental lineages. The findings support the idea, according to particular aspect of the present invention, that a large set of cell-type-invariant PMDs dominate the hypomethylation landscape in most tissues.

According to additional aspects, working Example 4 below describes data showing that PMD hypomethylation emerges during embryonic development. The substantial similarity of PMD structure detected between ICMs, ESCs, embryonic (<8 weeks) stages, and post-natal samples, suggests that PMD hypomethylation begins at the earliest stages of development. This interpretation is strengthened by the observation that the degree of hypomethylation observed at the fetal and postnatal stages for each cell type largely mirror the lineage-specific hypomethylation rate within the same embryonic cell type.

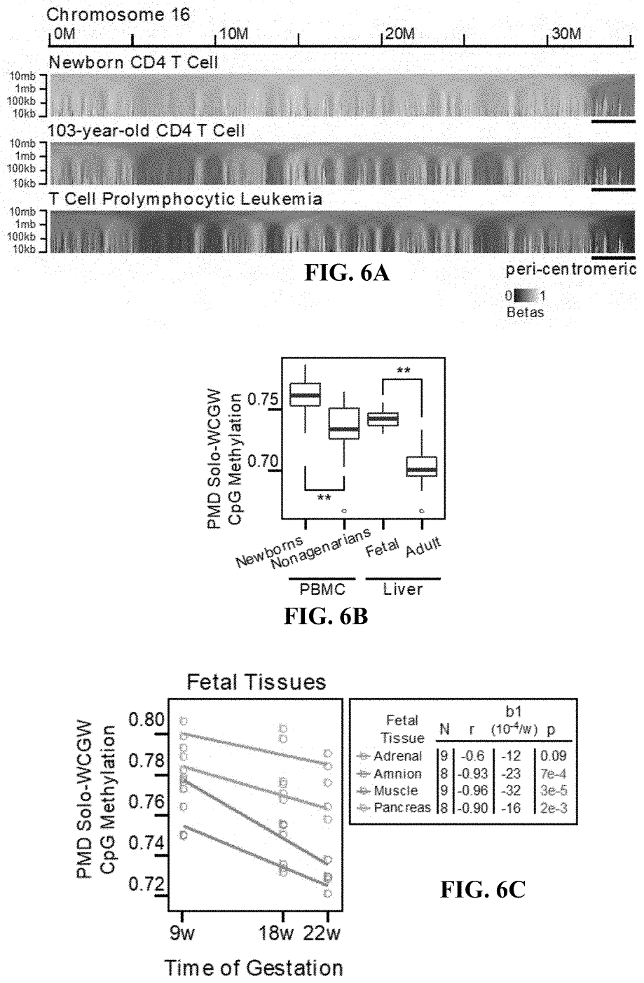

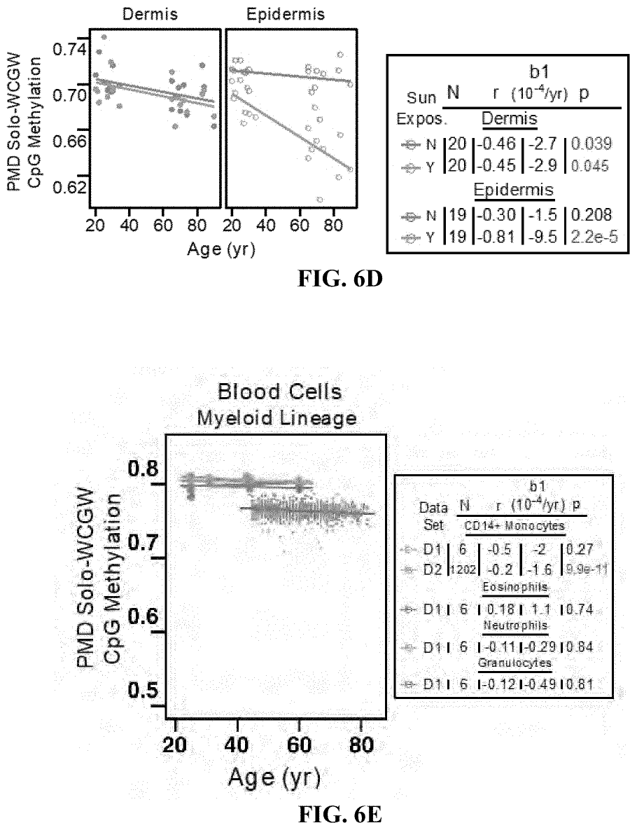

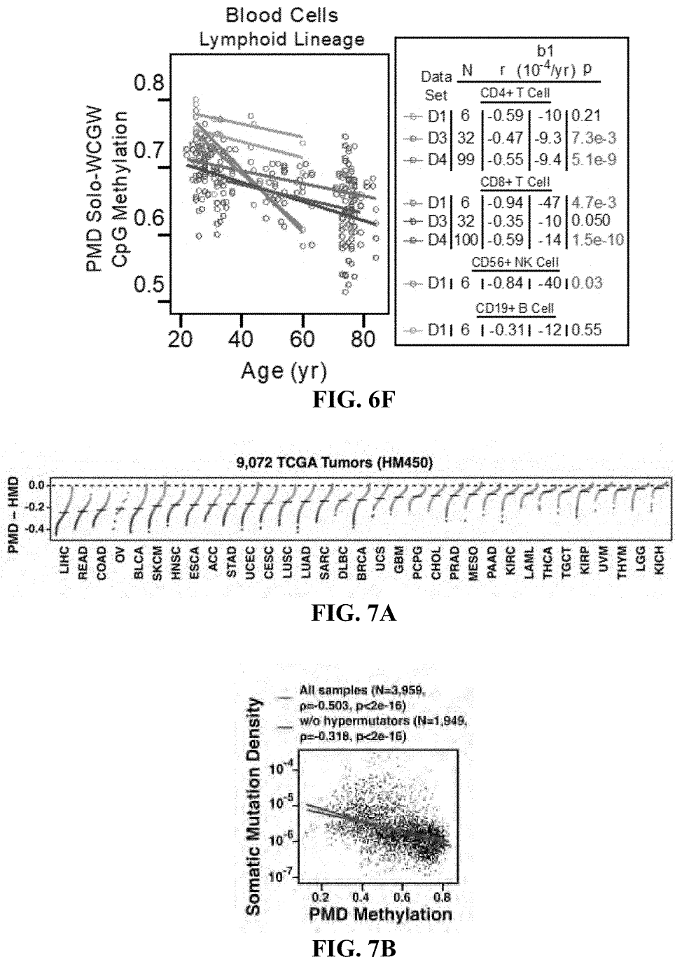

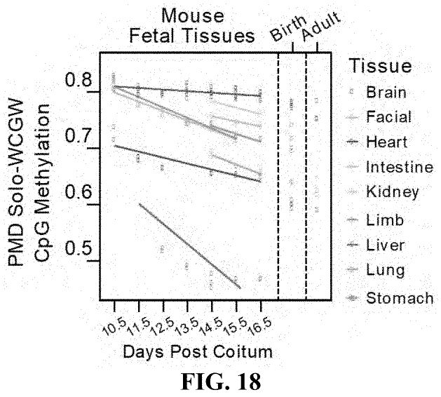

According to additional aspects, working Example 5 below describes data showing that PMD hypomethylation is associated with chronological age. A strong age association was evident from the WGBS profile of sorted CD4+ T cells from a newborn vs. those from a 103-year-old individual, with the latter being closer to a T cell-derived leukemia than to the newborn sample (FIG. 6A). Strikingly, fetal tissues from four different developmental lineages showed nearly linear accumulation of hypomethylation from 9 weeks post-gestation to 22 weeks post-gestation (FIG. 6C). Despite small sample sizes, this was statistically significant for 3 of the 4 fetal tissue types. A similar association was observed between PMD hypomethylation and gestational age in multiple mouse fetal tissue types (FIG. 18). The presently disclosed solo-WCGWs analysis revealed that both dermal and epidermal cells exhibited age-associated PMD hypomethylation without sun exposure, but that this process was dramatically accelerated specifically in epidermal cells upon sun exposure (FIG. 6D). This suggests that while PMD hypomethylation is a nearly universal process in aging, the degree of hypomethylation is a reflection of the complete mitotic history of the cell, including proliferation associated with normal development and tissue maintenance, plus additional cell turnover occurring as a consequence of environmental insults. Diverse hematopoietic cell types had a significant association between donor age and degree of hypomethylation, with the myeloid lineage (FIG. 6E) having a much slower rate of age-associated loss compared to the lymphoid lineage (FIG. 6F). This finding is consistent with the overall lower degree of methylation observed in myeloid cell types from WGBS data. While the rate of loss within the myeloid lineage was extremely low, the association to donor age was highly significant within the large human monocyte dataset (FIG. 6E).

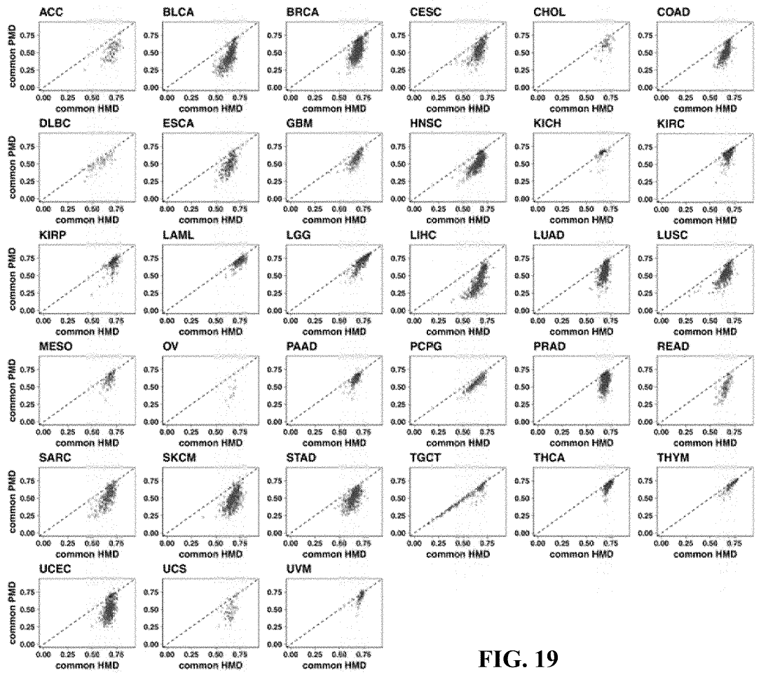

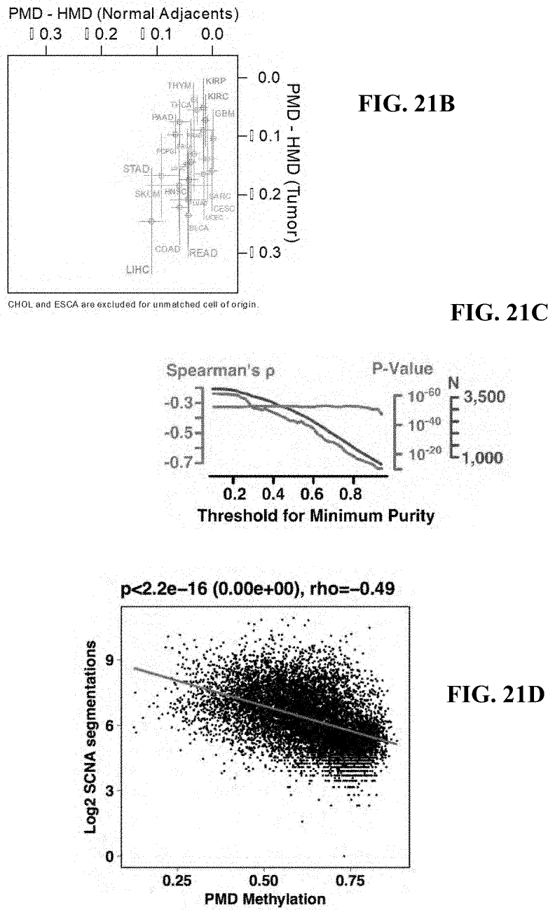

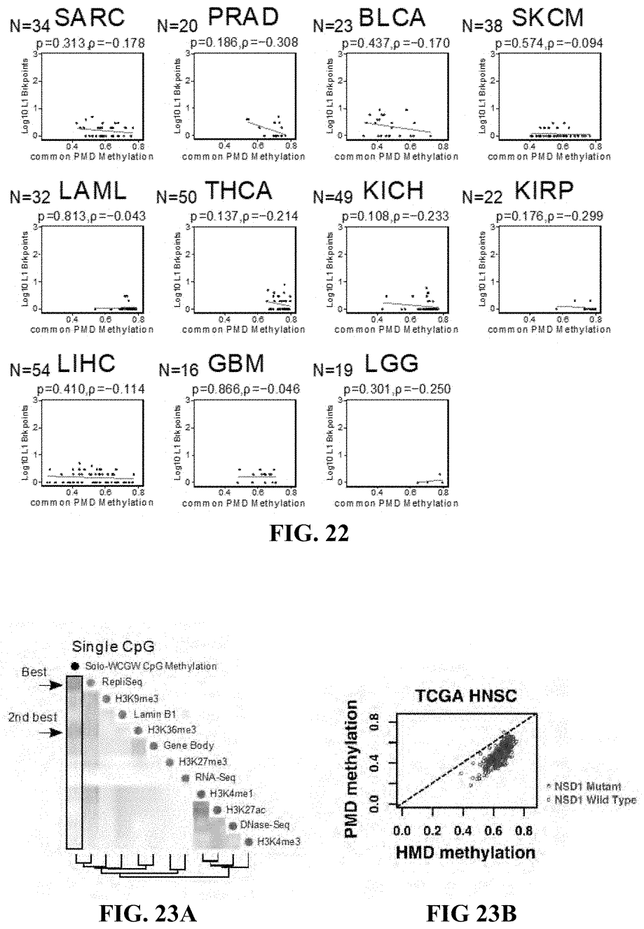

According to additional aspects, working Example 6 below describes data showing that PMD hypomethylation is linked to mitotic cell division in cancer. PMD hypomethylation was nearly universal but showed extensive variation both within and across cancer types. Comparison to 749 adjacent normals from TCGA showed that the relative degree of hypomethylation across cancer types was correlated with that of the disease-free tissue of origin (FIGS. 19-21). PMD hypomethylation was also associated with somatic copy number aberration density (FIG. 21D). Intriguingly, tumors with deeper PMD hypomethylation had more LINE-1 insertions in 8 of 9 cancer types, with the only exception being endometrial cancer (FIG. 7D; FIG. 22). According to particular aspects of the present invention, tumors highly proliferative at the time of specimen collection may also reflect an extensive history of past cell division. Supporting a link between ongoing cell proliferation and PMD hypomethylation, the genes with the greatest association to PMD hypomethylation were strongly enriched within a list of 350 cell-cycle dependent genes from Cyclebase (44) (FIG. 7F). Ranking tumor samples by their degree of PMD hypomethylation showed that this association involved most cell-cycle dependent genes across different mitotic stages (FIG. 7G). According to particular aspects of the present invention, all of the presently disclosed tumor mutation and expression results suggest cumulative mitotic cell divisions as the major driving force behind PMD hypomethylation accumulation.

According to additional aspects, working Example 7 below describes data showing that both replication timing and H3K36me3 were shown to affect methylation. IMR90 cells, for which there is publicly available data for all relevant histone and topological marks, was used to systematically analyze the presently disclosed solo-WCGW based PMD definition. This analysis confirmed that HMD/PMD structure coincided with nuclear architecture, as characterized by Hi-C A/B compartments, Lamin B1 distribution and replication timing (FIG. 8A). At the single CpG scale, Solo-WCGW CpG methylation was most strongly correlated with replication timing, followed by the histone mark H3K36me3 (FIG. 23A). A stratified analysis of all solo-WCGW CpGs in the genome (FIG. 8B-C) was performed, revealing that the 14% of Solo-WCGWs overlapping H3K36me3 were highly methylated, irrespective of position relative to gene annotations or replication timing (FIG. 8B, left). The remaining 86% of Solo-WCGWs (those not overlapping an H3K36me3 peak) had lower methylation across all contexts, but were strongly replication-timing dependent (FIG. 8B, right). Because most somatic cell types had detectably hypomethylated PMDs like IMR90 (and unlike H1), the presently disclosed observations support a model in which highly effective methylation maintenance at H3K36me3-marked regions is achieved through a process mediated by the direct recruitment of DNMT3B through its PWWP domain (45). Consistent with earlier observations (9), this H3K36me3-linked maintenance appears to act independently from the effect of replication timing on PMD methylation loss (FIG. 8D).

According to additional aspects, working Example 8 below describes the materials and methods used in the presently disclosed work, including whole genome bisulfite sequencing, external data, alignment and extraction of methyl-cytosine levels, genomic binning, definition of preliminary PMD/HMD domains. final definition of PMDs/HMDs based on standard deviation of solo-WCGW methylation, HM450 analysis, analysis of the IMR90 epigenome, rescaling based on PMD methylation, stratified analysis of solo-WCGW CpGs in the genome, statistics, data availability, code availability, and URLs).

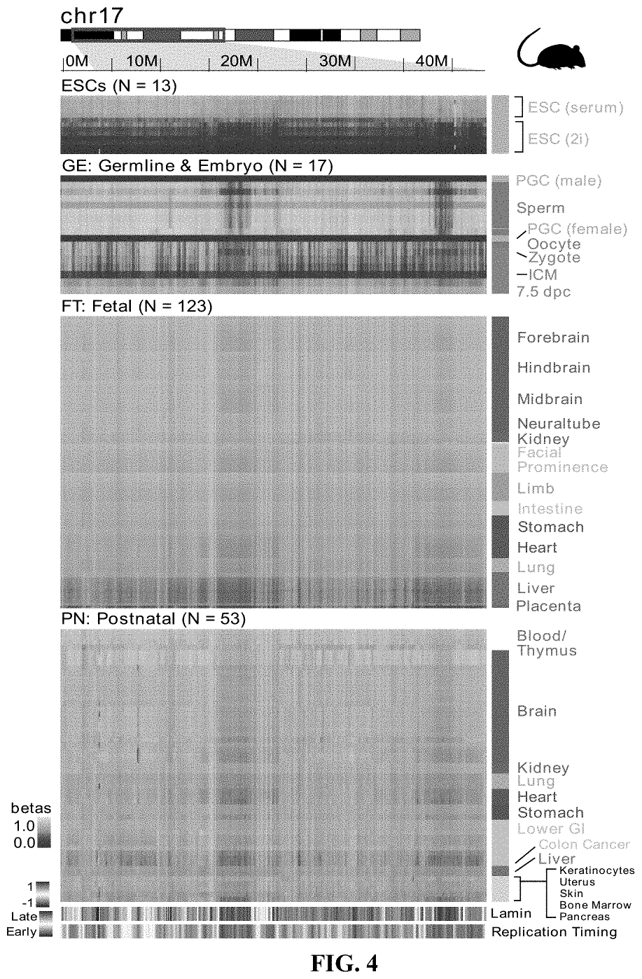

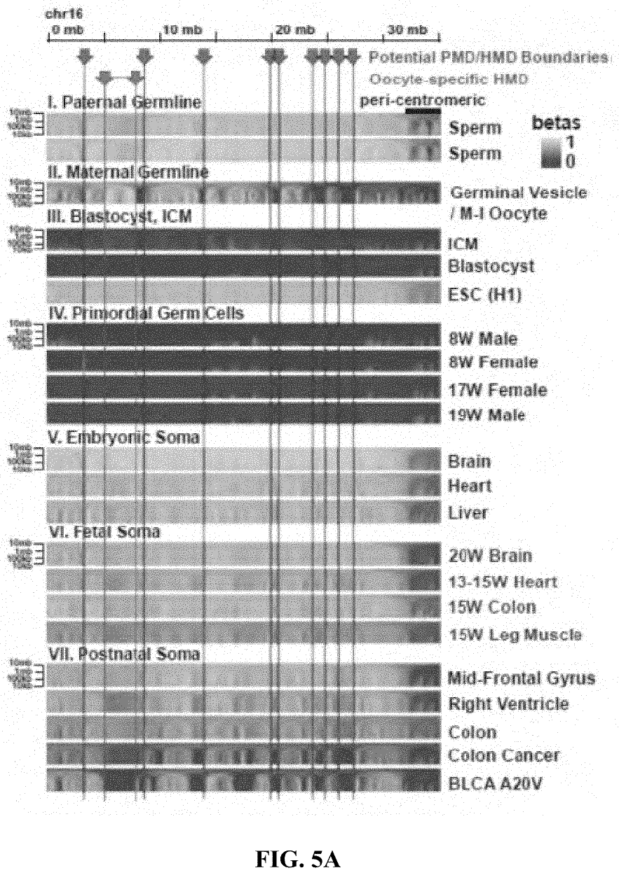

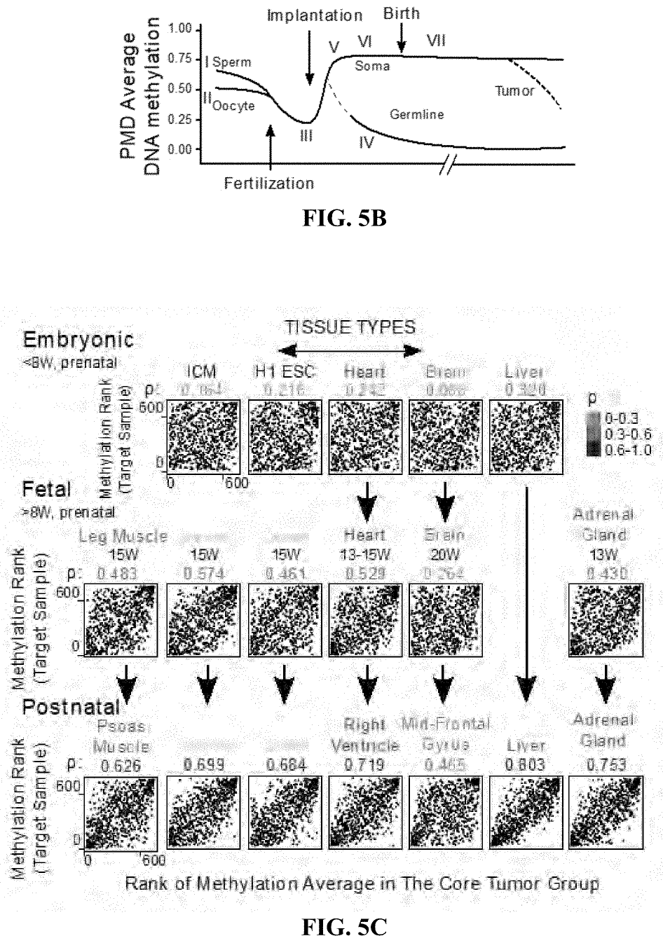

According to additional aspects, working Example 9 below describes data showing that PMD hypomethylation in immortalized cell lines was demonstrated using the solo-WCGW motif. PMD hypomethylation was observed in almost all cultured cell lines except for ESCs, iPSCs and their derived cell lines (FIG. 4 Group ESC). The stark contrast between the primary inner cell mass (ICM) sample and the heavily methylated hESCs suggests that cultured hESCs may reflect a later stage of post-implantation embryonic development, where expression of the DNMT3A and DNMT3B methyltransferases can help to maintain high levels of DNA methylation despite prolonged culture (FIG. 5A).

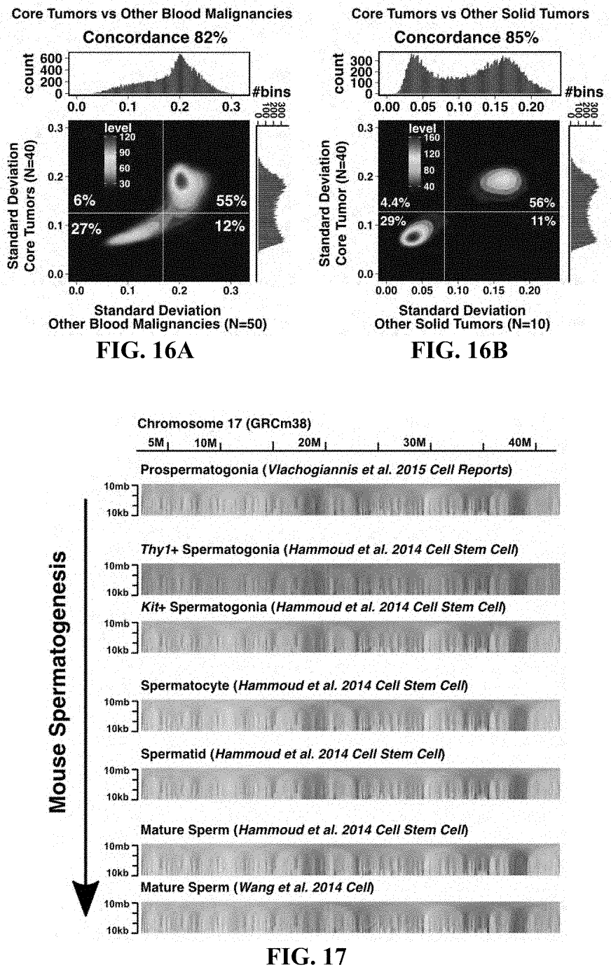

According to additional aspects, working Example 10 below describes data showing that improved analysis of HMD/PMD structure was obtained using the solo-WCGW motif. Cell-type invariant PMDs were useful for investigating general properties of methylation loss over time. PMDs were defined in the present work by exploiting the inherent variance in PMD hypomethylation levels across large cohorts of samples, which was the only cross-sample feature bimodally distributed between HMDs and PMDs. Under this definition, for example, the core tumor group (containing only solid tumors) had almost the same degree of shared PMDs with blood malignancies (82%) as it did with other solid tumors not from the core set (85%) (FIG. 16). The present focus on common PMDs, however, does not discount the importance of cell-type-specific PMDs. According to particular aspects of the present invention, incorporation of solo-WCGW sequence features can be used to improve current methods for such cell-type-specific PMD detection, including kernel-based (87), HMM-based (88) and multi-scale based (89), and methods for methylation array data (84). Explicitly modeling and subtracting PMD-related hypomethylation will reduce noise and enhance the ability to detect changes in TET-mediated demethylation processes affecting short-range elements such as promoters, enhancers, and insulators.

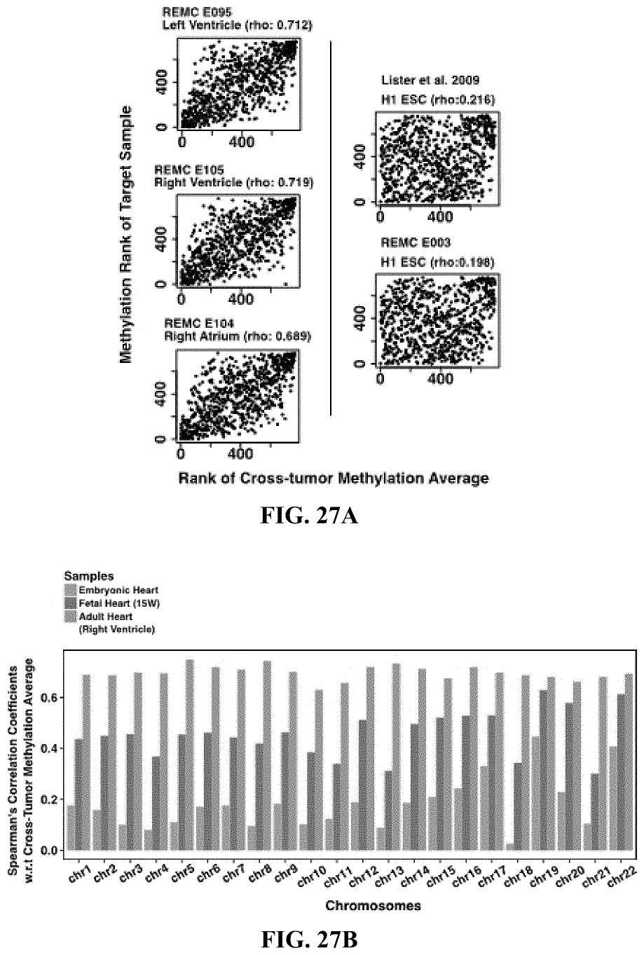

According to additional aspects, working Example 11 below describes data showing that the stability of rank-based correlation between methylomes was demonstrated using the solo-WCGW motif. A rank-based analysis of 792 genomic 100 kb bins from chromosome 16 (FIG. 5) was performed to measure the HMD/PMD structure in normal tissues at different developmental stages. The rank correlations had only minor variations between replica or closely related samples (FIG. 27A) and the patterns were stable when using bins from different chromosomes (FIG. 27B).

According to additional aspects, working Example 12 below discusses an alternative nuclear localization model (FIG. 8G) of PMD hypomethylation.

According to additional aspects, working Example 13 below assesses the relevance of the PMD sequence signature to somatic and germline mutational landscape.

To investigate any potential impact of the PMD sequence signature on introducing cytosine deamination mutations in the CpG dinucleotides, the relative proportion of somatic mutations that are within certain tetranucleotide sequence contexts and certain numbers of neighboring CpGs was studied. Somatic CpG to TpG mutations reported in an early gastric cancer whole-genome sequencing experiment was compared, and indeed confirmed that solo-WCGWs within late replicating PMDs had a lower CpG to TpG mutation rate compared with other sequence context (FIG. 24A). De novo CpG->TpG mutations reported in a study of 1,548 Icelandic trios were studied, and these de novo CpG->TpG mutations in the maternal germline were indeed found to be depleted at CpGs in the WCGW context and with low local CpG density (FIG. 24Bb). The standing distribution of human and mouse CpGs is also consistent with the hypothesis that tendency of losing methylation in solo-WCGW context in the germline may exert a protective role for these CpGs against deamination (FIGS. 24C and 24D).

According to additional aspects, working Example 14 below, certain specific sub-patterns that match the Solo-WCGW definition were found to be more predictive than the general definition, and DNA shape features were also found to be predictive. According to additional aspects, therefore, more specific definitions and structures within the general Solo-WCGW pattern are provided for tracking replication-associated DNA methylation loss.

According to additional aspects, working Example 15 below describes the materials and methods used in the presently disclosed Examples 16-18, including primary cell culture, DNA methylation assay, Beta calling, QA/NA Removal, and Solo-WCGW subsetting.

According to additional aspects, working Example 16 below describes using an elastic net modeling strategy to identify a 44 CpG model for predicting mitotic history with and between cell types.

According to additional aspects, working Example 17 below describes using an individual probe regression strategy to identify 75 correlated probes for all tissue types studied.

According to additional aspects, working Example 18 below describes a comparison to the results of using the elastic net modeling strategy and individual probe regression strategy.

According to additional aspects, working Example 19 below describes a comparison of the solo-WCGW mitotic clock to existing clocks, including conception, model building and application.

According to additional aspects, working Example 20 below, the disclosed methods for measuring and tracking replication-associated DNA methylation loss are broadly applicable, and additional, non-limiting exemplary applications are provided.

### Terms (Definitions)

Unless otherwise explained, all technical and scientific terms used herein have the same meaning as commonly understood by one of ordinary skill in the art to which this disclosure belongs.

It is to be understood that the methods and systems are not limited to specific methods, specific components, or to particular compositions. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. “On the order of” can mean approximately, a fraction thereof, or a multiple thereof.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. All ranges disclosed herein are inclusive and combinable (e.g., ranges of “up to 25%, or, more specifically 5% to 20%” is inclusive of the endpoints and all intermediate values of the ranges of “5% to 25%,” etc.).

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not.

The sequence “WCGW” as used herein refers to a CpG dinucleotide sequence flanked by either A or T (e.g., ACGA, ACGT, TCGT, TCGA). According to particular aspects of the present invention, preferred WCGW sequences are those located in sequence motifs (e.g., ≥22 bp) characterized by specific G/C content and/or having only one or a few CpG dinucletides. For example, preferred aspects of the present methods comprise determining a mean or average methylation value, or a value related thereto, for a plurality of genomic CpG dinucleotide sequences, wherein each such CpG dinucleotide is the sole CpG dinucleotide sequence within a n(x)WCpGWn(x) genomic DNA sequence motif, wherein W=A or T, n=A or G or C or T, and wherein x≥9, to provide a measure of cellular replication-associated DNA methylation loss. In preferred aspects, xis a value selected from the group consisting of at least 9, at least 14, at least 19, at least 24, at least 29, at least 34, at least 39, at least 44, at least 49, at least 54, at least 59, about 34, 34±25, 34±15, or x is a value in a range selected from the group consisting of about 9-49, 9-99, 9-149, 9-199, 14-49, 14-99, 14-149, 14-199, 19-49, 19-99, 19-149, 19-199, 24-49, 24-99, 24-149, 24-199, 29-49, 29-99, 29-149, 29-199, 34-49, 34-99, 34-149, 34-199, 39-49, 39-99, 39, 149, 39-199, 44-49, 44-99, 44-149, 44-199, 49-99, 49-149, 49-199, 54-99, 54-149, 54-199, 59-99, 59-149, 59-199 and any subranges of the preceding ranges. Preferably, x is 34 (or about 34), or 34±25 (e.g., in the range of 9-59) or 34±15 (e.g., in the range of 19-49).

“Solo-WCGW” refers to a n(x)WCpGWn(x) genomic DNA sequence motif wherein the CpG dinucleotide of the WCGW sequence is the sole CgG dinucleotide sequence in the n(x)WCpGWn(x) genomic DNA sequence motif, wherein W, n and x are defined as in the preceding paragraph. Preferred solo-WCGW genomic DNA sequence motifs are those wherein x is 34 (or about 34), or 34±15 (e.g., in the range of 19-49), however less favored aspects of the methods may include x in a value range selected from 9 to 199 as described in the preceding paragraph.

In particular aspects, the Solo-WCGW motif may comprise the sequence n(x−1)mWCpGWGn(x−1), and wherein W=A or T, n=A or G or C or T, m=C or A, and x≥9 (with x varying as describe above in the preceding paragraphs). In the methods, the Solo-WCGW motif may comprise the sequence n(x−1)CWCpGWGn(x−1), and wherein W=A or T, n=A or G or C or T, and x≥9 (with x varying as describe above in the preceding paragraphs).

Exemplary human and mouse n(x)WCpGWn(x) genomic DNA sequence motif species are provided in Tables 4-7 below.

In particular, less favored, aspects of the methods, the n(x)WCpGWn(x) genomic DNA sequence motif may comprise 1 or 2 CpG dinucleotide sequences in addition to the CpG dinucleotide sequence of the WCGW sequence. In such aspects, x is a value selected from the group consisting of at least 9, at least 14, at least 19, at least 24, at least 29, at least 34, at least 39, at least 44, at least 49, at least 54, at least 59, about 34, 34±25, 34±15, or x is a value in a range selected from the group consisting of about 9-49, 9-99, 9-149, 9-199, 14-49, 14-99, 14-149, 14-199, 19-49, 19-99, 19-149, 19-199, 24-49, 24-99, 24-149, 24-199, 29-49, 29-99, 29-149, 29-199, 34-49, 34-99, 34-149, 34-199, 39-49, 39-99, 39-149, 39-199, 44-49, 44-99, 44-149, 44-199, 49-99, 49-149, 49-199, 54-99, 54-149, 54-199, 59-99, 59-149, 59-199 and any ranges or subranges of the preceding ranges. In particular of such aspects, x is 34 (or about 34), or 34±25 (e.g., in the range of 9-59) or 34±15 (e.g., in the range of 19-49).

For purposes of the presently disclosed methods, in the context of the various above-described n(x)WCpGWn(x) genomic DNA sequence motifs, certain instances of the motif are more predictive (e.g., for tracking replication-associated DNA methylation loss) than others. In our analysis, Solo-WCGWs (as described above) in the contexts ACGA, TCGA, and ACGT are not equally predictive for tracking replication-associated DNA methylation loss.

As used herein, “condition or state” of a test cell or tissue sample means the health of a cell or tissue, including, for example, the condition or state of a normal (healthy) cell or tissue, a diseased cell or tissue, and/or a cell or tissue showing some signs indicative of a diseased state. In one example, the condition or state are signs indicative of the beginning of a diseased state and/or the progression or advancement towards a diseased state. The “condition or state” of a test cell or tissue sample also includes the type of cell or tissue, for example, the developmental stage of a particular cell or tissue type (embryonic, fetal, neonatal, adult), and the differentiated type of cell of tissue, for example, a liver cell, lung cell, brain cell.

As used herein, the term “effective cell division” or “effective cell divisions” means the process of dividing a parent cell into two new identical daughter cells, each daughter cell including the same number of chromosomes and genetic content as that of the parent cell. In one aspect, effective cell division may refer to the number of nuclear divisions when a eukaryotic cell reproduces during maintenance or growth.

As used herein, “determining the number of effective cell divisions” means determining the number of cells present after effective cell division(s). In one aspect, in the in vitro environment, the number of cells present after division(s) of a test cell can be determined by serially measuring the growth of the cell culture with a count slide (or hemacytometer) and a microscope, or with a spectrophotometer. In another aspect, stains are used to distinguish viable from non-viable cells to account for rates of cell death.

In one aspect, as used with Examples 15-18 below, the number of effective cell divisions may be determined according to the following methods. Primary cells are maintained under pro-mitotic conditions using optimal media formulations as recommended by the vendor (Coriell). The neonatal fibroblast lines (AG21859, AG21839) are cultured in 1:1 Ham's F12: Dulbecco Modified Eagle's Medium, with 2 mM L-glutamine, 15% v/v fetal bovine serum (FBS), and 1% v/v penicillin-streptomycin. The adult fibroblast line (AG16146) is cultured in Eagle's Minimum Essential Medium with Earle's salts, 1% v/v non-essential amino acids, 10% FBS v/v, and 1% v/v penicillin-streptomycin. The adult vascular smooth muscle line (AG21546) is cultured in Medium 199 in Earl BSS, with 2 mM L-glutamine, 10% FBS v/v, 0.02 mg/ml Endothelial Cell Growth Supplement, 0.05 mg/ml Heparin, and 1% v/v penicillin-streptomycin. Culture dishes are first coated with sterile gelatin (0.1% w/v) before seeding; this facilitates attachment and growth. The adult endothelial line (AG11182) is cultured under identical conditions to the vascular smooth muscle cell line (AG11546) except 15% v/v FBS is included. All primary cell lines are maintained at 37° C. at 5% CO2. Media is aspirated and replaced every 2-3 days. Replicative senescence is defined qualitatively as the inability to reach confluence at two weeks following the most recent passaging event, or >60% non-viable cells as quantified below.

Cells are counted using an automated cell counter (BioRad TC20). Briefly, 10 ul of a suspension of cells are retained at each passage. An equal volume (10 ul) of 0.40% Trypan Blue Dye is added to and gently mixed with the cell suspension. The addition of Trypan Blue Dye allows for detection of the live/dead cell fraction; dead cells are stained and live cells are not. Ten microliters of the stained cell suspension is applied to both chambers of a double-sided hemocytometer/counting slide. Both sides are read by an automated cell counter (BioRad TC20) and the average live/dead cell counts is calculated.

Population doubling level (PDL) is a standard method for quantifying mitoses within a population, given the initial seeding density and the final cell count at harvest. PDL for a given passage is calculated as followed:

$$PDL = 3.32 x \frac{{log}_{10} final viable cell count}{{log}_{10} starting viable cell count}$$

This is a derivative equation of the binary fission equation: x=2ⁿ wherein x=final cell count and n=number of population doublings. The multiplier 3.32 is introduced by converting from

$${log}_{2} x to {log}_{10} x , e . g . 3.32 = \frac{1}{{log}_{10} 2} .$$

To calculate the total mitotic history, the sum of total PDLs (from passage 1 onward) is taken:

Total PDL=Σ_{passage 1}^{passage n}PDL

The vendor (Coriell) may provide a starting PDL for primary cell lines that are established in their facilities; this is also included in the cumulative PDL.

In another aspect, in an in vivo environment, the number of cells present after cell division(s) can be determined by serially measuring the change in volume of a cell mass of a test cell or cells, or test cell tissue that has been grafted onto the animal, e.g., a mouse or other rodent.

As used herein “conditions for the test cell to divide” means conditions for effective cell division; and such conditions can be provided either in an in vitro environment or an in vivo environment. In vitro, in one embodiment, the conditions for a test cell to divide may include a culture plate containing a solid or liquid media or agar. In one aspect, conditions for encouraging a test cell to divide in vitro in the media/agar include providing a nutrient-rich broth in the media/agar along with, in some instances, antibiotics to promote cell growth; and providing temperature conditions favorable for cell growth (for example, 37° C.). In vivo, in one embodiment, the conditions for a test cell to divide may include providing an animal (e.g., a mouse, rat, or other animal) and grafting one or more test cells, or cell tissue, onto the animal. In one aspect, conditions for encouraging a test cell to divide in vivo include providing food, water and nutrients to the animal and, in some instances, antibiotics to promote growth of the animal; and temperature conditions favorable for growth of the animal (for example, 23° C.).

As used herein, “cell passaging” or “passaging” is a process for subculturing cells under physiological and environmental conditions to keep the cells alive for periods of time, sometimes extended periods of time. And as used herein, “passage number” or “cell passage” means the number of times a cell culture has been subcultured (harvested and transferred) into daughter cell cultures.

As used herein, “timepoint” or “timepoints” means the moment in time when a particular action occurs, for example, the transfer of cells to a new cell culture plate in cell passaging.

In one aspect, the method described herein provide for statistical methods to estimate of the probability of a degree of association between variables; and statistical significance can be expressed, in terms of p-value. As used herein, in one aspect, “statistically significant” means a p-value that is less than 0.05 or, alternatively is less than 0.01, 0.005, or 0.001.

The term “mitotic clock” means a series of similar events which occur in a DNA replication-dependent manner. One example of a mitotic clock is the loss of a small amount of DNA following each round of DNA replication due to the inability of DNA polymerase to fully replicate chromosome ends (telomeres). Other mitotic clocks are described hereinbelow in the Examples. As used herein, “mitotic clock” means a change (e.g. increase) in the DNA hypomethylation level with each round of DNA replication.

As used herein “cell mass” means a mass or grouping of cells that originate from a parent cell.

Another aspect is a method for developing a mitotic clock, including (a) identifying a test cell for which a determination of a mitotic clock is desired; (b) providing conditions for the test cell to divide; (c) determining the number of effective cell divisions in the test cell at one or more timepoints; (d) using data processing apparatus to obtain CpG dinucleotide sequence methylation data for genomic DNA derived from the test cell at the timepoints, wherein the genomic DNA comprises highly methylated domains (HMD) and partially methylated domains (PMD), wherein each such CpG dinucleotide is the sole CpG dinucleotide sequence within a n(x)WCpGWn(x) genomic DNA sequence motif (Solo-WCGW motif) of at least one PMD, and wherein W=A or T, n=A or G or C or T, and x≥9; (e) using the data processing apparatus to determine, based on the CpG dinucleotide sequence methylation data, a mean or average CpG dinucleotide methylation value or a value related thereto at each of the timepoints for a plurality of Solo-WCGW motif sequences of the at least one PMDs, to provide a measure of cellular replication-associated DNA methylation loss at each of the timepoints; (f) using the data processing apparatus to correlate the effective cell divisions at each of the timepoints with the measure of cellular replication-associated DNA methylation loss at each of the timepoints; and (g) if the correlation is statistically significant, identifying the measure of cellular replication-associated DNA methylation loss as a mitotic clock.

In some aspects, data processing apparatus is used to implement various aspects of the inventive method. For instance, the user may provide data input or selections to software being executed by the data processing apparatus. In some aspects of the present inventive methods, data processing apparatus is used because of the need for computing power to manipulate and analyze the large amount of data associated with measuring replication-associated DNA methylation loss. More specifically, it would not be humanly practical to digest and calculate replication-associated DNA methylation loss without errors. Using data processing apparatus, instead of a human, to perform repeated calculations, the calculations would be systematically accurate and reliable; an aspect of considerable importance to discerning cellular replicative/mitotic history, mitotic turnover rate, chronological age of a cell or tissue, increased risk for conditions associated with excessive replicative turnover or aging, identification of subjects for increased surveillance, cancer screening, forensic analysis, etc.

Implementations of the subject matter and the functional operations described in this specification can be implemented in digital electronic circuitry, or in computer software, firmware, or hardware, including the structures disclosed in this specification and their structural equivalents, or in combinations of one or more of them. Moreover, subject matter described in this specification may be implemented as one or more computer program products, i.e., one or more modules of computer program instructions encoded on a computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The terms “data processing apparatus”, “computing device” and “computing processor” encompass all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as an application, program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

One or more aspects of the disclosure can be implemented in a computing system that includes a backend component, e.g., as a data server, or that includes a middleware component, e.g., an application server, or that includes a frontend component, e.g., a client computer having a graphical user interface or a Web browser through which a user can interact with an implementation of the subject matter described in this specification, or any combination of one or more such backend, middleware, or frontend components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

The human and mouse Genome Assemblies GRCh37 and GRCm38 used for the present work are summarized below in Tables 2 and 3, respectively.

Exemplary, representative human and mouse n(x)WCpGWn(x) genomic DNA sequence motif species, wherein W=A or T, n=A or G or C or T, and wherein x=35 are provided below in Tables 4 and 5 (human) and Tables 6 and 7 (mouse).

Tables 8 and 9 list exemplary probes with extension base targeting CpG dinucleotide sequences in the respective exemplary human Solo-WCGW motif sequences listed in Tables 4 and 5, respectively.

Tables 10 and 11 list exemplary probes with extension base targeting CpG dinucleotide sequences in the respective exemplary mouse Solo-WCGW motif sequences listed in Tables 6 and 7, respectively.

Table 12 lists primary human cells obtained from multiple tissues and donors.

Table 13 lists 44 CpGs and coefficients selected by elastic net regression of solo-WCGW CpG beta values from serial primary cell culture to standardized population doubling level.

Table 14 is a summary of predictive performance of various methylation clocks on training dataset from primary cells.

Tables 15A-B list the CpGs in a 44-CpG model for predicting mitotic history within and between cell types.

Tables 16A-B list a subset of 75 strongly correlated CpGs for all tissue types studied.

TABLE 2
Human Genome Assembly GRCh37

| Chromosome | Total length (bp) | GenBank accession | RefSeq accession |
|---|---|---|---|
| 1 | 249,250,621 | CM000663.1 | NC_000001.10 |
| 2 | 243,199,373 | CM000664.1 | NC_000002.11 |
| 3 | 198,022,430 | CM000665.1 | NC_000003.11 |
| 4 | 191,154,276 | CM000666.1 | NC_000004.11 |
| 5 | 180,915,260 | CM000667.1 | NC_000005.9 |
| 6 | 171,115,067 | CM000668.1 | NC_000006.11 |
| 7 | 159,138,663 | CM000669.1 | NC_000007.13 |
| 8 | 146,364,022 | CM000670.1 | NC_000008.10 |
| 9 | 141,213,431 | CM000671.1 | NC_000009.11 |
| 10 | 135,534,747 | CM000672.1 | NC_000010.10 |
| 11 | 135,006,516 | CM000673.1 | NC_000011.9 |
| 12 | 133,851,895 | CM000674.1 | NC_000012.11 |
| 13 | 115,169,878 | CM000675.1 | NC_000013.10 |
| 14 | 107,349,540 | CM000676.1 | NC_000014.8 |
| 15 | 102,531,392 | CM000677.1 | NC_000015.9 |
| 16 | 90,354,753 | CM000678.1 | NC_000016.9 |
| 17 | 81,195,210 | CM000679.1 | NC_000017.10 |
| 18 | 78,077,248 | CM000680.1 | NC_000018.9 |
| 19 | 59,128,983 | CM000681.1 | NC_000019.9 |
| 20 | 63,025,520 | CM000682.1 | NC_000020.10 |
| 21 | 48,129,895 | CM000683.1 | NC_000021.8 |
| 22 | 51,304,566 | CM000684.1 | NC_000022.10 |
| X | 155,270,560 | CM000685.1 | NC_000023.10 |
| Y | 59,373,566 | CM000686.1 | NC_000024.9 |

General

| Assembly name | GRCh37 |
|---|---|
| Release date | 2009 Feb. 27 |
| Assembly type | haploid-with-alt-loci |
| Release type | major |
| Assembly units | 10 |
| Total bases | 3,137,144,693 |
| Total non-N bases | 2,897,293,955 |
| Primary assembly N50 | 46,395,641 |

Regions

| | |
|---|---|
| Total regions | 7 |
| Regions with alternate loci | 3 |
| Regions with FIX patches | 0 |
| Regions with NOVEL patches | 0 |
| Regions as PAR | 4 |

Alternate Loci and Patches

| | |
|---|---|
| Alternate loci | 9 |
| Alternate loci aligned to primary assembly | 9 |
| FIX patches | 0 |
| FIX patches aligned to primary assembly | 0 |
| NOVEL patches | 0 |
| NOVEL patches aligned to primary assembly | 0 |

TABLE 3
Mouse Genome Assembly GRCm38

| Chromosome | Total length (bp) | GenBank accession | RefSeq accession |
|---|---|---|---|
| 1 | 195,471,971 | CM000994.2 | NC_000067.6 |
| 2 | 182,113,224 | CM000995.2 | NC_000068.7 |
| 3 | 160,039,680 | CM000996.2 | NC_000069.6 |
| 4 | 156,508,116 | CM000997.2 | NC_000070.6 |
| 5 | 151,834,684 | CM000998.2 | NC_000071.6 |
| 6 | 149,736,546 | CM000999.2 | NC_000072.6 |
| 7 | 145,441,459 | CM001000.2 | NC_000073.6 |
| 8 | 129,401,213 | CM001001.2 | NC_000074.6 |
| 9 | 124,595,110 | CM001002.2 | NC_000075.6 |
| 10 | 130,694,993 | CM001003.2 | NC_000076.6 |
| 11 | 122,082,543 | CM001004.2 | NC_000077.6 |
| 12 | 120,129,022 | CM001005.2 | NC_000078.6 |
| 13 | 120,421,639 | CM001006.2 | NC_000079.6 |
| 14 | 124,902,244 | CM001007.2 | NC_000080.6 |
| 15 | 104,043,685 | CM001008.2 | NC_000081.6 |
| 16 | 98,207,768 | CM001009.2 | NC_000082.6 |
| 17 | 94,987,271 | CM001010.2 | NC_000083.6 |
| 18 | 90,702,639 | CM001011.2 | NC_000084.6 |
| 19 | 61,431,566 | CM001012.2 | NC_000085.6 |
| X | 171,031,299 | CM001013.2 | NC_000086.7 |
| Y | 91,744,698 | CM001014.2 | NC_000087.7 |

General

| Assembly name | GRCm38 |
|---|---|
| Release date | 2012 Jan. 9 |
| Assembly type | haploid-with-alt-loci |
| Release type | major |
| Assembly units | 16 |
| Total bases | 2,793,712,140 |
| Total non-N bases | 2,714,420,385 |
| Primary assembly N50 | 54,517,951 |

Regions

| | |
|---|---|
| Total regions | 72 |
| Regions with alternate loci | 70 |
| Regions with FIX patches | 0 |
| Regions with NOVEL patches | 0 |
| Regions as PAR | 2 |

Alternate Loci and Patches

| | |
|---|---|
| Alternate loci | 99 |
| Alternate loci aligned to primary assembly | 92 |
| FIX patches | 0 |
| FIX patches aligned to primary assembly | 0 |
| NOVEL patches | 0 |
| NOVEL patches aligned to primary assembly | 0 |

TABLE 4
Exemplary human n₍ₓ₎WCpGWn₍ₓ₎ genomic DNA sequence motifs,
wherein W = A or T, n = A or G or C or T, and x = 35. The 40 randomly
selected motif sequences are for common (shared between/among
cell/tissue types) PMD solo-WCGW CpGs, each in an arm of a chromosome
(4 chromosomes have only 1 arm).The exemplary motif sequences cover
35 bp upstream and 35 bp downstream of the target CpG, which in each
case is surrounded by square brackets. The respective SEQ ID NOS
are shown to right of each sequence in the last column. The human
reference sequence version is GRCh37. Specific chromosome accession
numbers can be found at https:
//www.ncbi.nlm.nih.gov/grc/human/data?asm=GRCh37.

| | | | | | | Sequence (5′ |
|---|---|---|---|---|---|---|
| | sequence | sequence | | | | to 3′); (SEQ |
| chromosome | begin | end | arm | CpG begin | CpG end | ID NOS) |
| chr1 | 5696956 | 5697027 | chr1p | 5696991 | 5696992 | AAATATTGGCTA |
| | | | | | | TTATTATTTTTA |
| | | | | | | TCACACCATCT[ |
| | | | | | | CG]TGAGTCTCA |
| | | | | | | TCATCTCATGAA |
| | | | | | | ATAGTGCATGAG |
| | | | | | | AA (SEQ ID |
| | | | | | | NO: 1) |
| chr1 | 217414200 | 217414271 | chr1q | 217414235 | 217414236 | GTTTCAGTGGTG |
| | | | | | | GGATCATGTCTT |
| | | | | | | TATCAGAAGCT[ |
| | | | | | | CG]TGAAGGAAT |
| | | | | | | GTTGCTTTTCTT |
| | | | | | | AGTCATGTAGGA |
| | | | | | | AC (SEQ ID |
| | | | | | | NO: 2) |
| chr10 | 19690339 | 19690410 | chr10p | 19690374 | 19690375 | AGCAGTTTGTAT |
| | | | | | | AAACACAAATAA |
| | | | | | | TAGGAAGTAAT[ |
| | | | | | | CG]AATTGAAAA |
| | | | | | | CTAATCCAAAAC |
| | | | | | | TGCTTTTTGAAT |
| | | | | | | GG (SEQ ID |
| | | | | | | NO: 3) |
| chr10 | 55000655 | 55000726 | chr10q | 55000690 | 55000691 | AGGTGGGAGAAA |
| | | | | | | CTCTTCAGGCCA |
| | | | | | | AGAGTTTGAGA[ |
| | | | | | | CG]AGCCTGGGC |
| | | | | | | AACATAGCAAGA |
| | | | | | | CCCTATCTCTAT |
| | | | | | | AA (SEQ ID |
| | | | | | | NO: 4) |
| chr11 | 15065192 | 15065263 | chr11p | 15065227 | 15065228 | TGGTGAAAAGGG |
| | | | | | | AATGGAAATTGG |
| | | | | | | ATGTAAGGATA[ |
| | | | | | | CG]AGTTTCCTT |
| | | | | | | TTTTTTTTTTTT |
| | | | | | | TTGAGACAGAGT |
| | | | | | | AT (SEQ ID |
| | | | | | | NO: 5) |
| chr11 | 56180625 | 56180696 | chr11q | 56180660 | 56180661 | ATTCCTAGAAAA |
| | | | | | | CTGTATTAAACT |
| | | | | | | GATTGCTAGCA[ |
| | | | | | | CG]TATGTGTAT |
| | | | | | | GGATTCACTGTG |
| | | | | | | GGACTTGTACAG |
| | | | | | | AC (SEQ ID |
| | | | | | | NO: 6) |
| chr12 | 17187586 | 17187657 | chr12p | 17187621 | 17187622 | TTTTCCCTTTAT |
| | | | | | | ACCAAGAGGATG |
| | | | | | | TCTGATTAACT[ |
| | | | | | | CG]ATGTATAAA |
| | | | | | | AGGACTGATAAC |
| | | | | | | AAAAATAAGCAT |
| | | | | | | CA (SEQ ID |
| | | | | | | NO: 7) |
| chr12 | 127631492 | 127631563 | chr12q | 127631527 | 127631528 | GGGTGGATTGCT |
| | | | | | | TGAGCTCAAGAA |
| | | | | | | TTCAAGACCAA[ |
| | | | | | | CG]TGGGCAGCA |
| | | | | | | TAGCAAGACTCC |
| | | | | | | CTACAAAAAAAA |
| | | | | | | TA (SEQ ID |
| | | | | | | NO: 8) |
| chr13 | 70647232 | 70647303 | chr13q | 70647267 | 70647268 | CACATGCACATG |
| | | | | | | TATGTTTATTGC |
| | | | | | | AGCACTATTCA[ |
| | | | | | | CG]ATAGCAGAC |
| | | | | | | TTGGAACCAACC |
| | | | | | | CAAATGTCCATC |
| | | | | | | AA (SEQ ID |
| | | | | | | NO: 9) |
| chr14 | 97515326 | 97515397 | chr14q | 97515361 | 97515362 | GAGTTCATTCCC |
| | | | | | | CATCCAGTTAGG |
| | | | | | | TCAAGTTAGAA[ |
| | | | | | | CG]AGGGTTGCC |
| | | | | | | ATCCAGTTAGGT |
| | | | | | | CAAGTTAAAATG |
| | | | | | | AG (SEQ ID |
| | | | | | | NO: 10) |
| chr15 | 88363768 | 88363839 | chr15q | 88363803 | 88363804 | CCTTCCACTGAT |
| | | | | | | AACCATCAAGGT |
| | | | | | | AACATTGCAAA[ |
| | | | | | | CG]TGTTAGACT |
| | | | | | | ATGGCATAAAGG |
| | | | | | | CAACCACAGGTA |
| | | | | | | CA (SEQ ID |
| | | | | | | NO: 11) |
| chr16 | 17056693 | 17056764 | chr16p | 17056728 | 17056729 | GGCCAAGGCAGG |
| | | | | | | CAGATCACTTGA |
| | | | | | | GGTCAGGAGTT[ |
| | | | | | | CG]AGATCAGTC |
| | | | | | | TAGCCAACATGG |
| | | | | | | TGAAACCCAGTC |
| | | | | | | TC (SEQ ID |
| | | | | | | NO: 12) |
| chr16 | 59014585 | 59014656 | chr16q | 59014620 | 59014621 | GTCCCAGAGATT |
| | | | | | | CTGGTATGTTGT |
| | | | | | | GTCTTTGTTCT[ |
| | | | | | | CG]TTGGTTTCA |
| | | | | | | AAGAGCATCTTT |
| | | | | | | ATTTCTGCTTTC |
| | | | | | | AT (SEQ ID |
| | | | | | | NO: 13) |
| chr17 | 21763952 | 21764023 | chr17p | 21763987 | 21763988 | TCTCCTCCTAGA |
| | | | | | | TTATATAAAAAG |
| | | | | | | ATTGTATTCCA[ |
| | | | | | | CG]TGCTGAATC |
| | | | | | | AAAACACAGTTA |
| | | | | | | ACTTGGTGAGAT |
| | | | | | | CA (SEQ ID |
| | | | | | | NO: 14) |
| chr17 | 75530197 | 75530268 | chr17q | 75530232 | 75530233 | CCTGCACTTCCT |
| | | | | | | GGCCCTCCATGC |
| | | | | | | TTGGGCATGGA[ |
| | | | | | | CG]TGTGATATG |
| | | | | | | GTTTGGCTGTGT |
| | | | | | | CCCCACCCAAAT |
| | | | | | | CT (SEQ ID |
| | | | | | | NO: 15) |
| chr18 | 1029417 | 1029488 | chr18p | 1029452 | 1029453 | ACATGTGCCATG |
| | | | | | | TTGGTTTGCTGC |
| | | | | | | ACCCATCAACT[ |
| | | | | | | CG]TCATTTACA |
| | | | | | | TTAGGTATTTCT |
| | | | | | | CCTAACACTATC |
| | | | | | | CC (SEQ ID |
| | | | | | | NO: 16) |
| chr18 | 70768819 | 70768890 | chr18q | 70768854 | 70768855 | GTCAGAGTGCTT |
| | | | | | | GTGCCCAAAACT |
| | | | | | | AAGTCATACCA[ |
| | | | | | | CG]TACTTAAGT |
| | | | | | | ACACAGATCTTA |
| | | | | | | GAGTCAGAGTGC |
| | | | | | | TT (SEQ ID |
| | | | | | | NO: 17) |
| chr19 | 21460219 | 21460290 | chr19p | 21460254 | 21460255 | CCCAGCCTTAGG |
| | | | | | | GTGTCCTTTTTA |
| | | | | | | TACTTTGTTTT[ |
| | | | | | | CG]TTAACAGTG |
| | | | | | | TCAAAAATTAGT |
| | | | | | | TGGCTTTAAGTA |
| | | | | | | TT (SEQ ID |
| | | | | | | NO: 18) |
| chr19 | 57379969 | 57380040 | chr19q | 57380004 | 57380005 | CCATTTTGTGTA |
| | | | | | | AAATCTGCCATG |
| | | | | | | GACAATATGTA[ |
| | | | | | | CG]TGAATGAAC |
| | | | | | | ATGGCTATGTTC |
| | | | | | | CACATTATTTTG |
| | | | | | | GG (SEQ ID |
| | | | | | | NO: 19) |
| chr2 | 60084641 | 60084712 | chr2p | 60084676 | 60084677 | GTAACTTAACAC |
| | | | | | | AATAGATGTTTA |
| | | | | | | TTTCTTACTCA[ |
| | | | | | | CG]TAAAGTCTA |
| | | | | | | ATAGGTGCCAAG |
| | | | | | | ACAGATAAGGTT |
| | | | | | | CT (SEQ ID |
| | | | | | | NO: 20) |
| chr2 | 142005802 | 142005873 | chr2q | 142005837 | 142005838 | ATTTAGACAAAG |
| | | | | | | GTATATTCAGCC |
| | | | | | | TGTTTTATGTA[ |
| | | | | | | CG]AAGCACTGT |
| | | | | | | ACTGATCCCTGC |
| | | | | | | AGAAGACAAAAT |
| | | | | | | CA (SEQ ID |
| | | | | | | NO: 21) |
| chr20 | 23054904 | 23054975 | chr20p | 23054939 | 23054940 | AGCTGTGTGCTG |
| | | | | | | GAGGCTGCCAGT |
| | | | | | | GCTCAACAAAT[ |
| | | | | | | CG]TGCTTGCAC |
| | | | | | | TTTTCACTGTGC |
| | | | | | | TCAGGTGAAGTA |
| | | | | | | CA (SEQ ID |
| | | | | | | NO: 22) |
| chr20 | 49807131 | 49807202 | chr20q | 49807166 | 49807167 | TGCCCAGGTCTG |
| | | | | | | GCCTCTTGTTTC |
| | | | | | | AAGTCACAGCT[ |
| | | | | | | CG]TTGAAAACA |
| | | | | | | TTAAAAAAAAAA |
| | | | | | | AAAACAAACCTT |
| | | | | | | GA (SEQ ID |
| | | | | | | NO: 23) |
| chr21 | 10493977 | 10494048 | chr21p | 10494012 | 10494013 | ACAAAAATTCAT |
| | | | | | | CAGATTTAATAA |
| | | | | | | AGTTGTCTATT[ |
| | | | | | | CG]AAGATAGGG |
| | | | | | | ACTTTTTTCTTT |
| | | | | | | TTTAAAAATTAA |
| | | | | | | AT (SEQ ID |
| | | | | | | NO: 24) |
| chr21 | 14898104 | 14898175 | chr21q | 14898139 | 14898140 | AGGATGGCTGGG |
| | | | | | | CTCCAGTGTCTC |
| | | | | | | TGGAGTGGCTT[ |
| | | | | | | CG]AGTCCACTG |
| | | | | | | CTCCTGGAAGGC |
| | | | | | | TTCATCCCATTG |
| | | | | | | GC (SEQ ID |
| | | | | | | NO: 25) |
| chr22 | 49713189 | 49713260 | chr22q | 49713224 | 49713225 | AGATATGACTGG |
| | | | | | | AAAACATTTTCT |
| | | | | | | CCCATTGTGTA[ |
| | | | | | | CG]TGTCTTTTC |
| | | | | | | ACTTACTTGGTG |
| | | | | | | ACATCCTTTAGA |
| | | | | | | GC (SEQ ID |
| | | | | | | NO: 26) |
| chr3 | 19776288 | 19776359 | chr3p | 19776323 | 19776324 | CACATTGTCAAA |
| | | | | | | ATTGGTGGTGGG |
| | | | | | | TGAGAAACAGT[ |
| | | | | | | CG]TGGGTTCTA |
| | | | | | | GTTCATCTTTAT |
| | | | | | | GAATTCCCATTT |
| | | | | | | GT (SEQ ID |
| | | | | | | NO: 27) |
| chr3 | 137050701 | 137050772 | chr3q | 137050736 | 137050737 | CCCCATGACCTA |
| | | | | | | GTCACCTCCCCA |
| | | | | | | AAGGCCCCAGT[ |
| | | | | | | CG]ACTTGGGAA |
| | | | | | | TTAGGATTTCAA |
| | | | | | | CCTATACATTTT |
| | | | | | | GG (SEQ ID |
| | | | | | | NO: 28) |
| chr4 | 32808198 | 32808269 | chr4p | 32808233 | 32808234 | ATATAAGCAGGC |
| | | | | | | AGAAAAATGTGA |
| | | | | | | AAAGAGAAACA[ |
| | | | | | | CG]TCTAGCTGC |
| | | | | | | CCAGTATACATC |
| | | | | | | TTTCTCCCATGC |
| | | | | | | TG (SEQ ID |
| | | | | | | NO: 29) |
| chr4 | 117062707 | 117062778 | chr4q | 117062742 | 117062743 | CAAAGTCATTTT |
| | | | | | | TAATTATAAACT |
| | | | | | | TTGAATATGTT[ |
| | | | | | | CG]TATTTATTT |
| | | | | | | AGTTATTTAATG |
| | | | | | | CTTATTTAAAAA |
| | | | | | | TG (SEQ ID |
| | | | | | | NO: 30) |
| chr5 | 10037651 | 10037722 | chr5p | 10037686 | 10037687 | CTACAAACCAAG |
| | | | | | | CACACCAAGGAT |
| | | | | | | TTCTGGAGCCA[ |
| | | | | | | CG]AGAAGTGGA |
| | | | | | | GCAAGAAAGAGG |
| | | | | | | CATTGGTTCATG |
| | | | | | | AA (SEQ ID |
| | | | | | | NO: 31) |
| chr5 | 164978207 | 164978278 | chr5q | 164978242 | 164978243 | GAGTGCAGCCAT |
| | | | | | | TTTAAAGTATCA |
| | | | | | | AGCCAGGTGTT[ |
| | | | | | | CG]TAACAGGCA |
| | | | | | | CTTCATAAGTGG |
| | | | | | | AATATTTTATTT |
| | | | | | | TG (SEQ ID |
| | | | | | | NO: 32) |
| chr6 | 18974109 | 18974180 | chr6p | 18974144 | 18974145 | GAGGAGACTTTT |
| | | | | | | GATATTGTTCTA |
| | | | | | | TTTATCTTTAT[ |
| | | | | | | CG]TCACATTTT |
| | | | | | | TTCAGGCAGTAA |
| | | | | | | CTATATGTAAAA |
| | | | | | | GA (SEQ ID |
| | | | | | | NO: 33) |
| chr6 | 96253280 | 96253351 | chr6q | 96253315 | 96253316 | CCACACTACTCA |
| | | | | | | AAGTAGCTGTTC |
| | | | | | | CCCAAACTGTT[ |
| | | | | | | CG]TTACCCTTA |
| | | | | | | CACTAAGAGATA |
| | | | | | | AGAAGCTTGATC |
| | | | | | | CA (SEQ ID |
| | | | | | | NO: 34) |
| chr7 | 37490418 | 37490489 | chr7p | 37490453 | 37490454 | AAAAAAGAAAAA |
| | | | | | | AAAGTAGTCTTA |
| | | | | | | TAGATTAATTA[ |
| | | | | | | CG]TAATTAACC |
| | | | | | | ATTAGCAAACAC |
| | | | | | | AATACAGCCTGA |
| | | | | | | GA (SEQ ID |
| | | | | | | NO: 35) |
| chr7 | 131497504 | 131497575 | chr7q | 131497539 | 131497540 | AGATCAAGACCA |
| | | | | | | TCCTGGCCAACA |
| | | | | | | TGGTGAAACCT[ |
| | | | | | | CG]TCTCTACTA |
| | | | | | | AAAATACAAAAA |
| | | | | | | TTAGCTGGGCAT |
| | | | | | | GG (SEQ ID |
| | | | | | | NO: 36) |
| chr8 | 21352316 | 21352387 | chr8p | 21352351 | 21352352 | CACTCCTCCCAG |
| | | | | | | ACACAAGAGCTA |
| | | | | | | GTCAATGGTGT[ |
| | | | | | | CG]TGTGTCCCT |
| | | | | | | TCAAGGCAAATA |
| | | | | | | CTACTTGTAATA |
| | | | | | | GT (SEQ ID |
| | | | | | | NO: 37) |
| chr8 | 73088640 | 73088711 | chr8q | 73088675 | 73088676 | TAAGGTTCATTG |
| | | | | | | TGGGCCATCTTA |
| | | | | | | GAGGCTATCTA[ |
| | | | | | | CG]AGTGGATCA |
| | | | | | | TTACTTTTTATT |
| | | | | | | ATCATTATTTAT |
| | | | | | | TT (SEQ ID |
| | | | | | | NO: 38) |
| chr9 | 26513962 | 26514033 | chr9p | 26513997 | 26513998 | AGCCCAGCTAAG |
| | | | | | | TTTTTATTATTC |
| | | | | | | TTTTGTAGACA[ |
| | | | | | | CG]TGATCTTGC |
| | | | | | | TATGTTGCCCAG |
| | | | | | | GCTGGTCTTAAA |
| | | | | | | CA (SEQ ID |
| | | | | | | NO: 39) |
| chr9 | 121162709 | 121162780 | chr9q | 121162744 | 121162745 | CCTAATCCAATA |
| | | | | | | GTACTGGTGTCC |
| | | | | | | TTATAAGAAGA[ |
| | | | | | | CG]AGATTAGGA |
| | | | | | | CAGAGACACCTA |
| | | | | | | CAGAAGGAAGGC |
| | | | | | | TG (SEQ ID |
| | | | | | | NO: 40) |

TABLE 5
Exemplary human n(ₓ)WCpGWn(ₓ) genomic DNA sequence motifs, wherein
W = A or T, n = A or G or C or T, and x = 35. The 40 exemplary motif
sequences, randomly selected intergenic CpGs (H3K36me3 primarily exits
only at gene bodies), are for common (shared between/among cell/tissue
types) PMD solo-WCGW CpGs, each in an arm of a chromosome (4
chromosomes have only 1 arm). The exemplary motif sequences cover
35 bp upstream and 35 bp downstream of the target CpG, which in each
case is surrounded by square brackets. The respective SEQ ID NOS are
shown to the right of each sequence in the last column. The human
reference sequence version is GRCh37. Specific chromosome accession
numbers can be found at https:
//ncbi.nlm.nih.gov/grc/human/data?asm=GRCh37.

| | | | | | | Sequence (5′ |
|---|---|---|---|---|---|---|
| | sequence | sequence | | | | to 3′); (SEQ |
| chromosome | begin | end | arm | CpG begin | CpG end | ID NOS) |
| chr1 | 104551650 | 104551721 | chr1p | 104551685 | 104551686 | TGATATCCCCTTTA |
| | | | | | | TCATTTTTTATTGT |
| | | | | | | GTCTATT[CG]ATT |
| | | | | | | TTTCTCTCTTTTCT |
| | | | | | | TCTTTATTAGTCTG |
| | | | | | | GCTA (SEQ ID |
| | | | | | | NO: 41) |
| chr1 | 218995293 | 218995364 | chr1q | 218995328 | 218995329 | TTCTACCAGAGGTA |
| | | | | | | CAAAGAGGAGCTGG |
| | | | | | | TACCATT[CG]TTC |
| | | | | | | TGAAACTATTCCAG |
| | | | | | | TCAATAGAAAGAGA |
| | | | | | | GGGA (SEQ ID |
| | | | | | | NO: 42) |
| chr10 | 7185785 | 7185856 | chr1Op | 7185820 | 7185821 | CTGGGTTCAAGCAA |
| | | | | | | TCCTCTTGCCTCAG |
| | | | | | | CCTCCCT[CG]TAG |
| | | | | | | CTGAAACTACAGGC |
| | | | | | | ATATGCCACCATGC |
| | | | | | | CCAA (SEQ ID |
| | | | | | | NO: 43) |
| chr10 | 127072911 | 127072982 | chr10q | 127072946 | 127072947 | TTAGAGTTGCCAGA |
| | | | | | | GTTCTTGCACTGGC |
| | | | | | | TCTTTCT[CG]TCT |
| | | | | | | ATGTAGGCTGATGT |
| | | | | | | TCCTTTAATCTTTG |
| | | | | | | AAGT (SEQ ID |
| | | | | | | NO: 44) |
| chr11 | 25362076 | 25362147 | chr11p | 25362111 | 25362112 | GAGACAGGATCTCA |
| | | | | | | CTACATTACCCAGG |
| | | | | | | CTGGTCT[CG]AAC |
| | | | | | | TCTTGGCCTCAAGT |
| | | | | | | GATCCTCCTGCCTC |
| | | | | | | AGCC (SEQ ID |
| | | | | | | NO: 45) |
| chr11 | 134588646 | 134588717 | chr11q | 134588681 | 134588682 | AGTATTGATACCCC |
| | | | | | | TGCTCTCTTTTGGT |
| | | | | | | TATTATT[CG]TAT |
| | | | | | | AAACTATCCTTTTT |
| | | | | | | TATACTTTCACTTT |
| | | | | | | CAAC (SEQ ID |
| | | | | | | NO: 46) |
| chr12 | 34249312 | 34249383 | chr12p | 34249347 | 34249348 | GTGTGTATATATAT |
| | | | | | | GTGTGTGTGTATAT |
| | | | | | | ATACACA[CG]TAT |
| | | | | | | ATATATATATTTAA |
| | | | | | | CTGATTCTTGTGCC |
| | | | | | | TTAG (SEQ ID |
| | | | | | | NO: 47) |
| chr12 | 60734392 | 60734463 | chr12q | 60734427 | 60734428 | ATTTCAATGCATAA |
| | | | | | | AACTAAGAAAGTAG |
| | | | | | | ATCAAGA[CG]ATA |
| | | | | | | ATACAATTTTCAGT |
| | | | | | | TGTATATTTTTGTT |
| | | | | | | TTAG (SEQ ID |
| | | | | | | NO: 48) |
| chr13 | 109105511 | 109105582 | chr13q | 109105546 | 109105547 | AACAACCTGGGCAA |
| | | | | | | CATGGTGAAACTCT |
| | | | | | | GTCTCTA[CG]AAA |
| | | | | | | AAAAAAAAAAATTA |
| | | | | | | GCTGGATGTGGTGG |
| | | | | | | TGTG (SEQ ID |
| | | | | | | NO: 49) |
| chr14 | 29622409 | 29622480 | chr14q | 29622444 | 29622445 | AAGTATCTTATTAA |
| | | | | | | TATTTTTAAAATAC |
| | | | | | | TTGATTA[CG]TGT |
| | | | | | | TAAAATGATGGTAT |
| | | | | | | TTTGAATATACTGG |
| | | | | | | ATTA (SEQ ID |
| | | | | | | NO: 50) |
| chr15 | 46873411 | 46873482 | chr15q | 46873446 | 46873447 | ACATACACCATTGA |
| | | | | | | AATAGACAAATGTT |
| | | | | | | ACTTTTT[CG]TAC |
| | | | | | | CTACCCCTATTCCT |
| | | | | | | CTAAGTACCTGTTG |
| | | | | | | TTAA (SEQ ID |
| | | | | | | NO: 51) |
| chr16 | 26585447 | 26585518 | chr16p | 26585482 | 26585483 | CAGGCTGATGGAAA |
| | | | | | | CATGACATGGAGTT |
| | | | | | | GGCCTGA[CG]TTG |
| | | | | | | CTGACTTTGAAAAT |
| | | | | | | GGAGAAAGGGGCCA |
| | | | | | | AGAG (SEQ ID |
| | | | | | | NO: 52) |
| chr16 | 61515568 | 61515639 | chr16q | 61515603 | 61515604 | CCTGTAGGCAAGCA |
| | | | | | | TAAGAAATGAGCAG |
| | | | | | | CTACTAA[CG]TTT |
| | | | | | | GAAATCCTTTGCTA |
| | | | | | | TCCCATGCAAAGTT |
| | | | | | | ACAT (SEQ ID |
| | | | | | | NO: 53) |
| chr17 | 5400427 | 5400498 | chr17p | 5400462 | 5400463 | AGTAGGGAGATATG |
| | | | | | | TCATCACATATTCC |
| | | | | | | TGGGATA[CG]TAA |
| | | | | | | ACTATAACTCAAAC |
| | | | | | | TATATAAGAGGAAA |
| | | | | | | ATTG (SEQ ID |
| | | | | | | NO: 54) |
| chr17 | 50429052 | 50429123 | chr17q | 50429087 | 50429088 | TTTTTGCTATTGTG |
| | | | | | | AATAGTGCTGCAAT |
| | | | | | | AAACATA[CG]TGT |
| | | | | | | GCATGTGTCTTTAT |
| | | | | | | TGTAGCATGATTTA |
| | | | | | | TAAT (SEQ ID |
| | | | | | | NO: 55) |
| chr18 | 11199564 | 11199635 | chr18p | 11199599 | 11199600 | GTTATTTCAGTAAC |
| | | | | | | ACTTGTGTTTATTG |
| | | | | | | CAACTGA[CG]TGA |
| | | | | | | TTGCAGGAGCTGCA |
| | | | | | | CAGGGCACTTGTCC |
| | | | | | | ATCC (SEQ ID |
| | | | | | | NO: 56) |
| chr18 | 51151401 | 51151472 | chr18q | 51151436 | 51151437 | AAGTATTGTTCTTA |
| | | | | | | AGAAATGTTCAGTC |
| | | | | | | TGTTCAA[CG]ATT |
| | | | | | | TGAGCCCCTTTCTA |
| | | | | | | TTGACTCTCCAGGA |
| | | | | | | GTCA (SEQ ID |
| | | | | | | NO: 57) |
| chr19 | 14976670 | 14976741 | chr19p | 14976705 | 14976706 | ACAGTCAAATATGC |
| | | | | | | CCCTTCTTAAAAAC |
| | | | | | | AAACAAA[CG]AAC |
| | | | | | | AGACAAACAAATCC |
| | | | | | | CTCTCTTCAGTGTA |
| | | | | | | TATC (SEQ ID |
| | | | | | | NO: 58) |
| chr19 | 42017439 | 42017510 | chr19q | 42017474 | 42017475 | TGGATATTAGAAAA |
| | | | | | | AATATCACAAGGGG |
| | | | | | | GTGTATA[CG]ACT |
| | | | | | | CCTGAGATATTGGG |
| | | | | | | AGTAACATCATTCT |
| | | | | | | CTCC (SEQ ID |
| | | | | | | NO: 59) |
| chr2 | 81964316 | 81964387 | chr2p | 81964351 | 81964352 | AGGACCACCTATCC |
| | | | | | | AAGACTATGGGAGG |
| | | | | | | CCTGAGA[CG]ATT |
| | | | | | | GCAGAACATCTGCT |
| | | | | | | AGTATAAACTTCAA |
| | | | | | | GAAT (SEQ ID |
| | | | | | | NO: 60) |
| chr2 | 117648329 | 117648400 | chr2q | 117648364 | 117648365 | ATGTTAGCTATAGG |
| | | | | | | ATTTCCATATATGG |
| | | | | | | CCTTTAT[CG]TGT |
| | | | | | | TGTGGTACATTCCT |
| | | | | | | TCTATACCTAATTT |
| | | | | | | GTTC (SEQ ID |
| | | | | | | NO: 61) |
| chr20 | 19107540 | 19107611 | chr20p | 19107575 | 19107576 | GGCATTATGTAAGA |
| | | | | | | GTCAAATTTTATTC |
| | | | | | | CTCTCCA[CG]AAG |
| | | | | | | ATATCCAGTTTTCC |
| | | | | | | TAACACTATTTATT |
| | | | | | | GAAG (SEQ ID |
| | | | | | | NO: 62) |
| chr20 | 51415270 | 51415341 | chr20q | 51415305 | 51415306 | CCTGGGACAGCCTG |
| | | | | | | GGTTTTGTTTCTCC |
| | | | | | | TTCCTTT[CG]AAG |
| | | | | | | CAGAATGTTCTTCA |
| | | | | | | AAGCTTTTCCCAGT |
| | | | | | | GAGT (SEQ ID |
| | | | | | | NO: 63) |
| chr21 | 10417751 | 10417822 | chr21p | 10417786 | 10417787 | CCATTTATGACAAT |
| | | | | | | ATGGATGAATCTAG |
| | | | | | | AGGACAT[CG]TGG |
| | | | | | | TAAGTGAAATAAGC |
| | | | | | | CAGACACAGAAAGA |
| | | | | | | CAAG (SEQ ID |
| | | | | | | NO: 64) |
| chr21 | 15360193 | 15360264 | chr21q | 15360228 | 15360229 | TCATCAATCACCAC |
| | | | | | | TGTTTCAGTGCAGA |
| | | | | | | ACATTTT[CG]TCT |
| | | | | | | TCCCAAAAAGAAAC |
| | | | | | | CCCTCAGTAATCAC |
| | | | | | | TCCC (SEQ ID |
| | | | | | | NO: 65) |
| chr22 | 20689045 | 20689116 | chr22q | 20689080 | 20689081 | TGGGATTCAGTTTT |
| | | | | | | TGAAATGAAACACT |
| | | | | | | GAGCCTT[CG]ATG |
| | | | | | | ACCTTCCTGTACAT |
| | | | | | | GTGAAAGCACACCT |
| | | | | | | GTCT (SEQ ID |
| | | | | | | NO: 66) |
| chr3 | 26257765 | 26257836 | chr3p | 26257800 | 26257801 | CTCACATGGTGCCC |
| | | | | | | TGCACTGCCAAGAC |
| | | | | | | AAGTGAA[CG]ATA |
| | | | | | | CAGTAAGGATGGCT |
| | | | | | | AAAGGTGACCTCAG |
| | | | | | | AAAC (SEQ ID |
| | | | | | | NO: 67) |
| chr3 | 103794890 | 103794961 | chr3q | 103794925 | 103794926 | ATATTTTTAAAAGC |
| | | | | | | ATAAATATTTAGGC |
| | | | | | | ATACTAA[CG]ATA |
| | | | | | | GTCAGATATAAGTC |
| | | | | | | ATGAACAGACAAGC |
| | | | | | | TGAA (SEQ ID |
| | | | | | | NO: 68) |
| chr4 | 32434655 | 32434726 | chr4p | 32434690 | 32434691 | AAGAGATGGGTAGA |
| | | | | | | ATAGAAACAACTTG |
| | | | | | | AAAAACA[CG]TTT |
| | | | | | | TAAGATATCATCTA |
| | | | | | | TGAGAGCTTCCCCA |
| | | | | | | ACTT (SEQ ID |
| | | | | | | NO: 69) |
| chr4 | 96567228 | 96567299 | chr4q | 96567263 | 96567264 | TGACTCCACCAAGG |
| | | | | | | CAAGGAAGTCATCA |
| | | | | | | AAAGGGA[CG]TGG |
| | | | | | | GGAGTGTGGGGAAA |
| | | | | | | AAATACATAAATCA |
| | | | | | | TGGG (SEQ ID |
| | | | | | | NO: 70) |
| chr5 | 23294691 | 23294762 | chr5p | 23294726 | 23294727 | GAGATGTGAGGTGT |
| | | | | | | CATTCTATTCATCA |
| | | | | | | TGTTCTT[CG]TTG |
| | | | | | | CTTGAATACTCTCA |
| | | | | | | GCATTTGTTTTCTG |
| | | | | | | GAAA (SEQ ID |
| | | | | | | NO: 71) |
| chr5 | 105641660 | 105641731 | chr5q | 105641695 | 105641696 | AAGAAACTCCAGCA |
| | | | | | | TATTTACATCTTTT |
| | | | | | | ATGTCTA[CG]ATC |
| | | | | | | CACTCACTTTCAGA |
| | | | | | | GTTTCCAAAGACTG |
| | | | | | | AATT (SEQ ID |
| | | | | | | NO: 72) |
| chr6 | 23619619 | 23619690 | chr6p | 23619654 | 23619655 | CATTGTCTGTTTTT |
| | | | | | | AAATTTGAGATAAA |
| | | | | | | ATTGTCA[CG]AAA |
| | | | | | | ATATAAGACAAACA |
| | | | | | | GGGAAATCTAATTT |
| | | | | | | TCTG (SEQ ID |
| | | | | | | NO: 73) |
| chr6 | 68712701 | 68712772 | chr6q | 68712736 | 68712737 | TCCCCATTCTCCTC |
| | | | | | | TCATATAAGGCTAC |
| | | | | | | CACAGAA[CG]TAT |
| | | | | | | TTTCTAGGGCCCTC |
| | | | | | | CATCTTTTGATTCC |
| | | | | | | CTAA (SEQ ID |
| | | | | | | NO: 74) |
| chr7 | 12304413 | 12304484 | chr7p | 12304448 | 12304449 | AATAGTTTAATGGT |
| | | | | | | TATTATACAGATAT |
| | | | | | | GTTTTAT[CG]TTT |
| | | | | | | TCTTGGAGAATGTT |
| | | | | | | GACTATTTTAGCTT |
| | | | | | | TCAA (SEQ ID |
| | | | | | | NO: 75) |
| chr7 | 142541482 | 142541553 | chr7q | 142541517 | 142541518 | TAACTGGAGAACAC |
| | | | | | | ACTTATTACTCATA |
| | | | | | | AAGCAGA[CG]AAG |
| | | | | | | CAAAAGTAGACATT |
| | | | | | | TGACATATAATAAA |
| | | | | | | ACAA (SEQ ID |
| | | | | | | NO: 76) |
| chr8 | 23821444 | 23821515 | chr8p | 23821479 | 23821480 | TAGTCCATCAGTTA |
| | | | | | | TTCAGTAGCCTAAT |
| | | | | | | TTTGATT[CG]AAT |
| | | | | | | GCACTTCACTGGTT |
| | | | | | | TAGTACCCAGGTCA |
| | | | | | | TTGC (SEQ ID |
| | | | | | | NO: 77) |
| chr8 | 127068714 | 127068785 | chr8q | 127z068749 | 127068750 | GTCACAGGTCCTCA |
| | | | | | | TGAGAATTGGAGGG |
| | | | | | | GACAAGA[CG]TCC |
| | | | | | | AAATCATATCAAAA |
| | | | | | | CTTGACAGAGTTTT |
| | | | | | | CATT (SEQ ID |
| | | | | | | NO: 78) |
| chr9 | 13856747 | 13856818 | chr9p | 13856782 | 13856783 | TTTCTTACTACAAA |
| | | | | | | TTTTCCTGTCATTT |
| | | | | | | CCTATTT[CG]ACC |
| | | | | | | TCTTTTATCTAAGC |
| | | | | | | CTGGAATGCAGTCA |
| | | | | | | GCAC (SEQ ID |
| | | | | | | NO: 79) |
| chr9 | 78293755 | 78293826 | chr9q | 78293790 | 78293791 | GCAAGGATGTCTCC |
| | | | | | | TCTCACACTCCTTT |
| | | | | | | TCAATAT[CG]TAC |
| | | | | | | TAGAAGTTCTAGCT |
| | | | | | | GATACAATAAGACA |
| | | | | | | AGAA (SEQ ID |
| | | | | | | NO: 80) |

TABLE 6
Exemplary mouse n₍ₓ₎>WCpGWn₍ₓ₎ genomic DNA
sequence motifs, wherein W = A or T, n = A
or G or C or T, and x = 35. The 19 randomly
chosen motif sequences are for common
(shared between/among cell/tissue types)
PMD solo-WCGW CpGs. The exemplary motif
sequences cover 35 bp upstream and 35 bp
downstream of the target CpG, which in each
case is surrounded by square brackets. The
respective SEQ ID NOS are shown to right
of each sequence in the last column. The
mouse reference version is GRCm38.
Specific chromosome accession numbers
can be found at https: //www.ncbi.
nlm.nih.gov/grc/mouse/data?asm=GRCm38.

| | | | | | | Sequence |
|---|---|---|---|---|---|---|
| chromo- | sequence | sequence | | | | (5′ to 3′); |
| some | begin | end | arm | CpG begin | CpG end | (SEQ ID NOS) |
| chr1 | 19259467 | 19259538 | chr1q | 19259502 | 19259503 | TGATCTACTCATG |
| | | | | | | CAGAAGGCAGGCC |
| | | | | | | TGCAAGTAT[CG] |
| | | | | | | TAGCTACACAGAG |
| | | | | | | TAAAACCAACATC |
| | | | | | | CAGCAATAA |
| | | | | | | (SEQ ID |
| | | | | | | NO: 81) |
| chr10 | 23645214 | 23645285 | chr10q | 23645249 | 23645250 | TAGTGGAGCATGT |
| | | | | | | ATCCTTATTACAT |
| | | | | | | CCCTTATTA[CG] |
| | | | | | | AGATAGCATTTGA |
| | | | | | | AATGTAAATGAAG |
| | | | | | | AAAATATCT |
| | | | | | | (SEQ ID |
| | | | | | | NO: 82) |
| chr11 | 28831037 | 28831108 | chr11q | 28831072 | 28831073 | CCTATCATATGCC |
| | | | | | | TGAAAAGCACTTA |
| | | | | | | CAACAGACT[CG] |
| | | | | | | AGTTGCTCTTGAC |
| | | | | | | TTTGTCCTACTAC |
| | | | | | | ACTTGCTTC |
| | | | | | | (SEQ ID |
| | | | | | | NO: 83) |
| chr12 | 10029631 | 10029702 | chr12q | 10029666 | 10029667 | GCTATAACATATT |
| | | | | | | CAGAGGGTAAGTC |
| | | | | | | CCATATTTT[CG] |
| | | | | | | TGTTTCTAATCAA |
| | | | | | | TGATGAGAGAATA |
| | | | | | | AAGACTCCT |
| | | | | | | (SEQ ID |
| | | | | | | NO: 84) |
| chr13 | 22908617 | 22908688 | chr13q | 22908652 | 22908653 | AAACAAATTCAAA |
| | | | | | | GACAAAAACCACA |
| | | | | | | TGATCATCT[CG] |
| | | | | | | TTAGATGCAGAAA |
| | | | | | | AAGCATTTGACAA |
| | | | | | | GATCCAACA |
| | | | | | | (SEQ ID |
| | | | | | | NO: 85) |
| chr14 | 36346214 | 36346285 | chr14q | 36346249 | 36346250 | GATTTCAGAGGAA |
| | | | | | | AACACTTTCTCTG |
| | | | | | | TCTTGTACT[CG] |
| | | | | | | TCCAGGTGATAAA |
| | | | | | | CTCCTACTTTGAA |
| | | | | | | ATCCTATTG |
| | | | | | | (SEQ ID |
| | | | | | | NO: 86) |
| chr15 | 26717633 | 26717704 | chr15q | 26717668 | 26717669 | CATGTCTTTCTCA |
| | | | | | | TTAGTTGTTAAGA |
| | | | | | | AATTGTCTT[CG] |
| | | | | | | TTCTGCATACAAT |
| | | | | | | TTGGCCACTAAAA |
| | | | | | | ATTGCATCA |
| | | | | | | (SEQ ID |
| | | | | | | NO: 87) |
| chr16 | 84244385 | 84244456 | chr16q | 84244420 | 84244421 | AATTCTAAGGGGC |
| | | | | | | AAAGTGTCCACAC |
| | | | | | | TTTGGTCTT[CG] |
| | | | | | | TTCTTCTTGAGTT |
| | | | | | | TCATGTGTTTTGC |
| | | | | | | AAATTGTAT |
| | | | | | | (SEQ ID |
| | | | | | | NO: 88) |
| chr17 | 61018970 | 61019041 | chr17q | 61019005 | 61019006 | TAAAAATAGGCTT |
| | | | | | | TTTAAGGTTAAGA |
| | | | | | | AAATCCTTT[CG] |
| | | | | | | TAAAATTGAGGTT |
| | | | | | | GATTTATCCAGAG |
| | | | | | | TCTAGAAAC |
| | | | | | | (SEQ ID |
| | | | | | | NO: 89) |
| chr18 | 26745680 | 26745751 | chr18q | 26745715 | 26745716 | ATACATGAGGACA |
| | | | | | | TTTAGCTTCTCTT |
| | | | | | | TTGGGTCTT[CG] |
| | | | | | | ATTTTATTTCAAT |
| | | | | | | GATCAACCTGTCT |
| | | | | | | GTTTCTGTA |
| | | | | | | (SEQ ID |
| | | | | | | NO: 90) |
| chr19 | 12225274 | 12225345 | chr19q | 12225309 | 12225310 | AACTTTTAGATTG |
| | | | | | | TTTATTTGTGTCT |
| | | | | | | GGAGACATT[CG] |
| | | | | | | ATTTTACCACACA |
| | | | | | | GCACCTTCTTTTC |
| | | | | | | CTTCATCAT |
| | | | | | | (SEQ ID |
| | | | | | | NO: 91) |
| chr2 | 55655906 | 55655977 | chr2q | 55655941 | 55655942 | TTTATTCACAGGG |
| | | | | | | ATTACTTCTTTTC |
| | | | | | | CTTTATCTA[CG] |
| | | | | | | TTTCTGTGAATGT |
| | | | | | | CTTTAATATTTTT |
| | | | | | | ATACTTCTA |
| | | | | | | (SEQ ID |
| | | | | | | NO: 92) |
| chr3 | 78067268 | 78067339 | chr3q | 78067303 | 78067304 | CTGACCTCCACTT |
| | | | | | | TAGTCAGCTCTTG |
| | | | | | | GCTCAAGCA[CG] |
| | | | | | | TACCACTGTGAAA |
| | | | | | | GCAAAACAGATGG |
| | | | | | | TCAGTAAGT |
| | | | | | | (SEQ ID |
| | | | | | | NO: 93) |
| chr4 | 93285296 | 93285367 | chr4q | 93285331 | 93285332 | TCTGTAAGAGGTC |
| | | | | | | ATCTTTTACACTA |
| | | | | | | AATAGAATT[CG] |
| | | | | | | TTCCTGATTTTAA |
| | | | | | | GCAAACTACTGTA |
| | | | | | | GCCAAAGCC |
| | | | | | | (SEQ ID |
| | | | | | | NO: 94) |
| chr5 | 78825073 | 78825144 | chr5q | 78825108 | 78825109 | GCAATCACCATCA |
| | | | | | | AAATTCCAACTCA |
| | | | | | | ATTCTTCAA[CG] |
| | | | | | | AATTAGAAAGAGC |
| | | | | | | AATCTGCAAATTC |
| | | | | | | ATCTGGAAC |
| | | | | | | (SEQ ID |
| | | | | | | NO: 95) |
| chr6 | 36083383 | 36083454 | chr6q | 36083418 | 36083419 | TGAGTTTCATGTG |
| | | | | | | TTTAGGAAATTGT |
| | | | | | | ATCTTATAT[CG] |
| | | | | | | TGGGTATCCTAGG |
| | | | | | | TTTTGGGCTAGTA |
| | | | | | | TCCACTTAT |
| | | | | | | (SEQ ID |
| | | | | | | NO: 96) |
| chr7 | 93705931 | 93706002 | chr7q | 93705966 | 93705967 | TTCTTTTCTGTTA |
| | | | | | | TTATCTTTTGAAG |
| | | | | | | GGCTGGATT[CG] |
| | | | | | | TGGAAAGATAATG |
| | | | | | | TGTGAATTTTGTT |
| | | | | | | TTGTAGTGG |
| | | | | | | (SEQ ID |
| | | | | | | NO: 97) |
| chr8 | 62873386 | 62873457 | chr8q | 62873421 | 62873422 | ACTCTAGCAAGCC |
| | | | | | | TGTCTTAGCATTA |
| | | | | | | GTTATGCAAfCG |
| | | | | | | TCAACTGGCCTCA |
| | | | | | | AAGTTACTGAGAT |
| | | | | | | TTGCTGCAG |
| | | | | | | (SEQ ID |
| | | | | | | NO: 98) |
| chr9 | 23741611 | 23741682 | chr9q | 23741646 | 23741647 | GCTTTACAAGGTA |
| | | | | | | AGTCTGGCCTTGA |
| | | | | | | ACTTTCTAA[CG] |
| | | | | | | AAATTCAAGACAG |
| | | | | | | TCTATCAGAAGTA |
| | | | | | | AAGTGGGGA |
| | | | | | | (SEQ ID |
| | | | | | | NO: 99) |

TABLE 7
Exemplary mouse n(x)WCpGWn(x) genomic DNA sequence motifs,
wherein W = A or T, n = A or G or C or T, and x = 35. The 19
exemplary motif sequences, represent randomly selected
intergenic CpGs (H3K36me3 primarily exists only at gene
bodies), are for common (shared between/among cell/tissue
types) PMD solo-WCGW CpGs. The exemplar motif sequences
cover 35 bp upstream and 35 bp downstream of the target
CpG, which in each case is surrounded by square brackets.
The respective SEQ ID NOS are shown to right of each
sequence in the last column. The mouse reference version
is GRCm38. Specific chromosome accession numbers can be found
at https: //www.ncbi.nlm.nih.gov/grc/mouse/data?asm=GRCm38.

| | | | | | | Sequence |
|---|---|---|---|---|---|---|
| | | | | | | (5′ to 3′); |
| chromo- | sequence | sequence | | | | (SEQ ID |
| some | begin | end | arm | CpG begin | CpG end | NOS) |
| chr1 | 101103624 | 101103695 | chr1q | 101103659 | 101103660 | TTTTCAGGTAC |
| | | | | | | TTCTCAGCCAT |
| | | | | | | TTGGTATTCCT |
| | | | | | | CA[CG]TGAGA |
| | | | | | | ATTCTTTGTTT |
| | | | | | | AGCTCTGAGCA |
| | | | | | | CAATTTTT |
| | | | | | | (SEQ ID |
| | | | | | | NO: 100) |
| chr10 | 102702261 | 102702332 | chr10q | 102702296 | 102702297 | ATCAAATAAGT |
| | | | | | | CACTTTACATC |
| | | | | | | TCTTCCCTGGT |
| | | | | | | AA[CG]ACTAC |
| | | | | | | AAAATTCCATA |
| | | | | | | CTTCTAAGAGC |
| | | | | | | CACAGAGA |
| | | | | | | (SEQ ID |
| | | | | | | NO: 101) |
| chr11 | 24964066 | 24964137 | chr11q | 24964101 | 24964102 | ATAAATGTGGA |
| | | | | | | ATTATATGTAC |
| | | | | | | atataaatgga |
| | | | | | | TA[CG]TTATC |
| | | | | | | CAAATTAAAAA |
| | | | | | | TTCAAGACCCA |
| | | | | | | AGAAATAC |
| | | | | | | (SEQ ID |
| | | | | | | NO: 102) |
| chr12 | 48091061 | 48091132 | chr12q | 48091096 | 48091097 | ATTCCAGATAA |
| | | | | | | ATTTGCAGATT |
| | | | | | | GCCCTTTCTAA |
| | | | | | | TT[CG]TTGAA |
| | | | | | | GAATTGAGTTG |
| | | | | | | GAATTTTGATG |
| | | | | | | GGGATTGT |
| | | | | | | (SEQ ID |
| | | | | | | NO: 103) |
| chr13 | 11139090 | 11139161 | chr13q | 11139125 | 11139126 | GCAATACCCAT |
| | | | | | | CAAAATTCCAA |
| | | | | | | ATCAATTCTTC |
| | | | | | | AA[CG]AATTA |
| | | | | | | GAAGGAGCAAT |
| | | | | | | TTGCAAATTCA |
| | | | | | | TCTGGAAT |
| | | | | | | (SEQ ID |
| | | | | | | NO: 104) |
| chr14 | 106494444 | 106494515 | chr14q | 106494479 | 106494480 | ATGCTACTTTT |
| | | | | | | GTGCTACTTCA |
| | | | | | | GCATTCATTTT |
| | | | | | | AA[CG]TTTTC |
| | | | | | | TTCAACTTTCT |
| | | | | | | TAATGTTTGTT |
| | | | | | | TCTCAAAG |
| | | | | | | (SEQ ID |
| | | | | | | NO: 105) |
| chr15 | 50051643 | 50051714 | chr15q | 50051678 | 50051679 | AATCTCAAGAT |
| | | | | | | AAAATATAAAA |
| | | | | | | TTGTACTCCAA |
| | | | | | | TT[CG]TTTGT |
| | | | | | | CAAGAGAACAT |
| | | | | | | AAATTCAAGCA |
| | | | | | | ATGCTCCC |
| | | | | | | (SEQ ID |
| | | | | | | NO: 106) |
| chr16 | 53374953 | 53375024 | chr16q | 53374988 | 53374989 | AATAGAATATT |
| | | | | | | CATCCCCAATG |
| | | | | | | CATTCTTAAGA |
| | | | | | | CT[CG]TGATA |
| | | | | | | TTAGTGAGAAA |
| | | | | | | AATATAGTATG |
| | | | | | | GAAGACTC |
| | | | | | | (SEQ ID |
| | | | | | | NO: 107) |
| chr17 | 94074535 | 94074606 | chr17q | 94074570 | 94074571 | AAAATACTTCT |
| | | | | | | AGCTATTTATT |
| | | | | | | GCTGTGCCTCA |
| | | | | | | AA[CG]ATCCT |
| | | | | | | AAAACAT GACA |
| | | | | | | ACATAAAACAG |
| | | | | | | CAGCATTT |
| | | | | | | (SEQ ID |
| | | | | | | NO: 108) |
| chr18 | 19222623 | 19222694 | chr18q | 19222658 | 19222659 | TCATACCAGTG |
| | | | | | | taaaatatagt |
| | | | | | | TGTGCAAAAAT |
| | | | | | | AT[CG]TTTGT |
| | | | | | | CATCTGTCTCT |
| | | | | | | AAAATTCCTAT |
| | | | | | | TATGACAA |
| | | | | | | (SEQ ID |
| | | | | | | NO: 109) |
| chr19 | 51173190 | 51173261 | chr19q | 51173225 | 51173226 | GGTGCACAGAA |
| | | | | | | CAGGAGCTTTG |
| | | | | | | CATATAAACTC |
| | | | | | | AA[CG]TGGTG |
| | | | | | | GT GACAACAGG |
| | | | | | | CAAAATCCTTG |
| | | | | | | AAAAGGAC |
| | | | | | | (SEQ ID |
| | | | | | | NO: 110) |
| chr2 | 57738394 | 57738465 | chr2q | 57738429 | 57738430 | CTACCCTACCC |
| | | | | | | CCTACACACAC |
| | | | | | | ACACACACACA |
| | | | | | | CA[CG]AGAGA |
| | | | | | | GAGAGAGAGAG |
| | | | | | | AGAGGGAGAGA |
| | | | | | | GAGAGAGA |
| | | | | | | (SEQ ID |
| | | | | | | NO: 111) |
| chr3 | 91837912 | 91837983 | chr3q | 91837947 | 91837948 | AGAGCATTATG |
| | | | | | | CACCTTTAAAC |
| | | | | | | ATTTGTTCTCT |
| | | | | | | CA[CG]ACCCT |
| | | | | | | TCATTTTGGTA |
| | | | | | | ACACTTAAACA |
| | | | | | | CTTGATGT |
| | | | | | | (SEQ ID |
| | | | | | | NO: 112) |
| chr4 | 13603340 | 13603411 | chr4q | 13603375 | 13603376 | CTACCACAGTC |
| | | | | | | ATTTTTATAAA |
| | | | | | | GGACATGGTCT |
| | | | | | | GT[CG]AGTAA |
| | | | | | | CCAACTTTGCA |
| | | | | | | TCCATTCAGCA |
| | | | | | | TGCCTTTC |
| | | | | | | (SEQ ID |
| | | | | | | NO: 113) |
| chr5 | 56958316 | 56958387 | chr5q | 56958351 | 56958352 | AATGAAATAAA |
| | | | | | | AGTCCATGTCC |
| | | | | | | TACCTTAAAAG |
| | | | | | | GA[CG]TAGTC |
| | | | | | | TTGAATAAACA |
| | | | | | | AACATTTAAAA |
| | | | | | | GACACATA |
| | | | | | | (SEQ ID |
| | | | | | | NO: 114) |
| chr6 | 20895739 | 20895810 | chr6q | 20895774 | 20895775 | TTTAAAGTGAA |
| | | | | | | TCTCTAACAAT |
| | | | | | | ATTTAGAATGA |
| | | | | | | AT[CG]AAATT |
| | | | | | | CAGTCAAACTA |
| | | | | | | ATGAAGCCTGA |
| | | | | | | GATACAAA |
| | | | | | | (SEQ ID |
| | | | | | | NO: 115) |
| chr7 | 8795790 | 8795861 | chr7q | 8795825 | 8795826 | AATTATCTTAT |
| | | | | | | AGAGGAGAAAG |
| | | | | | | TAGAGAAGAGT |
| | | | | | | CT[CG]AAGAT |
| | | | | | | ATTGGCACAAG |
| | | | | | | GGAAAACTTCC |
| | | | | | | TGAACTAC |
| | | | | | | (SEQ ID |
| | | | | | | NO: 116) |
| chr8 | 96443670 | 96443741 | chr8q | 96443705 | 96443706 | TTTAAAACTGA |
| | | | | | | ACTGAACTGCT |
| | | | | | | AATATCCTGAC |
| | | | | | | AA[CG]AATAT |
| | | | | | | TGAACTTGTAC |
| | | | | | | CCAAAGAGCTG |
| | | | | | | TTTCTAAA |
| | | | | | | (SEQ ID |
| | | | | | | NO: 117) |
| chr9 | 79360236 | 79360307 | chr9q | 79360271 | 79360272 | TAATTTAAAAA |
| | | | | | | ACTGAAAGAAA |
| | | | | | | CTAAGAAAAAA |
| | | | | | | AA[CG]TGAGG |
| | | | | | | AATGTATATAT |
| | | | | | | atatatatata |
| | | | | | | TATATATA |
| | | | | | | (SEQ ID |
| | | | | | | NO: 118) |

TABLE 8
Exemplary probes with extension base targeting
CpG dinucleotide sequences in the exemplary
human Solo-WCGW motif sequences listed in
Table 4 above. Note that the 3′ “C” of
the probe sequence corresponds to the “C”
of the CpG of the respective Solo-WCGW
sequences in Table 4 above.

| chromo- | probe sequence | |
|---|---|---|
| some | (5′ to 3′) | SEQ ID NOS |
| chr1 | AAATATTAACTATTATTA | SEQ ID NO: 119 |
| | TTTTTATCACACCATCTC | |
| chr1 | ATTTCAATAATAAAATCA | SEQ ID NO: 120 |
| | TATCTTTATCAAAAACTC | |
| chr10 | AACAATTTATATAAACAC | SEQ ID NO: 121 |
| | AAATAATAAAAAATAATC | |
| chr10 | AAATAAAAAAAACTCTTC | SEQ ID NO: 122 |
| | AAACCAAAAATTTAAAAC | |
| chr11 | TAATAAAAAAAAAATAAA | SEQ ID NO: 123 |
| | AATTAAATATAAAAATAC | |
| chr11 | ATTCCTAAAAAACTATAT | SEQ ID NO: 124 |
| | TAAACTAATTACTAACAC | |
| chr12 | TTTTCCCTTTATACCAAA | SEQ ID NO: 125 |
| | AAAATATCTAATTAACTC | |
| chr12 | AAATAAATTACTTAAACT | SEQ ID NO: 126 |
| | CAAAAATTCAAAACCAAC | |
| chr13 | CACATACACATATATATT | SEQ ID NO: 127 |
| | TATTACAACACTATTCAC | |
| chr14 | AAATTCATTCCCCATCCA | SEQ ID NO: 128 |
| | ATTAAATCAAATTAAAAC | |
| chr15 | CCTTCCACTAATAACCAT | SEQ ID NO: 129 |
| | CAAAATAACATTACAAAC | |
| chr16 | AACCAAAACAAACAAATC | SEQ ID NO: 130 |
| | ACTTAAAATCAAAAATTC | |
| chr16 | ATCCCAAAAATTCTAATA | SEQ ID NO: 131 |
| | TATTATATCTTTATTCTC | |
| chr17 | TCTCCTCCTAAATTATAT | SEQ ID NO: 132 |
| | AAAAAAATTATATTCCAC | |
| chr17 | CCTACACTTCCTAACCCT | SEQ ID NO: 133 |
| | CCATACTTAAACATAAAC | |
| chr18 | ACATATACCATATTAATT | SEQ ID NO: 134 |
| | TACTACACCCATCAACTC | |
| chr18 | ATCAAAATACTTATACCC | SEQ ID NO: 135 |
| | AAAACTAAATCATACCAC | |
| chr19 | CCCAACCTTAAAATATCC | SEQ ID NO: 136 |
| | TTTTTATACTTTATTTTC | |
| chr19 | CCATTTTATATAAAATCT | SEQ ID NO: 137 |
| | ACCATAAACAATATATAC | |
| chr2 | ATAACTTAACACAATAAA | SEQ ID NO: 138 |
| | TATTTATTTCTTACTCAC | |
| chr2 | ATTTAAACAAAAATATAT | SEQ ID NO: 139 |
| | TCAACCTATTTTATATAC | |
| chr20 | AACTATATACTAAAAACT | SEQ ID NO: 140 |
| | ACCAATACTCAACAAATC | |
| chr20 | TACCCAAATCTAACCTCT | SEQ ID NO: 141 |
| | TATTTCAAATCACAACTC | |
| chr21 | ACAAAAATTCATCAAATT | SEQ ID NO: 142 |
| | TAATAAAATTATCTATTC | |
| chr21 | AAAATAACTAAACTCCAA | SEQ ID NO: 143 |
| | TATCTCTAAAATAACTTC | |
| chr22 | AAATATAACTAAAAAACA | SEQ ID NO: 144 |
| | TTTTCTCCCATTATATAC | |
| chr3 | CACATTATCAAAATTAAT | SEQ ID NO: 145 |
| | AATAAATAAAAAACAATC | |
| chr3 | CCCCATAACCTAATCACC | SEQ ID NO: 146 |
| | TCCCCAAAAACCCCAATC | |
| chr4 | ATATAAACAAACAAAAAA | SEQ ID NO: 147 |
| | ATATAAAAAAAAAAACAC | |
| chr4 | CAAAATCATTTTTAATTA | SEQ ID NO: 148 |
| | TAAACTTTAAATATATTC | |
| chr5 | CTACAAACCAAACACACC | SEQ ID NO: 149 |
| | AAAAATTTCTAAAACCAC | |
| chr5 | AAATACAACCATTTTAAA | SEQ ID NO: 150 |
| | ATATCAAACCAAATATTC | |
| chr6 | AAAAAAACTTTTAATATT | SEQ ID NO: 151 |
| | ATTCTATTTATCTTTATC | |
| chr6 | CCACACTACTCAAAATAA | SEQ ID NO: 152 |
| | CTATTCCCCAAACTATTC | |
| chr7 | AAAAAAAAAAAAAAAATA | SEQ ID NO: 153 |
| | ATCTTATAAATTAATTAC | |
| chr7 | AAATCAAAACCATCCTAA | SEQ ID NO: 154 |
| | CCAACATAATAAAACCTC | |
| chr8 | CACTCCTCCCAAACACAA | SEQ ID NO: 155 |
| | AAACTAATCAATAATATC | |
| chr8 | TAAAATTCATTATAAACC | SEQ ID NO: 156 |
| | ATCTTAAAAACTATCTAC | |
| chr9 | AACCCAACTAAATTTTTA | SEQ ID NO: 157 |
| | TTATTCTTTTATAAACAC | |
| chr9 | CCTAATCCAATAATACTA | SEQ ID NO: 158 |
| | TAATCCTTATAAAAAAAC | |

TABLE 9
Exemplary probes with extension base targeting
CpG dinucleotide sequences in the exemplary
human Solo-WCGW motif sequences listed in
Table 5 above. Note that the 3′ “C” of the
probe sequence corresponds to the “C” of the
CpG of the respective Solo-WCGW sequences
in Table 5 above. Respective SEQ ID NOS
are in the right column.

| chromo- | probe sequence | |
|---|---|---|
| some | (5′ to 3′) | SEQ ID NOS |
| chr1 | TAATATCCCCTTTATCAT | SEQ ID NO: 159 |
| | TTTTTATTATATCTATTC | |
| chr1 | TTCTACCAAAAATACAAA | SEQ ID NO: 160 |
| | AAAAAACTAATACCATTC | |
| chr10 | CTAAATTCAAACAATCCT | SEQ ID NO: 161 |
| | CTTACCTCAACCTCCCTC | |
| chr10 | TTAAAATTACCAAAATTC | SEQ ID NO: 162 |
| | TTACACTAACTCTTTCTC | |
| chr11 | AAAACAAAATCTCACTAC | SEQ ID NO: 163 |
| | ATTACCCAAACTAATCTC | |
| chr11 | AATATTAATACCCCTACT | SEQ ID NO: 164 |
| | CTCTTTTAATTATTATTC | |
| chr12 | ATATATATATATATATAT | SEQ ID NO: 165 |
| | ATATATATATATACACAC | |
| chr12 | ATTTCAATACATAAAACT | SEQ ID NO: 166 |
| | AAAAAAATAAATCAAAAC | |
| chr13 | AACAACCTAAACAACATA | SEQ ID NO: 167 |
| | ATAAAACTCTATCTCTAC | |
| chr14 | AAATATCTTATTAATATT | SEQ ID NO: 168 |
| | TTTAAAATACTTAATTAC | |
| chr15 | ACATACACCATTAAAATA | SEQ ID NO: 169 |
| | AACAAATATTACTTTTTC | |
| chr16 | CAAACTAATAAAAACATA | SEQ ID NO: 170 |
| | ACATAAAATTAACCTAAC | |
| chr16 | CCTATAAACAAACATAAA | SEQ ID NO: 171 |
| | AAATAAACAACTACTAAC | |
| chr17 | AATAAAAAAATATATCAT | SEQ ID NO: 172 |
| | CACATATTCCTAAAATAC | |
| chr17 | TTTTTACTATTATAAATA | SEQ ID NO: 173 |
| | ATACTACAATAAACATAC | |
| chr18 | ATTATTTCAATAACACTT | SEQ ID NO: 174 |
| | ATATTTATTACAACTAAC | |
| chr18 | AAATATTATTCTTAAAAA | SEQ ID NO: 175 |
| | ATATTCAATCTATTCAAC | |
| chr19 | ACAATCAAATATACCCCT | SEQ ID NO: 176 |
| | TCTTAAAAACAAACAAAC | |
| chr19 | TAAATATTAAAAAAAATA | SEQ ID NO: 177 |
| | TCACAAAAAAATATATAC | |
| chr2 | AAAACCACCTATCCAAAA | SEQ ID NO: 178 |
| | CTATAAAAAACCTAAAAC | |
| chr2 | ATATTAACTATAAAATTT | SEQ ID NO: 179 |
| | CCATATATAACCTTTATC | |
| chr20 | AACATTATATAAAAATCA | SEQ ID NO: 180 |
| | AATTTTATTCCTCTCCAC | |
| chr20 | CCTAAAACAACCTAAATT | SEQ ID NO: 181 |
| | TTATTTCTCCTTCCTTTC | |
| chr21 | CCATTTATAACAATATAA | SEQ ID NO: 182 |
| | ATAAATCTAAAAAACATC | |
| chr21 | TCATCAATCACCACTATT | SEQ ID NO: 183 |
| | TCAATACAAAACATTTTC | |
| chr22 | TAAAATTCAATTTTTAAA | SEQ ID NO: 184 |
| | ATAAAACACTAAACCTTC | |
| chr3 | CTCACATAATACCCTACA | SEQ ID NO: 185 |
| | CTACCAAAACAAATAAAC | |
| chr3 | ATATTTTTAAAAACATAA | SEQ ID NO: 186 |
| | ATATTTAAACATACTAAC | |
| chr4 | AAAAAATAAATAAAATAA | SEQ ID NO: 187 |
| | AAACAACTTAAAAAACAC | |
| chr4 | TAACTCCACCAAAACAAA | SEQ ID NO: 188 |
| | AAAATCATCAAAAAAAAC | |
| chr5 | AAAATATAAAATATCATT | SEQ ID NO: 189 |
| | CTATTCATCATATTCTTC | |
| chr5 | AAAAAACTCCAACATATT | SEQ ID NO: 190 |
| | TACATCTTTTATATCTAC | |
| chr6 | CATTATCTATTTTTAAAT | SEQ ID NO: 191 |
| | TTAAAATAAAATTATCAC | |
| chr6 | TCCCCATTCTCCTCTCAT | SEQ ID NO: 192 |
| | ATAAAACTACCACAAAAC | |
| chr7 | AATAATTTAATAATTATT | SEQ ID NO: 193 |
| | ATACAAATATATTTTATC | |
| chr7 | TAACTAAAAAACACACTT | SEQ ID NO: 194 |
| | ATTACTCATAAAACAAAC | |
| chr8 | TAATCCATCAATTATTCA | SEQ ID NO: 195 |
| | ATAACCTAATTTTAATTC | |
| chr8 | ATCACAAATCCTCATAAA | SEQ ID NO: 196 |
| | AATTAAAAAAAACAAAAC | |
| chr9 | TTTCTTACTACAAATTTT | SEQ ID NO: 197 |
| | CCTATCATTTCCTATTTC | |
| chr9 | ACAAAAATATCTCCTCTC | SEQ ID NO: 198 |
| | ACACTCCTTTTCAATATC | |

TABLE 10
Exemplary probes with extension base targeting
CpG dinucleotide sequences in the exemplary
mouse Solo-WCGW motif sequences listed in
Table 6 above. Note that the 3′ “C” of the
probe sequence corresponds to the “C” of the
CpG of the respective Solo-WCGW sequences
in Table 6 above.

| chromo- | | |
|---|---|---|
| some | probe sequence | SEQ ID NO |
| chr1 | TAATCTACTCATACAAAA | SEQ ID NO: 199 |
| | AACAAACCTACAAATATC | |
| chr10 | TAATAAAACATATATCCT | SEQ ID NO: 200 |
| | TATTACATCCCTTATTAC | |
| chr11 | CCTATCATATACCTAAAA | SEQ ID NO: 201 |
| | AACACTTACAACAAACTC | |
| chr12 | ACTATAACATATTCAAAA | SEQ ID NO: 202 |
| | AATAAATCCCATATTTTC | |
| chr13 | AAACAAATTCAAAAACAA | SEQ ID NO: 203 |
| | AAACCACATAATCATCTC | |
| chr14 | AATTTCAAAAAAAAACAC | SEQ ID NO: 204 |
| | TTTCTCTATCTTATACTC | |
| chr15 | CATATCTTTCTCATTAAT | SEQ ID NO: 205 |
| | TATTAAAAAATTATCTTC | |
| chr16 | AATTCTAAAAAACAAAAT | SEQ ID NO: 206 |
| | ATCCACACTTTAATCTTC | |
| chr17 | TAAAAATAAACTTTTTAA | SEQ ID NO: 207 |
| | AATTAAAAAAATCCTTTC | |
| chr18 | ATACATAAAAACATTTAA | SEQ ID NO: 208 |
| | CTTCTCTTTTAAATCTTC | |
| chr19 | AACTTTTAAATTATTTAT | SEQ ID NO: 209 |
| | TTATATCTAAAAACATTC | |
| chr2 | TTTATTCACAAAAATTAC | SEQ ID NO: 210 |
| | TTCTTTTCCTTTATCTAC | |
| chr3 | CTAACCTCCACTTTAATC | SEQ ID NO: 211 |
| | AACTCTTAACTCAAACAC | |
| chr4 | TCTATAAAAAATCATCTT | SEQ ID NO: 212 |
| | TTACACTAAATAAAATTC | |
| chr5 | ACAATCACCATCAAAATT | SEQ ID NO: 213 |
| | CCAACTCAATTCTTCAAC | |
| chr6 | TAAATTTCATATATTTAA | SEQ ID NO: 214 |
| | AAAATTATATCTTATATC | |
| chr7 | TTCTTTTCTATTATTATC | SEQ ID NO: 215 |
| | TTTTAAAAAACTAAATTC | |
| chr8 | ACTCTAACAAACCTATCT | SEQ ID NO: 216 |
| | TAACATTAATTATACAAC | |
| chr9 | ACTTTACAAAATAAATCT | SEQ ID NO: 217 |
| | AACCTTAAACTTTCTAAC | |

Exemplary probes with extension base targeting
CpG dinucleotide sequences in the exemplary
mouse Solo-WCGW motif sequences listed in
Table 7 above. Note that the 3′ “C” of the
probe sequence corresponds to the “C” of the
CpG of the respective Solo-WCGW sequences in
Table 7 above. Respective SEQ ID NOS
are in the right column.

| chromo- | | |
|---|---|---|
| some | probe sequence | SEQ ID NO |
| chr1 | TTTTCAAATACTTCTCAA | SEQ ID NO: 218 |
| | CCATTTAATATTCCTCAC | |
| chr10 | ATCAAATAAATCACTTTA | SEQ ID NO: 219 |
| | CATCTCTTCCCTAATAAC | |
| chr11 | ATAAATATAAAATTATAT | SEQ ID NO: 220 |
| | ATACATATAAATAAATAC | |
| chr12 | ATTCCAAATAAATTTACA | SEQ ID NO: 221 |
| | AATTACCCTTTCTAATTC | |
| chr13 | ACAATACCCATCAAAATT | SEQ ID NO: 222 |
| | CCAAATCAATTCTTCAAC | |
| chr14 | ATACTACTTTTATACTAC | SEQ ID NO: 223 |
| | TTCAACATTCATTTTAAC | |
| chr15 | AATCTCAAAATAAAATAT | SEQ ID NO: 224 |
| | AAAATTATACTCCAATTC | |
| chr16 | AATAAAATATTCATCCCC | SEQ ID NO: 225 |
| | AATACATTCTTAAAACTC | |
| chr17 | AAAATACTTCTAACTATT | SEQ ID NO: 226 |
| | TATTACTATACCTCAAAC | |
| chr18 | TCATACCAATATAAAATA | SEQ ID NO: 227 |
| | TAATTATACAAAAATATC | |
| chr19 | AATACACAAAACAAAAAC | SEQ ID NO: 228 |
| | TTTACATATAAACTCAAC | |
| chr2 | CTACCCTACCCCCTACAC | SEQ ID NO: 229 |
| | ACACACACACACACACAC | |
| chr3 | AAAACATTATACACCTTT | SEQ ID NO: 230 |
| | AAACATTTATTCTCTCAC | |
| chr4 | CTACCACAATCATTTTTA | SEQ ID NO: 231 |
| | TAAAAAACATAATCTATC | |
| chr5 | AATAAAATAAAAATCCAT | SEQ ID NO: 232 |
| | ATCCTACCTTAAAAAAAC | |
| chr6 | TTTAAAATAAATCTCTAA | SEQ ID NO: 233 |
| | CAATATTTAAAATAAATC | |
| chr7 | AATTATCTTATAAAAAAA | SEQ ID NO: 234 |
| | AAAATAAAAAAAAATCTC | |
| chr8 | TTTAAAACTAAACTAAAC | SEQ ID NO: 235 |
| | TACTAATATCCTAACAAC | |
| chr9 | TAATTTAAAAAACTAAAA | SEQ ID NO: 236 |
| | AAAACTAAAAAAAAAAAC | |

TABLE 12
Characterization primary cells used in solo-WCGW mitotic clock construction.
Reported PDL is a measure of mitotic age in culture only, as reported by
biobank vendor (Coriell). Standardized PDL is a mathematical estimate of the actual
mitotic age of each cell type, reflecting mitotic history in and before cell culture.

| Coriell | | | Reported | Standardized | | |
|---|---|---|---|---|---|---|
| ID | Cell type | Donor age | PDL | PDL | Sex | Race |
| AG21859 | Skin fibroblast | Neonate (0 y) | 6.82 | 26.0 | Male | Caucasian |
| AG21839 | Skin fibroblast | Neonate (0 y) | 5.39 | [5.39] | Male | Not reported |
| AG16146 | Skin fibroblast | Adult (31 y) | 4 | 43.15 | Male | Caucasian |
| AG11182 | Vein endothelial cell | Adolescent | 5.91 | 47.17 | Male | Caucasian |
| | (Iliac) | (15 y) | | | | |
| AG11546 | Vein smooth muscle cell | Adult (19 y) | 26 | 16.65 | Male | Caucasian |
| | (Iliac) | | | | | |

TABLE 13
44 CpGs and coefficients selected by elastic net regression of
solo-WCGW CpG beta values from serial primary cell culture to
standardized population doubling level. Four tissues and five donors are
represented across 116 timepoints to generate this multi-tissue model.

| CpG Marker | Coefficient |
|---|---|
| (Intercept) | 83.0126509 |
| cg00633815 | −0.5518149 |
| cg00756431 | 8.81719933 |
| cg02392915 | −4.0598453 |
| cg02593932 | 15.3483584 |
| cg04293275 | −10.14431 |
| cg05380830 | 1.72139531 |
| cg05625027 | −5.648398 |
| cg07158237 | −19.239856 |
| cg08457479 | −0.0091438 |
| cg08566792 | −0.0684508 |
| cg08707225 | −0.0981587 |
| cg08777703 | −5.5918972 |
| cg09763729 | −4.4732931 |
| cg10299521 | −4.5195526 |
| cg11558212 | −0.0069268 |
| cg12423387 | 1.60682734 |
| cg12441123 | −0.0068909 |
| cg14235511 | −5.7077285 |
| cg14874516 | 2.53000325 |
| cg15328937 | −8.764524 |
| cg15699514 | −0.4109342 |
| cg15853512 | −12.493757 |
| cg15868178 | 15.5166784 |
| cg16776291 | −1.1776387 |
| cg16940826 | −0.1209694 |
| cg17330885 | −0.0104335 |
| cg17858719 | −0.0338121 |
| cg19558170 | −4.0437772 |
| cg22031606 | −5.4113509 |
| cg22509480 | −3.0327514 |
| cg22531284 | −0.7221717 |
| cg22962360 | 3.55864073 |
| cg23127532 | −5.0212504 |
| cg23260202 | −1.0239884 |
| cg23260554 | −0.5037005 |
| cg24092773 | −1.8329249 |
| cg24305861 | −0.1232256 |
| cg24306397 | 0.28567637 |
| cg24707643 | −6.6319206 |
| cg24759892 | −1.2915068 |
| cg25129056 | −9.9425957 |
| cg25439479 | 0.82235261 |
| cg25576497 | −1.5276623 |
| cg26550001 | −5.6363962 |

TABLE 14
Summary of predictive performance of various methylation clocks on training
dataset from primary cells. Correlation across cultures is to observe PDL except
for the elastic net model, where correlation is to standardized PDL. Cross-culture
correlations include all observed timepoints (n = 116) for all cultures (n = 5).
1334/353 DNAm Age probes are present on the EPIC array, possibly affecting
predictive ability.

| | Elastic | | | Skin & | | |
|---|---|---|---|---|---|---|
| | net | Overlapping | | Blood | | |
| | solo- | individual | DNAm | DNAm | | |
| | WCGW | regression | Age | Age | PhenoAge | epiTOC |
| | mitotic | solo-WCGW | (Horvath | (Horvath | (Levine | (Yang |
| Model | clock* | miotic clock | 2013) | 2018) | 2018) | 2016) |
| Number of | 44 | 75 | 353¹ | 391 | 513 | 385 |
| probes | | | | | | |
| Cross-culture | 0.976 | −0.549 | 0.200 | −0.0444 | 0.594 | 0.577 |
| correlation to | | | | | | |
| PDL | | | | | | |
| (standardized | | | | | | |
| PDL when | | | | | | |
| implicated*) | | | | | | |
| AG21859 | 0.986 | −0.992 | 0.863 | 0.734 | 0.814 | 0.843 |
| correlation | | | | | | |
| AG21839 | 0.987 | −0.989 | 0.925 | 0.941 | 0.887 | 0.950 |
| correlation | | | | | | |
| AG16146 | 0.936 | −0.968 | 0.935 | −0.872 | −0.940 | 0.420 |
| correlation | | | | | | |
| AG11182 | 0.925 | −0.977 | 0.657 | 0.751 | 0.646 | 0.402 |
| correlation | | | | | | |
| AG11546 | 0.955 | −0.982 | −0.205 | 0.802 | −0.716 | 0.198 |
| correlation | | | | | | |

TABLES 15A-B. 44-CpG model. The human reference sequence version is GRCh37 (hg19). Specific chromosome accession numbers can be found at https://www. ncbi.nlm.nih.gov/grc/human/data?asm=GRCh37.

TABLE 15A

| SEQ | | | | | | | |
|---|---|---|---|---|---|---|---|
| ID | | chromo- | sequence | sequence | | EPIC Array | Regression |
| No. | Composite ID | some | begin | end | arm | ProbeID | coefficient |
| SEQ | cg00633815_chr1_ | chr1 | 165400618 | 165400689 | chr1q | cg00633815 | −0.551814925 |
| ID | 165400653 | | | | | | |
| 239 | | | | | | | |
| SEQ | cg09763729_chr1_ | chr1 | 176796254 | 176796325 | chr1q | cg09763729 | −4.473293112 |
| ID | 176796289 | | | | | | |
| 240 | | | | | | | |
| SEQ | cg16940826_chr1_ | chr1 | 225083851 | 225083922 | chr1q | cg16940826 | −0.120969431 |
| ID | 225083886 | | | | | | |
| 241 | | | | | | | |
| SEQ | cg23260554_chr1_ | chr1 | 2934461 | 2934532 | chr1p | cg23260554 | −0.503700469 |
| ID | 2934496 | | | | | | |
| 242 | | | | | | | |
| SEQ | cg25576497_chr1_ | chr1 | 176601233 | 176601304 | chr1q | cg25576497 | −1.527662339 |
| ID | 176601268 | | | | | | |
| 243 | | | | | | | |
| SEQ | cg04293275_chr10_ | chr10 | 9710731 | 9710802 | chr10p | cg04293275 | −10.14430992 |
| ID | 9710766 | | | | | | |
| 244 | | | | | | | |
| SEQ | cg15699514_chr10_ | chr10 | 10704495 | 10704566 | chr10p | cg15699514 | −0.410934183 |
| ID | 10704530 | | | | | | |
| 245 | | | | | | | |
| SEQ | cg23127532_chr10_ | chr10 | 20164010 | 20164081 | chr10p | cg23127532 | −5.021250405 |
| ID | 20164045 | | | | | | |
| 246 | | | | | | | |
| SEQ | cg23260202_chr11_ | chr11 | 70705799 | 70705870 | chr11q | cg23260202 | −1.023988438 |
| ID | 70705834 | | | | | | |
| 247 | | | | | | | |
| SEQ | cg25129056_chr11_ | chr11 | 30141899 | 30141970 | chr11p | cg25129056 | −9.942595724 |
| ID | 30141934 | | | | | | |
| 248 | | | | | | | |
| SEQ | cg24305861_chr12_ | chr12 | 99564237 | 99564308 | chr12q | cg24305861 | −0.123225571 |
| ID | 99564272 | | | | | | |
| 249 | | | | | | | |
| SEQ | cg08777703_chr13_ | chr13 | 72199271 | 72199342 | chr13q | cg08777703 | −5.591897217 |
| ID | 72199306 | | | | | | |
| 250 | | | | | | | |
| SEQ | cg11558212_chr13_ | chr13 | 22809965 | 22810036 | chr13q | cg11558212 | −0.00692676 |
| ID | 22810000 | | | | | | |
| 251 | | | | | | | |
| SEQ | cg24759892_chr13_ | chr13 | 93141655 | 93141726 | chr13q | cg24759892 | −1.291506763 |
| ID | 93141690 | | | | | | |
| 252 | | | | | | | |
| SEQ | cg08566792_chr14_ | chr14 | 83721955 | 83722026 | chr14q | cg08566792 | −0.068450829 |
| ID | 83721990 | | | | | | |
| 253 | | | | | | | |
| SEQ | cg24092773_chr14_ | chr14 | 95327800 | 95327871 | chr14q | cg24092773 | −1.832924922 |
| ID | 95327835 | | | | | | |
| 254 | | | | | | | |
| SEQ | cg17330885_chr15_ | chr15 | 54055554 | 54055625 | chr15q | cg17330885 | −0.010433494 |
| ID | 54055589 | | | | | | |
| 255 | | | | | | | |
| SEQ | cg19558170_chr15_ | chr15 | 84624456 | 84624527 | chr15q | cg19558170 | −4.043777176 |
| ID | 84624491 | | | | | | |
| 256 | | | | | | | |
| SEQ | cg02392915_chr16_ | chr16 | 49437418 | 49437489 | chr16q | cg02392915 | −4.05984532 |
| ID | 49437453 | | | | | | |
| 257 | | | | | | | |
| SEQ | cg17858719_chr16_ | chr16 | 13636246 | 13636317 | chr16q | cg17858719 | −0.033812107 |
| ID | 13636281 | | | | | | |
| 258 | | | | | | | |
| SEQ | cg14874516_chr18_ | chr18 | 5630915 | 5630986 | chr18p | cg14874516 | 2.530003254 |
| ID | 5630950 | | | | | | |
| 259 | | | | | | | |
| SEQ | cg02593932_chr2_ | chr2 | 154728272 | 154728343 | chr2q | cg02593932 | 15.34835844 |
| ID | 154728307 | | | | | | |
| 260 | | | | | | | |
| SEQ | cg15328937_chr2_ | chr2 | 7212053 | 7212124 | chr2p | cg15328937 | −8.764523985 |
| ID | 7212088 | | | | | | |
| 261 | | | | | | | |
| SEQ | cg08457479_chr20_ | chr20 | 4424914 | 4424985 | chr20p | cg08457479 | −0.009143777 |
| ID | 4424949 | | | | | | |
| 262 | | | | | | | |
| SEQ | cg12441123_chr20_ | chr20 | 51818094 | 51818165 | chr20q | cg12441123 | −0.00689095 |
| ID | 51818129 | | | | | | |
| 263 | | | | | | | |
| SEQ | cg22962360_chr20_ | chr20 | 21818144 | 21818215 | chr20p | cg22962360 | 3.558640734 |
| ID | 21818179 | | | | | | |
| 264 | | | | | | | |
| SEQ | cg05380830_chr21_ | chr21 | 39710207 | 39710278 | chr21q | cg05380830 | 1.721395312 |
| ID | 39710242 | | | | | | |
| 265 | | | | | | | |
| SEQ | cg10299521_chr21_ | chr21 | 31595983 | 31596054 | chr21q | cg10299521 | −4.519552552 |
| ID | 31596018 | | | | | | |
| 266 | | | | | | | |
| SEQ | cg08707225_chr22_ | chr22 | 25107754 | 25107825 | chr22q | cg08707225 | −0.098158705 |
| ID | 25107789 | | | | | | |
| 267 | | | | | | | |
| SEQ | cg07158237_chr3_ | chr3 | 76181385 | 76181456 | chr3p | cg07158237 | −19.23985624 |
| ID | 76181420 | | | | | | |
| 268 | | | | | | | |
| SEQ | cg15868178_chr3_ | chr3 | 120501293 | 120501364 | chr3q | cg15868178 | 15.51667837 |
| ID | 120501328 | | | | | | |
| 269 | | | | | | | |
| SEQ | cg05625027_chr4_ | chr4 | 113735418 | 113735489 | chr4q | cg05625027 | −5.648398027 |
| ID | 113735453 | | | | | | |
| 270 | | | | | | | |
| SEQ | cg14235511_chr4_ | chr4 | 139710165 | 139710236 | chr4q | cg14235511 | −5.707728482 |
| ID | 139710200 | | | | | | |
| 271 | | | | | | | |
| SEQ | cg22031606_chr4_ | chr4 | 62303518 | 62303589 | chr4q | cg22031606 | −5.411350865 |
| ID | 62303553 | | | | | | |
| 272 | | | | | | | |
| SEQ | cg00756431_chr5_ | chr5 | 168777641 | 168777712 | chr5q | cg00756431 | 8.81719933 |
| ID | 168777676 | | | | | | |
| 273 | | | | | | | |
| SEQ | cg15853512_chr5_ | chr5 | 42565316 | 42565387 | chr5p | cg15853512 | −12.49375667 |
| ID | 42565351 | | | | | | |
| 274 | | | | | | | |
| SEQ | cg16776291_chr5_ | chr5 | 38672093 | 38672164 | chr5p | cg16776291 | −1.177638664 |
| ID | 38672128 | | | | | | |
| 275 | | | | | | | |
| SEQ | cg12423387_chr7_ | chr7 | 130871924 | 130871995 | chr7q | cg12423387 | 1.606827344 |
| ID | 130871959 | | | | | | |
| 276 | | | | | | | |
| SEQ | cg22531284_chr7_ | chr7 | 132104867 | 132104938 | chr7q | cg22531284 | −0.722171739 |
| ID | 132104902 | | | | | | |
| 277 | | | | | | | |
| SEQ | cg24306397_chr7_ | chr7 | 93718644 | 93718715 | chr7q | cg24306397 | 0.285676368 |
| ID | 93718679 | | | | | | |
| 278 | | | | | | | |
| SEQ | cg22509480_chr8_ | chr8 | 130400740 | 130400811 | chr8q | cg22509480 | −3.032751399 |
| ID | 130400775 | | | | | | |
| 279 | | | | | | | |
| SEQ | cg24707643_chr8_ | chr8 | 133507611 | 133507682 | chr8q | cg24707643 | −6.631920581 |
| ID | 133507646 | | | | | | |
| 280 | | | | | | | |
| SEQ | cg25439479_chr8_ | chr8 | 92971526 | 92971597 | chr8q | cg25439479 | 0.822352611 |
| ID | 92971561 | | | | | | |
| 281 | | | | | | | |
| SEQ | cg26550001_chr8_ | chr8 | 94247480 | 94247551 | chr8q | cg26550001 | −5.636396176 |
| ID | 94247515 | | | | | | |
| 282 | | | | | | | |
| | | | | | | (Intercept) | 83.01265089 |

Table 15B

| SEQ ID | CpG | CpG | Sequence |
|---|---|---|---|
| No. | begin | end | (5′ to 3′) |
| SEQ ID | 165400653 | 165400654 | AGACTCTTCTGAGGCCCTGG |
| 239 | | | GGGCTGTGACATTTA[CG]AG |
| | | | GCCAATGTATACCTTGAGTCT |
| | | | GTTACTAAGATA |
| SEQ ID | 176796289 | 176796290 | TATTCCATATTATGGACAGCC |
| 240 | | | AGTTCTGTTCTTCT[CG]TTC |
| | | | ATATTGCTTGAACTCAACTCC |
| | | | TACTTGGTCCT |
| SEQ ID | 225083886 | 225083887 | CTTGCAGTCAAGTTGAAGAAC |
| 241 | | | CAGTGAATGACAGC[CG]TTG |
| | | | CAGGTGGGTTTCAGAAACTCC |
| | | | CTGAGAATCTC |
| SEQ ID | 2934496 | 2934497 | GTGGCTCTTAAACCCACTGGA |
| 242 | | | TCTTCTCAGTGGCC[CG]TGG |
| | | | TGCCAGCCCCAGACAGTGGCC |
| | | | AGGCCTCCTTG |
| SEQ ID | 176601268 | 176601269 | GGTAGATGGTTTAGGAAGACA |
| 243 | | | GTGAAGATTTTCAC[CG]TGA |
| | | | AGGAAATGGAGAAAGATGCTT |
| | | | GTTAGAGATAT |
| SEQ ID | 9710766 | 9710767 | GGGGATTCTTCTTTTCTGATG |
| 244 | | | GCCTTTAGAATGAG[CG]TTG |
| | | | GATCTTCCTGGGTCTCAAGCC |
| | | | TGCAGGCTTTG |
| SEQ ID | 10704530 | 10704531 | AGAGATTTGCAGGCATGGTAG |
| 245 | | | GCAGATGAGGAAGC[CG]TGA |
| | | | CAAAAGGGAAATTTGTGTGCC |
| | | | TAAGAAGTCTC |
| SEQ ID | 20164045 | 20164046 | AAGGTGCAAAAATTAAATCAT |
| 246 | | | GCATGCAAAGCAGT[CG]TAG |
| | | | GTGCTCCATAGTATGTGGTTA |
| | | | GCCTTATAATG |
| SEQ ID | 70705834 | 70705835 | GTCAAGTCCCTGCCCTTGAAT |
| 247 | | | GTGGTTTGACCTCC[CG]AAG |
| | | | TGAGAAAACATGCCAGGAAGC |
| | | | TTGTTACCCAC |
| SEQ ID | 30141934 | 30141935 | TTTTTCTCACTATGGCATGCA |
| 248 | | | CCTAATCCTTGGTC[CG]TGA |
| | | | CTGCTAAAGCAGTAGATTTCT |
| | | | ATGGCCCTTTG |
| SEQ ID | 99564272 | 99564273 | TCTCATGGTTTTATTTGAAGC |
| 249 | | | TGAAATGAAATAGC[CG]TGA |
| | | | AAAAAGCACTGTAACTTAGAG |
| | | | CTATCTCAATC |
| SEQ ID | 72199306 | 72199307 | ATGACTACTGTAGACACTCTT |
| 250 | | | AAATTCCCTGTCAA[CG]TTT |
| | | | CATTATAGCAGCATCATCTGT |
| | | | TTGAAAATATA |
| SEQ ID | 22810000 | 22810001 | TGCAGAGGACATGGGCTTCCT |
| 251 | | | CATCACTGATGCCA[CG]AGC |
| | | | TCCTCATGGGTAGACAGGACC |
| | | | CTGCCAGTGAC |
| SEQ ID | 93141690 | 93141691 | CAGTAAATACATCATGTGTCA |
| 252 | | | GATATTGATGAGAC[CG]TGG |
| | | | AGAAGAATTAGGCAAGGTAAT |
| | | | TTGCATAAAAA |
| SEQ ID | 83721990 | 83721991 | CCTGAAGCCCATAAGTCATCT |
| 253 | | | CATTAGTATACAAA[CG]TAG |
| | | | TATTATGCCATTACTTTTAAT |
| | | | GGCAAAAACCA |
| SEQ ID | 95327835 | 95327836 | GTGGGAAGTCACTAACACTGA |
| 254 | | | GGGAGAAATGGTCA[CG]TCA |
| | | | TGAGAGCATCACAAAGAGGTG |
| | | | AGGTCACAGGT |
| SEQ ID | 54055589 | 54055590 | ACTGTAAGATCATTCACCCTA |
| 255 | | | ACTCATTCCACTTT[CG]ACA |
| | | | TCCTGTTACTTCCAGTATTGT |
| | | | TTATTCCTTCC |
| SEQ ID | 84624491 | 84624492 | GTCACCCAGGAGCTAGGACCT |
| 256 | | | GGCATGGGGGCTTC[CG]ACT |
| | | | CTGCCCAGTGCACTGTCTGTG |
| | | | GCTGAGCTTGT |
| SEQ ID | 49437453 | 49437454 | GTTGGCCAGGCTTAGCTGAGC |
| 257 | | | TAGGCTGGAGTTAC[CG]TCT |
| | | | GCAGTCAGCTAGTGGGTTAAC |
| | | | TGGGTCTGGCT |
| SEQ ID | 13636281 | 13636282 | GGAATCATCAGGAAGCTCCTG |
| 258 | | | TGGGACAGATAACA[CG]TGT |
| | | | TCATTGTATAGGTGAGGGAGC |
| | | | TAAGGTTCAGA |
| SEQ ID | 5630950 | 5630951 | GTGGAGGGAAGGGAGAGGCTA |
| 259 | | | TGATAAATGTCCCT[CG]TGT |
| | | | GCCTTAAGGGGACCTGGTAAC |
| | | | TTGGTTTCTTT |
| SEQ ID | 154728307 | 154728308 | GGAGCAGGGAGGGAGGAGGGC |
| 260 | | | TGGGGGTGCTGGTT[CG]TAA |
| | | | ATGATACTAGCCCAGTGAGAG |
| | | | GCCTCCAGGCT |
| SEQ ID | 7212088 | 7212089 | GAAATTCCTCCTGGAACTCCA |
| 261 | | | GTGTCTGCTCCTAC[CG]ACA |
| | | | GGCTCCAGCCCACCCTAAGGA |
| | | | TTTTGGATTTG |
| SEQ ID | 4424949 | 4424950 | ACTCAGCAATTCCTTGCTAAG |
| 262 | | | ACTTACAGATAGCC[CG]TAC |
| | | | TGGTGGCTGTTCCAGATATCT |
| | | | TCTCTCTTATT |
| SEQ ID | 51818129 | 51818130 | AGATCCTTAATTTTCTAACAT |
| 263 | | | CAGCAAAGTCCCTT[CG]TCA |
| | | | CATAAACTGACATTCACAGGT |
| | | | TCTGGACATTC |
| SEQ ID | 21818179 | 21818180 | GAAGTGACTGAGACCAGATGA |
| 264 | | | TCACCACTGGGCAC[CG]TGG |
| | | | TCTCTGTAGCAGGCTCAGGGA |
| | | | GCCCAGGGTTG |
| SEQ ID | 39710242 | 39710243 | AGGAATATGACTTTGTGGCAA |
| 265 | | | ATGCTTTAACTTGG[CG]TAA |
| | | | GAGCTAAGTCTGGCATTGCTG |
| | | | CAATTGAATGG |
| SEQ ID | 31596018 | 31596019 | TATTTCTTGTTCTTATCTTTC |
| 266 | | | TTTTTCTCTGACCT[CG]TTC |
| | | | CAGATATCTTTAGAGTTGCTG |
| | | | CTATGGGGAGC |
| SEQ ID | 25107789 | 25107790 | AAGTATGTGCCCTTTATCCTC |
| 267 | | | CTGGACATGAGCAG[CG]ACT |
| | | | TTTTTTTTTTTTTTTTTTTTT |
| | | | TTTGAGATGGT |
| SEQ ID | 76181420 | 76181421 | CATTCTTCTAGGATCAAATTG |
| 268 | | | TGGCAATAGGAGAG[CG]TGC |
| | | | TACAGGGCAGCTCTTTGCTGC |
| | | | AGTGTTGCAGA |
| SEQ ID | 120501328 | 120501329 | TGGTAAACCCTTAGGAAGAAA |
| 269 | | | TTAGAAAAACATGG[CG]TAA |
| | | | GACAAGAAGTCTCTGTGAAGG |
| | | | GTTGAAGAGTG |
| SEQ ID | 113735453 | 113735454 | AAGTGTTAATTACCTAATGAA |
| 270 | | | CAATAACTCAGCCA[CG]AGA |
| | | | GAAATATTCAGTATGTTATTT |
| | | | ACTGGAGAAGG |
| SEQ ID | 139710200 | 139710201 | GAGCAGAGATTCTGGAGGAAC |
| 271 | | | TGATCCATTGAGCC[CG]TAG |
| | | | ATAGTGGGGCAAGAGCATTCC |
| | | | AGGCAGGAGAA |
| SEQ ID | 62303553 | 62303554 | TAACTCATGTTGTTTTCCCTG |
| 272 | | | CCTTGGAATTCTGC[CG]TCC |
| | | | TCCTCCCTCCCTCCCCTTGCA |
| | | | ACACTTACCCA |
| SEQ ID | 168777676 | 168777677 | AATGCAAAATGTGCAGTTCAG |
| 273 | | | GCTGGCAGAAGGAA[CG]AGG |
| | | | CTGGAATAGGAGCCAACAGGC |
| | | | TTATAATAATA |
| SEQ ID | 42565351 | 42565352 | CAGATCTGTATTCCTCATGAA |
| 274 | | | AATAAAACCTCTCT[CG]ACA |
| | | | CACTGTGTCCTTGTGGGTTTT |
| | | | TAGTTTTACTA |
| SEQ ID | 38672128 | 38672129 | ATAACATCCTGGAGGGGAACT |
| 275 | | | GACTCCTACAATGC[CG]AAA |
| | | | GAGATCTATACCAAGAACATG |
| | | | GCTCTCACAGA |
| SEQ ID | 130871959 | 130871960 | TGGCCTTCAGCATTGAACTAA |
| 276 | | | ATAAGCAGTCATGG[CG]AAG |
| | | | TGGCCAGAGGATTTGTTCAGT |
| | | | GTCATACTTGC |
| SEQ ID | 132104902 | 132104903 | GAGGGGATCCCCACCAACCTC |
| 277 | | | TTCCACACCTGCCC[CG]AGT |
| | | | CAAGGTCAAGTCCACATTGCT |
| | | | CCTGTGCCTCT |
| SEQ ID | 93718679 | 93718680 | TCTCTAGTAGCACCTCACATG |
| 278 | | | ACTAGTAAGCCCTT[CG]AAG |
| | | | GGGTATGCACACCATTGGATA |
| | | | CCCCTTCTCAA |
| SEQ ID | 130400775 | 130400776 | AAGCAATGACATTTGCCAAGA |
| 279 | | | GAAATGCTCAGGCC[CG]TCC |
| | | | TGTGGGCACTCATTGCTGCAT |
| | | | CATGAGAGGCC |
| SEQ ID | 133507646 | 133507647 | ATGAGAAGGTATGACATGAAC |
| 280 | | | TAAATGACATTTTT[CG]TCA |
| | | | TTCTGGCTGCTGTAGAGAGAA |
| | | | TGGAATAGAAG |
| SEQ ID | 92971561 | 92971562 | TGTCTTACTCTGTGGAACCTT |
| 281 | | | GCAAAAGTGAAGAA[CG]TTG |
| | | | AAGGGTTATTTAGGGCAGCTG |
| | | | GCTGATGTCAA |
| SEQ ID | 94247515 | 94247516 | CTGTGTATCAGTAAGTGGGTG |
| 282 | | | TGGGTGTGTATATT[CG]TGT |
| | | | GCATTTCAGTGTTTGTCTAAG |
| | | | TGTTTATGTGT |

TABLES 16A-B. 75-CpG Subset. The human reference sequence version is GRCh37 (hg19). Specific chromosome accession numbers can be found at https://www. ncbi.nlm.nih.gov/grc/human/data?asm=GRCh37.

TABLE 16A

| SEQ | | | | | | |
|---|---|---|---|---|---|---|
| ID | | chromo | sequence | sequence | | |
| No. | Composite ID | some | begin | end | arm | ProbeID |
| SEQ | cg10696969_chr1_ | chr1 | 3104006 | 3104077 | chr1p | cg10696969 |
| ID | 3104041 | | | | | |
| 283 | | | | | | |
| SEQ | cg14649362_chr1_ | chr1 | 154721873 | 154721944 | chr1q | cg14649362 |
| ID | 154721908 | | | | | |
| 284 | | | | | | |
| SEQ | cg07230985_chr10_ | chr10 | 132281501 | 132281572 | chr10q | cg07230985 |
| ID | 132281536 | | | | | |
| 285 | | | | | | |
| SEQ | cg08666638_chr10_ | chr10 | 20071694 | 20071765 | chr10q | cg08666638 |
| ID | 20071729 | | | | | |
| 286 | | | | | | |
| SEQ | cg12950311_chr10_ | chr10 | 19886770 | 19886841 | chr10p | cg12950311 |
| ID | 19886805 | | | | | |
| 287 | | | | | | |
| SEQ | cg14752504_chr10_ | chr10 | 130093361 | 130093432 | chr10q | cg14752504 |
| ID | 130093396 | | | | | |
| 288 | | | | | | |
| SEQ | cg23127532_chr10_ | chr10 | 20164010 | 20164081 | chr10p | cg23127532 |
| ID | 20164045 | | | | | |
| 289 | | | | | | |
| SEQ | cg24385652_chr10_ | chr10 | 50329792 | 50329863 | chr10q | cg24385652 |
| ID | 50329827 | | | | | |
| 290 | | | | | | |
| SEQ | cg25079832_chr10_ | chr10 | 130277358 | 130277429 | chr10q | cg25079832 |
| ID | 130277393 | | | | | |
| 291 | | | | | | |
| SEQ | cg05616355_chr11_ | chr11 | 124480954 | 124481025 | chr11q | cg05616355 |
| ID | 124480989 | | | | | |
| 292 | | | | | | |
| SEQ | cg06988933_chr11_ | chr11 | 45699357 | 45699428 | chr11p | cg06988933 |
| ID | 45699392 | | | | | |
| 293 | | | | | | |
| SEQ | cg17425351_chr11_ | chr11 | 110843009 | 110843080 | chr11q | cg17425351 |
| ID | 110843044 | | | | | |
| 294 | | | | | | |
| SEQ | cg17434901_chr11_ | chr11 | 133913832 | 133913903 | chr11q | cg17434901 |
| ID | 133913867 | | | | | |
| 295 | | | | | | |
| SEQ | cg25415985_chr11_ | chr11 | 84881718 | 84881789 | chr11q | cg25415985 |
| ID | 84881753 | | | | | |
| 296 | | | | | | |
| SEQ | cg00171816_chr12_ | chr12 | 99227017 | 99227088 | chr12q | cg00171816 |
| ID | 99227052 | | | | | |
| 297 | | | | | | |
| SEQ | cg06605459_chr12_ | chr12 | 117747371 | 117747442 | chr12q | cg06605459 |
| ID | 117747406 | | | | | |
| 298 | | | | | | |
| SEQ | cg27603605_chr12_ | chr12 | 126002485 | 126002556 | chr12q | cg27603605 |
| ID | 126002520 | | | | | |
| 299 | | | | | | |
| SEQ | cg10191005_chr14_ | chr14 | 102022911 | 102022982 | chr14q | cg10191005 |
| ID | 102022946 | | | | | |
| 300 | | | | | | |
| SEQ | cg11204152_chr14_ | chr14 | 72638659 | 72638730 | chr14q | cg11204152 |
| ID | 72638694 | | | | | |
| 301 | | | | | | |
| SEQ | cg15320156_chr14_ | chr14 | 97409269 | 97409340 | chr14q | cg15320156 |
| ID | 97409304 | | | | | |
| 302 | | | | | | |
| SEQ | cg05989248_chr15_ | chr15 | 100530615 | 100530686 | chr15q | cg05989248 |
| ID | 100530650 | | | | | |
| 303 | | | | | | |
| SEQ | cg06851885_chr15_ | chr15 | 84588876 | 84588947 | chr15q | cg06851885 |
| ID | 84588911 | | | | | |
| 304 | | | | | | |
| SEQ | cg07273980_chr15_ | chr15 | 81718778 | 81718849 | chr15q | cg07273980 |
| ID | 81718813 | | | | | |
| 305 | | | | | | |
| SEQ | cg08484383_chr15_ | chr15 | 80527983 | 80528054 | chr15q | cg08484383 |
| ID | 80528018 | | | | | |
| 306 | | | | | | |
| SEQ | cg09783969_chr15_ | chr15 | 100885966 | 100886037 | chr15q | cg09783969 |
| ID | 100886001 | | | | | |
| 307 | | | | | | |
| SEQ | cg17135920_chr15_ | chr15 | 94498977 | 94499048 | chr15q | cg17135920 |
| ID | 94499012 | | | | | |
| 308 | | | | | | |
| SEQ | cg25624874_chr15_ | chr15 | 92248328 | 92248399 | chr15q | cg25624874 |
| ID | 92248363 | | | | | |
| 309 | | | | | | |
| SEQ | cg04257915_chr17_ | chr17 | 11464392 | 11464463 | chr17p | cg04257915 |
| ID | 11464427 | | | | | |
| 310 | | | | | | |
| SEQ | cg05692077_chr17_ | chr17 | 9929997 | 9930068 | chr17p | cg05692077 |
| ID | 9930032 | | | | | |
| 311 | | | | | | |
| SEQ | cg22446777_chr17_ | chr17 | 33088658 | 33088729 | chr17q | cg22446777 |
| ID | 33088693 | | | | | |
| 312 | | | | | | |
| SEQ | cg05519376_chr18_ | chr18 | 5901049 | 5901120 | chr18p | cg05519376 |
| ID | 5901084 | | | | | |
| 313 | | | | | | |
| SEQ | cg10431939_chr18_ | chr18 | 35072525 | 35072596 | chr18q | cg10431939 |
| ID | 35072560 | | | | | |
| 314 | | | | | | |
| SEQ | cg11467777_chr18_ | chr18 | 6368486 | 6368557 | chr18p | cg11467777 |
| ID | 6368521 | | | | | |
| 315 | | | | | | |
| SEQ | cg24680171_chr18_ | chr18 | 44015495 | 44015566 | chr18q | cg24680171 |
| ID | 44015530 | | | | | |
| 316 | | | | | | |
| SEQ | cg25704768_chr18_ | chr18 | 11757290 | 11757361 | chr18p | cg25704768 |
| ID | 11757325 | | | | | |
| 317 | | | | | | |
| SEQ | cg20006624_chr19_ | chr19 | 53789914 | 53789985 | chr19q | cg20006624 |
| ID | 53789949 | | | | | |
| 318 | | | | | | |
| SEQ | cg22561329_chr19_ | chr19 | 57346699 | 57346770 | chr19q | cg22561329 |
| ID | 57346734 | | | | | |
| 319 | | | | | | |
| SEQ | cg00300216_chr2_ | chr2 | 6992664 | 6992735 | chr2p | cg00300216 |
| ID | 6992699 | | | | | |
| 320 | | | | | | |
| SEQ | cg01933248_chr2_ | chr2 | 418537 | 418608 | chr2p | cg01933248 |
| ID | 418572 | | | | | |
| 321 | | | | | | |
| SEQ | cg02337413_chr2_ | chr2 | 222708817 | 222708888 | chr2q | cg02337413 |
| ID | 222708852 | | | | | |
| 322 | | | | | | |
| SEQ | cg08970156_chr2_ | chr2 | 227947410 | 227947481 | chr2q | cg08970156 |
| ID | 227947445 | | | | | |
| 323 | | | | | | |
| SEQ | cg11033909_chr2_ | chr2 | 4875525 | 4875596 | chr2p | cg11033909 |
| ID | 4875560 | | | | | |
| 324 | | | | | | |
| SEQ | cg11742722_chr2_ | chr2 | 31352385 | 31352456 | chr2p | cg11742722 |
| ID | 31352420 | | | | | |
| 325 | | | | | | |
| SEQ | cg15020921_chr2_ | chr2 | 23436236 | 23436307 | chr2p | cg15020921 |
| ID | 23436271 | | | | | |
| 326 | | | | | | |
| SEQ | cg15328937_chr2_ | chr2 | 7212053 | 7212124 | chr2p | cg15328937 |
| ID | 7212088 | | | | | |
| 327 | | | | | | |
| SEQ | cg17586290_chr2_ | chr2 | 7247095 | 7247166 | chr2p | cg17586290 |
| ID | 7247130 | | | | | |
| 328 | | | | | | |
| SEQ | cg25995816_chr2_ | chr2 | 21539454 | 21539525 | chr2p | cg25995816 |
| ID | 21539489 | | | | | |
| 329 | | | | | | |
| SEQ | cg01416395_chr20_ | chr20 | 55806397 | 55806468 | chr20q | cg01416395 |
| ID | 55806432 | | | | | |
| 330 | | | | | | |
| SEQ | cg08041987_chr20_ | chr20 | 58250492 | 58250563 | chr20q | cg08041987 |
| ID | 58250527 | | | | | |
| 331 | | | | | | |
| SEQ | cg09010674_chr20_ | chr20 | 38659531 | 38659602 | chr20q | cg09010674 |
| ID | 38659566 | | | | | |
| 332 | | | | | | |
| SEQ | cg10249285_chr20_ | chr20 | 22795649 | 22795720 | chr20p | cg10249285 |
| ID | 22795684 | | | | | |
| 333 | | | | | | |
| SEQ | cg04556646_chr22_ | chr22 | 45310542 | 45310613 | chr22q | cg04556646 |
| ID | 45310577 | | | | | |
| 334 | | | | | | |
| SEQ | cg17584604_chr22_ | chr22 | 43705242 | 43705313 | chr22q | cg17584604 |
| ID | 43705277 | | | | | |
| 335 | | | | | | |
| SEQ | cg23059285_chr22_ | chr22 | 40121921 | 40121992 | chr22q | cg23059285 |
| ID | 40121956 | | | | | |
| 336 | | | | | | |
| SEQ | cg03383322_chr3_ | chr3 | 123094614 | 123094685 | chr3q | cg03383322 |
| ID | 123094649 | | | | | |
| 337 | | | | | | |
| SEQ | cg04791901_chr3_ | chr3 | 1293023 | 1293094 | chr3p | cg04791901 |
| ID | 1293058 | | | | | |
| 338 | | | | | | |
| SEQ | cg06916161_chr3_ | chr3 | 56468266 | 56468337 | chr3p | cg06916161 |
| ID | 56468301 | | | | | |
| 339 | | | | | | |
| SEQ | cg15428258_chr3_ | chr3 | 63391664 | 63391735 | chr3p | cg15428258 |
| ID | 63391699 | | | | | |
| 340 | | | | | | |
| SEQ | cg15739772_chr3_ | chr3 | 163497467 | 163497538 | chr3q | cg15739772 |
| ID | 163497502 | | | | | |
| 341 | | | | | | |
| SEQ | cg17817976_chr3_ | chr3 | 6573767 | 6573838 | chr3p | cg17817976 |
| ID | 6573802 | | | | | |
| 342 | | | | | | |
| SEQ | cg06507260_chr4_ | chr4 | 7531061 | 7531132 | chr4p | cg06507260 |
| ID | 7531096 | | | | | |
| 343 | | | | | | |
| SEQ | cg17322397_chr4_ | chr4 | 185065367 | 185065438 | chr4q | cg17322397 |
| ID | 185065402 | | | | | |
| 344 | | | | | | |
| SEQ | cg06772654_chr5_ | chr5 | 38048811 | 38048882 | chr5p | cg06772654 |
| ID | 38048846 | | | | | |
| 345 | | | | | | |
| SEQ | cg11180210_chr5_ | chr5 | 169787977 | 169788048 | chr5q | cg11180210 |
| ID | 169788012 | | | | | |
| 346 | | | | | | |
| SEQ | cg12216397_chr5_ | chr5 | 170020876 | 170020947 | chr5q | cg12216397 |
| ID | 170020911 | | | | | |
| 347 | | | | | | |
| SEQ | cg13721576_chr5_ | chr5 | 166730684 | 166730755 | chr5q | cg13721576 |
| ID | 166730719 | | | | | |
| 348 | | | | | | |
| SEQ | cg14045305_chr5_ | chr5 | 179545078 | 179545149 | chr5q | cg14045305 |
| ID | 179545113 | | | | | |
| 349 | | | | | | |
| SEQ | cg23683507_chr5_ | chr5 | 117931659 | 117931730 | chr5q | cg23683507 |
| ID | 117931694 | | | | | |
| 350 | | | | | | |
| SEQ | cg27629673_chr5_ | chr5 | 7462820 | 7462891 | chr5p | cg27629673 |
| ID | 7462855 | | | | | |
| 351 | | | | | | |
| SEQ | cg07436074_chr6_ | chr6 | 162071104 | 162071175 | chr6q | cg07436074 |
| ID | 162071139 | | | | | |
| 352 | | | | | | |
| SEQ | cg10988349_chr6_ | chr6 | 51861910 | 51861981 | chr6p | cg10988349 |
| ID | 51861945 | | | | | |
| 353 | | | | | | |
| SEQ | cg16305062_chr7_ | chr7 | 124716979 | 124717050 | chr7q | cg16305062 |
| ID | 124717014 | | | | | |
| 354 | | | | | | |
| SEQ | cg18929226_chr7_ | chr7 | 4207508 | 4207579 | chr7p | cg18929226 |
| ID | 4207543 | | | | | |
| 355 | | | | | | |
| SEQ | cg27230333_chr7_ | chr7 | 50266240 | 50266311 | chr7p | cg27230333 |
| ID | 50266275 | | | | | |
| 356 | | | | | | |
| SEQ | cg25184152_chr8_ | chr8 | 20831250 | 20831321 | chr8p | cg25184152 |
| ID | 20831285 | | | | | |
| 357 | | | | | | |

Table 16B

| SEQ ID | CpG | CpG | Sequence |
|---|---|---|---|
| No. | begin | end | (5′ to 3′) |
| SEQ ID | 3104041 | 3104042 | GGTCCTGTGTCTTGCCCACC |
| 283 | | | TGCTCTCCTGGTGGC[CG]T |
| | | | GGCTCTGGAGAAGTCCCCAG |
| | | | CCAGGTCCATGCTC |
| SEQ ID | 154721908 | 154721909 | TGCAGCCTCACCTAGGCAGG |
| 284 | | | GTTAGTGTGGGAAGG[CG]T |
| | | | GGGAATCACCCTGTGACCAA |
| | | | GAACAAAGAGGAAC |
| SEQ ID | 132281536 | 132281537 | TCCTCTCATATTCTAAATAG |
| 285 | | | CTGAGAAACAGCCTA[CG]T |
| | | | GCAGGTCAGTTGCACTGCAC |
| | | | TGTGTGTGATAGTG |
| SEQ ID | 20071729 | 20071730 | TTAACAGTAAAAATTCAACT |
| 286 | | | TCCTAACACTGGCCC[CG]T |
| | | | GAACATCTACATGTTCATTC |
| | | | CATTCTCATCCTCT |
| SEQ ID | 19886805 | 19886806 | ACACAGCCAAACTTGGAAAG |
| 287 | | | ACAAATAGTCATTGG[CG]A |
| | | | ATAAAGCAGAGATCTGGATT |
| | | | CAAGTGAAGTGAAG |
| SEQ ID | 130093396 | 130093397 | AACTTCCATTTCCTCAGTGG |
| 288 | | | CAGTTAACCACATTC[CG]T |
| | | | GCTCAGCACAGAGTATTTTT |
| | | | CTTATTGCAGAAAG |
| SEQ ID | 20164045 | 20164046 | AAGGTGCAAAAATTAAATCA |
| 289 | | | TGCATGCAAAGCAGT[CG]T |
| | | | AGGTGCTCCATAGTATGTGG |
| | | | TTAGCCTTATAATG |
| SEQ ID | 50329827 | 50329828 | AGGTCTGTCAGGACTCCACC |
| 290 | | | ATTTTGACATGACCC[CG]T |
| | | | TTTCCCCCACAATCCCCCTT |
| | | | CCAGGACCCCATTG |
| SEQ ID | 130277393 | 130277394 | GGGGTGGAAATGGTCAGGGT |
| 291 | | | AGACCCAAGAGAGCA[CG]A |
| | | | TGCCTGGATGATCAGTTTTT |
| | | | GTTAGTCAGTAGTT |
| SEQ ID | 124480989 | 124480990 | AAAGACTACTATGTAGGGTA |
| 292 | | | GGCAATCCCAGCTGGG[CG] |
| | | | TGGGACTCCATTCCCACTCC |
| | | | AAACCACAAAATGA |
| SEQ ID | 45699392 | 45699393 | AGCATCCTACAGCCCCACAA |
| 293 | | | GTACAGGCCCTTGTT[CG]A |
| | | | ATGTGTCTTACAAAAAGGAA |
| | | | TAAATGAAAATAAG |
| SEQ ID | 110843044 | 110843045 | TGAGCCATGGCACTTTTCCC |
| 294 | | | AATTCAATTTTCACT[CG]A |
| | | | AAACTCAAAGTGAGATAATT |
| | | | GCCTAGGCAAAACT |
| SEQ ID | 133913867 | 133913868 | GGCCCAGGTTGGGGGAAGCT |
| 295 | | | CCTCCACCAACCTGT[CG]T |
| | | | GAGCCATGCCCCTCCAGTCC |
| | | | ATCTGCTCCCACTC |
| SEQ ID | 84881753 | 84881754 | CACAGGTGGTAAAAAGAATT |
| 296 | | | TACCAAGACAGCTGT[CG]T |
| | | | AAAGAAAGGCAGGTTTGAGA |
| | | | AAGTAGGAAAATGC |
| SEQ ID | 99227052 | 99227053 | CGAGTGGTTAAGTCACCTAC |
| 297 | | | CCAAGAGCCAGCATG[CG]T |
| | | | GGCTCTGGGATTTGAATCAG |
| | | | ATTTGCCTGATTCC |
| SEQ ID | 117747406 | 117747407 | TTCACTGCAATGCAGAGGAT |
| 298 | | | GGGTTTGAAATTCAC[CG]A |
| | | | TTCCCTAGGGTTGCCCTGGC |
| | | | CTGGCCCATCAGCT |
| SEQ ID | 126002520 | 126002521 | TAAATTTGATTTATTTTTAA |
| 299 | | | ATTATTTTAATTTGC[CGTT |
| | | | AAATGGCCATTTGTGGCTGG |
| | | | TGGCCACAATATTG |
| SEQ ID | 102022946 | 102022947 | CTGGAAAGTCACCACCCAAC |
| 300 | | | CCACTCCTGATGCAG[CG]A |
| | | | GACCTGAGGAAGGGGCCAGA |
| | | | GATGCACAGGGTCA |
| SEQ ID | 72638694 | 72638695 | AGCTGAACTCTTAACCACAC |
| 301 | | | TGCTCTCCTGCAGGG[CG]A |
| | | | TGAGCTTGCCATGCCTCTTG |
| | | | GTCATTCCCTAAGG |
| SEQ ID | 97409304 | 97409305 | AGGGCATTTCAGCAGCATAC |
| 302 | | | TCAAGATTCTACAGA[CG]A |
| | | | CTAAGTAGCAGAGCCACAGT |
| | | | TTGAACCCAGGCAG |
| SEQ ID | 100530650 | 100530651 | ATACTAAGCTTTATTAACAT |
| 303 | | | CCAAGTAACTGTGTG[CG]T |
| | | | CCCTGTTTGGTTTTGGGGAA |
| | | | ACTGGACTGACAGC |
| SEQ ID | 84588911 | 84588912 | TAGTGGAGTACAAGAATTCC |
| 304 | | | TTTCTACAAATGGTA[CG]T |
| | | | GGGAACAAAGATTGCATTGG |
| | | | CCCACTATGGGCTC |
| SEQ ID | 81718813 | 81718814 | TTTATACCCAGTGATTCTGA |
| 305 | | | AGAAGGCAATAGAAC[CG]T |
| | | | GTGAGGAAAATGTAAAGGCA |
| | | | CCCTGCAATGTGGC |
| SEQ ID | 80528018 | 80528019 | CCTGGGCTGTTGCTCTTGGC |
| 306 | | | TCCATAAAGTTCTTA[CG]T |
| | | | GTAGTTCTGTAGTTATGACC |
| | | | CAGAACCAACTCCC |
| SEQ ID | 100886001 | 100886002 | TTGCTATTTGGGTTGTCTGT |
| 307 | | | TATATGCAGCCAAAC[CG]A |
| | | | CCCCTAACAGACACACATAT |
| | | | AGACAACTCCCATC |
| SEQ ID | 94499012 | 94499013 | CCCCTAGGGTTCTTAAAAGG |
| 308 | | | ATTCTATGAGTTATT[CG]T |
| | | | TGAAAGGGTTTGAATGAGTA |
| | | | CTGACCCATAGTAA |
| SEQ ID | 92248363 | 92248364 | GATAGCCTGCTGGTCCTAGG |
| 309 | | | AGAAGTATCAGAAGC[CG]T |
| | | | GGAGCAGAGCCACACCAGCC |
| | | | CTGTTGCAGATCCA |
| SEQ ID | 11464427 | 11464428 | ATGGAACAAGCAAAGCCACA |
| 310 | | | TCAATAGGCAAGTTC[CG]T |
| | | | AGCAGATAAAAGAGGCTTCT |
| | | | GGGGCTGGAACCTA |
| SEQ ID | 9930032 | 9930033 | GACCCAGCAGGGCTGGAGAC |
| 311 | | | TGGCAATTCACTCCC[CG]T |
| | | | CATGCCTTCCTGGTGGACAC |
| | | | CTGTTTAGGTGGGC |
| SEQ ID | 33088693 | 33088694 | CCTGGGTTCAAATCCCAGAG |
| 312 | | | TTGCCCTTTCTAGCC[CG]T |
| | | | GACCTCTGGGGAGCCACTTC |
| | | | ACCTCTCCAGGTGT |
| SEQ ID | 5901084 | 5901085 | GCAGCTAAGTGTGCCATTGA |
| 313 | | | CAGAGATGGTAAGAA[CG]T |
| | | | AGAGTGGGAAGGGGCCTTAA |
| | | | GGTACTTAATGCTC |
| SEQ ID | 35072560 | 35072561 | TTCCTGGTACCTTTTGAAGC |
| 314 | | | AGATGTTCTGCTGCC[CG]T |
| | | | GAGAGAGAGGCAGCTACAGA |
| | | | GCAGCTCATCATGT |
| SEQ ID | 6368521 | 6368522 | CCAAGGTCCCTGCTAAGCAC |
| 315 | | | TTTCCATGCATTAAC[CG]T |
| | | | GGAACTTCAAGACAACCCTG |
| | | | AGGTATAGGTATTA |
| SEQ ID | 44015530 | 44015531 | TCTGCTCCCAGCCACCCTCT |
| 316 | | | GGGCCAGATGGTCCC[CG]T |
| | | | GAGCCTGGTTCTAGCAATTA |
| | | | GCTCAGATATTACT |
| SEQ ID | 11757325 | 11757326 | ATCATCAGCCTTACAGGCCA |
| 317 | | | GGTGTGTCCAGACAC[CG]A |
| | | | AGCTTTGGAGGGTTCTAAGC |
| | | | AGTGGAGCCATGAG |
| SEQ ID | 53789949 | 53789950 | AAAGGGTTTCCCAGATACAG |
| 318 | | | AAGTTACACTCCAGC[CG]T |
| | | | TGTGTTTAGTACACTCTGGT |
| | | | TTGTCTATGAGCTC |
| SEQ ID | 57346734 | 57346735 | CTTACCTTCTTCCTACCTCA |
| 319 | | | ATCAGATGCCACTCA[CG]A |
| | | | TTCCCTTGCTCTAGGAATCC |
| | | | TGGATTTTCAGCTC |
| SEQ ID | 6992699 | 6992700 | ACTGTTTTCTCCTCTGTGCT |
| 320 | | | CTCAAAACCCTTTCT[CG]T |
| | | | GACTCTACTGAAAAACTCCT |
| | | | CATTGCAAATCAGA |
| SEQ ID | 418572 | 418573 | TTATAGAAAAGCAATATATT |
| 321 | | | TTGTAAAATGAATGA[CG]A |
| | | | ATGCTTCCATGTATCCAGGA |
| | | | AGAGTACTGTGTCC |
| SEQ ID | 222708852 | 222708853 | GATATCAATTCAAAGTCCCA |
| 322 | | | AATCTCATCTAAATC[CG]T |
| | | | CACTTCAAAAGTCCAAAGTC |
| | | | TCCTTGTCTCAGTC |
| SEQ ID | 227947445 | 227947446 | AGGGATAAGTTTGTGATGAA |
| 323 | | | AAAGGCATGGAAGTG[CG]T |
| | | | CCTGCTAAGGAAAGTTGATG |
| | | | AGCAGGAGAAGAGG |
| SEQ ID | 4875560 | 4875561 | TAAACAGTGTGATAAATTGT |
| 324 | | | GTGATTTAGTTCTGC[CG]T |
| | | | GGAGGAGAATATTCACCTGT |
| | | | GAGTAAGCAGGTAG |
| SEQ ID | 31352420 | 31352421 | CCAATTATCTGGGTGCCTTA |
| 325 | | | ATTAATCCACAGACC[CG]T |
| | | | GGCCTGATCTCCCTGAGATC |
| | | | CTAGGAAACAATAA |
| SEQ ID | 23436271 | 23436272 | GCATGAGGGATGTAAAGGTG |
| 326 | | | CATTGGAGATGATTT[CG]A |
| | | | TCAGCATTCTTTAAGATGTT |
| | | | GTTTACAAAGGCAA |
| SEQ ID | 7212088 | 7212089 | GAAATTCCTCCTGGAACTCC |
| 327 | | | AGTGTCTGCTCCTAC[CG]A |
| | | | CAGGCTCCAGCCCACCCTAA |
| | | | GGATTTTGGATTTG |
| SEQ ID | 7247130 | 7247131 | GGTTGTCCTAGAGATGCTGC |
| 328 | | | AGCTGTTGGCTGTGA[CG]T |
| | | | GGCTTACTCCATGTACAGGT |
| | | | GAATGTCAGAGATT |
| SEQ ID | 21539489 | 21539490 | GTTTCCAGTTGCCCTTCACA |
| 329 | | | CTGACTCTCCTTGGC[CG]T |
| | | | TGCTGCTGATGGGTCCATCC |
| | | | TTGGCCTACTTACC |
| SEQ ID | 55806432 | 55806433 | CTCTGAAAGCAGTGCTGCTA |
| 330 | | | TGAACATCACAGGAC[CG]T |
| | | | GTTTCATGCCTAGAAGTGGC |
| | | | ATTGTGCATTGCAG |
| SEQ ID | 58250527 | 58250528 | CAGGGGGCAACTACCTCTTC |
| 331 | | | ATAGCAAAGCTTCAT[CG]T |
| | | | TAAGTTCCTGGTTCTGGGCT |
| | | | ATTGTCCCTGTCTC |
| SEQ ID | 38659566 | 38659567 | TTTCAGGTCATTAAGGGCTT |
| 332 | | | TACTTATTTTGAATG[CG]T |
| | | | TTATTTTGACAACAATTAAT |
| | | | GGGTTTTGAGCAGA |
| SEQ ID | 22795684 | 22795685 | GCAGCTGGAGGAGATGGGAA |
| 333 | | | GGTGCAGGTTTGCCC[CG]T |
| | | | GATCTGCAGCACACAAGATC |
| | | | TGTGCCAGGGACTG |
| SEQ ID | 45310577 | 45310578 | ACATTCTATTTTTTTTCACT |
| 334 | | | GCCATGAGGCCCCTC[CG]T |
| | | | GGTGGATGGGGAAGGGGAAG |
| | | | GGGGTCTTCAGATG |
| SEQ ID | 43705277 | 43705278 | CTAGGTACTATGGTATGTGT |
| 335 | | | TTTACAAAGCTCATC[CG]T |
| | | | TGGCCTCTGCATCATCTCTG |
| | | | TCAAATAAGCACTG |
| SEQ ID | 40121956 | 40121957 | ACTGAAGTATGCATATGGAG |
| 336 | | | TTAGGTGTGCTTATG[CG]T |
| | | | GACTCAACTGTGTGTGGGTA |
| | | | GCAAGATCCATGTC |
| SEQ ID | 123094649 | 123094650 | GCAAGTGGATAGCTGAAAGG |
| 337 | | | CTGGGCAGAGTGACC[CG]A |
| | | | GGGCCTCATTTAGCCCTGGG |
| | | | TAGTGAATGCCTGT |
| SEQ ID | 1293058 | 1293059 | CAGCAATACTTTGACTCTGC |
| 338 | | | TAGATCCTATAATTC[CG]A |
| | | | ATCCTAACAACTACTCCTGT |
| | | | CCTTCTCCTGCTTC |
| SEQ ID | 56468301 | 56468302 | CCTTCTTGATGATGCCAAAC |
| 339 | | | TTTCTTCTGCACAGG[CG]T |
| | | | GGTACCATCTGCAAAGCATC |
| | | | AACTACTCAGTGAG |
| SEQ ID | 63391699 | 63391700 | ATTCAGTTTATTCTTACTGT |
| 340 | | | CCTGTAGAGAGGACA[CG]A |
| | | | GGATCAGAGAGGTTCAGTTT |
| | | | CTTGCCCAGAATCA |
| SEQ ID | 163497502 | 163497503 | GGAAGGCAGAAGTGGGTGTG |
| 341 | | | GAGGTTTCCCATGAG[CG]T |
| | | | TGGCTTATGTGATGCTTAAT |
| | | | TTTAGGTGACAACT |
| SEQ ID | 6573802 | 6573803 | AAGTTAAAAGGATGGTGAAG |
| 342 | | | ATAAGCATAGAAAGA[CG]A |
| | | | GGTTTGGCTAAGTAAAGGTT |
| | | | AAAGTTAAGGCTTG |
| SEQ ID | 7531096 | 7531097 | CATTTGATGCTGTTGTATTT |
| 343 | | | TTGCTTCTTTCCTTA[CG]T |
| | | | CCATCTGCCTCCTTCCATCT |
| | | | CCCCTCCTAGAACA |
| SEQ ID | 185065402 | 185065403 | TAATTTAATATGTGGGTACC |
| 344 | | | TACCTGGAGCCCTCT[CG]T |
| | | | TACTTTGCCAGGACTCCTCC |
| | | | CTCCAAATCTACCA |
| SEQ ID | 38048846 | 38048847 | CATGAGATGGGAGGAGCTTG |
| 345 | | | AGTAACTGAATGACC[CG]T |
| | | | GGAGCAGAGCCTGTCAGCCT |
| | | | CAAACACACTGTAC |
| SEQ ID | 169788012 | 169788013 | CCTGTGCTGGAGTTTGACAG |
| 346 | | | CAGTGACCAGCCAGA[CG]A |
| | | | CCTGGATGAGACAAGGGTCA |
| | | | GTGCAAACAAGACC |
| SEQ ID | 170020911 | 170020912 | AGAAAAAGAAGAGGATGCCT |
| 347 | | | GAGGTGGTGGGAAGA[CG]T |
| | | | AGGCTCTAGCTTCAGGTGAG |
| | | | CTTGGAAAAGTCAG |
| SEQ ID | 166730719 | 166730720 | GTGGGTCTGTATCTCCTTTT |
| 348 | | | CAATGTGAATATGTA[CG]A |
| | | | GACTATGAATAGCTAAGTAA |
| | | | AGGTGAAAAGTCCC |
| SEQ ID | 179545113 | 179545114 | TAAATGTGATCTGAGGCCAC |
| 349 | | | ATAAATAAAAGTATT[CG]T |
| | | | TTAGAATCAGGGAGGTGGAA |
| | | | GATCCTGTGTACCT |
| SEQ ID | 117931694 | 117931695 | CACACAGCCTCTCACAGTGG |
| 350 | | | TGTGGCCTGGACACC[CG]T |
| | | | TTCCTTCTCCTTTCTCAGGC |
| | | | TGCCCTATTCTTGG |
| SEQ ID | 7462855 | 7462856 | TTTATTTTAGTTCTTTTTCA |
| 351 | | | GTGTCAGGTGCTCAT[CG]T |
| | | | GGTGTAAATAACAATTCTGT |
| | | | GTTAGGCAGGTTTT |
| SEQ ID | 162071139 | 162071140 | CAGTCCCCAGAGGTCAAGTT |
| 352 | | | ATCTCAACCTACAGG[CG]T |
| | | | TCCAGATGATAACCCAGTAA |
| | | | TTTTGCAACAAAGG |
| SEQ ID | 51861945 | 51861946 | TGTGCTCATGAAAGACCCTT |
| 353 | | | TCATTCCCATGTGAT[CG]A |
| | | | ATAGGAAAGCAAGTAGGCCT |
| | | | AGAAGCTACTGACA |
| SEQ ID | 124717014 | 124717015 | GGGAATAATTTTGAAGAGTA |
| 354 | | | TAGGAAAATGATGAC[CG]A |
| | | | GAGAGGGGATAATTGTTAGA |
| | | | CTGATATCCTTGAG |
| SEQ ID | 4207543 | 4207544 | AGCCCAAGCTTGTACTGCAA |
| 355 | | | GGTGGCTGCAAGGCC[CG]A |
| | | | CCCAAATCTAGAGCCTGACC |
| | | | TTGACCTCATGGGT |
| SEQ ID | 50266275 | 50266276 | GAAAGTGTGCTCAGAGGTTT |
| 356 | | | GGATAATGCTCAAAC[CG]T |
| | | | AGCTTGGGTTTGAATTCTCA |
| | | | AAGAAAGTGCTTAA |
| SEQ ID | 20831285 | 20831286 | TGTCTCATTGAAACACATTG |
| 357 | | | CTCATTTATTCCTCT[CG]T |
| | | | CATCCTTTGAGACACAGTCA |
| | | | TTATTTTCCAGATG |

WGBS means Whole-Genome Bisulfite Sequencing as recognized in the art (6).

“TCGA” as referred to herein, means The Cancer Genome Atlas (TCGA). TCGA is supervised by the National Cancer Institute's Center for Cancer Genomics and the National Human Genome Research Institute funded by the US government. A three-year pilot project, begun in 2006, focused on characterization of three types of human cancers: glioblastoma multiforme, lung, and ovarian cancer. In 2009, it expanded into phase II, which planned to complete the genomic characterization and sequence analysis of 20-25 different tumor types by 2014. TCGA surpassed that goal, characterizing 33 cancer types including 10 rare cancers.

“Hi-C-defined heterochromatic compartment B” as used herein is as recognized in the art, for example, by Fortin, J.-P. & Hansen, K. D. (7).

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutations of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Although methods and materials similar or equivalent to those described herein can be used in the practice or testing of this disclosure, suitable methods and materials are described below. The term “comprises” means “includes.” The abbreviation, “e.g.” is derived from the Latin exempli gratis, and is used herein to indicate a non-limiting example. Thus, the abbreviation “e.g.” is synonymous with the term “for example.”

The present methods and systems may be understood more readily by reference to the following detailed description of preferred embodiments and the Examples included therein and to the Figures and their previous and following description.

### Example 1

(Solo-WCGW CpGs were Shown to be Prone to Hypomethylation)

This example describes definition and use of a Solo-WCGW sequence motif having substantial utility for measuring genomic DNA methylation loss.

TCGA tumors and adjacent normal samples were sequenced using paired-end WGBS at ˜15× sequence depth, to compile a set of 40 core tumor samples and 9 core normal samples (FIGS. 30-1 to 30-16 (Table 1) and working Example 8 below).

A set of shared PMDs and HMDs was initially defined across the majority of our 49 core sample set using an existing Hidden Markov Model-based (HMM-based) method, MethPipe27 (FIG. 9A; working Example 8 below). Previous studies have suggested that DNA methylation is associated with local sequence context, including local CpG density (28, 29) and nucleotides directly flanking the CpG (29). The shared MethPipe PMD set (excluding CpG islands) was used to determine local CpG density and tetranucleotide sequence contexts most predictive of DNA hypomethylation.

Specifically, FIGS. 9A-C show that using the solo-WCGW sequence motif a set of shared PMDs and HMDs was initially defined across the majority of the 49 core sample set using an existing Hidden Markov Model-based (HMM-based) method, MethPipe27. FIG. 9A shows PMD calls by methpipe on tumor and adjacent normal samples reported in this study (left) and cutoff for choosing shared MethPipe PMDs (Note that this only used here and in FIG. 1, the definition of PMDs were updated later based on cross tumor SDs) from these methpipe calls (right). FIG. 9B shows a Receiver Operating Characteristic (ROC) curve showing prediction power of hypomethylation tendency with different sizes of the sequence window in defining Solo-CpGs in human (N=26,752,698 CpGs). FIG. 9C shows methylation average of CpG dinucleotides in 10 tetranucleotide sequence context stratified by neighboring CpG number and genomic territory (PMD or HMD). Each panel includes 390 WGBS samples.

Low CpG density within windows of about +/−35 bp was found to be optimal for predicting PMD-specific hypomethylation (FIG. 9B). Additionally, CpGs flanked by an A or T (“W”) on both sides (WCGW tetranucleotides) were consistently more prone to DNA hypomethylation than those flanked by a C or G (“S”) on either (SCGW) or both (SCGS) sides (FIG. 1A; FIG. 9C). In colon tumors and adjacent normal tissues, low CpG density and the WCGW context contributed additively to hypomethylation (FIG. 1B, upper). The most hypomethylation-prone sequence context was at CpGs with the combination of zero neighboring CpGs (“solo”) and the WCGW motif. In two adjacent normal colon samples, only these solo-WCGW CpGs showed significant hypomethylation (FIG. 1B, upper). These same sequence dependencies were apparent in a colorectal tumor and normal colon tissue from mice (FIG. 1B, lower). Moreover, they were consistent within all other tumor and adjacent normal samples in the core set, using either the WGBS data (FIG. 10A1-A 3) or matched Illumina Infinium HumanMethylation450™ (HM450) microarray data (FIG. 10B1-B2). An additional 390 human and 206 mouse WGBS samples examined later exhibited the same pattern (FIGS. 11A and 11B), with the exception of three germ cell samples (FIG. 11C).

Specifically, FIGS. 10A1-A3 and B1-B2 show that the same sequence dependencies shown in FIG. 9, were consistent within all other tumor and adjacent normal samples in the core set, using either the WGBS data (FIG. 10A1-A3), or matched Illumina Infinium HumanMethylation450™ (HM450) microarray data (FIG. 10B1-B2). FIG. 10A 1-A3 shows Violin plots of CpG methylation in 24 sequence contexts for all 47 TCGA WGBS samples (39 tumors and 8 normals) reported in this study. Elements of the violin plots represent the DNA methylation beta value of each CpG. FIG. 10B1-B2 shows methylation distribution of CpGs in 24 sequence contexts from 27 matched HM450 data of the TCGA WGBS samples. Elements of the violin plots represents the DNA methylation beta value of each CpG.

Specifically, FIGS. 11A-C show that an additional 390 human and 206 mouse WGBS samples examined later exhibited the same hypomethylation pattern (FIG. 11A-B) as in FIGS. 9 and 10, with the exception of three germ cell samples (FIG. 11C). FIG. 11A shows methylation average of CpG dinucleotides in 24 sequence contexts (rows) of 390 WGBS samples; FIG. 11b shows methylation average of CpG dinucleotides in 24 sequence context (rows) of 206 mouse WGBS samples. FIG. 11c shows methylation distribution of CpG dinucleotides in 24 sequence contexts in one oocyte and two spermatozoa samples in human and in mouse respectively. N=26,752,698 CpGs for human and N=20,383,610 CpGs for mouse. Elements of the violin plots represent the DNA methylation beta value of each CpG in the specific sequence context.

Subsequent analyses were focused on solo-WCGWs, representing 13% of all CpGs in the human genome. While they represent only the extreme of a hypomethylation process that affects other CpGs, focusing on solo-WCGWs alone enhanced the signal of PMD/HMD structure, especially in normal adjacent tissues and weakly hypomethylated tumors such as COAD-3518 (FIG. 1C). The relatively shallow hypomethylation in COAD-3518 could not be attributed to a greater fraction of non-cancer cells in this sample, as the cancer cell fraction in this sample was estimated by molecular estimates (30; PMID 22544022) to be 80%, compared to 51% for the more strongly hypomethylated COAD-A00R; indicating that PMD depth was quantitative and driven by an independent property of the cancer cells.

Specifically, FIGS. 1A-C show that Solo-WCGW CpGs are prone to hypomethylation. In FIG. 1A, each genomic CpG dinucleotide was placed into one of four CpG density categories (0, 1, 2, or 3+, depending on the number of additional CpGs within a +/−35 bp window), and one of the three flanking nucleotide categories (SCGS, SCGW and WCGW, with “S” being C or G and “W” being A or T). Because CpGs are palindromic, WCGS and SCGW were combined. Each of the 4×3=12 possible contexts are shown as columns for CpGs within common HMDs (left) or common PMDs (right). In the illustrations, a star indicates the target CpGs, and solid circles indicate all neighboring CpGs within the window. The number of CpGs in each context is shown as a percentage of all genomic CpGs; for instance, the first column shows that 6% of all CpGs in the human genome are within HMDs, have 3+ flanking CpGs, and SCGS tetranucleotide context. The FIG. 1B Violin plots show beta value distributions for CpGs in each context, for five human tissues (two normal colon tissues and three colon tumors) and two mouse tissues (one normal colon tissue and one colon tumor). Violin color indicates mean beta value. Columns shaded orange and green indicate the most hypomethylation-resistant and most hypomethylation-prone categories, respectively. FIG. 1C shows average methylation values (non-overlapping 100-kb bins) across a 12-mb section of chr16p, for the human colon samples. Values were calculated using all CpGs (left), only hypomethylation resistant CpGs (orange, middle), or only Solo-WCGW CpGs (green, right). CpG islands were removed in all analyses.

In addition to enhancing the PMD/HMD signal in high coverage WGBS data, solo-WCGW CpGs allowed accurate PMD structure to be determined with average genomic read coverage as low as 0.05× in down-sampled bulk WGBS data (FIG. 12A), and in low-coverage single-cell WGBS data (31) (FIG. 12B), providing for an application for low coverage or single-cell WGBS studies.

Specifically, FIGS. 12A-B show that in addition to enhancing the PMD/HMD signal in high coverage WGBS data, solo-WCGW CpGs allowed accurate PMD structure to be determined with average genomic read coverage as low as 0.05× in down-sampled bulk WGBS data (FIG. 12A), and in low-coverage single-cell WGBS data (31) (FIG. 12B), providing for an application for low coverage or single-cell WGBS studies.

FIG. 12A is a heatmap showing DNA methylation beta value of chromosome 16p in 49 TCGA WGBS samples (40 tumors and 9 adjacent normal samples, including colorectal cancer and matched normal from Berman et al. 2012 Nature Genetics) downsampled from 1× to 0.01×. FIG. 12b is a heatmap showing DNA methylation beta value of chromosome 16p in 20 single-cell whole genome bisulfite sequencing (scWGBS) of HL60 cell line under vitamin D treatment as well as two bulk WGBS data sets of 50 ng (data from Farlik et al. 2015 Cell Reports, see also FIG. 29 (Table 1)).

### Example 2

(Most PMDs were Shown to be Shared Across Cancer and Normal Tissues)

Genomic plots of solo-WCGW CpG mean methylation revealed strong concordance between PMD locations in all samples in the core set (FIG. 2A). Comparing the average solo-WCGW methylation of the core tumors vs the core normal in multi-scale plots (FIG. 2B) confirmed that PMDs ranging from 100 kb to 5 mb (32) were mostly overlapping between tumors and normals, but less hypomethylated in the normals.

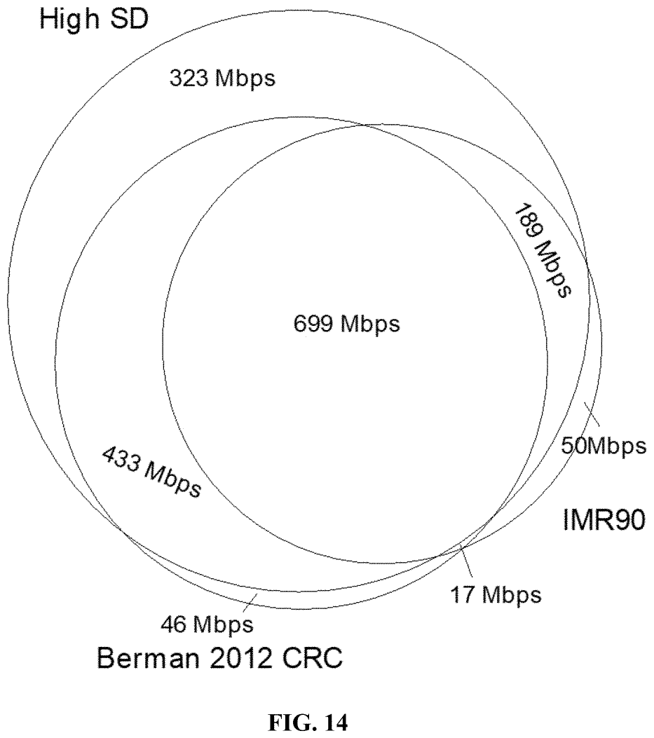

Given the high variability of solo-WCGW PMD hypomethylation across samples (FIG. 2A), the standard deviation (SD) of 100-kb bins across was compared across the core normal tissues and across core tumors, showing that PMDs had higher SD than HMDs within each group (FIG. 2C). Genome-wide, SD was bimodally distributed within 100-kb bins in both normal and tumor core groups (FIG. 2D), unlike mean methylation (FIG. 13) and all other features examined (not shown). While the highly variable nature of hypomethylation in PMDs has been noted previously (5, 7), it has not been used, or suggested for use as a method for identifying/characterizing PMDs. Using the bimodal SD peaks as a classifier resulted in a segmentation of the genome into HMDs and PMDs, with PMDs covering 63% of the genome in the core tumors (SD>0.125), and 66% of the genome in the core normals (SD>0.07). Strikingly, this method resulted in 100-kb bin classifications that were 83% concordant between the normal and tumor groups (FIG. 2D). These PMDs covered 95% of the base pairs in PMDs previously reported in colorectal cancer (6), and 93% of PMDs in the IMR90 fibroblast cell line (12) (FIG. 14). This SD-based classification of PMDs allowed for rescaling of methylation values for individual samples based on their sample-specific degree of PMD hypomethylation (FIGS. 2E-F), further illustrating the high degree of concordance in PMD/HMD structure across tumor and normal samples.

Specifically, FIGS. 2A-F show that most PMDs are shared across cancer and normal tissues. In FIG. 2A, average methylation values (non-overlapping 100-kb bins) for chr16p are shown for the core tumor/normal dataset. The “tumor” field indicates tumors (black) vs. adjacent normals, and “this study” field indicates samples that were newly sequenced as part of this study (black). Within both normal and tumor classes, tissue types are grouped and ordered by average methylation level of samples from the group. For instance, “endometrium” is the first normal group because it has the highest methylation among normal groups, and likewise for “GBM” among tumor groups. In FIG. 2B, average methylation across all normal (upper) or tumor samples (lower), was calculated for multiple window sizes from 10 kb to 10 mb (“multi-scale plot”). FIG. 2C shows standard deviation (SD) across all normal or tumor samples as multi-scale plots. FIG. 2D shows 100-kb SD values for the all non-overlapping genomic bins, plotted for tumors (red histogram, X-axis) vs. normals (blue histogram, Y-axis). Bimodal peaks for each were identified via a Gaussian mixture model, and cutoffs dividing low and high SD values are indicated by dashed lines for each axis. A scatter cloud shows the correlation between SD values between the tumors and normals, indicating the percentage of 100-kb bins falling into each of the four quadrants as well as Spearman's p. FIG. 2E shows an illustration of a method used to rescale each sample's methylation values based on genome-wide levels within a common set of PMDs (see working Example 8 herein). FIG. 2F shows the same data as FIG. 2A, but using rescaled methylation values.

Specifically, FIG. 13 shows that that there is an absence of bimodal distribution of cross-sample mean methylation for the core normal and tumor WGBS samples, whereas Genome-wide, SD was bimodally distributed within 100-kb bins in both normal and tumor core groups (FIG. 2D), unlike mean methylation (FIG. 13) and all other features examined (not shown).

Specifically, FIG. 14 shows that PMDs classified using the presently disclosed SD-based method covered 95% of the base pairs in PMDs previously reported in colorectal cancer (6), and 93% of PMDs in the IMR90 fibroblast cell line (12). FIG. 14 shows the overlap of PMD definition in this work with previous studies from colorectal cancer and IMR90 cell lines with overlapping area approximating numbers of overlapping base pairs.

### Example 3

(Most PMDs where Shown to be Shared Across Developmental Lineages)

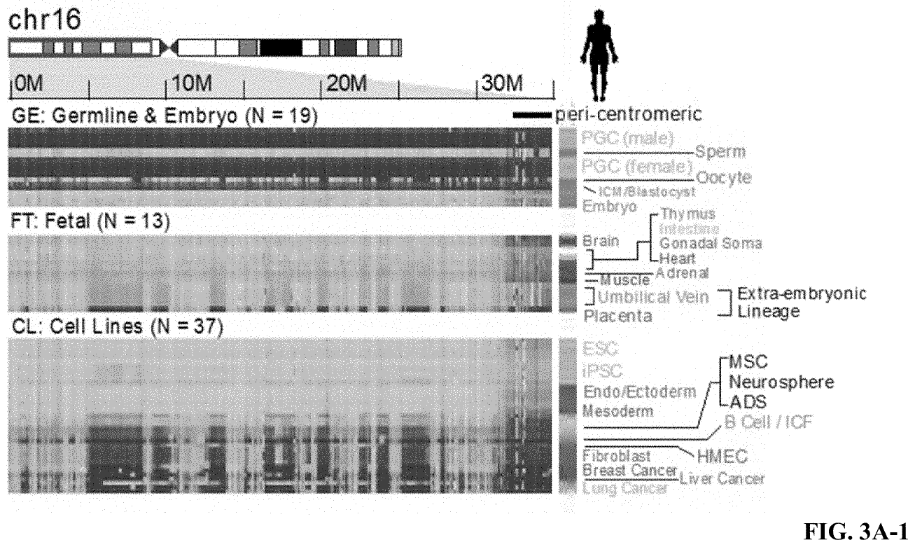

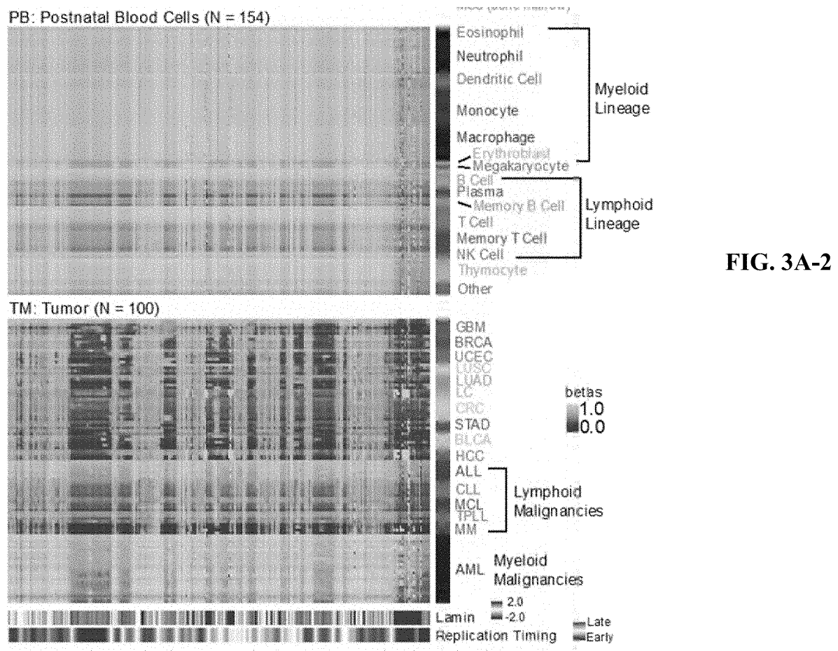

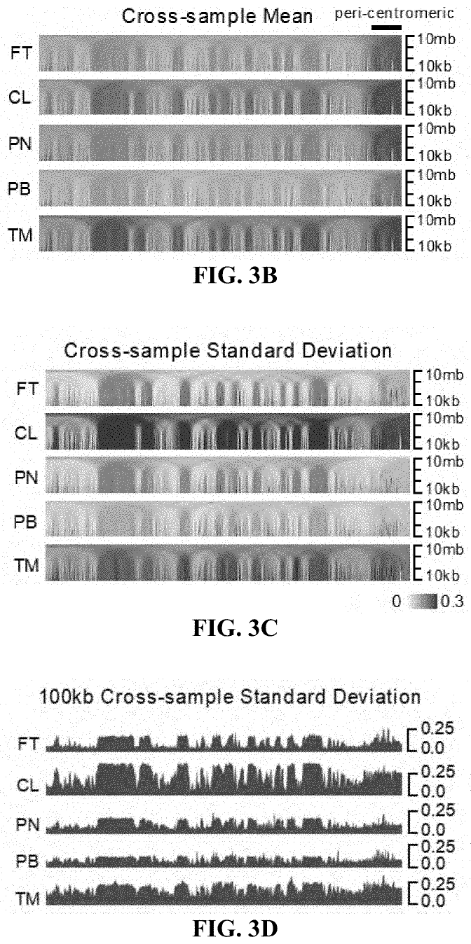

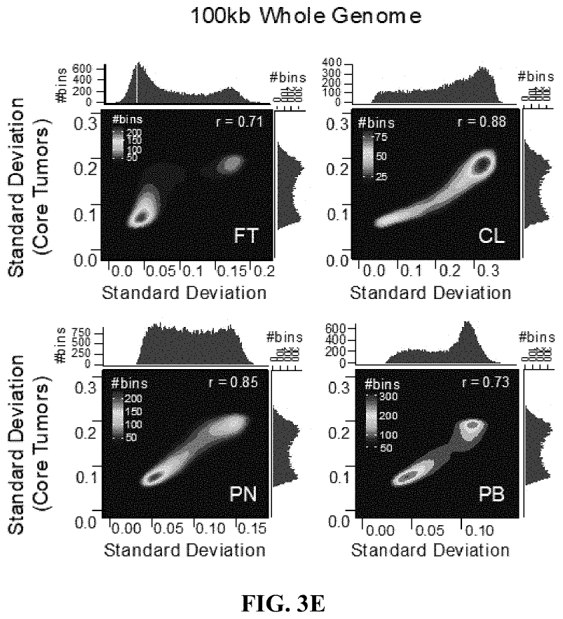

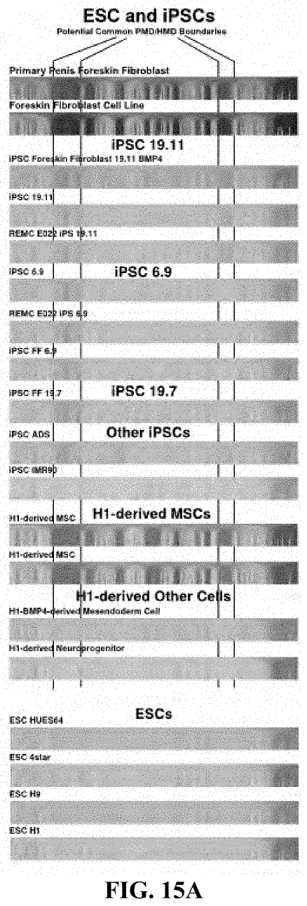

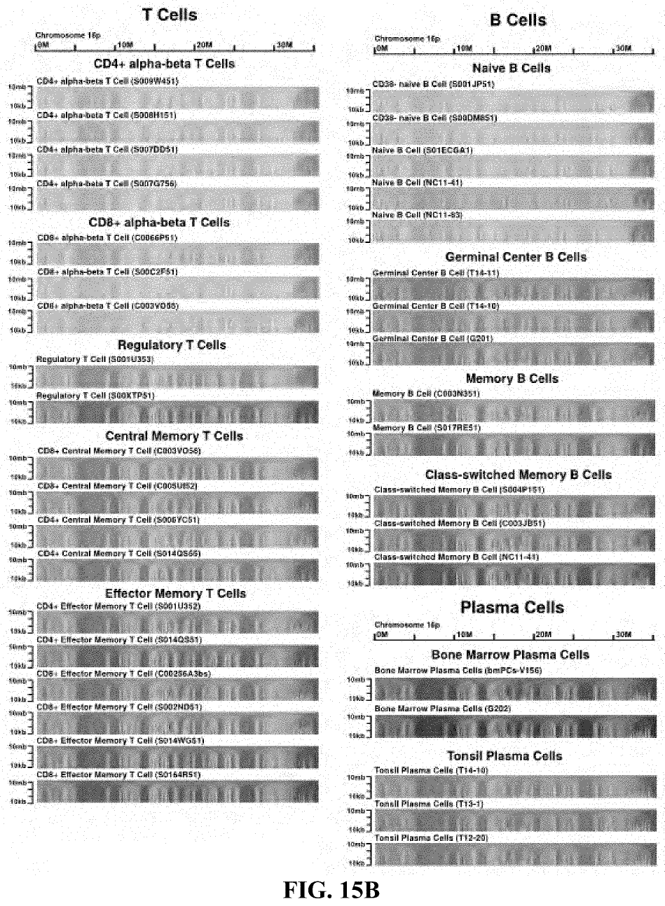

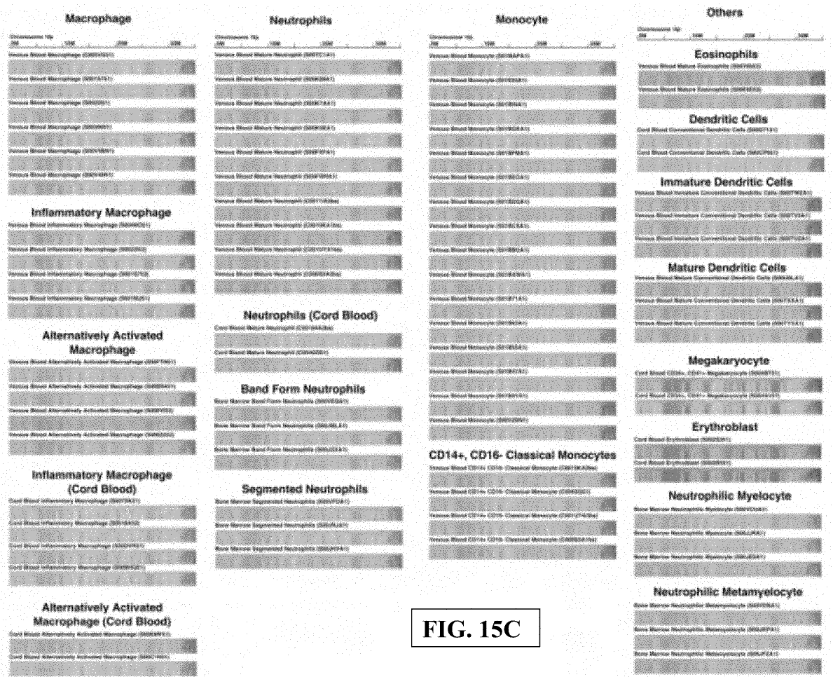

Solo-WCGW PMD structure was also investigated by combining our TCGA dataset with 343 previously published human and 206 mouse WGBS samples (FIGS. 30-1 to 30-16 (Table 1)), examining solo-WCGW methylation averages with human samples arranged into 6 groups (FIG. 3) and mouse samples into 4 groups (FIG. 4). As in the core set, the overall degree of hypomethylation varied widely, but PMD structure was largely shared for 5 of the 6 categories. Common PMDs overlapped lamina-associated regions (LADs) (33) and late replicating domains, as expected (FIG. 3A1-3A2 and FIG. 4, bottom). The germline and embryo (GE) category was the only exception, with only some samples sharing PMDs (FIG. 3A1-3A2, Group GE, FIG. 4, Group GE). Immortalized cell lines (cancer and non-cancer), with the exception of pluripotent embryonic cells, generally showed strongly hypomethylated PMDs that were shared with other groups (FIG. 3A1-3A2, Group CL, FIG. 4, Group ESC). More discussion on methylation maintenance in embryonic and induced pluripotent stem cells is given in working Example 9, and FIG. 15A.

In agreement with the TCGA tumor-adjacent “normal”, most disease-free post-natal tissues showed PMD structure shared with tumors and other groups (FIG. 3A1-3A2, Group PN and FIG. 4, Group PN). The normal human samples from Schultz et al. (25) made up the majority of non-brain samples in our PN group and clearly had shared PMDs in our solo-WCGW analysis, while the original analysis of Schultz et al. identified PMDs in only 3 of these 37 samples. Most brain samples in the PN group were from a different study (34), and these stood out as the one post-natal tissue type without clearly detectable PMDs in our analysis, possibly attributable to de novo DNA methylation in post-mitotic brain cells (34). Tissue types with high stem cell turnover (35) including liver, colon, skin, and pancreas displayed the strongest PMD hypomethylation.

All nucleated blood cell types showed shared PMD structure, in contrast to an earlier analysis of many of the same WGBS datasets (41) that found PMD hypomethylation to be limited to the lymphoid lineage (FIG. 3A1-3A2, Group PB). Both B cells and T cells could generally be divided into subgroups of strong vs. weak hypomethylation. Those subtypes having undergone antigen presentation and activation (e.g., memory B/T cells, regulatory T cells, germinal center B cells, and plasma cells) fell into the strongly hypomethylated class, while naive B and T cells fell into the weakly hypomethylated class, consistent with earlier reports showing that B and T cell hypomethylation increased during maturation (23, 24). However, unlike these earlier reports, the presently disclosed solo-WCGW analysis showed that PMD hypomethylation was already clearly evident by the naïve stage (FIG. 3A1-3A2 and FIG. 15B). Lymphocyte activation involves clonal expansion (proliferation of individual B/T cells to produce large numbers of daughter cells with the same antigen specificity) (36), and the dramatic hypomethylation that occurs after activation strengthens the notion that methylation loss accumulates during successive rounds of cell division (consistent with long term cultures (21)). The presently disclosed solo-WCGW analysis provided the first demonstration that PMDs occur across all cell types of the myeloid lineage and are largely shared with other cell types (FIG. 3A1-3A2 and FIG. 15C).

Specifically, FIGS. 15A-C show methylation maintenance in embryonic and induced pluripotent stem cells. FIG. 15A shows a multiscaled view of Solo-WCGW methylation in iPSC and ESC-derived cells, showing deep PMD in H1-derived MSCs and residual PMD in iPSCs. FIG. 15B shows a multiscale view of Solo-WCGW CpG methylation in T, B and plasma cells of different varieties, showing deep PMD hypomethylation in regulatory T cells, germinal center B cells, memory T, B cells and plasma cells. FIG. 15C shows a multiscale view of Solo-WCGW methylation in myeloid cells, showing deeper PMD in megakaryocytes and erythroblasts.

The tumor group (TM) consisted of 50 solid tumors (largely lmade up of the 40 core tumors shown previously), plus 50 hematopoietic malignancies (FIG. 3A1-3A2, Group TM). Interestingly, while hematopoietic tumors had more strongly hypomethylated PMDs than normal hematopoietic samples, they generally followed the trend established by their developmental origin: those derived from myeloid cells (AML) had shallower PMDs than those derived from lymphoid cells (CLL, MCL, TPLL, MM) (one-way Wilcoxon test, p=9.69e-7). The notable exception among lymphoid-derived tumors was ALL, which had hypomethylation levels similar to normal lymphoid cells. The lower degree of hypomethylation in ALL (derived from childhood cases) may reflect the generally lower degree of hypomethylation in cells from younger individuals, a topic investigated below.

For five of the six cell type groups (excluding group “GE”), mean methylation across samples in the group (FIG. 3B), as well as SD (FIG. 3C-D), revealed largely shared PMD structure. SD was bimodally distributed across the genome in all five groups (FIG. 3E), and could thus be used to define PMD regions. For all of the five sample groups, the majority of PMDs defined by high-SD bins were substantially overlapping PMDs defined earlier from the core tumor group (FIG. 3E and FIG. 16). For example, 82% of high-SD bins were overlapping between the post-natal non-blood group (PN) and the core tumor group, and 84% were overlapping between the post-natal blood group (PB) and the core tumor group. The findings support the idea, according to particular aspect of the present invention, that a large set of cell-type-invariant PMDs dominate the hypomethylation landscape in most tissues.

Specifically, FIGS. 3A-E show that most PMDs are shared across developmental lineages in humans. In FIG. 3A1-3A2, average solo-WCGW methylation levels were plotted along chromosome 16p for 390 WGBS samples, organized into 6 groups: Germline and preimplantation embryo (GE). Post-implantation embryonic/fetal samples (FT), grouped first by embryonic vs. extra-embryonic, then by average methylation. Cell lines (CL). Post-natal non-blood normal tissue samples (PN). Post-natal blood-derived samples (PB). Primary tumors (TM). Within each of the 6 groups, samples were organized by cell type (labeled with color codes). Lamin B1 signal and replication timing of IMR90 lung fibroblast are shown below methylation heatmaps (bottom). FIG. 3B shows mean methylation levels within each of the 5 major groups (excluding group GE), plotted as in FIG. 2B. FIG. 3C shows SD within each of the 5 major groups, plotted as in FIG. 2C. FIG. 3D shows SDs for the 100-kb scale alone. FIG. 3E shows the distribution of SD for all non-overlapping 100-kb genomic bins across all samples of the core tumor group (from FIG. 3D) are plotted on the Y-axis, compared to each of four major groups (FT, CL, PN, and PB), shown on the X-axis. Group GE is omitted due to lack of PMD structure.

Specifically, FIG. 4 shows that most PMDs are shared across developmental lineages in mouse. Average solo-WCGW methylation levels were plotted along a 40 representative 30-mb regions of chromosome 17 in mouse. 206 WGBS samples are organized into four groups: Embryonic Stem Cells (ESC); Germline and embryos (GE); Fetal tissues (FT); Postnatal tissues (PN); and Grouping and ordering of samples were performed as described in FIG. 3. Lamin and replication timing are shown on the bottom of the heatmap. Lamin A DamID from wild type mouse ESCs were downloaded from GEO with accession GSE6268369. Replication timing of day 9 differentiated ESCs were downloaded from GEO with accession GSE1798370.

### Example 4

(PMD Hypomethylation was Shown to Emerge During Embryonic Development))

The presence of PMD hypomethylation in multiple fetal tissue types led to further investigation of solo-WCGW methylation in gametes and early developmental stages (FIG. 5A-C). Human sperm was highly methylated, with little discernable PMD structure aside from the peri-centromeric region (FIG. 5A, Group I), while mouse methylomes displayed consistent PMD structures throughout spermatogenesis (FIG. 17). Human germinal vesicle oocytes had deep PMD hypomethylation (FIG. 5A, Group II), although a subset of PMD boundaries appeared to differ from somatic tissues. The rapid and global demethylation that occurs within the Inner Cell Mass (ICM) is thought to be an active process, attributable to a different mechanism than PMD-associated hypomethylation (37). Interestingly, while ICM and blastocyst samples were strongly de-methylated, they did retain weak PMDs with boundaries resembling those of oocytes rather than those of later somatic cell types (FIG. 5A, Group III). Primordial germ cells (PGCs), which are set aside from the soma soon after implantation, showed an even more extreme erasure of DNA methylation than blastocysts, precluding any discernable PMD structure (FIG. 5A, Group IV).

Embryonic somatic tissues (FIG. 5A, Group V) were rapidly re-methylated genome-wide, and PMD structure could not be readily resolved, in contrast to more mature fetal samples (FIG. 5A, Group VI). Tissues sampled at different developmental stages revealed a progressive emergence of PMD/HMD structure along organismal development (FIG. 5C). This analysis revealed a substantial degree of similarity between PMD structure in brain tissues and PMD structure in other lineages, something that was not apparent from genomic plots. The substantial similarity of PMD structure detected between ICMs, ESCs, embryonic (<8 weeks) stages, and post-natal samples, suggests that PMD hypomethylation may begin at the earliest stages of development. This interpretation is strengthened by the observation that the degree of hypomethylation observed at the fetal and postnatal stages for each cell type largely mirror the lineage-specific hypomethylation rate within the same embryonic cell type.

Specifically, FIGS. 5A-C show that PMD hypomethylation emerges during embryonic development. In FIG. 5A, multi-scale solo-WCGW average plots are shown for samples divided into seven developmental stages, as diagrammed in FIG. 5B: paternal (I) and maternal (II) germ cells, implantation-related tissues (III), primordial germ cells (IV), embryonic soma (V), fetal soma (VI) and postnatal soma (VII). FIG. 5C shows rank-based analysis of the 792 genomic 100-kb bins from chr16, comparing methylation ranks of the core tumors (Y-axis) to each developmental sample (X-axis), with each axis going from a rank of 1 (lowest methylation) to the rank of the highest methylation (excluding bins with missing value from either of the samples). Greater correlations (indicated by the Spearman's correlation coefficient ρ) indicated stronger HMD/PMD structure.

Specifically, FIG. 17 shows a multiscaled view of chromosome 17 (3-43Mbp) Solo-WCGW methylation in different stages of mouse spermatogenesis from prospermatogonia to mature sperm.

### Example 5

(PMD Hypomethylation was Shown to be Associated with Chronological Age)

To investigate the link between PMD-associated hypomethylation and cumulative numbers of cell divisions, the question as to whether solo-WCGW methylation level within common PMDs was associated with donor age in different primary cell types was tested. A strong age association was evident from the WGBS profile of sorted CD4+ T cells from a newborn vs. those from a 103-year-old individual, with the latter being closer to a T cell-derived leukemia than to the newborn sample (FIG. 6A). To investigate age-related properties within larger studies only performed using the HM450 platform, we used the common PMDs derived from all WGBS samples to define a standard set of solo-WCGW PMD probes represented on HM450 (working Example 8 below). In these larger studies, PBMC samples from newborns had significantly less PMD hypomethylation than those from elderly donors (FIG. 6B left), and fetal liver samples had significantly less PMD hypomethylation than adult liver samples (FIG. 6B, right). Strikingly, fetal tissues from four different developmental lineages showed nearly linear accumulation of hypomethylation from 9 weeks post-gestation to 22 weeks post-gestation (FIG. 6C). Despite small sample sizes, this was statistically significant for 3 of the 4 fetal tissue types. A similar association was observed between PMD hypomethylation and gestational age in multiple mouse fetal tissue types (FIG. 18).

Specifically, FIG. 18 shows the association of average PMD solo-WCGW CpG methylation with gestational age in mouse WGBS data sets stratified by tissue types.

An earlier study used the HM450 platform to investigate the effects of environmental (UV) exposure on PMD hypomethylation in human skin samples (26). While the earlier study described PMD hypomethylation as only occurring within the sun-exposed samples of the epidermal layer, the presently disclosed re-analysis of solo-WCGWs revealed that both dermal and epidermal cells exhibited age-associated PMD hypomethylation without sun exposure, but that this process was dramatically accelerated specifically in epidermal cells upon sun exposure (FIG. 6D). This suggests that while PMD hypomethylation is a nearly universal process in aging, the degree of hypomethylation is a reflection of the complete mitotic history of the cell, including proliferation associated with normal development and tissue maintenance, plus additional cell turnover occurring as a consequence of environmental insults.

HM450 datasets showed that diverse hematopoietic cell types had a significant association between donor age and degree of hypomethylation, with the myeloid lineage (FIG. 6E) having a much slower rate of age-associated loss compared to the lymphoid lineage (FIG. 6F). This finding is consistent with the overall lower degree of methylation observed in myeloid cell types from WGBS data. While the rate of loss within the myeloid lineage was extremely low, the association to donor age was highly significant within the large human monocyte dataset (FIG. 6E). This finding contradicts an earlier analysis based on many of the same samples, which found that monocytes lacked PMD hypomethylation and age-associated hypomethylation (24).

Specifically, FIGS. 6A-F show that PMD hypomethylation is associated with chronological age. In FIG. 6A, multi-scale solo-WCGW average plots are shown for newborn CD4 T cell, 103-year old CD4 T cell (GSE31438) and T cell prolymphocytic Leukemia (BLUEPRINT accession S016KWU1). FIGS. 6B-F show a summarization of average PMD hypomethylation in HM450-based samples, by averaging beta values for 6,214 solo-WCGW probes mapped to common PMDs (see working Example 8 below). Peripheral Blood Mononuclear Cell (PBMC) in newborns and nonagenarians (left, from GSE30870, p=8.8e−5, one-way Wilcoxon Rank Sum test), and disease-free fetal and adult liver tissue (right, from GSE61278). Center lines of the box plots indicate median, and the lower and upper bounds indicate lower and upper quartiles. The lower and upper whiskers indicate smallest and largest methylation values. **p<=0.001 from Wilcoxon Rank Sum test. FIGS. c-f show HM450-based solo-WCGW averages vs. age for individual donors for several tissue types. N is the number of donors/samples, r is Pearson's product moment correlation, b1 is the estimated rate of methylation loss, and p is the p-value based on Pearson correlation test. FIG. 6C shows four fetal tissue types during three pre-natal time points (from GSE56515). FIG. 6D shows sun-exposed and sun-protected dermis and epidermis (from GSE51954). FIG. 6E shows sorted blood cells of the myeloid lineage (D1: GSE35069; D2: GSE56046). FIG. 6F shows sorted blood cells of lymphoid lineage (D1: GSE35069; D3: GSE71955; D4: GSE59065).

### Example 6

(PMD Hypomethylation was Shown to be Linked to Mitotic Cell Division in Cancer)

The landscape of cancer hypomethylation in 9,072 tumors from 33 cancer types included in TCGA, was next studied using the HM450 solo-WCGWs located within common PMDs (FIG. 7A). PMD hypomethylation was nearly universal but showed extensive variation both within and across cancer types. Comparison to 749 adjacent normals from TCGA showed that the relative degree of hypomethylation across cancer types was correlated with that of the disease-free tissue of origin (FIGS. 19-21). This association was reduced in cancer types for which the normal adjacent specimens contained low fractions of relevant cell types representing putative cells of origin for the tumor.

Specifically, FIG. 19 shows the Solo-WCGW methylation average in common HMD and common PMD in 9,072 TCGA tumor samples from 33 tumor types.

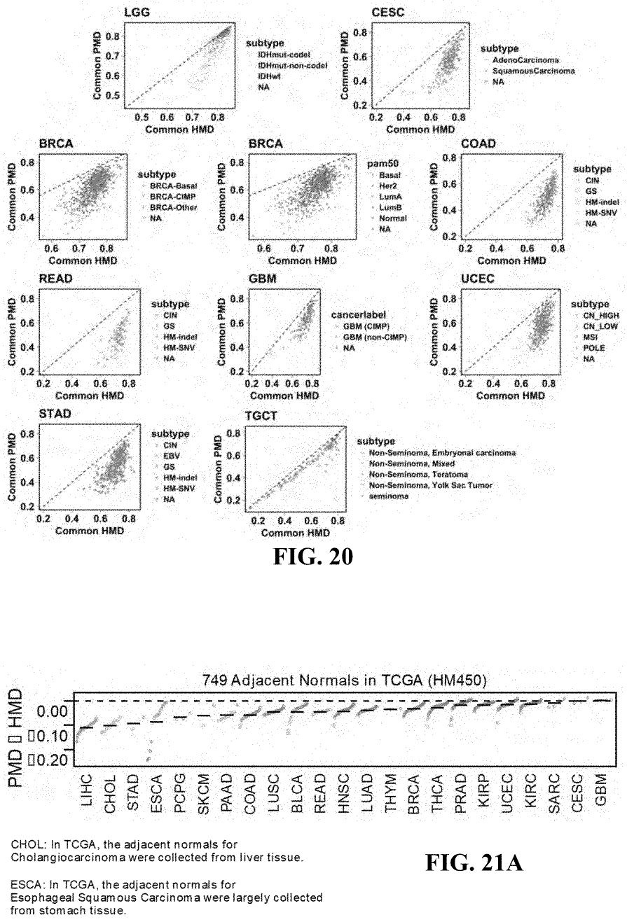

Specifically, FIG. 20 shows subtype-stratification of Solo-WCGW methylation average in common HMD and common PMD in TCGA tumor samples from 10 cancer types.

Specifically, FIG. 21A-D shows that within TCGA tumors, higher genome-wide somatic mutation densities were found to be significantly associated with deeper PMD hypomethylation, suggesting that mitotic turnover may underlie both somatic mutation and PMD hypomethylation (FIG. 7B). This association was consistent using different purity thresholds (FIG. 13C), indicating that it was not the result of confounding due to differential detection sensitivity related to purity. PMD hypomethylation was also associated with somatic copy number aberration density (FIG. 21D). FIG. 21a shows the difference of PMD and HMD methylation average of 6,214 Solo-WCGW probes in 749 adjacent normal samples assayed in TCGA on HM450 platform. FIG. 21B shows a comparison of normal (N=749) vs tumor (N=9,072) HMD-PMD methylation based on Solo-WCGW CpGs in 33 cancer types in TCGA with lines indicate standard deviation. The sample sizes are: ACC(N=80); BLCA(N=419); BRCA(N=799); CESC(N=309); CHOL(N=36); COAD(N=316); DLBC(N=48); ESCA(N=186); GBM(N=153); HNSC(N=530); KICH(N=66); KIRC(N=325); KIRP(N=276); LAML(N=194); LGG(N=534); LIHC(N=380); LUAD(N=475); LUSC(N=372); MESO(N=87); OV(N=10); PAAD(N=185); PCPG(N=184); PRAD(N=503); READ(N=99); SARC(N=265); SKCM(N=474); STAD(N=396); TGCT(N=156); THCA(N=515); THYM(N=124); UCEC(N=439); UCS(N=57); UVM(N=80); The sample sizes for normals are: BLCA(N=21); BRCA(N=98); CESC(N=3); CHOL(N=9); COAD(N=38); ESCA(N=16); GBM(N=2); HNSC(N=50); KIRC(N=160); KIRP(N=45); LIHC(N=50); LUAD(N=32); LUSC(N=43); PAAD(N=10); PCPG(N=3); PRAD(N=50); READ(N=7); SARC(N=4); SKCM(N=2); STAD(N=2); THCA(N=56); THYM(N=2); UCEC(N=46); The mean of each data set is used to measure the center. FIG. 21 c shows the Spearman's correlation coefficient (for the analysis in FIG. 7B), shown as a function of minimum purity threshold from 0.1 to 0.95 (hypermutators excluded; working Example 8). PMD hypomethylation in TCGA tumors was captured by the average DNA methylation beta values of common PMD HM450 probes. FIG. 21D shows the correlation between PMD methylation (average DNA methylation beta value of HM450 common PMD probes) and the number of Somatic Copy Number Aberration (SCNA) in TCGA tumor sample (N=9454).

Somatic mutation events are known to display mitotic clock-like properties (38). Within TCGA tumors, higher genome-wide somatic mutation densities were found to be significantly associated with deeper PMD hypomethylation, suggesting that mitotic turnover may underlie both somatic mutation and PMD hypomethylation (FIG. 7B). This association was consistent using different purity thresholds (FIG. 21C), indicating that it was not the result of confounding due to differential detection sensitivity related to purity.

PMD hypomethylation was also associated with somatic copy number aberration density (FIG. 21D). Activation and insertion of LINE-1 endogenous retro-transposable elements is a common event in human cancer and can induce structural alterations, copy number alterations, and induction of oncogenes (39-41). Using somatic LINE-1 insertions identified from Whole Genome Sequencing (WGS) of TCGA tumors (41), LINE-1 insertion breakpoints were found herein to be preferentially enriched in PMD regions (FIG. 7C), in agreement with an earlier study (39). Intriguingly, tumors with deeper PMD hypomethylation had more LINE-1 insertions in 8 of 9 cancer types, with the only exception being endometrial cancer (FIG. 7D; FIG. 22). While the mechanisms controlling LINE-1 insertion density in cancer are not well understood, they may be stochastically linked to the number of cell divisions (like SNVs), and/or require de-repression of “hot” LINE-1 elements, a process which may be linked to DNA hypomethylation (42, 43).

Specifically, FIG. 22 shows the association of LINE-1 break points and PMD methylation (characterized by average of HM450 probes in common PMDs). Rho is Spearman's correlation coefficient. P-value was calculated using algorithm AS89 implemented in the R software.

According to particular aspects of the present invention, tumors highly proliferative at the time of specimen collection may also reflect an extensive history of past cell division. Using TCGA samples with matched gene expression data, the 60 genes most strongly associated with PMD hypomethylation were identified, and it was determined that these genes were most enriched in Gene Ontology functional terms associated with proliferation and mitotic cell division (FIG. 7E). In further support of this link between ongoing cell proliferation and PMD hypomethylation, the genes with the greatest association to PMD hypomethylation were strongly enriched within a list of 350 cell-cycle dependent genes from Cyclebase (44) (FIG. 7F). Ranking tumor samples by their degree of PMD hypomethylation showed that this association involved most cell-cycle dependent genes across different mitotic stages (FIG. 7G). Remarkably, proliferative tumors had deep PMD hypomethylation despite having higher levels of both DNMT1 and DNMT3A/B, which are expressed as part of a general DNA replication program (working Example 10). The most hypomethylated tumors also had high expression of UHRF1 (a contributor to DNMT1 methylation maintenance activity), underscoring that PMD hypomethylation accumulates despite strong expression of the DNA methylation maintenance machinery. The question of whether overexpression of TET genes, which participate in active DNA demethylation, might contribute to PMD hypomethylation was also investigated. None of the three TET genes were highest in the tumors with strongly hypomethylated PMDs, indicating that TET enzymes are not responsible for DNA methylation loss in PMD regions (in contrast to promoters and CpG islands, where extensive evidence exists for TET-mediated demethylation). According to particular aspects of the present invention, all of the presently disclosed tumor mutation and expression results suggest cumulative mitotic cell divisions as the major driving force behind PMD hypomethylation accumulation.

Specifically, FIGS. 7A-G show that PMD hypomethylation is linked to mitotic cell division in cancer. FIG. 7A shows PMD-HMD solo-WCGW methylation difference for 9,072 tumors from TCGA HM450 data. Each sample is ordered within cancer type by PMD-HMD difference, and cancer types are ordered by average PMD-HMD difference. FIG. 7B shows PMD methylation (X-axis) vs. somatic mutation density (Y-axis) for all 3,959 high purity TCGA cases (purity>=0.7), with Spearman's p indicated. The blue line represents the regression line for all samples, while the red regression line excludes “hypermutator” samples (Online Methods). FIG. 7C shows density of somatic LINE-1 insertions (violin plot elements) in non-overlapping 1-mb genomic bins (N=3,053), stratified by percent of bin overlapping common PMDs (only cases with whole-genome sequencing are included). FIG. 7D shows PMD methylation (X-axis) vs. LINE-1 insertion counts (Y-axis) for nine TCGA cancer types having substantial LINE-1 insertion counts. * (p<0.05) and **(p<=0.01) indicate Spearman's test significance. FIG. 7E shows the 10 most significantly enriched Gene Ontology (GO) terms for the 60 genes with the most strongly correlated expression vs. PMD hypomethylation in TCGA tumors, showing fold enrichment (grey) and false discovery rate (olive). Fib. 7F shows Gene Set Enrichment Analysis (GSEA) for 350 cell-cycle-dependent genes from Cyclebase (44), ranking all genes according to degree of expression vs. PMD hypomethylation correlation. FIG. 7G shows normalized expression (Z-scores) of cell-cycle-dependent genes from Cyclebase (categorized by cell cycle phase) in 3,414 high purity TCGA tumor samples (purity>=0.7), ordered by PMD-HMD methylation difference.

### Example 7

(Both Replication Timing and H3K36Me3 were Shown to Affect Methylation)

The one cell type with publicly available data for all relevant histone and topological marks, IMR90, was used to systematically analyze the presently disclosed solo-WCGW based PMD definition. This analysis confirmed previous findings (6, 7) that HMD/PMD structure coincided with nuclear architecture, as characterized by Hi-C A/B compartments, Lamin B1 distribution and replication timing (FIG. 8A). At the single CpG scale, Solo-WCGW CpG methylation was most strongly correlated with replication timing, followed by the histone mark H3K36me3 (FIG. 23A).

Specifically, FIG. 23 shows that head and neck squamous cell carcinomas with NSD1 mutations, which exhibit significant reductions in H3K36me2 and H3K36me3 levels (57), have substantial loss of DNA methylation in the HMD compartment. FIG. 23A shows Spearman correlation coefficients of Solo-WCGW CpG methylation and 10 other epigenomic features of IMR90 fibroblast at single CpG scale. Samples were hierarchically clustered based on distances defined by 1-abs(rho). The dendrogram of clustering is shown on the bottom with arrow indicating the best and the 2nd best correlator with Solo-WCGW CpG. FIG. 23B shows PMD vs HMD methylation average of Solo-WCGW HM450 probes in TCGA HNSC tumors showing NSD1 wild types and mutants.

The de novo methyltransferase DNMT3B has recently been shown to be guided to transcribed gene bodies via a direct interaction with the H3K36 methylation mark (45). Active genes marked by H3K36me3 are overwhelmingly located in early replicating regions, and it has been suggested that both active transcription of gene bodies and early replication timing contribute to differential methylation throughout the genome (9). To disentangle the contributions of H3K36me3 and replication timing to genome-wide DNA methylation levels and PMDs, a stratified analysis of all solo-WCGW CpGs in the genome (FIG. 8B-C) was performed, revealing that the 14% of Solo-WCGWs overlapping H3K36me3 were highly methylated, irrespective of position relative to gene annotations or replication timing (FIG. 8B, left). The remaining 86% of Solo-WCGWs (those not overlapping an H3K36me3 peak) had lower methylation across all contexts, but were strongly replication-timing dependent (FIG. 8B, right). In IMR90 cells, the degree of methylation maintenance associated with early replication timing was even greater than the degree associated with H3K36me3 (FIG. 8B, right). The relative contribution of replication timing vs. H3K36me3 was reversed in the H1 (hESC) cell line (FIG. 8C), a cell type with exceptionally high DNMT3A/B activity that makes them one of the few cell types able to survive loss of Dnmt1 function (46, 47). Because most somatic cell types had detectably hypomethylated PMDs like IMR90 (and unlike H1), the presently disclosed observations support a model in which highly effective methylation maintenance at H3K36me3-marked regions is achieved through a process mediated by the direct recruitment of DNMT3B through its PWWP domain (45). Consistent with earlier observations (9), this H3K36me3-linked maintenance appears to act independently from the effect of replication timing on PMD methylation loss (FIG. 8d).

Specifically, FIGS. 8A-G show that replication timing and H3K36me3 contribute independently to methylation maintenance. FIG. 8A shows a multi-scale plot of chr16p showing similarity between solo-WCGW methylation and other chromatin marks in the IMR90 fibroblast cell line. Fib. 8B shows the average methylation level of all genomic solo-WCGWs in IMR90, stratified by (1) overlap with H3K36me3 peaks (left vs. right), (2) context relative to gene annotations (“Genic” vs. “Intergenic”), and (3) Repli-seq replication timing bin (red, yellow, light blue, dark blue). For Solo-WCGWs residing within +1-10 kb of an annotated gene (Genic), meta-gene plots show methylation averages in relation to the Transcription Start Site (TSS) and the Transcription Termination Site (TTS). For all other Solo-WCGWs (Intergenic), each replication timing group is shown as a single violin plot. FIG. 8C shows the same representation of data plotted for the H1 hESC cell line (using Repli-chip data rather than Repli-seq). FIG. 8D is a schematic summary, showing Solo-WCGW CpG methylation loss primarily determined by replication timing domain but locally protected by H3K36me3. FIG. 8E shows a schematic model illustrating DNMT1 processivity favoring dense CpGs and leading to incomplete re-methylation of Solo CpGs. FIG. 8F shows a schematic illustration of the “re-methylation timing model” where genomic regions synthesized earlier in S-phase (HMDs) spend more time exposed to methylation maintenance machinery and thus more complete methylation maintenance than PMDs. FIG. 8G shows an illustration of the relationship between major determinants of hypomethylation and 3D nuclear topology, with Lamina Associated Domains (LADs) occupying a distinct heterochromatic nuclear compartment.

### Example 8

(Materials and Methods)

Whole Genome Bisulfite Sequencing.

Cases for the WGBS assay were selected from 8 of the most common cancer types (Lung squamous cell carcinoma, Lung adenocarcinoma, Breast, Colorectal, Endometrial, Stomach, Bladder, Glioblastoma). For at least one tumor from each cancer type, we also sequenced its adjacent histologically normal tissue; for the rest, only the tumor was profiled. These samples were combined with one tumor and matched normal colon cancer pair from an earlier study (6), yielding a core set of 40 well characterized tumors and 9 adjacent normal samples (FIGS. 30-1 to 30-16 (Table 1)). These tumors and normal samples are referred to as core tumors and core normals in the text. Paired-End WGBS-PE protocol was adapted from earlier developed protocols (6). Briefly, sample genomic DNA (2 μg) was sonicated using a Diagenode Bioruptor and size selected to a range of 400-500 bp. Sodium bisulfate conversion of all DNA samples was performed using the EZ DNA Methylation Kit (Zymo Research). All libraries are quality controlled by Agilent Bioanalyzer examination and quantified using the Kapa Biosystems kit. Cluster generation and paired-end sequencing are performed according to Illumina guidelines for the HiSeq 2000, utilizing the latest version reagents and software updates.

External Data.

The external human WGBS data consists of 19 germ cells and pre-implantation embryonic tissues, 13 post-implantation embryonic and fetal tissues, 37 cell lines, 59 non-blood normal primary tissues (including normal adjacent tissues of tumors as well as disease-free samples), 154 blood or blood component samples, 11 solid tumors and 50 blood malignancies (FIGS. 30-1 to 30-16 (Table 1)). The 206 mouse WGBS data sets are constituted by 13 ES cells, 17 germ cells and embryonic tissues, 123 primary fetal tissues and 53 primary postnatal normal samples. Human postnatal normals were retrieved from Roadmap Epigenomics Project (see working Example 8, under “URLs”). Sorted blood WGBS and blood malignancies were downloaded from the BLUEPRINT epigenome project (see working Example 8, under “URLs”). Mouse fetal WGBS samples were downloaded from the ENCODE project (see URLs). Other postnatal and fetal WGBS samples were downloaded from MethBase (27). For MethBase samples, only data sets that passed the Q/C standard of the Database were included. The relevant citations and sources of the WGBS data sets used in the presently disclosed work are shown in FIGS. 30-1 to 30-16 (Table 1). HM450 datasets and the corresponding meta-information used for age association were obtained from Gene Expression Omnibus by downloading the following datasets: GSE30870, GSE35069, GSE56046, GSE59065, GSE51954, GSE61278, GSE56515. Mutation prevalence for TCGA tumor samples were obtained from the Broad Institute TCGA Genome Data Analysis Center (2016): MutSigCV v0.9 cross-sample somatic mutation rate estimates (Jan. 28, 2016 release). Tumors that have POLE or APOBEC family mutations, or classified as with microsatellite instability, were annotated to be hypermutator tumors. When hypermutator samples were excluded, samples without annotation were also excluded. Numbers of somatic LINE-1 insertions in 1-mb bins were downloaded from an earlier report (41).

Alignment and Extraction of Methyl-Cytosine Levels.

Reads were aligned to the genome (build GRCh37) using BSmap (71) under the following parameters “−p 27 −s 16 −v 10 −q 2

-A
AATGATACGGCGACCACCGAGATCTACACTCTTTCCCTACACGA
CGCTCTTCCGATCT
-A
AGATCGGAAGAGCGTCGTGTAGGGAAAGAGTGTAGATCTCGGTG
GTCGCCGTTCATT

(3′-end adapter SEQ ID NOS:237 and 238, respectively). Duplicated reads were marked using Picard tools (see URLs, version 1.38). DNA methylation rates and SNP information were called using Bis-SNP (72), using the default easy-run procedure (see URLs). Bis-SNP allows for distinguishing a C->T mutation from bisulfite conversion by investigating the complementary strand. CpGs with fewer than 10 reads' coverage were excluded from analysis.

Genomic Binning.

To show megabase-scale HMD/PMD structures, a 100-kb window size was chosen so that the segments would contain a sufficient number of solo-WCGWs to give reliable methylation averages (FIG. 25, and see working Example 11), without losing resolution to detect the majority of PMD positions, which fall within PMDs of 500 kb or greater (6).

Specifically, FIG. 25 shows first decile of the number of solo-WCGW CpGs in windows of different sizes that were used to segment the whole genome.

Definition of Preliminary PMD/HMD Domains Based on all CpGs.

WGBS was used at ˜15× coverage to profile methylation patterns of 40 tumors (39 new TCGA samples and one from a prior study (6)) from 8 of the most common cancer types, and tumors were selected on the basis of high cancer cell content (FIGS. 30-1 to 30-16 (Table 1)). For one case from each of the 8 cancer types, profiled both the tumor and adjacent normal tissue was profiled; for the rest, only the tumor was profiled. Most of our tumor samples had a high degree of hypomethylation, so an existing HMM based tool, MethPipe (27) using a window size setting of 10 kb, was first used to identify PMDs in each sample individually (FIG. 9a). While the fraction of the genome covered by PMDs in different samples differed by two to three folds (FIG. 9b), there was sufficient overlap to define a shared MethPipe PMD set of 417 PMDs (covering 13% of the genome) that was shared among at least 21 of the 30 tumors. As a comparison group, we defined a shared MethPipe HMD (highly methylated domain) set that was not covered by PMDs in any tumor sample, and included 830 regions (covering 32% of the genome).

Final Definition of PMDs/HMDs Based on Standard Deviation of Solo-WCGW Methylation.

Every 100-kb bins are dichotomized into PMD/HMD using a Gaussian mixture model (implemented in the R package mixtools) based on cross-sample SD of beta values from our core tumor samples (N=40). The Gaussian mixture model assumes two subpopulations of 100-kb bins—those located in PMDs with higher cross-sample SDs and those located in HMDs with lower cross-sample SDs. The final threshold of cross-sample SD for classifying PMDs from HMDs is determined to be 0.125. The more conservative sets of “common PMDs” and “common HMDs” are defined by the criteria that SD>0.15 and SD<0.10 respectively. Overlap of PMD boundaries of two samples were measured in the percentage of 100-kb bins identified as both in PMDs and in HMDs in the two samples respectively. The mouse PMDs/HIMDs were defined in the same way using 32 postnatal non-brain WGBS samples (FIGS. 30-1 to 30-16 (Table 1)). The SD threshold for classifying PMDs from HMDs in mouse is determined to be 0.09.

HM450 Analysis.

For TCGA HM450 data sets, raw IDATs were preprocessed by first applying background subtraction (73) and then linear dye-bias correction matching the signal intensities of the two detection channels. Probe signals with detection p-value<0.05, as well as probes overlapping common SNPs and putative repetitive elements which cause potential cross-hybridization were then masked (74). For external data sets where raw IDATs were unavailable, processed beta values downloaded from GEO were used. Based on WGBS analysis, HM450 probes were classified according to the number of neighboring CpGs and the tetranucleotide sequence context. Only probes targeting solo-WCGW CpGs are retained. Also removed were probes falling into annotated CpG Islands, or those unmethylated (beta<0.2) in at least 20 of the 749 matched normal tissue samples included in TCGA. This resulted in 6,214 probes in common PMDs and 9,040 probes in common HMDs. Four letter acronyms for cancer types were taken following the official TCGA nomenclature. The difference of methylation between the mean methylation of solo-WCGW probes located in common PMDs and those in common HMDs was used to measure the degree of PMD-associated DNA hypomethylation in each sample. This method avoids confounding in the case of cancer types derived from globally de-methylated cell types such as primordial germ cells (FIGS. 20-21).

Analysis of the IMR90 Epigenome.

Features are clustered using 1−|ρ| as distance where r is the Spearman's correlation coefficient. Centromeres are excluded from IMR90 analysis. IMR90 epigenome data was downloaded from the ENCODE project data center (accessions listed in FIGS. 30-1 to 30-16 (Table 1)). Wavelet-transformed signals for replication timing were downloaded from GEO (GSM923447) (75). Histone mark signal was quantified using percentage of base overlaps of each window with gapped peaks downloaded from the Roadmap Epigenome Consortium. Gene bodies were extracted from GENCODE transcript annotation version 26. Base overlap was used as the gene body signal. RNA-seq signal is log 2 transformed number of reads overlapping with each window using bedtools (76). Only the protein-coding gene annotation from the HAVANA team was used for genic analysis in FIG. 8d. Intergenic regions exclude all transcript annotation from all sources. Solo-WCGW CpGs LaminB1 ChIP and HiC data were downloaded from GEO under the accession GSE53331 and GSE35156, respectively.

Rescaling Based on PMD Methylation.

The distribution of methylation values within common PMD 100-kb bins was calculated. The top and bottom 20% of this distribution was trimmed for each sample, setting low values to 0 and high values to 1, and linearly rescaled all values between 20% and 80% to the range [0,1] (FIG. 2E). The same genomic region of chr16p is visualized in FIG. 2F.

Stratified Analysis of Solo-WCGW CpGs in the Genome.

The Solo-WCGW CpGs were first classified (FIG. 8b-c) by their overlap with H3K36me3 into H3K36me3-positive (left) and H3K36me3-negative (right) categories, then by relative position to gene structures and placement in one of the four replication timing bins quartiles (colors, with threshold≤40, (40,60], (60,75],>75 for IMR90 Repli-Seq and ≤−0.5, (−0.5,0.4], (0.4,1.15],>1.15 for H1 Repli-ChIP). For Solo-WCGWs residing within +1-10 kb of an annotated gene, metagene plots (FIG. 8B-C) were used to show average methylation levels across all genes in relation to the Transcription Start Site (TSS) and the Transcription Termination Site (TTS). For all other Solo-WCGWs (intergenic), the distribution of methylation values was shown together for each replication timing group as a single violin plot.

Statistics.

Except for when described explicitly in the text, P-values for two-group comparison were calculated using one-tailed Wilcoxon's Rank Sum test. Correlation coefficients were computed with Spearman's method, with the exact P-values calculated in R using algorithm AS (89), otherwise via asymptotic t-approximation when exact computation was not feasible.

Data availability.

The WGBS data (incorporated by reference herein) is available in Genome Data Commons (GDC) under the TCGA project with IDs and file names shown in FIGS. 30-1 to 30-16 (Table 1).

Code availability.

Our customized work flow for preprocessing WGBS sequencing data is freely accessible (see under URLs below; incorporated by reference herein).

URLs.

Roadmap Epigenomics data is downloaded from ftp://ftp.ncbi. nlm.nih.gov/pub/geo/DATA/roadmapepigenomics/. BLUEPRINT epigenome project data is downloaded from ftp://ftp.ebi.ac.uk/pub/databases/blueprint/. ENCODE data project is downloaded from www.encodeproject.org. The Bis-SNP easy run procedure is detailed at http://people.csail.mit. edu/dnaase/bissnp2011/stepByStep.html. The entire customized work flow ECWorkflows is hosted and freely available at https://github. com/uec/ECWorkflows. Picard tools was downloaded from http://broadinstitute. github. io/picard.

### Example 9

(PMD Hypomethylation in Immortalized Cell Lines was Demonstrated Using the Solo-WCGW Motif)

According to particular aspects, PMD hypomethylation was observed in almost all cultured cell lines except for ESCs, iPSCs and their derived cell lines (FIG. 4 Group ESC). Interesting observations included: 1) hESCs (including H1, H9 and HUES64 and 4star) and most hESC-derived progenitor cells were heavily methylated without visually detectable PMD, most likely due to hyperactivity of DNMT3B (77, 78). The stark contrast between the primary ICM sample and the heavily methylated hESCs suggests that cultured hESCs may reflect a later stage of post-implantation embryonic development, where expression of the DNMT3A and DNMT3B methyltransferases can help to maintain high levels of DNA methylation despite prolonged culture (FIG. 5A). 2) Two H1-derived Mesenchymal Stem Cells (MSCs) showed clear PMD structure (FIG. 15a). 3) iPSCs, also with active DNMT3B (79) and with very little loss of PMD methylation in most samples, had residual trace PMDs in some samples (e.g., the 19.11 cell line) with respect to fore-skin fibroblasts from which they originated (FIG. 15A).

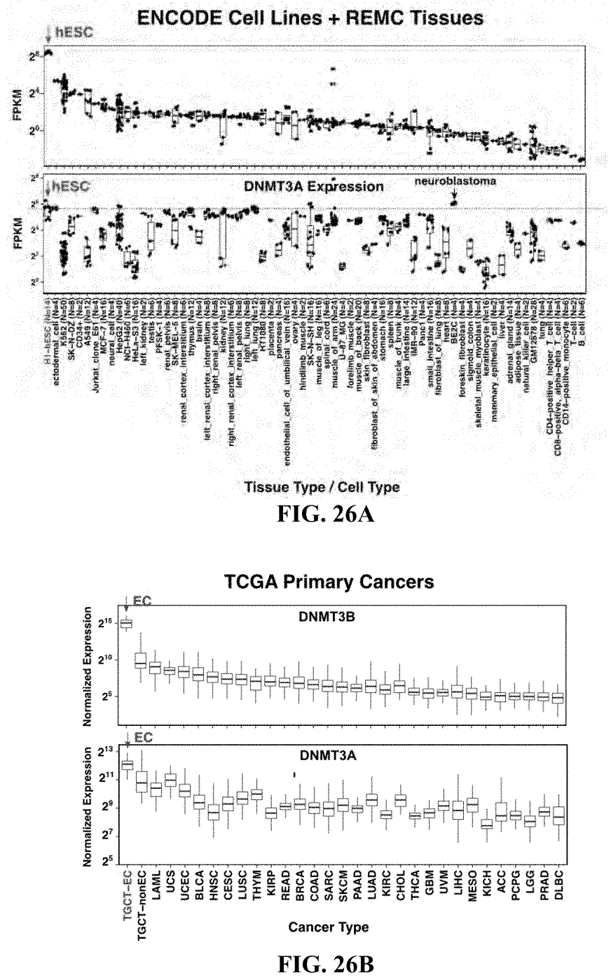

Note that although both ESCs and the proliferative tumors were high in the expression of DNMT3s compared to other normal tissues of non-embryonic origin, the level of expression in ESCs was higher than the most proliferative tumors. For example, the expression of DNMT3B in H1 hESC was higher than other cancer cell lines and primary tissues assayed in the ENCODE project by over ten-fold (FIG. 26A). Embryonic Carcinoma, sharing a similar early embryonic origin with ESCs, also had the highest expression of both DNMT3A and DNMT3B compared to other cancer types in TCGA (FIG. 26B). Like hESCs, these embryonic carcinomas did not manifest strong PMD structures either (FIG. 20). Since DNMTs are part of a large DNA replication program, the high DNMT3s in most proliferative tumors are passively driven by the fast cell turn-over of the cancer cells, while ESCs actively express DNMT3s to maintaining their pluripotency. This explains the seemingly contradictory observations of a strong PMD structure in the proliferative tumors and lack of PMD structure in ESCs, despite both having high DNMT3s. This is supported by the high expression of other replication program component genes (such as UHRF1 and other cell cycle dependent genes) in the highly proliferating tumors with severe PMD hypomethylation (FIG. 7G).

Specifically, FIGS. 26A-B show mRNA expression of DNMT3A and DNMT3B. Expression of DNMT3B in H1 hESC was higher than other cancer cell lines and primary tissues assayed in the ENCODE project by over ten-fold (FIG. 26A). Embryonic Carcinoma, sharing a similar early embryonic origin with ESCs, also had the highest expression of both DNMT3A and DNMT3B compared to other cancer types in TCGA (FIG. 26B). FIG. 26A shows mRNA expression of DNMT3A and DNMT3B in ENCODE cell lines and Roadmap Epigenome Consortium (REMC) primary tissues (each data point corresponds to the expression level for a cell line or primary tissue type). FIG. 26b shows mRNA expression of DNMT3A and DNMT3B in all TCGA cancer types with TGCT split into tumors of the embryonic origin (TGCT-EC) and non-embryonic origin (TGCT-nonEC). The figures show elevated DNMT3B expression in hESCs and embryonic carcinomas compared to other tissues and cancers by over an order of magnitude. Each data point in the box plot represents the normalized expression level for a cancer sample. Samples sizes for all cancer types are: ACC(N=79); BLCA(N=427); BRCA(N=1218); CESC(N=310); CHOL(N=45); COAD(N=329); DLBC(N=48); GBM(N=174); HNSC(N=566); KICH(N=91); KIRC(N=606); KIRP(N=101); LAML(N=173); LGG(N=534); LIHC(N=424); LUAD(N=576); LUSC(N=554); MESO(N=87); OV(N=266); PAAD(N=183); PCPG(N=187); PRAD(N=550); READ(N=105); SARC(N=265); SKCM(N=473); TGCT(N=156); THCA(N=572); THYM(N=122); UCEC(N=201); UCS(N=57); UVM(N=80).

### Example 10

(Improved Analysis of HMD/PMD Structure was Demonstrated Using the Solo-WCGW Motif)

The primary focus of the present disclosure has been on cell-type invariant PMDs, which were useful for investigating general properties of methylation loss over time. The 49% of the genome we identified as occurring within “Common PMDs” (using the SD>0.15 method) contains essentially all of the cell-type-invariant PMD regions that applicants identified previously (84). PMDs were defined in the present work by exploiting the inherent variance in PMD hypomethylation levels across large cohorts of samples, which was the only cross-sample feature bimodally distributed between HMDs and PMDs. Under this definition, for example, the core tumor group (containing only solid tumors) had almost the same degree of shared PMDs with blood malignancies (82%) as it did with other solid tumors not from the core set (85%) (FIG. 16). The power of this method might not apply to sample cohorts with little variation in hypomethylation levels, but it worked well for all the sample groups we examined here.

Specifically, FIGS. 16A-B show that for five sample groups, the majority of PMDs defined by high-SD bins were substantially overlapping PMDs defined earlier from the core tumor group (FIG. 3E). Distribution of cross-sample SDs for solo-WCGW methylation in all genomic 100 kb bins of the core tumor group (studied in FIG. 2B-C) are plotted on Y-axis, against SD distribution from 50 other blood malignancies (FIG. 16a); and 10 other solid tumors (FIG. 16B), plotted on X-axis. The figure shows the concordance of SD-based PMD definitions based on the core tumors and other tumors.

The present focus on common PMDs does not discount the importance of cell-type-specific PMDs. The work of applicant's group and others showed that about 25% of PMDs were cell-type specific (80, 81), and the present results here do not conflict with that. Others have established that cell-type specific cancer PMDs can be associated with gene expression differences, and distinguish different molecular subtypes of medulloblastoma and Atypical Teratoid/Rhabdoid tumors (81-83). Work from Fortin and Hansen showed that these cell-type-specific PMD differences corresponded to cell-type-specific topological domain and chromatin structure differences using Hi-C and DNase data from the same cell lines (84).

Deep PMD hypomethylation was observed in the methylome of T cells from a 103-year-old individual (FIG. 6A). Interestingly, in a previous study the hypomethylation patterns could not be conclusively called as PMDs even for the 103 year-old sample, likely due to the noise introduced by CpGs other than solo-WCGWs (86). According to particular aspects of the present invention, incorporation of solo-WCGW sequence features can be used to improve current methods for such cell-type-specific PMD detection, including kernel-based (87), HMM-based (88) and multi-scale based (89), and methods for methylation array data (84). Explicitly modeling and subtracting PMD-related hypomethylation will reduce noise and enhance the ability to detect changes in TET-mediated demethylation processes affecting short-range elements such as promoters, enhancers, and insulators.

While the discovery of solo-WCGW CpGs is a significant advance, the ability to detect differential PMDs in normal cell types with low levels of methylation loss, will remain a challenge. This is an important challenge to tackle, as it may allow the identification of PMD-associated cell-of-origin markers in cancer, which can be combined with mutational-signature-based cell-of-origin markers (85). PMD domain structure can also act as a useful proxy for 3D topological changes and other chromatin features in clinical disease samples where Hi-C or other direct mapping methods are not feasible due to the quantity or quality of intact chromatin available. PMDs also mark regions of gene silencing, and thus can help to infer the gene expression history of the cells being sampled. For instance, Hovestadt et al. showed that PMDs in medulloblastoma tumors reflected subtype-specific expression silencing in normal brain precursor cells (90).

### Example 11

(Stability of Rank-Based Correlation Between Methylomes was Demonstrated Using the Solo-WCGW Motif])

A rank-based analysis of 792 genomic 100 kb bins from chromosome 16 (FIG. 5) was performed to measure the HMD/PMD structure in normal tissues at different developmental stages. The rank correlations had only minor variations between replica or closely related samples (FIG. 27A) and the patterns were stable when using bins from different chromosomes (FIG. 27B).

Specifically, FIG. 27a shows rank correlation between three closely-related heart tissues and two replica of H1 ESC from different studies showing the magnitude of variation; N=792 non-overlapping 100 kbp genomic windows in chromosome 16. FIG. 27B shows order of Spearman's correlation in different chromosomes between the core tumor samples and the heart tissue samples from three different developmental stages.

### Example 12

(Alternative Explanation of PMD Hypomethylation)

While the present analysis supports replication timing as the most strongly associated genomic determinant of PMD methylation loss, replication timing is in practice very tightly linked to the Hi-C compartment “B” and the nuclear lamina based on applicants' work and the work of others (90, 91, 92). While the re-methylation window model is mechanistically attractive, we cannot rule out an alternative nuclear localization model (FIG. 8G), where methylation loss is due to compositional differences between the two nuclear compartments independent of replication timing, including differential activity of DNMTs or other chromatin regulatory factors. Indeed, various proteins are known to be regulated at the level of sub-nuclear compartment localization, such as TRIM28 (KAP-1) (93). It should be noted that the link between DNMT3B and H3K36me3 has been primarily described in mouse ES cells, which express a different isoform of Dnmt3b. Therefore, it remains possible that other DNMTs also contribute to the high methylation levels within early replicating regions. DNMT3A would be such a candidate, given that early replicating regions become hypomethylated upon Dnmt3a loss in a mouse lung cancer model (94). Recent work suggests that the heterochromatin and euchromatin nuclear compartments have a physical barrier created by liquid heterochromatin droplets formed by HP1-mediated phase separation (95, 96).

### Example 13

(Relevance of the PMD Sequence Signature to Somatic and Germline Mutational Landscape was Assessed)

To investigate any potential impact of the PMD sequence signature on introducing cytosine deamination mutations in the CpG dinucleotides, the relative proportion of somatic mutations that are within certain tetranucleotide sequence contexts and certain numbers of neighboring CpGs was studied. Somatic CpG to TpG mutations reported in an early gastric cancer whole-genome sequencing experiment was compared, and indeed confirmed that solo-WCGWs within late replicating PMDs had a lower CpG to TpG mutation rate compared with other sequence context (FIG. 24A). However, we also observed higher somatic mutation density overall in PMDs compared to HMDs, confirming earlier reports (97), possibly due to compensating effect from transcription-coupled DNA repair (98). More systematic investigation incorporating differential repair efficiencies will be necessary to investigate the effects solo-WCGW hypomethylation may have in shaping the single nucleotide mutational signatures observed in cancer and in evolution.

While only a limited number of samples were available for gametogenesis, dramatic PMD hypomethylation was observed in at least one germline cell type, the Germinal Vesicle, M-I Oocyte (FIG. 5B). This opens the possibility that local sequence determinants, HMD/PMD structure, or H3K36me3 distribution may play a role in methylation-sensitive deamination rates in the germline, and thereby help shape genome evolution. We studied de novo CpG->TpG mutations reported in a study of 1,548 Icelandic trios were studied, and these de novo CpG->TpG mutations in the maternal germline were indeed found to be depleted at CpGs in the WCGW context and with low local CpG density (FIG. 24B). The trend is not as apparent in paternal de novo mutations, consistent with lack of strong PMD structure in sperm (FIG. 5B). The standing distribution of human and mouse CpGs is also consistent with the hypothesis that tendency of losing methylation in solo-WCGW context in the germline may exert a protective role for these CpGs against deamination (FIGS. 24C and 24D). Such mechanisms have been proposed for other mutational processes (99), and the well-defined genomic constraints on the hypomethylation process described here will allow these types of analysis.

Specifically, FIGS. 24A-D show evidence supporting a model wherein hypomethylated solo-WCGWs within late replicating PMDs are protected from deamination and thus have a lower CpG to TpG mutation rate for both somatic mutations (from tumor sequencing) and de novo mutations in the human germline (from whole-genome trio sequencing). FIG. 24A shows the Impact of CpG dinucleotide PMD/HMD location, flanking CpG density and tetranucleotide sequence context on somatic mutation rate in 100 gastric cancer WGS24. FIG. 24B shows the impact of CpG dinucleotide sequence context on de novo germline mutation rates estimated from 1,548 Icelandic trios (25). FIG. 24C shows genomic CpG distribution stratified by PMD/HMD, flanking CpG density and sequence context in human. FIG. 24D shows genomic CpG distribution stratified by PMD/HMD, flanking CpG density and sequence context in mouse.

### Example 14

(Certain Specific Sub-Patterns that Match the Solo-WCGW Definition were Found to be More Predictive than the General Definition, and DNA Shape Features were Also Found to be Predictive)

Above, working Example 1 demonstrates that the Solo-WCGW motif is highly predictive of PMD methylation loss across a large number of cell types and across mammalian species. Formally, Solo-WCGW is defined as n(x)WCpGWn(x), where a series of x positions on either side can match any base n (A,C,T, or G) but none can match a CG dinucleotide. According to particular additional aspects of the present invention that we have demonstrated, much of the predictive value (for replication-associated methylation loss) is captured by this general pattern. However, this pattern represents a large number of actual sequence instances (using the preferred definition of x=34, there are approximately 3 million unique individual matching sequences in the human genome), and thus we investigated if it is possible to define sub-patterns that may further improve the predictive value, and that be used to prioritize sequences used in, for example, biomedical tests and other methods described herein. An exemplary covariance analysis was performed that supports the presence of such sub-patterns, as described below.

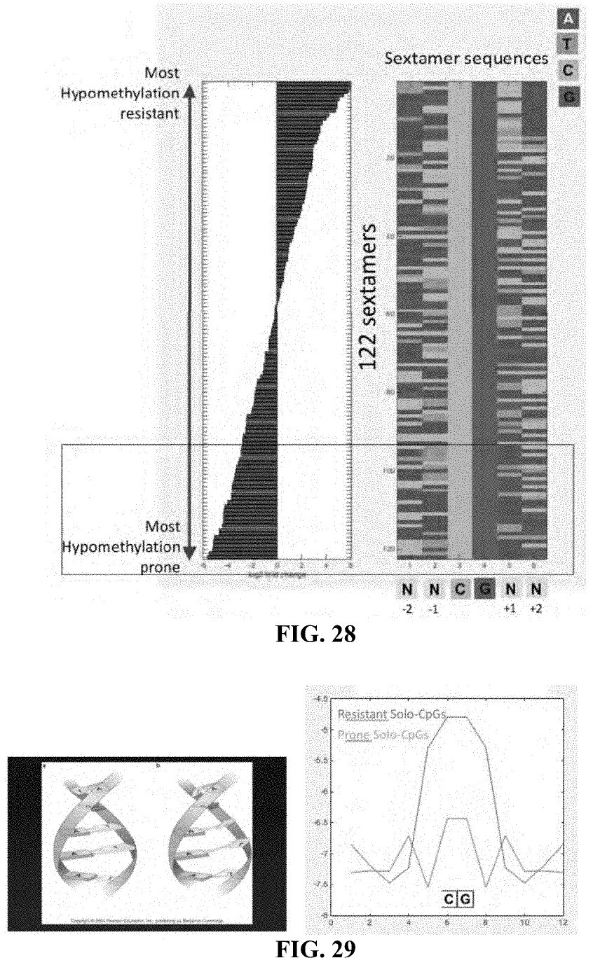

In the analysis, we started with the set of all Solo-CpGs (n(35)CpGn(35)) that fell within each common PMD as described above, and then compared the similarity of each Solo-CpG to all others within the common PMD using covariance across samples in our human WGBS set, described above. Hypomethylation prone Solo-CpGs were found to have high average covariance with other Solo-CpGs within the same PMD, and we defined those with average covariance greater than or equal to the 85th percentile of covariance for all Solo-CpGs in all common PMDs in the genome as “hypomethylation prone”. Those with covariances less than or equal to the 5th percentile of all values, with average methylation across all samples of >0.7, were defined as “hypomethylation resistant”. We then calculated the ratio of hypomethylation resistant to hypomethylation prone frequencies for all sextanucleotide Solo-CpG sequences (matching the pattern “NNCGNN”), and sorted sequences from those most resistant to those most prone, as shown in FIG. 28. As expected, the most hypomethylation prone sequences match the pattern WCGW, confirming our definition of Solo-WCGW as the predominant predictor of replication-associated hypomethylation. However, we also observed a tendency for the sequence pattern CWCGWG (or mWCGWG, where m=C or A) to be even more prone than the more general WCGW sequence in the context of the Solo-WCGW motif This is consistent with art-recognized knowledge that many DNA-binding proteins and protein complexes have recognition specificities that span 4-10 nucleotides. While this is an initial covariance finding that can be further validated using the larger datasets available on Infinium Human Methylation platforms, it indicates that the Solo-WCGW pattern that we have fully validated in multiple datasets, likely represents a lower bound in terms of predicting replication-associated hypomethylation. Thus, the covariance analysis refinements to the Solo-WCGW pattern can be used for prioritization of sequences to use in biomedical tests, and other applications disclosed herein.

In addition to DNA sequence patterns, DNA secondary structure or “DNA shape” is known in the art to play a role in the binding efficiency of chromatin modifying proteins, and may thus also be useful for defining sub-patterns of the Solo-WCGW pattern that can be used for prioritization of sequences to use, for example, in biomedical tests and other methods to improve the accuracy of replication-associated hypomethylation prediction. We have used the same hypomethylation resistant vs. hypomethylation prone analysis described in the last paragraph, to investigate the association of DNA shape, using the tool DNAShapeRTM (102). By comparing DNA shape in the most hypomethylation resistant vs. most hypomethylation prone Solo-CpGs, we determined that one particular DNA shape, “propeller twist” was specifically low in the hypomethylation prone Solo-CpGs, as shown in FIG. 29. This indicates that shape information can be used to further improve the set of Solo-WCGW instances chosen to predict replication-associated methylation loss.

Specifically, FIG. 29 shows, according to particular exemplary aspects, that DNA shape features were also found to be predictive of replication-associated DNA methylation loss. The upper panel shows a generic illustration (taken from 2004 Pearson Education, Inc., publishing as Bnjamin Cummings) of a propeller twist that results from bond rotation. The lower panel compares to extent of propeller twist at the CpG dinucleotide found in hypomethylation resistant Solo-WCGW motif sequences, to that found in hypomethylation prone Solo-WCGW motif sequences. Specifically, hypomethylation prone Solo-WCGW motif sequences were found to have a lower propeller twist DNA shape relative to hypomethylation resistant Solo-WCGW motif sequences.

### Example 15

(Materials and Methods for Examples 16-18)

Primary Cell Culture.

Primary human cells obtained from multiple tissues and donors (n=5, Table 12), as facilitated by biobank Coriell, were serially-cultured until replicative senescence. At each passaging, or replating, of cells, cell count and viability was measured to calculate population doubling level (PDL), the metric for observed mitotic history. DNA was extracted from cells at each timepoint (n=116).

DNA Methylation Assay.

Bisulfite-converted DNA was applied to an Illumina HumanMethylation EPIC microarray and fluorescence was measured aboard an Illumina iScan at probes sensitive to methylation status at >850,000 CpGs in the human genome. Other DNA methylation assays can be substituted for the EPIC array, such as other Illumina methylation arrays or whole genome bisulfate sequencing.

Beta Calling.

Using the sesame package (103) in statistical software R, raw fluorescence intensities were normalized to out-of-band fluorescence intensity (73) before beta value calculation. Beta value is the measure of degree of methylation at a given CpG dinucleotide; a beta value of 1 reflects complete methylation and 0 reflects complete unmethylation. Beta-calling of Illumina 450K and EPIC arrays is supported by sesame; other upstream methylation analyses will have different processing requirements.

Qa/Na Removal.

Specific samples and probes which exhibited consistently poor performance, as determined by NA/missing values returned on >5% of CpGs or samples, respectively, were removed. NA probe filtering stringency of the test set shown from hereafter was complete to ensure a most-reproducible probe set: probes with ≥1 NA (n=279,797) were removed, although differing applications may allow more relaxed filtering.

Solo-WCGW Subsetting.

Following sample and probe removal, probes were filtered to include only solo-WCGW CpGs in common PMDs (n=26,732 on EPIC microarray, n=9,711 following complete NA removal). Solo-WCGW identity is based on profiling of human genome build 19 (hg19); a full manifest is available at http://zwdzwd.io/pmd/soloWCGW_inCommonPMDshg19.bed.gz. Sequence positions may differ slightly by genome build.

### Example 16

(Elastic Net Modeling Strategy)

PDL Standardization.

Elastic net regression (ENR) was applied via the glmnet package in R across individual donor cultures, regressing against observed PDL in culture. Glmnet settings were mostly default; alpha was set to 0.5 (to achieve ENR) with gaussian distribution. A linear model was automatically selected. The mitotically youngest donor culture was AG21839, a neonatal foreskin fibroblast cell line. To standardize PDL and allow for development of a multi-tissue mitotic clock, starting PDLs from all other cell lines were normalized to the ENR model built from AG21839 (Table 12, ‘Standardized PDL’). Delta PDL was added to adjusted starting PDL for the following timepoints.

Multi-Tissue ENR Modeling.

Using prefiltered beta values from all cultures with standardized PDL, ENR was again performed using the same settings as above.

10-Fold Cross Validation and Probe Reduction.

To select the number of CpGs allowed in the model and control for potential overfitting, 10-fold cross validation was performed on the model. Lambda was set at lambda minimum+1 standard deviation, resulting in 44 CpGs included in this model (Table 13).

Model Performance.

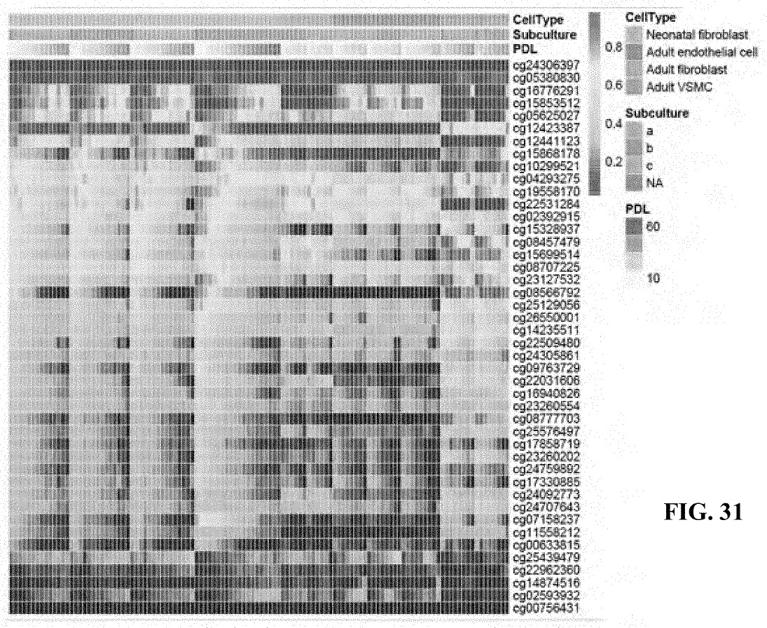

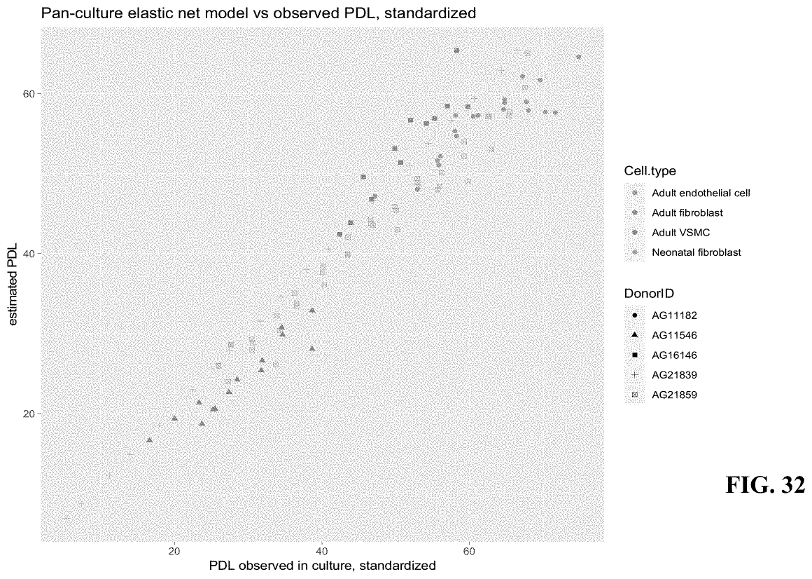

A heatmap of beta values at the selected CpGs across advancing PDL shows consistent hypomethylation across donors, cell types, and subcultures (FIG. 31). Predictive performance of the generated clock is shown for individual cultures (FIG. 32, r2≥0.970, cor≥0.925); across all cultures r2=0.9975 and correlation=0.976. Predictive performance of this model compared to other methylation clocks is shown in Table 14.

Suggested Use:

The elastic net regression strategy produced a robust 44-CpG model for predicting mitotic history within and between cell types (Tables 15A-B).

### Example 17

(Individual Probe Regression Strategy)

Simple linear regression was applied individually to each prefiltered probe.

Regression coefficients r and r2 from all primary cell cultures were compared.

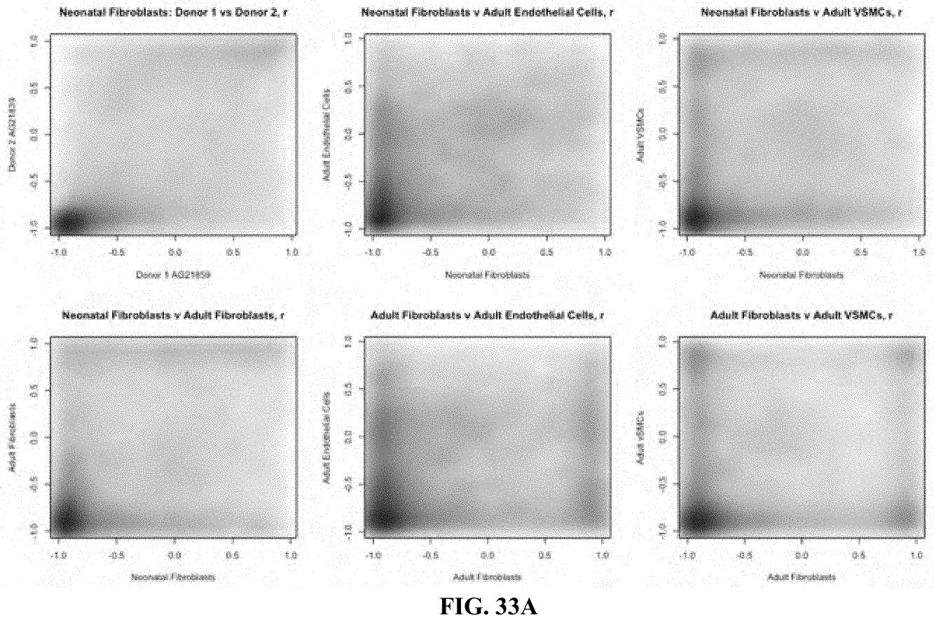

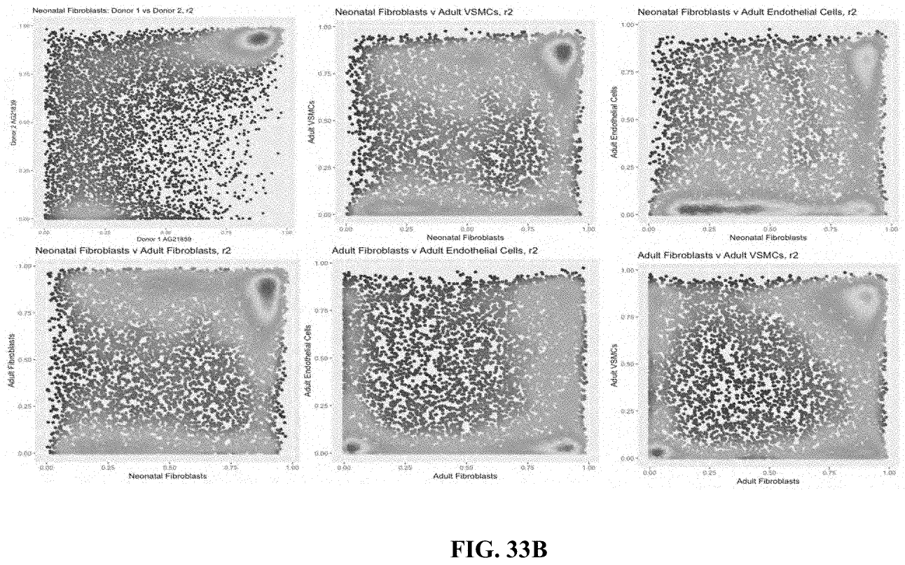

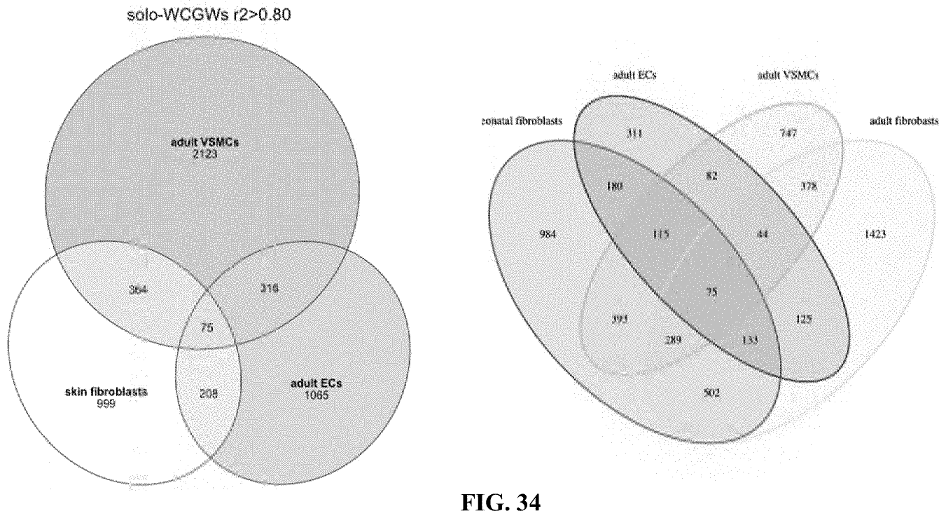

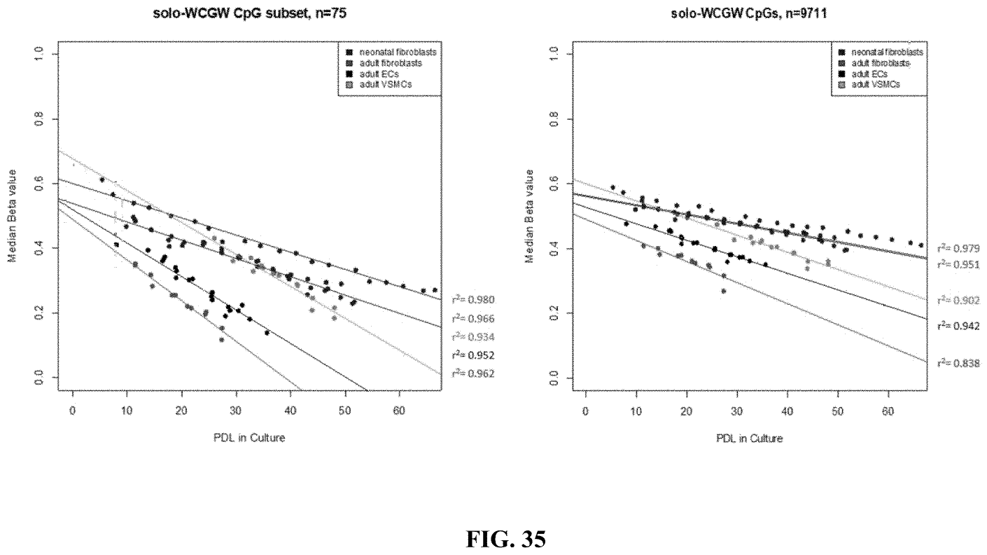

Density plots of regression coefficients r and r2 (FIGS. 33A and 33B, respectively) show a consistently strongly correlated group of probes shared across cell types, donors, and donor age. This group was extracted by filtering only the probes which met the following criteria in all cultures: r2>0.80 (FIG. 34). The resulting group of 75 CpGs showed markedly-improved predictive performance over solo-WCGWs altogether, particularly for cultures from adult donors (FIG. 35).

Model Performance:

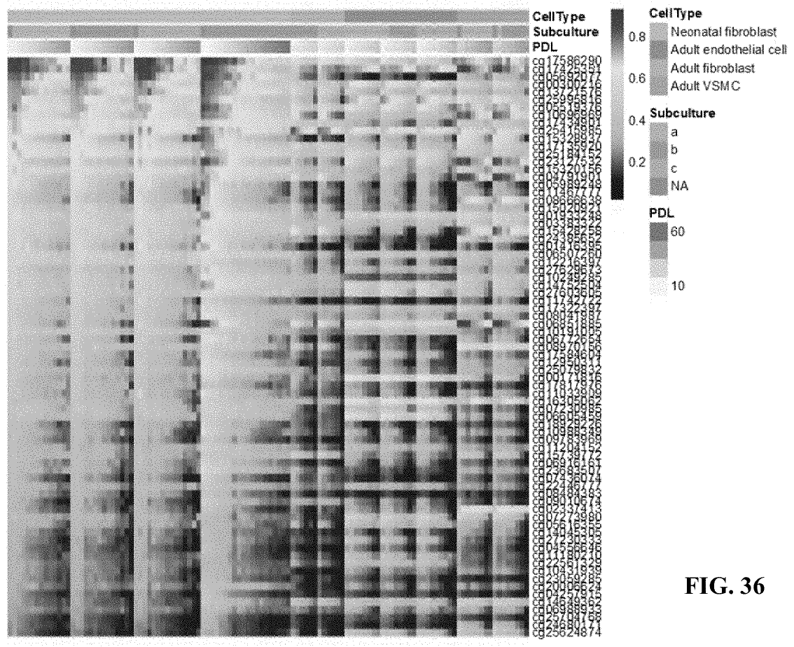

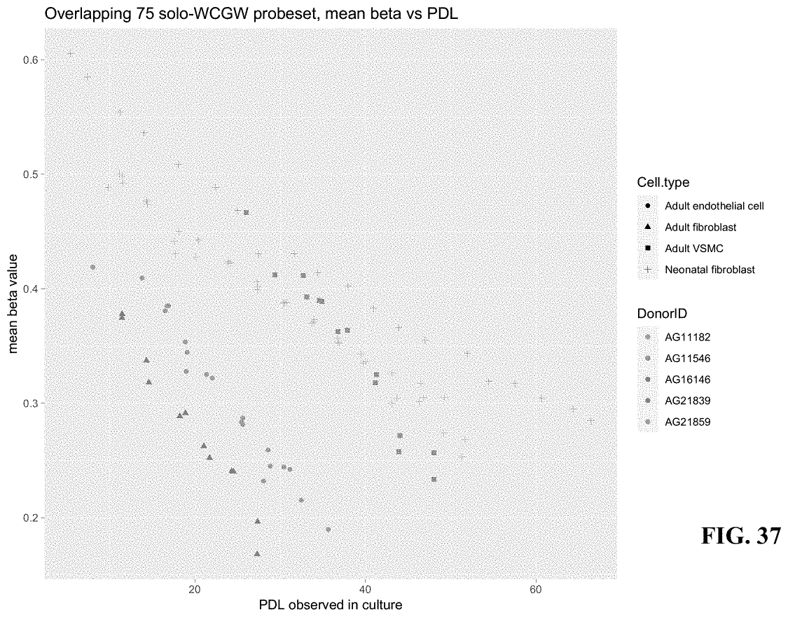

A heatmap of the selected CpGs across advancing PDL shows consistent hypomethylation across donors, cell types, and subcultures (FIG. 36). The mean beta value of the selected CpGs is plotted against observed PDL (FIG. 37). Overall correlation for unstandardized PDL is poor (−0.549) but individual culture correlations<−0.977. Predictive performance of this model compared to other methylation clocks is shown in Table 3.

Suggested Use:

The individual probe regression strategy, yielding a subset of 75 (Tables 16A-B) strongly correlated probes for all tissue types studied, offers an immediate refinement of the solo-WCGW signature. When beta values of these CpGs are weighted equally, robust intra-cell-type mitotic history comparisons are possible.

### Example 18

(Elastic Net Model Versus Individual Regression Model)

While both are highly predictive, the probe landscapes of the two mitotic clocks are rather distinct. There are only two overlapping CpG between the sets, cg15328937 and cg23127532; both are negatively correlated in both models. Nine and 35 CpGs of the elastic net model are positively and negatively correlated with mitotic age, respectively. Regression coefficients for the elastic net model range from −19.24−15.52; the intercept is 83.01. For the individual regression model, all CpGs are equally-weighted by taking the mean, but each cell type has a different intercept, ranging from 0.500 for AG16146 to 0.738 for AG11546, and slope, ranging from −0.005 for AG21839 to −0.011 for AG16146. Whereas the elastic net model places multi-tissue-type mitotic history on the same scale, the individual regression model's cell-specific slope/intercept values likely reflect slight differences in rates of solo-WCGW hypomethylation across tissue type and age.

### Example 19

(Comparison to Existing Clocks)

Comparison to Hannum Clock.

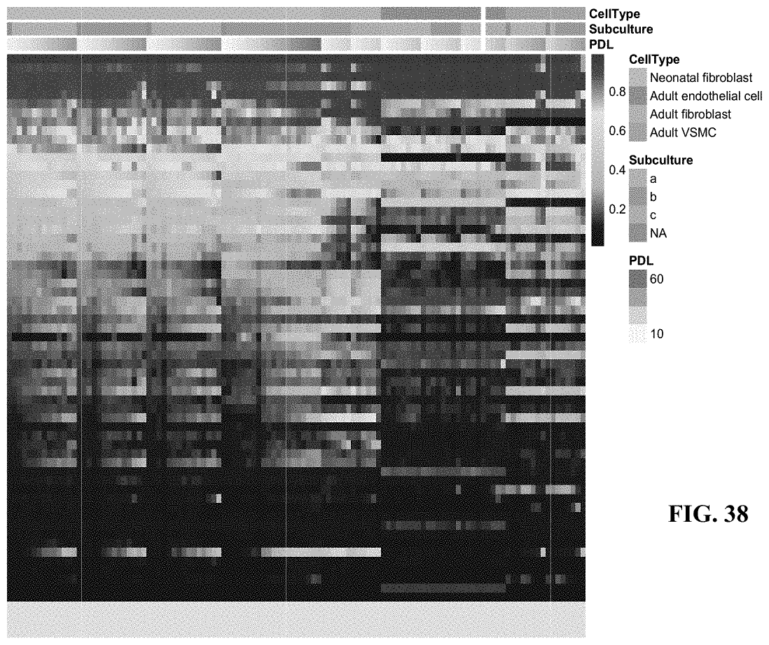

Hannum pioneered the modern methylation clock with a 71-CpG model (58) that predicts chronological age with high accuracy (>90% accuracy with mean error of several years) in whole blood samples in adults. In addition to introducing a high-performing methylation clock, to produce it Hannum et all implemented elastic net regression (104) via the glmnet package (105) in statistical software R. Elastic net regression (ENR) combines Lasso and ridge regression techniques to reduce both the number of variables and the relative contribution of each variable to a multivariate model, in which the number of potential variables vastly outnumbers the observations. It has since proven to be adept at modeling methylation clocks while controlling for overfitting. Definitively limiting its adoption, Hannum's clock performs poorly in non-blood samples and in blood samples from children; the composition of white blood cells and resulting methylation patterns changes dramatically during development. Three of the 71 CpGs are solo-WCGWs; none of these are present in the solo-WCGW clock. A heatmap of beta values at Hannum CpGs is shown in FIG. 38.

Comparison of DNAm Age.

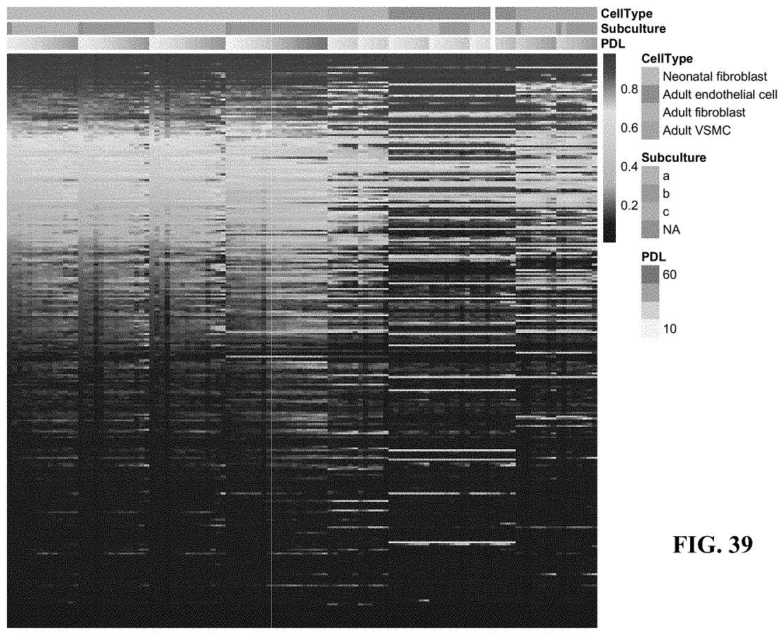

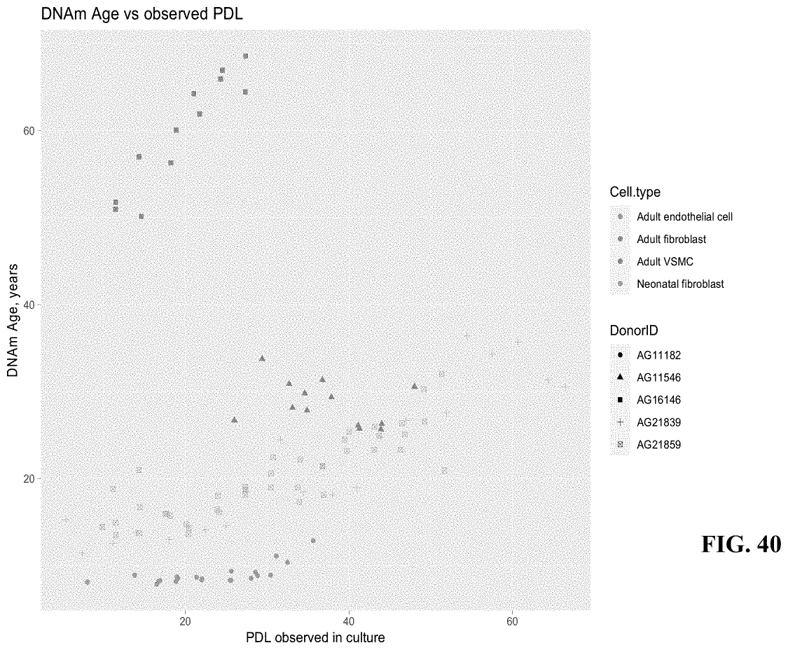

The most widely-applied methylation clock, ‘DNAm Age,’ (59) predicts chronological age with high accuracy in most human tissues. Elastic net regression was applied across a large dataset of Illumina Infinuim HumanMethylation 27K and 450K BeadChip array data from apparently-healthy human tissues of different chronological ages to mathematically select 353 CpGs and individual coefficients for each CpG. The weighted average of coefficient-multiplied beta values at these CpGs estimates chronological age with high accuracy across most tissues. Of the 353 CpGs, 193 are positively and 160 are negatively correlated with chronological age. DNAm Age was developed to perform well on multiple tissues with extremely variable mitotic capacities (e.g. brain and liver) so it is unsurprising that there is no overlap between it and the solo-WCGW clocks, however, three of the 353 CpGs are solo-WCGWs in common PMDs. A heatmap of beta values at DNAm Age CpGs is shown in FIG. 39; a plot of DNAm Age vs PDL by cell type is shown in FIG. 40.

Comparison to Skin & Blood Clock.

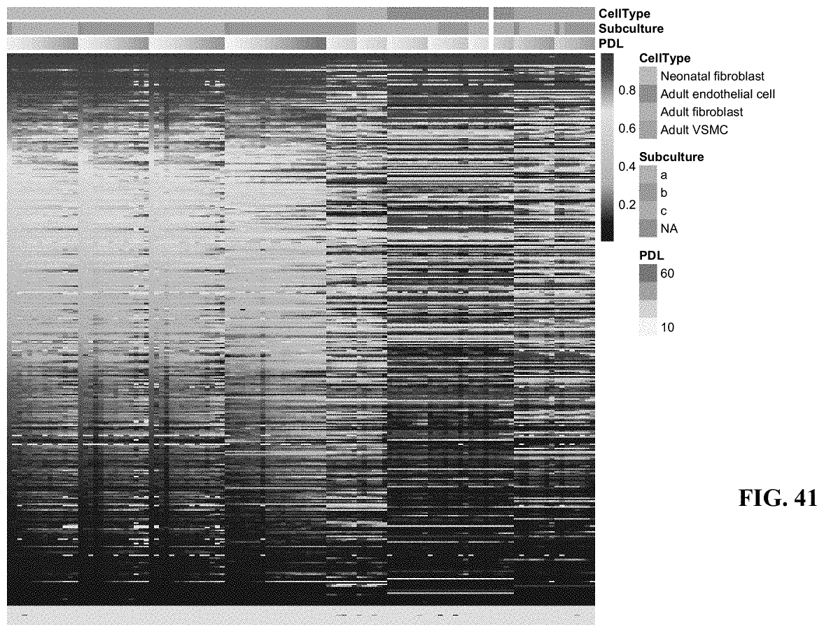

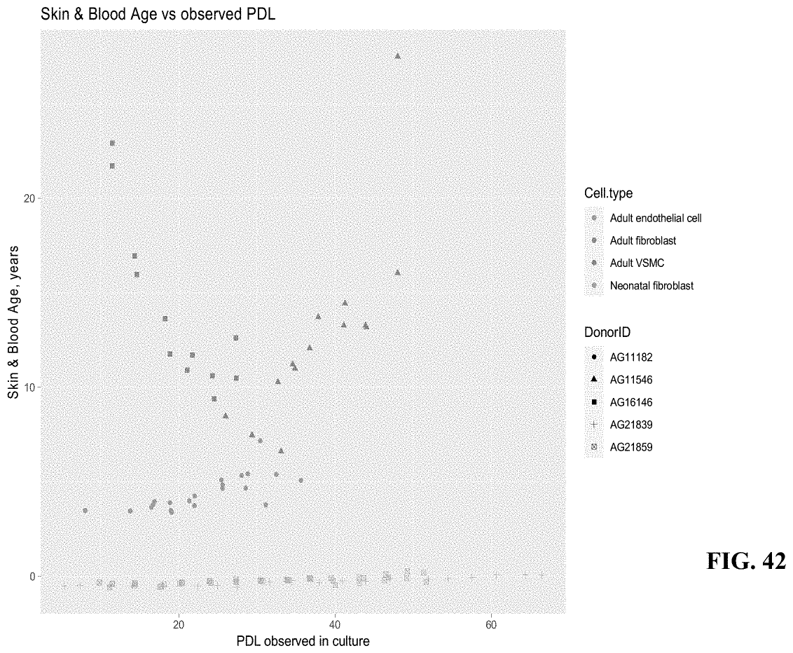

Despite high performance across most tissues, DNAm Age predictability underperformed on skin and blood samples. For clinical and forensic applications, skin and blood tissues are amongst the easiest to collect and thus the application of DNAm Age was limited. To remedy this, Horvath developed a similar ‘Skin & Blood Clock’ (106) which shares 60 CpGs (of 391) with DNAm Age. Six of these CpGs are solo-WCGWs, although there is no overlap of these probes with the three solo-WCGWs in DNAm Age. Again, there is no probe overlap between the solo-WCGW clocks and the Skin & Blood clock. A heatmap of beta values at Skin & Blood Clock CpGs is shown in FIG. 41; a graph of Skin & Blood Age vs PDL by cell type is shown in FIG. 42.

Comparison to DNAm PhenoAge.

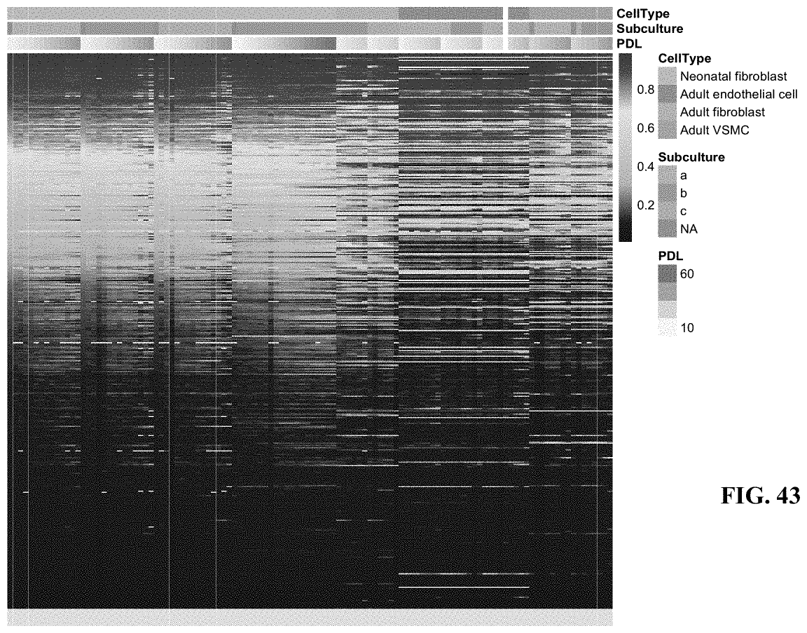

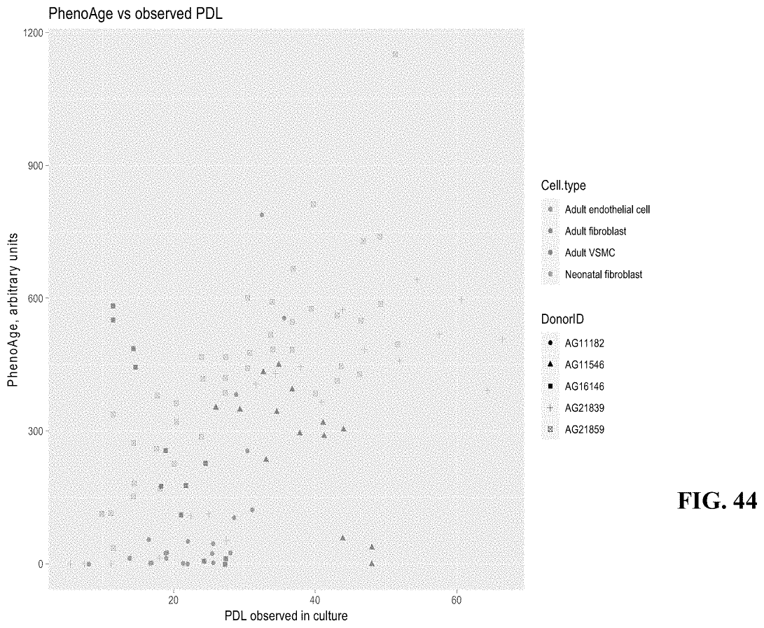

The ‘DNAm PhenoAge’ methylation clock (107) was trained not to predict chronological age of tissues but to predict all-cause mortality, or ‘phenotypic age,’ as defined by a panel of biomarkers. Using the same mathematical parameters as Horvath's chronological methylation clocks, ENR produced 513 CpGs, of which 57 overlap with DNAm Age and 41 overlap with the Skin & Blood Clock (20 are shared by all 3 models, albeit with differing weights). Four of these CpGs are solo-WCGWs, however none of these are probes within the solo-WCGW clocks. A heatmap of beta values at PhenoAge CpGs is shown in FIG. 43; a graph of PhenoAge (in relative units) vs PDL by cell type is shown in FIG. 44.

Comparison to EpiTOC′ Mitotic-Like Methylation Clock.

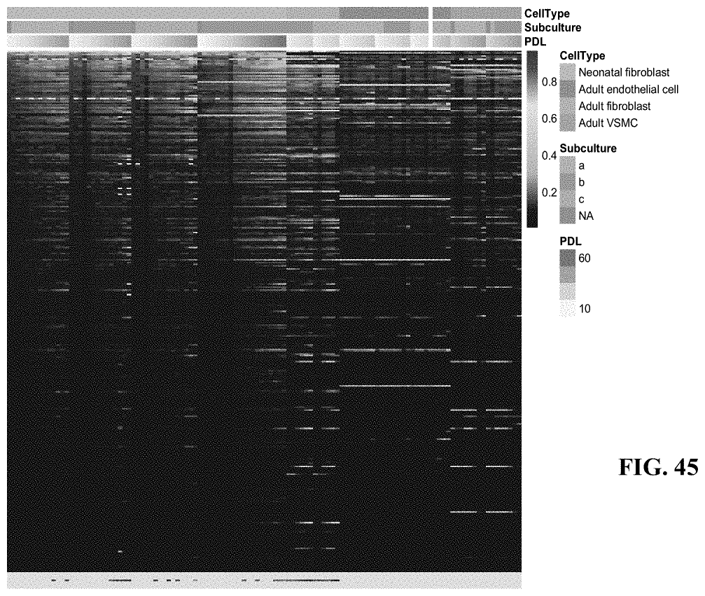

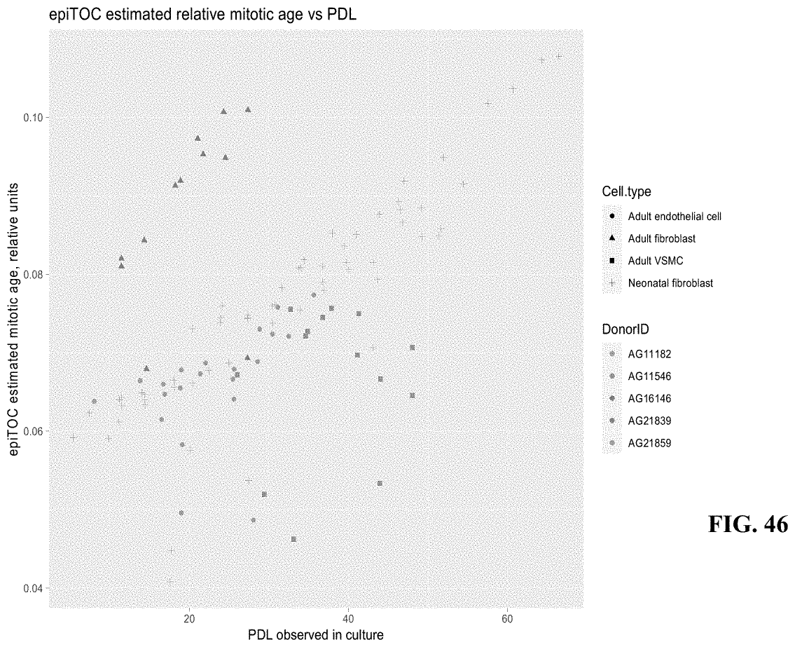

More comparable in developmental strategy and in application to the solo-WCGW clock is the ‘epiTOC’ mitotic-like methylation clock (108). Whereas DNAm Age, the Skin & Blood Clock, and DNAm PhenoAge were unsupervised in their construction, instead solely relying on glmnet-powered ENR and 10-fold cross validation to select probes and coefficients, Yang et al prefiltered CpGs based on the observation that polycomb target CpGs gain methylation with advancing age in a seemingly mitotic-capacity-driven manner. PRC2 polycomb target CpGs (109) were subsetted from the large whole blood dataset Hannum cultivated, and only CpGs that were unmethylated in fetal tissues and gained methylation over advancing chronological age in the training set were considered for the model: 385 CpGs remained. The epiTOC model was not built on ENR but takes the untransformed mean of the beta values at these 385 CpGs to estimate relative mitotic age. This model was trained solely off whole blood samples yet its authors have applied it to multiple tissues. None of the 385 epiTOC CpGs are present in DNAm Age, Skin & Blood, DNAm PhenoAge, or the solo-WCGW clocks. Indeed, none of the epiTOC probes are solo-WCGWs; this is likely a product of preselecting only PRC2-target CpGs. A heatmap of beta values at epiTOC CpGs is shown in FIG. 45; a graph of epiTOC mitotic age (relative units) vs PDL by cell type is shown in FIG. 46.

The solo-WCGW mitotic clock of the present invention is the first model to estimate mitotic age with high accuracy in primary cell culture (Table 3). Relative mitotic age estimation and comparisons between same-tissue samples can be performed with either the elastic net model or the independent regression model. Cross-tissue mitotic age comparisons (e.g. directly comparing skin tissue to vascular smooth muscle tissue) and absolute mitotic history can be estimated with the elastic net model and not the independent regression model. The construction of the solo-WCGW clock is unique in that it is the first of its kind to be trained from serial cell culture data. This feature gives the clock increased sensitivity—down to individual population doublings—over other methylation clocks which estimate age in years (with mixed success on cell culture data, see FIGS. 39-42) or relative mitotic age in arbitrary units (with little success on cell culture data, see FIGS. 45-46). Additionally, the solo-WCGW mitotic clock is unique in that it combines a well-characterized biological premise—mitosis-associated hypomethylation at solo-WCGW CpGs—with powerful multivariate regression techniques.

According to additional aspects, therefore, more specific definitions within the general Solo-WCGW pattern are provided for prioritization of sequences used in biomedical tests and other methods disclosed herein to track replication-associated DNA methylation loss.

### Example 20

(Additional Exemplary Methods)

Particular aspects of the present invention, provide, but are not limited to the following exemplary methods:

A method for determining chronological age, or accelerated chronological age of a cell or tissue sample of a test subject, comprising:

collecting cell and tissue samples, sort cells if necessary;

extracting DNA;

performing bisulfate conversion and library preparation (e.g., sonicate DNA, PCR amplification);

measuring beta*values (e.g., using 1000 probes with the extension base targeting solo-WCGW CpGs);

computing a score by taking the average of these solo-WCGW CpG beta values;

using the score as an indication of mitotic age;

computing a calibration curve by looking at the mitotic age score computed above in a population in a range of chronological ages; and

for test individuals, interpolating the chronological age to compare the standard mitotic age with the test mitotic age to determine if there is accelerated aging.

(*The Beta-value is the ratio of the methylated probe intensity and the overall intensity (sum of methylated and unmethylated probe intensities; e.g., see Du, Pan, et al., BMC Bioinformatics 2010; 11:587; doi 10.1186/1471-2105-11-587, (incorporated by reference herein).

A method for determining the mitotic turnover history of a cell, comprising:

collecting/immortalizing a primary cell line (e.g., lymphoblastoid cell line or other tissues);

passing the cell line to certain passage numbers;

extracting DNA for each cell with a certain passage number, and performing bisulfate conversion and library preparation;

calibrating the passage number against solo-WCGW beta value averages (e.g., using 1000 probes with the extension base targeting solo-WCGW CpGs); and

for test samples, interpolating the passage number using the measured solo-WCGW value averages.

A method of measuring excessive replicative turnover history in cancer by comparing to matched normal cell-type of origin, comprising:

collecting, for each tumor, a normal cell type of origin;

deriving a passage number calibration curve using the method above;

interpolating the passage number of the tumor cells; and

comparing the passage number of the tumors with the normal.

A method for measuring increased risk of a subject for conditions associated with excessive replicative turnover or aging (e.g., cancer, neurodegenerative disease, cardiovascular disease, progeria etc.), comprising:

collecting relevant tissues/cell types from affected individuals and disease-free controls;

measuring the passage number using the method described above, wherein the passage number is associated with the disease onset and age; and

calibrating the risk for the corresponding disease using the determined passage number of the relevant cells.

A method for identifying subjects for increased surveillance and screening, comprising:

collecting cell-free circulating DNA from patients or test individuals and disease-free controls;

performing bisulfite conversion and library preparation;

computing a mitotic replicative score by averaging the solo-WCGW CpG beta values (e.g., using 1000 probes with the extension base targeting solo-WCGW CpGs); and

identifying subjects in need of increased surveillance and screening if their mitotic replicative score is significantly higher than disease-free controls.

A method for forensic analysis, comprising:

collecting tissue from the crime scene;

extracting DNA and performing bisulfite conversion;

measuring solo-WCGW CpG methylation average in the extracted DNA (e.g., using 1000 probes with the extension base targeting solo-WCGW CpGs); and

computing a chronological age using a matched cell type using the method outlined above.

### REFERENCES

References cited with respect to working Examples 1-7, and incorporated herein by reference for their respective teachings:
1. Ehrlich, M. & Wang, R. Y. 5-Methylcytosine in eukaryotic DNA. Science 212, 1350-7 (1981).
2. Feinberg, A. P. & Vogelstein, B. Hypomethylation distinguishes genes of some human cancers from their normal counterparts. Nature 301,89-92 (1983).
3. Gama-sosa, M. A. et al. The 5-methykytosine content of DNA from human tumors. Nucleic Acids Res. 11,6883-6894 (1983).
4. Goelz, S., Vogelstein, B. & Feinberg, A. Hypomethylation of DNA from benign and malignant human colon neoplasms. Science (80-.). 228,187-190 (1985).
5. Hansen, K. D. et al. Increased methylation variation in epigenetic domains across cancer types. Nat. Genet. 43,768-775 (2011).
6. Berman, B. P. et al. Regions of focal DNA hypermethylation and long-range hypomethylation in colorectal cancer coincide with nuclear lamina-associated domains. Nat. Genet. 44, 40-46 (2012).
7. Fortin, J.-P. & Hansen, K. D. Reconstructing A/B compartments as revealed by Hi-C using long-range correlations in epigenetic data. Genome Biol. 16, 180 (2015).
8. Weber, M. et al. Chromosome-wide and promoter-specific analyses identify sites of differential DNA methylation in normal and transformed human cells. Nat. Genet. 37, 853-62 (2005).
9. Aran, D., Toperoff, G., Rosenberg, M. & Hellman, A. Replication timing-related and gene body-specific methylation of active human genes. Hum. Mol. Genet. 20, 544 670-680 (2011).
10. Bergman, Y. & Cedar, H. DNA methylation dynamics in health 545 and disease. Nat. Struct. Mol. Biol. 20, 274-281 (2013).
11. Quante, T. & Bird, A. Do short, frequent DNA sequence motifs mould the epigenome? Nat. Rev. Mol. Cell Biol. 17, 257-62 (2016).
12. Lister, R. et al. Human DNA methylomes at base resolution show widespread epigenomic differences. Nature 462, 315-322 (2009).
13. Timp, W. et al. Large hypomethylated blocks as a universal defining epigenetic alteration in human solid tumors. Genome Med 6, 61 (2014).
14. Hovestadt, V. et al. Decoding the regulatory landscape of medulloblastoma using DNA methylation sequencing. Nature 510, 537-541 (2014).
15. Baylin, S. & Bestor, T. H. Altered methylation patterns in cancer cell genomes: Cause or consequence? Cancer Cell 1, 299-305 (2002).
16. Brennan, K. & Flanagan, J. M. Is there a link between genome-wide hypomethylation in blood and cancer risk? Cancer Prev. Res. (Phila). 5, 1345-57 (2012).
17. Ehrlich, M. et al. Amount and distribution of 5-methylcytosine in human DNA from different types of tissues of cells. Nucleic Acids Res. 10, 2709-21 (1982).
18. Lister, R. et al. Hotspots of aberrant epigenomic reprogramming in human induced pluripotent stem cells. Nature 471, 68-73 (2011).
19. Hansen, K. D. et al. Large-scale hypomethylated blocks associated with Epstein-Barr virus-induced B-cell immortalization. Genome Res. 24, 177-184 (2014).
20. Landan, G. et al. Epigenetic polymorphism and the stochastic formation of differentially methylated regions in normal and cancerous tissues. Nat. Genet. 44, 1207-1214 (2012).
21. Shipony, Z. et al. Dynamic and static maintenance of epigenetic memory in pluripotent and somatic cells. Nature 513, 115-119 (2014).
22. Schroeder, D. I. et al. The human placenta methylome. Proc. Natl. Acad. Sci. U.S.A. 110, 6037-42 (2013).
23. Kulis, M. et al. Whole-genome fingerprint of the DNA methylome during human B cell differentiation. Nat. Genet. 47, 746-56 (2015).
24. Durek, P. et al. Epigenomic Profiling of Human CD4(+) T Cells Supports a Linear Differentiation Model and Highlights Molecular Regulators of Memory Development. Immunity 45, 1148-1161 (2016).
25. Schultz, M. D. et al. Human body epigenome maps reveal noncanonical DNA methylation variation. Nature 523, 212-6 (2015).
26. Vandiver, A. R. et al. Age and sun exposure-related widespread genomic blocks of hypomethylation in nonmalignant skin. Genome Biol. 16, 80 (2015).
27. Song, Q. et al. A reference methylome database and analysis pipeline to facilitate integrative and comparative epigenomics. PLoS One 8, e81148 (2013).
28. Edwards, J. R. et al. Chromatin and sequence features that define the fine and gross structure of genomic methylation patterns. Genome Res. 20, 972-80 (2010).
29. Gaidatzis, D. et al. DNA Sequence Explains Seemingly Disordered Methylation Levels in Partially Methylated Domains of Mammalian Genomes. PLoS Genet. 10, (2014).
30. Carter, S. L. et al. Absolute quantification of somatic DNA alterations in human cancer. Nat. Biotechnol. 30, 413-421 (2012).
31. Farlik, M. et al. DNA Methylation Dynamics of Human Hematopoietic Stem Cell Differentiation. Cell Stem Cell 19, 808-822 (2016).
32. Knijnenburg, T. a et al. Multiscale representation of genomic signals. Nat. Methods 11, 689-94 (2014).
33. Guelen, L. et al. Domain organization of human chromosomes revealed by mapping of nuclear lamina interactions. Nature 453, 948-51 (2008).
34. Lister, R. et al. Global Epigenomic Reconfiguration During Mammalian Brain Development. Science 341, 629-643 (2013).
35. Tomasetti, C. & Vogelstein, B. Variation in cancer risk among tissues can be explained by the number of stem cell divisions. Science (80-.). 347, 78-81 (2015).
36. Burnet, F. M. A modification of Jerne's theory of antibody production using the concept of clonal selection. CA. Cancer J. Clin. 26, 119-21 (1976).
37. Wu, H. & Zhang, Y. Reversing DNA methylation: Mechanisms, genomics, and biological functions. Cell 156, 45-68 (2014).
38. Alexandrov, L. B. et al. Clock-like mutational processes in human somatic cells. Nat. Genet. 47, 1402-7 (2015).
39. Lee, E. et al. Landscape of Somatic Retrotransposition in Human Cancers. Science (80-.). 337, 967-971 (2012).
40. Tubio, J. M. C. et al. Extensive transduction of nonrepetitive DNA mediated by L1 retrotransposition in cancer genomes. Science (80-.). 345, 1251343-1251343 (2014).
41. Rodriguez-Martin, B. et al. Pan-cancer analysis of whole genomes reveals driver rearrangements promoted by LINE-1 retrotransposition in human tumours. bioRKiv 179705 (2017). doi:10.1101/179705
42. Iskow, R. C. et al. Natural mutagenesis of human genomes by endogenous retrotransposons. Cell 141, 1253-1261 (2010).
43. Howard, G., Eiges, R., Gaudet, F., Jaenisch, R. & Eden, A. Activation and transposition of endogenous retroviral elements in hypomethylation induced tumors in mice. Oncogene 27, 404-8 (2008).
44. Santos, A., Wernersson, R. & Jensen, L. J. Cyclebase 3.0: A multi-organism database on cell-cycle regulation and phenotypes. Nucleic Acids Res. 43, D1140-D1144 (2015).
45. Baubec, T. et al. Genomic profiling of DNA methyltransferases reveals a role for DNMT3B in genic methylation. Nature 520, 243-7 (2015).
46. Li, E., Bestor, T. H. & Jaenisch, R. Targeted mutation of the DNA methyltransferase gene results in embryonic lethality. Cell 69, 915-26 (1992).
47. Li, Z. et al. Distinct roles of DNMT1-dependent and DNMT1-independent methylation patterns in the genome of mouse embryonic stem cells. Genome Biol. 16, 115 (2015).
48. Jones, P. a & Liang, G. Rethinking how DNA methylation patterns are maintained. Nat. Rev. Genet. 10, 805-811 (2009).
49. Hermann, A., Goyal, R. & Jeltsch, A. The Dnmt1 DNA-(cytosine-05)-methyltransferase methylates DNA processively with high preference for hemimethylated target sites. J. Biol. Chem. 279, 48350-9 (2004).
50. Flynn, J., Azzam, R. & Reich, N. DNA binding discrimination of the murine DNA cytosine-05 methyltransferase. J. Mol. Biol. 279, 101-16 (1998).
51. Bashtrykov, P., Ragozin, S. & Jeltsch, A. Mechanistic details of the DNA recognition by the Dnmt1 DNA methyltransferase. FEBS Lett. 586, 1821-1823 (2012).
52. Johann, P. D. et al. Atypical Teratoid/Rhabdoid Tumors Are Comprised of Three Epigenetic Subgroups with Distinct Enhancer Landscapes. Cancer Cell 29, 379-393 (2016).
53. Liang, G. et al. Cooperativity between DNA methyltransferases in the maintenance methylation of repetitive elements. Mol. Cell. Biol. 22, 480-91 (2002).
54. Schermelleh, L. et al. Dynamics of Dnmt1 interaction with the replication machinery and its role in postreplicative maintenance of DNA methylation. Nucleic Acids Res. 35, 4301-12 (2007).
55. Neri, F. et al. Intragenic DNA methylation prevents spurious transcription initiation. Nature 543, 72-77 (2017).
56. Jones, P. A. The DNA methylation paradox. Trends Genet. 15, 34-7 (1999).
57. Papillon-Cavanagh, S. et al. Impaired H3K36 methylation defines a subset of head and neck squamous cell carcinomas. Nat. Genet. 49, 180-185 (2017).
58. Hannum, G. et al. Genome-wide Methylation Profiles Reveal Quantitative Views of Human Aging Rates. Mol. Cell 49, 359-367 (2013).
59. Horvath, S. DNA methylation age of human tissues and cell types. Genome boil 14, R115 (2013).
60. Slieker, R. C. et al. Age-related accrual of methylomic variability is linked to fundamental ageing mechanisms. Genome Biol. 17, 191 (2016).
61. Knight, A. K. et al. An epigenetic clock for gestational age at birth based on blood methylation data. Genome Biol. 17, 206 (2016).
62. Walsh, C. P., Chaillet, J. R. & Bestor, T. H. Transcription of IAP endogenous retroviruses is constrained by cytosine methylation. Nat. Genet. 20, 116-7 (1998).
63. Bourc'his, D. & Bestor, T. H. Meiotic catastrophe and retrotransposon reactivation in male germ cells lacking Dnmt3L. Nature 431, 96-99 (2004).
64. Trinh, B. N., Long, T. I., Nickel, A. E., Shibata, D. & Laird, P. W. DNA methyltransferase deficiency modifies cancer susceptibility in mice lacking DNA mismatch repair. Mol. Cell. Biol. 22, 2906-17 (2002).
65. Eden, A. Chromosomal Instability and Tumors Promoted by DNA Hypomethylation. Science (80-. 669). 300, 455-455 (2003).
66. Ehrlich, M. DNA hypomethylation in cancer cells. Epigenomics 1, 239-259 (2009).
67. Solyom, S. et al. Pathogenic orphan transduction created by a nonreference LINE-1 retrotransposon. Hum. Mutat. 33, 369-371 (2012).
68. Helman, E. et al. Somatic retrotransposition in human cancer revealed by whole 674 genome and exome sequencing. Genome Res. 24, 1053-63 (2014).
69. Amendola, M. & van Steensel, B. Nuclear lamins are not required for lamina676 associated domain organization in mouse embryonic stem cells. EMBO Rep. 16, 610-7 (2015).
70. Hiratani, I. et al. Genome-wide dynamics of replication timing revealed by in vitro models of mouse embryogenesis. Genome Res. 20, 155-69 (2010).

References cited with respect to working Example 8, and incorporated herein by reference for their respective teachings:
71. Xi, Y. & Li, W. BSMAP: whole genome bisulfite sequence MAPping program. BMC Bioinformatics 10, 232 (2009).
72. Liu, Y., Siegmund, K. D., Laird, P. W. & Berman, B. P. Bis-SNP: Combined DNA methylation and SNP calling for Bisulfite-seq data. Genome Biol. 13, R61 (2012).
73. Triche, T. J., Weisenberger, D. J., Van Den Berg, D., Laird, P. W. & Siegmund, K. D. Low-level processing of Illumina Infinium DNA Methylation BeadArrays. Nucleic Acids Res. 41, (2013).
74. Zhou, W., Laird, P. W. P. W. & Shen, H. Comprehensive characterization, annotation and innovative use of Infinium DNA methylation BeadChip probes. Nucleic Acids Res. 45, e22 (2017).
75. Hansen, R. S. et al. Sequencing newly replicated DNA reveals widespread plasticity in human replication timing. Proc. Natl. Acad. Sci. U S. A. 107, 139-44 (2010).
76. Quinlan, A. R. & Hall, I. M. BEDTools: A flexible suite of utilities for comparing genomic features. Bioinformatics 26, 841-842 (2010).

References cited with respect to working Examples 9-13, and incorporated herein by reference for their respective teachings:
77. Okano, M., Bell, D. W., Haber, D. A. & Li, E. DNA methyltransferases Dnmt3a and Dnmt3b are essential for de novo methylation and mammalian development. Cell 99, 247-257 (1999).
78. Laurent, L. et al. Dynamic changes in the human methylome during differentiation. Genome Res. 20, 320-31 (2010).
79. Pawlak, M. & Jaenisch, R. De novo DNA methylation by Dnmt3a and Dnmt3b is dispensable for nuclear reprogramming of somatic cells to a pluripotent state. Genes Dev. 25, 1035-1040 (2011).
80. Lister, R. et al. Human DNA methylomes at base resolution show widespread epigenomic differences. Nature 462, 315-322 (2009).
81. Berman, B. P. et al. Regions of focal DNA hypermethylation and long-range hypomethylation in colorectal cancer coincide with nuclear lamina-associated domains. Nat. Genet. 44, 40-46 (2012).
82. Hovestadt, V. et al. Decoding the regulatory landscape of medulloblastoma using DNA methylation sequencing. Nature 510, 537-541 (2014).
83. Johann, P. D. et al. Atypical Teratoid/Rhabdoid Tumors Are Comprised of Three Epigenetic Subgroups with Distinct Enhancer Landscapes. Cancer Cell 29, 379-393 (2016).
84. Fortin, J.-P. & Hansen, K. D. Reconstructing A/B compartments as revealed by Hi-C using long-range correlations in epigenetic data. Genome Biol. 16, 180 (2015).
85. Polak, P. et al. Cell-of-origin chromatin organization shapes the mutational landscape of cancer. Nature 518, 360-364 (2015).
86. Vandiver, A. R. et al. Age and sun exposure-related widespread genomic blocks of hypomethylation in nonmalignant skin. Genome Biol. 16, 80 (2015).
87. Hansen, K. D., Langmead, B. & Irizarry, R. a. BSmooth: from whole genome bisulfite sequencing reads to differentially methylated regions. Genome Biol. 13, R83 (2012).
88. Song, Q. et al. A reference methylome database and analysis pipeline to facilitate integrative and comparative epigenomics. PLoS One 8, e81148 (2013).
89. Knijnenburg, T. a et al. Multiscale representation of genomic signals. Nat. Methods 11, 689-94 (2014).
90. Shipony, Z. et al. Dynamic and static maintenance of epigenetic memory in pluripotent and somatic cells. Nature 513, 115-119 (2014).
91. Hansen, R. S. et al. Sequencing newly replicated DNA reveals widespread plasticity in human replication timing. Proc. Natl. Acad. Sci. U S. A. 107, 139-44 (2010).
92. Pope, B. D. et al. Topologically associating domains are stable units of replication-timing regulation. Nature 515, 402-405 (2014).
93. Iyengar, S. & Farnham, P. J. KAP1 protein: An enigmatic master regulator of the genome. J. Biol. Chem. 286, 26267-26276 (2011).
94. Raddatz, G., Gao, Q., Bender, S., Jaenisch, R. & Lyko, F. Dnmt3a Protects Active Chromosome Domains against Cancer-Associated Hypomethylation. PLoS Genet. 8, e 1003146 (2012).
95. Strom, A. R. et al. Phase separation drives heterochromatin domain formation. Nature 547, 241-245 (2017).
96. Larson, A. G. et al. Liquid droplet formation by HP1α suggests a role for phase separation in heterochromatin. Nature 547, 236-240 (2017).
97. Lawrence, M. S. et al. Mutational heterogeneity in cancer and the search for new cancer-associated genes. Nature 499, 214-8 (2013).
98. Hanawalt, P. C. & Spivak, G. Transcription-coupled DNA repair: two decades of progress and surprises. Nat. Rev. Mol. Cell Biol. 9, 958-70 (2008).
99. Kenigsberg, E. et al. The mutation spectrum in genomic late replication domains shapes mammalian GC content. Nucleic Acids Res. 44, 4222-4232 (2016).

100. Wang, K. et al. Whole-genome sequencing and comprehensive molecular profiling identify new driver mutations in gastric cancer. Nat. Genet. 46, 573-582 (2014).

101. Jónsson, H. et al. Parental influence on human germline de novo mutations in 1,548 trios from Iceland. Nature 549, 519-522 (2017).
102. Chiu, T P, et al., DNAshapeR: an R/Bioconductor package for DNA shape prediction and feature encoding. Bioinformatics. 15; 32(8):1211-3 (2016). doi: 10.1093/bioinformatics/btv735. Epub 2015 Dec. 14.
103. Zhou, W., Triche, T J, Laird, P W, & Shen, H. SeSAMe: reducing artifactual detection of DNA methylation by Infinium BeadChips in genomic deletions. Nuc Acids Res. 46(20):e123 (2018).
104. Zou, H. & Hastie, T. Regularization and variable selection via the elastic net. J. R. Statist. Soc. 67(2), 301-320 (2005).
105. Friedman, J., et al., Regularization Paths for Generalized Linear Models via Coordinate Descent. J. Statist. Software 33(1), 1-22 (2010).
106. Horvath, S., Oshima, J., Martin, G M, et al. Epigenetic clock for skin and blood cells applied to Hutchinson Gilford Progeria Syndrome and ex vivo studies. Aging 10(7): 1758-1775 (2018).
107. Levine, M E, Lu, AT, Quach, A., et al. An epigenetic biomarker of aging for lifespan and healthspan. Aging 10(4):573-591 (2018).
108. Yang, Z., et al. Correlation of an epigenetic mitotic clock with cancer risk. Genome Biol. 17(1):205 (2016).
109. Beerman, I., et al. Proliferation-dependent alterations of the DNA methylation landscape underlie hematopoietic stem cell aging. Cell Stem Cell 12(4):413-25 (2013).

The references cited above are incorporated herein by reference for their respective teachings.

What is claimed is:

1. A method, comprising:
a) identifying a test cell or tissue sample for which a determination of replication-associated genomic DNA methylation loss is desired;
b) obtaining, at data processing apparatus, CpG dinucleotide sequence methylation data for genomic DNA derived from the test cell or test tissue sample, wherein the genomic DNA comprises highly methylated domains (HMD) and partially methylated domains (PMD), wherein each such CpG dinucleotide is the sole CpG dinucleotide sequence within a n₍ₓ₎WCpGWn₍ₓ₎ genomic DNA sequence motif (Solo-WCGW motif) of at least one PMD, and wherein W=A or T, n=A or G or C or T, and x≥9;
c) determining, at the data processing apparatus, based on the CpG dinucleotide sequence methylation data, a mean or average CpG dinucleotide methylation value, or a value related thereto, for a plurality of Solo-WCGW motif sequences of the at least one PMDs, to provide a measure of cellular replication-associated DNA methylation loss, wherein the provided measure of replication-associated DNA methylation loss reflects a cumulative number of cell divisions or mitotic history; and
d) based on the provided measure of replication-associated DNA methylation loss, reaching a conclusion, at the data processing apparatus, as to a condition or state of the test cell or tissue sample.

2. The method of claim 2, wherein obtaining the genomic CpG dinucleotide sequence methylation data comprises excluding at the data processing apparatus, from a larger set of genomic CpG methylation data, methylation data of CpG dinucleotide sequences not within the Solo-WCGW motif sequences of the at least one PMD.

3. The method of claim 1, wherein obtaining the genomic CpG dinucleotide sequence methylation data comprises excluding at the data processing apparatus, from a larger set of genomic CpG methylation data, methylation data of non-intergenic Solo-WCGW motif sequences of the at least one PMD.

4. The method of claim 1, wherein obtaining the genomic CpG dinucleotide sequence methylation data comprises excluding at the data processing apparatus, from a larger set of genomic CpG methylation data, methylation data of H3K36me3 histone marked Solo-WCGW motif sequences or Solo-WCGW motif sequences falling in transcribed gene bodies of the at least one PMD.

5. The method of claim 1, wherein the plurality of Solo-WCGW motif sequences of the at least one PMDs are located at one or more PMDs of a single chromosome.

6. The method of claim 1, wherein the plurality of Solo-WCGW motif sequences of the at least one PMDs are located between or among multiple chromosomes.

7. The method of claim 1, wherein x is a value selected from the group consisting of at least 9, at least 14, at least 19, at least 24, at least 29, at least 34, at least 39, at least 44, at least 49, at least 54, and at least 59.

8. The method of claim 1, wherein x is a value in a range selected from the group consisting of about 9-49, 9-99, 9-149, 9-199, 14-49, 14-99, 14-149, 14-199, 19-49, 19-99, 19-149, 19-199, 24-49, 24-99, 24-149, 24-199, 29-49, 29-99, 29-149, 29-199, 34-49, 34-99, 34-149, 34-199, 39-49, 39-99, 39-149, 39-199, 44-49, 44-99, 44-149, 44-199, 49-99, 49-149, 49-199 54-99, 54-149, 54-199, 59-99, 59-149, 59-199, and any subranges of the preceding ranges.

9. The method of claim 1, wherein x is 34±25 (e.g., in the range of 9-59, or wherein x is 34±15 (e.g., in the range of 19-49).

10. The method of claim 1, wherein x is 34 or about 34.

11. The method of claim 1, wherein the Solo-WCGW motif comprises the sequence n₍ₓ₋₁₎mWCpGWGn₍ₓ₋₁₎, and wherein W=A or T, n=A or G or C or T, m=C or A, and x≥9.

12. The method of claim 1, wherein the Solo-WCGW motif comprises the sequence n₍ₓ₋₁₎CWCpGWGn₍ₓ₋₁₎, and wherein W=A or T, n=A or G or C or T, and x≥9.

13. The method of claim 1, wherein the at least one PMD is characterized, at least in part, by late replication timing and/or nuclear lamina localization and/or Hi-C-defined heterochromatic compartment B.

14. The method of claim 1, wherein the at least one PMD is, at least in part, defined by assessing, at the data processing apparatus, the CpG dinucleotide sequence methylation data of the Solo-WCGW motif sequences.

15. The method of claim 1, wherein the at least one PMD is, at least in part, defined by assessing, at the data processing apparatus, the standard deviation (SD) of the CpG dinucleotide sequence methylation data of the Solo-WCGW motif sequences across a set of samples, and/or by assessing, at the data processing apparatus, the covariance between multiple Solo-WCGW motif sequences across a set of samples.

16. The method of claim 15, wherein the SD of solo-WCGW PMD hypomethylation is bimodally distributed within 100-kb bins.

17. The method of claim 1, wherein the at least one PMD comprises a common PMD shared between or among a plurality of different cell or tissue types, or is a cell-type invariant PMD.

18. The method of claim 1, wherein the at least one PMD comprises a common PMD shared between or among normal and cancer cell or tissue types.

19. The method of claim 1, wherein the at least one PMD comprises a common PMD shared between most healthy mammalian tissue types starting from fetal development.

20. The method of claim 1, wherein the at least one PMD comprises a cell-type-specific PMD.

21. The method of claim 1, wherein the replication-associated DNA methylation loss reflects a cell-type specific replicative/mitotic turnover rate.

22. The method of claim 21, further comprising inferring the presence of genomic DNA of a highly replicative target cell type within a sample containing genomic DNA of multiple cell types, based on a target cell-type specific rate of replication-associated DNA methylation loss.

23. The method of claim 1, wherein the cumulative number of cell divisions, or the mitotic history, is from an early stage of embryonic development.

24. The method of claim 1, wherein the replication-associated DNA methylation loss reflects the chronological age of the cell or tissue sample.

25. The method of claim 1, wherein the cell or tissue sample is a cancer cell or cancer tissue sample.

26. The method of claim 1, wherein the genomic DNA derived from a cell or tissue sample comprises genomic DNA derived from tissue biopsies, or cell-free DNA derived from blood or other non-invasive samples including but not limited to urine, stool, saliva, etc.

27. The method of claim 1, wherein the plurality of Solo-WCGW motif sequences of the at least one PMDs is a number selected from at least 5, at least 10, at least 100, at least 500, at least 1,000, at least 1,500, at least 2,000, at least 5000, and at least 10,000.

28. The method of claim 1, wherein obtaining CpG dinucleotide sequence methylation data comprises obtaining CpG dinucleotide sequence methylation data from less than a complete genomic read.

29. The method of claim 1, wherein obtaining CpG dinucleotide sequence methylation data is from the genomic DNA of a single cell.

30. The method of claim 1, wherein the amount of replication-associated DNA methylation loss varies between cell types or tissue types, reflecting a cell-type or tissue-type specific rate of replication-associated DNA methylation loss.

31. The method of claim 1, wherein the plurality of Solo-WCGW motif sequences of the at least one PMDs, comprise hypomethylation prone Solo-WCGW sequence motifs selected to minimize propeller twist DNA shape.

32. A method for identification of replication-associated DNA methylation loss of a target cell type in a sample containing genomic DNA of multiple cell types, comprising:
a) identifying a test sample containing genomic DNA of multiple cell types including genomic DNA of a target cell type; and
b) determining, at data processing apparatus, for the genomic DNA from the test sample, replication-associated DNA methylation loss according to the method of claim 1, wherein the at least one PMD comprises a target cell-type specific PMD to provide a measure of target cell-type specific replication-associated DNA methylation loss.

33. The method of claim 32, wherein the presence of genomic DNA of the target cell is identified at the data processing apparatus, based on the presence of the target cell-type specific replication-associated DNA methylation loss.

34. The method of claim 32, wherein the at least one PMD comprises a cell-type specific PMD for the target cell type, and for each of other cell types of the sample to provide a measure of cell-type specific replication-associated DNA methylation loss for the target cell, and for each of the other cell types of the sample.

35. The method of claim 34, wherein the presence of the genomic DNA of the multiple cells types is identified at the data processing apparatus, based on the presence of the respective cell-type specific replication-associated DNA methylation losses.

36. The method of claim 35, further comprising identification, at the data processing apparatus, of the most hypomethylated cell types in the sample.

37. The method of claim 32, wherein the genomic DNA comprises genomic DNA derived from tissue biopsies, or cell-free DNA derived from blood or other non-invasive samples including but not limited to urine, stool, saliva, etc.

38. A method for providing a measure of a mitotic history/age of a cell or tissue sample, comprising:
a) identifying a test cell or tissue sample for which a determination of mitotic history/age is desired; and
b) determining, at data processing apparatus, for genomic DNA from the test cell or the test tissue sample, replication-associated DNA methylation loss according to the method of claim 1 to provide a measure of mitotic history/age for the test cell or test tissue (test mitotic age).

39. The method of claim 38, further comprising comparing, at the data processing apparatus, the measure of mitotic history/age of the test cell or test tissue determined in step b) with one or more control mitotic history/age values obtained, using the same method used in step b), for genomic DNA of a normal matched cell/tissue having a known replicative history, and assigning a mitotic history/age to the test cell or the test tissue.

40. The method of claim 39, wherein the normal matched cell/tissue having a known replicative history comprises a primary cell line or an immortalized primary cell line, for which mitotic history/age has been calibrated with respect to passage number using the method of claim 1.

41. The method of claim 38, wherein the determined mitotic history/age of the cell or the tissue is a cell type-specific or tissue type-specific mitotic history/age.

42. A method for determining a chronological age of a cell or tissue sample, comprising:
a) identifying a test cell or tissue sample for which a determination of chronological age is desired;
b) determining, at data processing apparatus, for genomic DNA from the test cell or the test tissue sample, replication-associated DNA methylation loss according to the method of claim 1 to provide a measure of mitotic history/age for the test cell or test tissue (test mitotic age); and
c) determining a chronological age for the test cell or test tissue by comparing, at data processing apparatus, the test mitotic age with one or more control mitotic age values obtained, using the same method used in a), for genomic DNA of a normal, cell-matched and/or tissue-matched control population calculated, at the data processing apparatus, over a chronological age range, and assigning a chronological age to the test cell or the test tissue.

43. The method of claim 42, wherein the actual chronological age of the test cell or test sample is known and is less than the chronological age determined in step b), providing a measure of accelerated aging.

44. The method of claim 42, wherein the method is part of a forensic analysis.

45. A method for determining increased risk for conditions associated with excessive replicative turnover or aging, comprising:
a) identifying a test cell or tissue sample for which a determining increased risk for conditions associated with excessive replicative turnover or aging is desired;
b) measuring, at data processing apparatus, for genomic DNA from the test cell or the test tissue sample having a known chronological age, replication-associated DNA methylation loss according to the method of claim 1 to provide a measure of mitotic age for the test cell or test tissue (test mitotic age); and
c) determining that there is an increased risk for conditions associated with excessive replicative turnover or aging by comparing, at the data processing apparatus, the test mitotic age with control mitotic age values obtained, using the same method used in a), for the genomic DNA of a normal, cell-matched or tissue-matched control population having the same chronological age as the test cell or test tissue, and finding, at the data processing apparatus, that the test mitotic age is greater than the aged-matched control mitotic age.

46. The method of claim 45, wherein the condition associated with excessive replicative turnover or aging is selected from the group consisting of cancer, neurodegenerative disease, cardiovascular disease, gastrointestinal disease, auto-immune diseases and progeria.

47. A method for determining increased risk of a subject for conditions associated with excessive replicative turnover or aging, comprising:
a) determining, at data processing apparatus, replication-associated genomic DNA methylation loss for a test cell or test tissue of a test subject;
b) comparing, at the data processing apparatus, the replication-associated genomic DNA methylation loss determined in a) with that of an age-matched normal control cell or tissue; and
c) based on the comparison in part b), concluding, at the data processing apparatus, that a subject having greater replication-associated genomic DNA methylation loss compared to that of the age-matched control is a subject having an increased risk for conditions associated with excessive replicative turnover or aging,
wherein the replication-associated genomic DNA methylation loss is determined by the method of claim 1.

48. The method of claim 47, wherein the condition associated with excessive replicative turnover or aging is selected from the group consisting of cancer, neurodegenerative disease, cardiovascular disease, gastrointestinal disease, auto-immune diseases and progeria.

49. A method of assessing methylation maintenance in stem cells, comprising:
identifying a test stem cell sample;
determining, at data processing apparatus, a measure of replication-associated genomic DNA methylation loss by the method of claim 1; and
based on the measure of replication-associated genomic DNA methylation loss, concluding, at the data processing apparatus, the degree of methylation maintenance by comparison with a normal control stem cell value.

50. The method of claim 49, wherein the stem cell is selected from the group consisting of embryonic stem cells (ESC), induced pluripotent stem cells (iPSC) and mesenchymal stem cells (MSCs).

51. A method for structurally defining a partially methylated domain (PMD) of genomic DNA, comprising:
a) identifying a genomic DNA for which at least one PMD structural determination is desired;
b) obtaining, at data processing apparatus, CpG dinucleotide sequence methylation data for the genomic DNA, wherein each such CpG dinucleotide is the sole CpG dinucleotide sequence within a n₍ₓ₎WCpGWn₍ₓ₎ genomic DNA sequence motif (Solo-WCGW motif) of at least one PMD, and wherein W=A or T, n=A or G or C or T, and x≥9; and
c) determining, at the data processing apparatus, a PMD structure based on the CpG dinucleotide sequence methylation data.

52. The method of claim 51, wherein the at least one PMD is, at least in part, defined by assessing, at the data processing apparatus, the standard deviation (SD) of the CpG dinucleotide sequence methylation data of the Solo-WCGW motif sequences.

53. The method of claim 52, wherein the SD of solo-WCGW PMD hypomethylation is bimodally distributed within 100-kb bins.

54. A method for developing a mitotic clock, comprising:
a) identifying a test cell for which a determination of a mitotic clock is desired;
b) providing conditions for the test cell to divide;
c) determining the number of effective cell divisions in the test cell at one or more timepoints;
d) obtaining, at data processing apparatus, CpG dinucleotide sequence methylation data for genomic DNA derived from the test cell at the timepoints, wherein the genomic DNA comprises highly methylated domains (HMD) and partially methylated domains (PMD), wherein each such CpG dinucleotide is the sole CpG dinucleotide sequence within a n₍ₓ₎WCpGWn₍ₓ₎ genomic DNA sequence motif (Solo-WCGW motif) of at least one PMD, and wherein W=A or T, n=A or G or C or T, and x≥9;
e) based on the CpG dinucleotide sequence methylation data, determining, at the data processing apparatus, a mean or average CpG dinucleotide methylation value or a value related thereto at each of the timepoints for a plurality of Solo-WCGW motif sequences of the at least one PMDs, to provide a measure of cellular replication-associated DNA methylation loss at each of the timepoints;
f) correlating, at the data processing apparatus, the effective cell divisions at each of the timepoints with the measure of cellular replication-associated DNA methylation loss at each of the timepoints; and
g) if the correlation from the correlating step is statistically significant, identifying the measure of cellular replication-associated DNA methylation loss as a mitotic clock.

55. The method of claim 54, wherein the correlating step includes calculating regression at the data processing apparatus.

56. The method of claim 55, wherein the regression calculation is determined by an elastic net regression model or an independent regression model.

57. The method of claim 54, wherein the each of the one or more timepoints is a cell passage in vitro.

58. The method of claim 57, wherein the test cell is passaged to certain passage numbers, and wherein the timepoints are the passages numbers.

59. The method of claim 58, further comprising, extracting DNA at each passage number and performing bisulfite conversion and library preparation.

60. The method of claim 59, further comprising, at the data processing apparatus, determining a passage number calibration curve.

61. The method of claim 54, wherein the conditions are in an animal and wherein the test cell divides to form a cell mass.

62. The method of claim 61, wherein the determining step includes measuring the volume of the cell mass at the one or more timepoints, and wherein an increase in the volume of the cell mass at the timepoints reflects an increase in the number of effective cell divisions.
